(19) United States
(12) Patent Application Publication
(10) Pub. No.: US 2026/0250251 A1
(43) Pub. Date: Aug. 27, 2026

(54) HETEROCYCLIC COMPOUNDS AS PI3Ka INHIBITORS

(71) Applicant: Pikavation Therapeutics, Inc., Wilmington, DE (US)

(72) Inventors: Chao Qi, West Chester, PA (US); Jun Pan, Media, PA (US); Liangxing Wu, Wilmington, DE (US); Wenqing Yao, Chadds Ford, PA (US)

(21) Appl. No.: 18/872,438

(22) PCT Filed: Jun. 8, 2023
(86) PCT No.: PCT/US2023/024811

§ 371 Date: Dec. 6, 2024

Related U.S. Application Data
(60) Provisional application No. 63/351,043, filed on Jun. 10, 2022.

(51) Int. Cl. C07D 231/56; A61K 31/416; A61K 31/4178; A61K 31/4192; A61K 31/437; A61K 31/4439; A61K 31/454; A61K 31/4745; A61K 31/4985; A61K 31/506; A61K 31/519; A61K 31/5377; A61K 31/5517; C07D 401/04; C07D 401/06; C07D 401/12; C07D 403/04; C07D 403/06; C07D 403/12; C07D 405/06; C07D 405/14; C07D 409/12; C07D 471/04; C07D 471/06; C07D 487/04; C07D 487/06

(52) U.S. Cl. CPC C07D 231/56; A61K 31/416; A61K 31/4178; A61K 31/4192; A61K 31/437; A61K 31/4439; A61K 31/454; A61K 31/4745; A61K 31/4985; A61K 31/506; A61K 31/519; A61K 31/5377; A61K 31/5517; C07D 401/04; C07D 401/06; C07D 401/12; C07D 403/04; C07D 403/06; C07D 403/12; C07D 405/06; C07D 405/14; C07D 409/12; C07D 471/04; C07D 471/06; C07D 487/04; C07D 487/06

(57) ABSTRACT

The present application provides heterocyclic compounds of formula (I) that modulate the activity of the PI3Ka, which are useful in the treatment of various diseases, including cancer.

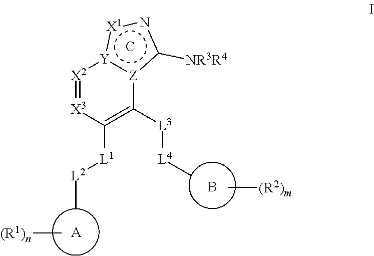

### TECHNICAL FIELD

The present disclosure provides heterocyclic compounds as well as their pharmaceutical compositions that modulate the activity of PI3Kα and are useful in the treatment of various diseases related to PI3Kα, including cancer.

### BACKGROUND

In the past few decades, signal transduction events have been studied to demonstrate critical roles in regulating almost all aspects of biological responses. Aberrant activation of the signaling pathways regulating cell survival and proliferation is commonly observed in many human cancers. The phosphoinositide 3-kinases (PI3Ks) signaling pathway is documented to be one of the highly mutated pathways in human cancers (Vogelstein et al., Science, 2013, 339(6127), 1546-1558). The PI3K signaling pathway regulates cell survival and proliferation. Increased activity of this pathway is associated with tumor progression and resistance to cancer therapies (Fusco et al., Front Oncol., 2021, 11, 644737).

PI3Ks belong to a lipid kinase family which catalyzes the phosphorylation of lipids contained in or associated with cell membranes. The PI3K family has fifteen kinases with distinct substrates, expression pattern, and modes of regulation. The class-I PI3Ks (p110α, p110β, p110δ, and p110γ) are typically activated by tyrosine receptor kinases or G-protein coupled receptors to generate PIP3, which activates downstream effectors of Akt, mTOR, or Rho GTPases (Fruman et al., Nat. Rev. Drug Discov., 2014, 13(2), 140-156)

Genetic mutations in the gene coding for PI3Kα are hotspot point mutations within helical and kinase domains, such as E542K, E545K, and H1047R. These mutations have been observed to occur in many cancer types such as lung, stomach, endometrial, ovarian, bladder, breast, colon, brain, prostate, and skin cancers. Because these gain-of-function mutations in PI3Kα are associated with tumor progression, targeting this pathway may provide valuable therapeutic opportunities (Courtney et al., J Clin. Oncol., 2010, 28 (6), 1075-1083). While multiple inhibitors of PI3Ks have been developed (for example, taselisib, alpelisib, buparlisib and others), these molecules inhibit multiple PI3K isoforms. These “pan-PI3K” inhibitors have encountered major hurdle in the clinical development due to inability to achieve the required level of target inhibition in tumors while avoiding toxicity in cancer patients (Fruman et al., Nat. Rev. Drug Discov., 2014, 13(2), 140-156). The toxicity of PI3K inhibitors is dependent on their isoform selectivity profile. Inhibition of PI3Kα is associated with hyperglycemia and rash, while inhibition of PI3Kδ or PI3Kγ is associated with diarrhea, myelosuppression, and transaminitis (Hanker et al., Cancer Discov., 2019, 9(4), 482-491). Therefore, selective inhibitors of PI3Kα may increase the therapeutic window, enabling sufficient target inhibition in the tumor while avoiding dose-limiting toxicity in cancer patients. However, given the central role of PI3Kα in regulating glucose homeostasis and other critical physiological process, current PI3Kα selective inhibitors, which are equally potent to wild-type and mutant PI3Kα, often cause hyperglycemia and/or hyperinsulinemia (Busaidy et al., J. Clin. Oncol., 2012, 30, 2919-2928). In summary, developing inhibitors with enhanced selectivity for mutant PI3Kα against wild-type PI3Kα would be able to overcome the problem of compensatory insulin production and hyperglycemia.

### SUMMARY

The present disclosure provides, inter alia, compounds of Formula I.

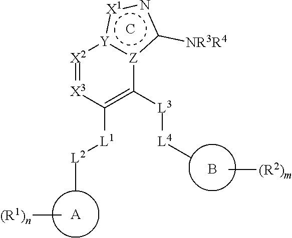

or pharmaceutically acceptable salts thereof, wherein constituent members are defined herein.

The present disclosure further provides a pharmaceutical composition comprising a compound of the disclosure, or a pharmaceutically acceptable salt thereof, and a pharmaceutically acceptable carrier or excipient.

The present disclosure further provides methods of inhibiting PI3Kα activity, comprising contacting the PI3Kα with a compound described herein, or a pharmaceutically acceptable salt thereof.

The present disclosure further provides methods of treating a disease or a disorder associated with PI3Kα in a patient by administering to the patient a therapeutically effective amount of a compound of the disclosure, or a pharmaceutically acceptable salt thereof.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

### DETAILED DESCRIPTION

The present disclosure provides a compound of Formula I:

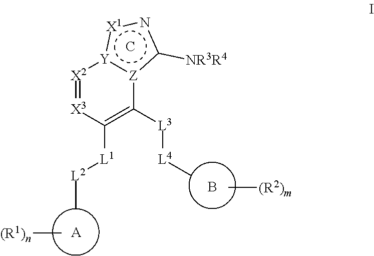

or a pharmaceutically acceptable salt thereof, wherein:
X¹ is CR⁵, O, N, or NR⁶;
X² is CR⁷ or N;
X³ is CR⁸ or N;
Y is C or N;
Z is C or N;
n is 0, 1, 2, 3, 4, 5, or 6;
m is 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is a 5-membered heteroaryl having 2 to 3 heteroatoms as ring members selected from O and N;
L¹ and L³ are each independently selected from C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)-C₁₋₄ alkyl-, -(5-6 membered heteroarylene)-C₁₋₄ alkyl-, —O—, —S—, —N(R^{L})—, —C(O)—, —C(O)N(R^{L})—, —N(R^{L})C(O)—, —N(R^{L})C(O)N(R^{L})—, —N(R^{L})C(O)O—, —OC(O)N(R^{L})—, —S(O)₂—, —N(R^{L})S(O)₂—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)-C₁₋₄ alkyl, and (5-6 membered heteroarylene)-C₁₋₄ alkyl of L¹ and L³ are each optionally substituted with 1, 2, 3, or 4 independently selected R^{G} substituents;
L² and L⁴ are each independently selected from bond, C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)-C₁₋₄ alkyl-, -(5-6 membered heteroarylene)-C₁₋₄ alkyl-, —O—, —S—, —N(R^{L})—, —C(O)—, —C(O)N(R^{L})—, —N(R^{L})C(O)—, —N(R^{L})C(O)N(R^{L})—, —N(R^{L})C(O)O—, —OC(O)N(R^{L})—, —S(O)₂—, —N(R^{L})S(O)₂—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)-C₁₋₄ alkyl, and (5-6 membered heteroarylene)-C₁₋₄ alkyl of L² and L⁴ are each optionally substituted with 1, 2, 3, or 4 independently selected R^{G} substituents;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}C(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{c1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{b1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R¹ are each optionally substituted with 1, 2, 3, 4, 5, or 6 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1}, R^{b1}, R^{c1}, and R^{d1} are each optionally substituted with 1, 2, 3, 4, 5, or 6 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{1A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1A}, —SR^{a1A}, —NR^{c1A}R^{d1A}, —C(O)R^{a1A}, —C(O)OR^{a1A}, —C(O)NR^{c1A}R^{d1A}, —C(O)NR^{c1A}(OR^{a1A}), —OC(O)R^{a1A}, —OC(O)NR^{c1A}R^{d1A}, —OC(O)OR^{a1A}, —OS(O)₂R^{b1A}, —OS(O)₂NR^{c1A}R^{d1A}, —NR^{c1A}C(O)R^{a1A}, —NR^{c1A}C(O)OR^{a1A}, —NR^{c1A}C(O)NR^{c1A}R^{d1A}, —NR^{c1A}S(O)₂R^{b1A}, —NR^{c1A}S(O)₂NR^{c1A}R^{d1A}, —NR^{c1A}OR^{a1A}, —NR^{c1A}S(O)R^{b1A}, —NR^{c1A}S(O)NR^{c1A}R^{d1A}, —S(O)R^{b1A}, —S(O)₂R^{b1A}, —S(O)NR^{c1A}R^{d1A}, —S(O)₂NR^{c1A}R^{d1A}, —C(═NR^{e1A})R^{a1A}, —C(═NR^{e1A})NR^{c1A}R^{d1A}, —NR^{c1A}C(═NR^{e1A})R^{a1A}, —NR^{c1A}C(═NR^{e1A})NR^{c1A}R^{d1A}, —NR^{c1A}S(O)(═NR^{e1A})R^{b1A}, —NR^{c1A}S(O)(═NR^{e1A})NR^{c1A}R^{d1A}, —OS(O)(═NR^{e1A})R^{b1A}, —S(O)(═NR^{e1A})R^{b1A}, —S(O)(═NR^{e1A})NR^{c1A}R^{d1A}, —C(O)NR^{c1A}S(O)₂R^{b1A}, —C(O)NR^{c1A}S(O)₂NR^{c1A}R^{d1A}, —S(O)₂NR^{c1A}C(O)R^{b1A}, —NR^{c1A}S(O)NR^{c1A}C(O)R^{b1A}, and —P(O)R^{f1A}R^{g1A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{1A} are each optionally substituted with 1, 2, 3, 4, 5, or 6 independently selected R^{G} substituents;
each R^{a1A}, R^{b1A}, R^{c1A}, and R^{d1A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1A}, R^{b1A}, R^{c1A}, and R^{d1A} are each optionally substituted with 1, 2, 3, 4, 5, or 6 independently selected R^{G} substituents;
or, any R^{c1A} and R^{d1A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e1A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1A} and R^{g1A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR², —SR², —NR^{c2}R^{d2}, —C(O)R^{a2}, —C(O)OR^{a2}, —C(O)NR^{c2}R^{d2}, —C(O)NR^{c2}(OR^{a2}), —OC(O)R^{a2}, —OC(O)NR^{c2}R^{d2}, —OC(O)OR^{a2}, —OS(O)₂R^{b2}, —OS(O)₂NR^{c2}R^{d2}, —NR^{c2}C(O)R^{a2}, —NR^{c2}C(O)OR^{a2}, NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —OS(O)(═NR^{e2})R^{b2}, —S(O)(═NR^{e2})R^{b2}, —S(O)(═NR^{e2})NR^{c2}R^{d2}, —C(O)NR^{c2}S(O)₂R^{b2}, —C(O)NR^{c2}S(O)₂NR^{c2}R^{d2}, —S(O)₂NR^{c2}C(O)R^{b2}, —NR^{c2}S(O)NR^{c2}C(O)R^{b2}, and —P(O)R^{f2}R^{g2}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R² are each optionally substituted with 1, 2, 3, 4, 5, or 6 independently selected R^{2A} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a2}, R^{b2}, R^{c2}, and R^{d2} are each optionally substituted with 1, 2, 3, 4, 5, or 6 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{2A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a2A}, —SR^{a2A}, —NR^{c2A}R^{d2A}, —C(O)R^{a2A}, —C(O)OR^{a2A}, —C(O)NR^{c2A}R^{d2A}, —C(O)_{NR}^{c2A}(OR^{a2A}), —OC(O)R^{a2A}, —OC(O)NR^{c2A}R^{d2A}, —OC(O)OR^{a2A}, —OS(O)₂R^{b2A}, —OS(O)₂NR^{c2A}R^{d2A}, —NR^{c2A}C(O)R^{a2A}, —NR^{c2A}C(O)OR^{a2A}, —NR^{c2A}C(O)NR^{c2A}R^{d2A}, —NR^{c2A}S(O)₂R^{b2A}, —NR^{c2A}S(O)₂NR^{c2A}R^{d2A}, —NR^{c2A}OR^{a2A}, —R^{c2A}S(O)R^{b2A}, —NR^{c2A}S(O)NR^{c2A}R^{d2A}, —S(O)R^{b2A}, —S(O)₂R^{b2A}. —S(O)NR^{c2A}R^{d2A}, —S(O)₂NR^{c2A}R^{d2A}, —C(═NR^{e2A})R^{a2A}, —C(═NR^{e2A})NR^{c2A}R^{d2A}, —NR^{c2A}C(═NR^{e2A})R^{a2A}, —NR^{c2A}C(═NR^{e2A})NR^{c2A}R^{d2A}, —NR^{c2A}S(O)(═NR^{e2A})R^{b2A}, —NR^{c2A}S(O)(═NR^{e2A})NR^{c2A}R^{d2A}, —OS(O)(═NR^{e2A})R^{b2A}, —S(O)(═NR^{e2A})R^{b2A}, —S(O)(═NR^{e2A})NR^{c2A}R^{d2A}, —C(O)NR^{e2A}S(O)₂R^{b2A}, —C(O)NR^{c2A}S(O)₂NR^{c2A}R^{d2A}, —S(O)₂NR^{c2A}C(O)R^{b2A}, —NR^{c2A}S(O)NR^{c2A}C(O)R^{b2A}, and —P(O)R^{f2A}R^{g2A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{2A} are each optionally substituted with 1, 2, 3, 4, 5, or 6 independently selected R^{G} substituents;
each R^{a2A}, R^{b2A}, R^{c2A}, and R^{d2A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a2A}, R^{b2A}, R^{c2A}, and R^{d2A} are each optionally substituted with 1, 2, 3, 4, 5, or 6 independently selected R^{G} substituents;
or, any R^{c2A} and R^{d2A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e2A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f2A} and R^{g2A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R³ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a3}, —C(O)OR^{a3}, —C(O)NR^{c3}R^{d3}, —S(O)₂R^{b3}, —S(O)₂NR^{c3}R^{d3}, —S(O)(═NR^{e3})R^{b3}, and —S(O)(=NR^{e3})NR^{c3}R^{d3} wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R³ are each optionally substituted with 1, 2, 3, 4, 5, or 6 independently selected R^{G} substituents;
or, R³ and L³ together with the atoms to which they are attached form a 5-10 membered heterocycloalkyl group, wherein the 5-10 membered heterocycloalkyl group is optionally substituted with 1, 2, 3, 4, 5, or 6 independently selected R^{G} substituents;
each R^{a1}, R^{b3}, R^{c3}, and R^{d3} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a3}, R^{b3}, R^{c3}, and R^{d3} are each optionally substituted with 1, 2, 3, 4, 5, or 6 independently selected R^{G} substituents;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁴ are each optionally substituted with 1, 2, 3, 4, 5, or 6 independently selected R^{G} substituents;
R⁵ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{a5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{a5}, —S(O)₂NR^{c5}R^{d5}, —C(═NR^{e5})R^{a5}, —C(═NR^{e5})NR^{c5}R^{a5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁵ are each optionally substituted with 1, 2, 3, 4, 5, or 6 independently selected R^{SA} substituents;
each R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R^{a5}, R^{b5}, R^{c5}, and R^{d5} are each optionally substituted with 1, 2, 3, 4, 5, or 6 independently selected R^{5A} substituents;

or, any R^{c5} and R^{d5} attached to the same N atom, together with the N atom to which they are attached, form a 4-14 membered heterocycloalkyl group, wherein the 4-14 membered heterocycloalkyl group is optionally substituted with 1, 2, 3, 4, 5, or 6 independently selected R^{5A} substituents;

each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;

each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{5A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5A}, —SR^{a5A}, —NR^{c5A}R^{d5A}, —NO₂, —C(O)R^{a5A}, —C(O)OR^{a5A}, —C(O)NR^{c5A}R^{d5A}, —C(O)NR^{c5A}(OR^{a5A}), —OC(O)R^{a5A}, —OC(O)NR^{c5A}R^{d5A}, —OC(O)OR^{a5A}, —OS(O)₂R^{b5A}, —OS(O)₂NR^{c5A}R^{d5A}, —NR^{c5A}C(O)R^{a5A}, —NR^{c5A}C(O)OR^{a5A}, —NR^{c5A}C(O)NR^{c5A}R^{d5A}, —NR^{c5A}S(O)₂R^{b5A}, —NR^{c5A}S(O)₂NR^{c5A}R^{d5A}, —NR^{c5A}OR^{a5A}, —NR^{c5A}S(O)R^{b5A}, —NR^{c5A}S(O)NR^{c5A}R^{a5A}, —S(O)R^{b5A}, —S(O)₂R^{b5A}, —S(O)NR^{c5A}R^{a5A}, —S(O)₂NR^{c5A}R^{d5A}, —C(═NR^{e5A})R^{a5A}, —C(═NR^{e5A})NR^{c5A}R^{d5A}, —NR^{c5A}C(═NR^{e5A})R^{a5A}, —NR^{c5A}C(═NR^{e5A})NR^{c5A}R^{d5A}, —NR^{c5A}S(O)(═NR^{e5A})R^{b5A}, —NR^{c5A}S(O)(═NR^{e5A})NR^{c5A}R^{a5A}, —OS(O)(═NR^{e5A})R^{b5A}, —S(O)(═NR^{e5A})R^{b5A}, —S(O)(═NR^{e5A})NR^{c5A}R^{a5A}, —C(O)NR^{c5A}S(O)₂R^{b5A}. —C(O)NR^{c5A}S(O)₂NR^{c5A}R^{a5A}, —S(O)₂NR^{e5A}C(O)R^{b5A}, —NR^{e5A}S(O)NR^{e5A}C(O)R^{b5A}, and —P(O)R^{f5A}R^{g5A} wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{5A} are each optionally substituted with 1, 2, 3, 4, 5, or 6 independently selected R^{G} substituents;
each R^{a5A}, R^{b5A}, R^{c5A}, and R^{d5A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a5a}, R^{b5a}, R^{c5a}, and R^{d5A} are each optionally substituted with 1, 2, 3, 4, 5, or 6 independently selected R^{G} substituents;
or, any R^{c5A} and R^{d5A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e5A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5A} and R^{g5A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —C(O)R^{a6}, —C(O)OR^{a6}, —C(O)NR^{c6}R^{d6}, —C(O)NR^{c6}(OR^{a6}), —S(O)R^{b6}, —S(O)₂R^{b6}, —S(O)NR^{c6}R^{d6}, —S(O)₂NR^{c6}R^{d6}, —C(═NR^{e6})R^{a6}, —C(═NR^{e6})NR^{c6}R^{d6}, —S(O)(═NR^{e6})R^{b6}, —S(O)(═NR^{e6})NR^{c6}R^{d6}, —C(O)NR^{c6}S(O)₂R^{b6}, —C(O)NR^{c6}S(O)₂NR^{c6}R^{d6}, and —S(O)₂NR^{c6}C(O)R^{b6}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁶ are each optionally substituted with 1, 2, 3, 4, 5, or 6 independently selected R^{6A} substituents;
each R^{a6}, R^{b6}, R^{c6} and R^{d6} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a6}, R^{b6}, R^{c6} and R^{d6} are each optionally substituted with 1, 2, 3, 4, 5, or 6 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{6A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a6A}, —SR^{a6A}, —NR^{c6A}R^{d6A}, —NO₂, C(O)R^{a6A}, —C(O)OR^{a6A}, —C(O)NR^{c6A}R^{d6A}, —C(O)NR^{c6A}(OR^{a6A}), —OC(O)R^{a6A}, —OC(O)NR^{c6A}R^{d6A}, —OC(O)OR^{a6A}, —OS(O)₂R^{b6A}, —OS(O)₂NR^{c6A}R^{d6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)₂NR^{c6A}R^{d6A}, —NR^{c6A}OR^{a6A}, —NR^{c6A}S(O)R^{b6A}, —NR^{c6A}S(O)NR^{c6A}R^{a6A}, —S(O)R^{b6A}, —S(O)₂R^{b6A}, —S(O)NR^{c6A}R^{d6A}, —S(O)₂NR^{c6A}R^{d6A}, —C(═NR^{e6A})R^{a6A}, —C(═NR^{e6A})NR^{c6A}R^{d6A}, —NR^{c6A}C(═NR^{e6A})R^{a6A}, —NR^{c6A}C(═NR^{e6A})NR^{c6A}R^{d6A}, —NR^{c6A}S(O)(═NR^{e6A})R^{b6A}, —NR^{c6A}S(O)(═NR^{e6A})NR^{c6A}R^{d6A}, —OS(O)(═NR^{e6A})R^{b6A}, —S(O)(═NR^{e6A})R^{b6A}, —S(O)(═NR^{e6A})NR^{c6A}R^{d6A}, —C(O)NR^{c6A}S(O)₂R^{b6A}, —C(O)NR^{c6A}S(O)₂NR^{c6A}R^{d6A}, —S(O)₂NR^{c6A}C(O)R^{b6A}, —NR^{c6A}S(O)NR^{c6A}C(O)R^{b6A}, and —P(O)R^{f6A}R^{g6A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{6A} are each optionally substituted with 1, 2, 3, 4, 5, or 6 independently selected R^{G} substituents;
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a6A}, R^{b6A}, R^{c6A}, and R^{d6A} are each optionally substituted with 1, 2, 3, 4, 5, or 6 independently selected R^{G} substituents;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e6A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f6A} and R^{g6A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}, —NR^{c7}C(O)R^{a7}, —NR^{c7}C(O)OR^{a7}, —NR^{c7}C(O)NR^{c7}R^{d7}, —NR^{c7}S(O)₂R^{b7}, —NR^{c7}S(O)₂NR^{c7}R^{d7}, —NR^{c7}OR^{a7}, —NR^{c7}S(O)R^{b7}, —NR^{c7}S(O)NR^{c7}R^{d7}, —S(O)R^{b7}, —S(O)₂R^{b7}, —S(O)NR^{c7}R^{d7}, —S(O)₂NR^{c7}R^{d7}, —C(═NR^{c7})R^{a7}, —C(═NR^{c7})NR^{c7}R^{d7}, —NR⁷C(═NR^{e7})R^{a7}, —NR^{c7}C(═NR^{e7})NR^{c7}R^{d7}, —NR^{c7}S(O)(═NR^{e7})R^{b7}, —NR^{c7}S(O)(═NR^{e7})NR^{c7}R^{d7}, —OS(O)(═NR^{e7})R^{b7}, —S(O)(═NR^{e7})R^{b7}, —S(O)(═NR^{e7})NR^{c7}R^{d7}, —C(O)NR^{c7}S(O)₂R^{b7}, —C(O)NR^{c7}S(O)₂NR^{c7}R^{d7}, —S(O)₂NR^{c7}C(O)R^{b7}, —NR^{c7}S(O)NR^{c7}C(O)R^{b7}, and —P(O)R^{f7}R^{g7}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of Rare each optionally substituted with 1, 2, 3, 4, 5, or 6 independently selected R^{7A} substituents;
or, R⁵ and R⁷ together with the atoms to which they are attached form a C₅₋₁₄ cycloalkyl or 5-14 membered heterocycloalkyl group, wherein the C₅₋₁₄ cycloalkyl and 5-14 membered heterocycloalkyl group is optionally substituted with 1, 2, 3, 4, 5, or 6 independently selected R^{7A} substituents;
or, R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents;
each R^{a7}, R^{b7}, R^{c7}, and R^{d7} is independently selected from H, C₁₋₆ alkyl, C₁₋₆ haloalkyl, C₂₋₆ alkenyl, C₂₋₆ alkynyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R^{a7}, R^{b7}, R^{c7}, and R^{d7} are each optionally substituted with 1, 2, 3, 4, 5, or 6 independently selected R^{7A} substituents;
or, any R^{c7} and R^{d7} attached to the same N atom, together with the N atom to which they are attached, form a 4-14 membered heterocycloalkyl group, wherein the 4-14 membered heterocycloalkyl group is optionally substituted with 1, 2, 3, 4, 5, or 6 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7A}, —SR^{a7A}, —NR^{c7A}R^{d7A}, —NO₂, —C(O)R^{a7A}, —C(O)OR^{a7A}, —C(O)NR^{c7A}R^{d7A}, —C(O)NR^{c7A}(OR^{a7A}), —OC(O)R^{a7A}, —OC(O)NR^{c7A}R^{d7A}, —OC(O)OR^{a7A}, —OS(O)₂R^{b7A}, —OS(O)₂NR^{7A}R^{d7A}, —NR^{c7A}C(O)R^{a7A}, —NR^{c7A}C(O)OR^{a7A}, —NR^{c7A}C(O)NR^{c7A}R^{d7A}, —NR^{c7A}S(O)₂R^{b7A}, —NR^{c7A}S(O)₂NR^{c7A}R^{d7A}, —NR^{c7A}OR^{a7A}, —NR^{c7A}S(O)R^{b7A}, —NR^{c7A}S(O)NR^{c7A}R^{d7A}, —S(O)R^{b7A}, —S(O)₂R^{b7A}, —S(O)NR^{c7A}R^{d7A}, S(O)₂NR^{c7A}R^{d7A}, —C(═NR^{e7A})R^{a7A}, —C(═NR^{e7A})NR^{c7A}R^{d7A}, —NR^{c7A}C(═NR^{e7A})R^{a7A}, —NR^{c7A}C(═NR^{e7A})NR^{c7A}R^{d7A}, —NR^{c7A}S(O)(═NR^{e7A})R^{b7A}, —NR^{c7A}S(O)(═NR^{e7A})NR^{c7A}R^{d7A}, —OS(O)(═NR^{e7A})R^{b7A}, —S(O)(═NR^{e7A})R^{b7A}, —S(O)(═NR^{e7A})NR^{c7A}R^{d7A}, —C(O)NR^{c7A}S(O)₂R^{b7A}, —C(O)NR^{c7A}S(O)₂NR^{c7A}R^{d7A}, —S(O)₂NR^{c7A}C(O)R^{b7A}, —NR^{c7A}S(O)NR^{c7A}C(O)R^{b7A}, and —P(O)R^{f7A}R^{g7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{7B} substituents;
each R^{a7A}, R^{b7A}, R^{c7A}, and R^{d7A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7A}, R^{b7A}, R^{c7A}, and R^{d7A} are each optionally substituted with 1, 2, 3, 4, 5, or 6 independently selected R^{7B} substituents;
or, any R^{c7A} and R^{d7A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{7B} substituents;
each R^{e7A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7A} and R^{g7A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7B} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7B}, —SR^{a7B}, —NR^{c7B}R^{d7B}, —NO₂, —C(O)R^{a7B}, —C(O)OR^{a7B}, —C(O)NR^{c7B}R^{d7B}, —C(O)NR^{c7B}(OR^{a7B}), —OC(O)R^{a7B}, —OC(O)NR^{c7B}R^{d7B}, —OC(O)OR^{a7B}, —OS(O)₂R^{b7B}, —OS(O)₂NR^{c7B}R^{d7B}, —NR^{c7B}C(O)R^{a7B}, —NR^{c7B}C(O)OR^{a7B}, —NR^{c7B}C(O)NR^{c7B}R^{d7B}, —NR^{c7B}S(O)₂R^{b7B}, —NR^{c7B}S(O)₂NR^{c7B}R^{d7B}, —NR^{c7B}OR^{a7B}, —NR^{c7B}S(O)R^{b7B}, —NR^{c7B}S(O)NR^{c7B}R^{d7B}, —S(O)R^{b7B}, —S(O)₂R^{b7B}, —S(O)NR^{c7B}R^{d7B}, —S(O)₂NR^{c7B}R^{d7B}, —C(═NR^{e7B})R^{a7B}, —C(═NR^{e7B})NR^{c7B}R^{d7B}, —NR^{c7B}C(═NR^{e7B})R^{a7B}, —NR^{c7B}C(═NR^{e7B})NR^{c7B}R^{d7B}, —NR^{c7B}S(O)(═NR^{e7B})R^{b7B}, —NR^{c7B}S(O)(═NR^{e7B})NR^{c7B}R^{d7B}, —OS(O)(═NR^{e7B})R^{b7B}, —S(O)(═NR^{e7B})R^{b7B}, —S(O)(═NR^{e7B})NR^{c7B}R^{d7B}, —C(O)NR^{c7B}S(O)₂R^{b7B}, —C(O)NR^{c7B}S(O)₂NR^{c7B}R^{d7B}, —S(O)₂NR^{c7B}C(O)R^{b7B}, —NR^{c7B}S(O)NR^{c7B}C(O)R^{b7B}, and —P(O)R^{f7B}R^{g7B}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7B} are each optionally substituted with 1, 2, 3, 4, 5, or 6 independently selected R^{G} substituents;
each R^{a7B}, R^{b7B}, R^{c7B}, and R^{d7B} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7B}, R^{b7B}, R^{c7B}, and R^{d7B} are each optionally substituted with 1, 2, 3, 4, 5, or 6 independently selected R^{G} substituents;
or, any R^{c7B} and R^{d7B} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e7B} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7B} and R^{g7B} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁸ is selected from H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —O^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁸ are each optionally substituted with 1, 2, 3, 4, 5, or 6 independently selected R^{8A} substituents;
each R^{a8}, R^{b8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{b8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —NR^{c8A}S(O)₂NR^{c8A}R^{d8A}, —NR^{c8A}OR^{a8A}, —NR^{c8A}S(O)R^{b8A}, —NR^{c8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, —C(═NR^{e8A})R^{a8A}, —C(═NR^{e8A})NR^{c8A}R^{d8A}, —NR^{c8A}C(═NR^{e8A})R^{a8A}, —NR^{c8A}C(═NR^{e8A})NR^{c8A}R^{d8A}, —NR^{c8A}S(O)(═NR^{e8A})R^{b8A}, —NR^{c8A}S(O)(═NR^{e8A})NR^{c8A}R^{d8A}, —OS(O)(═NR^{e8A})R^{b8A}, —S(O)(═NR^{e8A})R^{b8A}, —S(O)(═NR^{e8A})NR^{c8A}R^{d8A}, —C(O)NR^{c8A}S(O)₂R^{b8A}, —C(O)NR^{c8A}S(O)₂NR^{c8A}R^{d8A}, —S(O)₂NR^{c8A}C(O)R^{b8A}, —NR^{c8A}S(O)NR^{c8A}C(O)R^{b8A}, and —P(O)R^{f8A}R^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{b8A}, R^{c8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered 10 heteroaryl)-C₁₋₄ alkyl of R^{a8A}, R^{b8A}, R^{c8A}, and R^{d8A} are each optionally substituted with 1, 2, 3, 4, 5, or 6 independently selected R^{G} substituents;
or, any R^{c8A} and R^{d8A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino

In some embodiments:
X¹ is CR⁵, O, N, or NR⁶;
X² is CR⁷ or N;
X³ is CR⁸ or N;
Y is C or N;
Z is C or N;
n is 0, 1, 2, 3, 4, 5, or 6;
mis 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is a 5-membered heteroaryl having 2 to 3 heteroatoms as ring members selected from O and N;
L¹ and L³ are each independently selected from C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)-C₁₋₄ alkyl-, -(5-6 membered heteroarylene)-C₁₋₄ alkyl-, —O—, —S—, —N(R^{L})—, —C(O)—, —C(O)N(R^{L})—, —N(R^{L})C(O)—, —N(R^{L})C(O)N(R^{L})—, —N(R^{L})C(O)O—, —OC(O)N(R^{L})—, —S(O)₂—, —N(R^{L})S(O)₂—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)-C₁₋₄ alkyl, and (5-6 membered heteroarylene)-C₁₋₄ alkyl of L¹ and L³ are each optionally substituted with 1, 2, 3, or 4 independently selected R^{G} substituents;
L² and L⁴ are each independently selected from bond, C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)-C₁₋₄ alkyl-, -(5-6 membered heteroarylene)-C₁₋₄ alkyl-, —O—, —S—, —N(R^{L})—, —C(O)—, —C(O)N(R^{L})—, —N(R^{L})C(O)—, —N(R^{L})C(O)N(R^{L})—, —N(R^{L})C(O)O—, —OC(O)N(R^{L})—, —S(O)₂—, —N(R^{L})S(O)₂—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)-C₁₋₄ alkyl, and (5-6 membered heteroarylene)-C₁₋₄ alkyl of L² and L⁴ are each optionally substituted with 1, 2, 3, or 4 independently selected R^{G} substituents;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}C(O)NR^{c1}R^{d1}, —NR^{c1}S(O)₂R^{b1}, —NR^{c1}S(O)₂NR^{c1}R^{d1}, —NR^{c1}OR^{d1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{cl}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{a1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R¹ are each optionally substituted with 1, 2, 3, 4, 5, or 6 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1}, R^{b1}, R^{c1}, and R^{d1} are each optionally substituted with 1, 2, 3, 4, 5, or 6 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{1A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1A}, —SR^{a1A}, —NR^{c1A}R^{d1A}, —C(O)R^{a1A}, —C(O)OR^{a1A}, —C(O)NR^{c1A}R^{d1A}, —C(O)NR^{c1A}(OR^{a1A}), —OC(O)R^{a1A}, —OC(O)NR^{c1A}R^{d1A}, —OC(O)OR^{a1A}, —OS(O)₂R^{b1A}, —OS(O)₂NR^{c1A}R^{d1A}, —NR^{c1A}C(O)R^{a1A}, —NR^{c1A}C(O)OR^{a1A}, —NR^{c1A}C(O)NR^{c1A}R^{d1A}, —NR^{c1A}S(O)₂R^{b1A}, —NR^{c1A}S(O)₂NR^{c1A}R^{d1A}, —NR^{c1A}OR^{a1A}, —NR^{c1A}S(O)R^{b1A}, —NR^{c1A}S(O)NR^{c1A}R^{d1A}, —S(O)R^{b1A}, —S(O)₂R^{b1A}, —S(O)NR^{c1A}R^{d1A}, —S(O)₂NR^{c1A}R^{d1A}, —C(═NR^{e1A})R^{a1A}, —C(═NR^{e1A})NR^{c1A}R^{d1A}, —NR^{c1A}C(═NR^{e1A})R^{a1A}, —NR^{c1A}C(═NR^{e1A})NR^{c1A}R^{d1A}, —NR^{c1A}S(O)(═NR^{e1A})R^{b1A}, —NR^{c1A}S(O)(═NR^{e1A})NR^{c1A}R^{d1A}, —OS(O)(═NR^{e1A})R^{b1A}, —S(O)(═NR^{e1A})R^{b1A}, —S(O)(═NR^{e1A})NR^{c1A}R^{d1A}, —C(O)NR^{c1A}S(O)₂R^{b1A}, —C(O)NR^{c1A}S(O)₂NR^{c1A}R^{d1A}, —S(O)₂NR^{c1A}C(O)R^{b1A}, —NR^{c1A}S(O)NR^{c1A}C(O)R^{b1A}, and —P(O)R^{f1A}R^{g1A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{1A} are each optionally substituted with 1, 2, 3, 4, 5, or 6 independently selected R^{G} substituents;
each R^{a1A}, R^{b1A}, R^{c1A}, and R^{d1A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1A}, R^{b1A}, R^{c1A}, and R^{d1A} are each optionally substituted with 1, 2, 3, 4, 5, or 6 independently selected R^{G} substituents;
or, any R^{c1A} and R^{d1A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e1A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1A} and R^{g1A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)R^{a2}, —OC(O)NR^{c2}R^{d2}, —OC(O)OR^{a2}, —OS(O)₂R^{b2}, —OS(O)₂NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{e2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR²C(═NR^{e2})R^{a2}, —NR²C(═NR^{e2})NR^{c2}R^{d2}, —NR²S(O)(═NR^{e2})R^{b2}, —NR^{e2}S(O)(═NR^{e2})NR^{c2}R^{d2}, —OS(O)(═NR^{e2})R^{b2}, —S(O)(═NR^{e2})R^{b2}, —S(O)(═NR^{e2})NR^{c2}R^{d2}, —C(O)NR^{c2}S(O)₂R^{b2}, —C(O)NR^{c2}S(O)₂NR^{c2}R^{d2}, —S(O)₂NR^{c2}C(O)R^{b2}, —NR²S(O)NR^{c2}C(O)R^{b2}, and —P(O)R^{f2}R^{g2}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R² are each optionally substituted with 1, 2, 3, 4, 5, or 6 independently selected R^{2A} substituents;
each R^{a2}, R^{b2}, R^{c2}, and R^{a2} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a2}, R^{b2}, R^{c2}, and R^{d2} are each optionally substituted with 1, 2, 3, 4, 5, or 6 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{2A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a2A}, —SR^{a2A}, —NR^{c2A}R^{d2A}, —C(O)R^{a2A}, —C(O)OR^{a2A}, —C(O)NR^{c2A}R^{d2A}, —C(O)NR^{c2A}(OR^{a2A}), —OC(O)R^{a2A}, —OC(O)NR^{c2A}R^{d2A}, —OC(O)OR^{a2A}, —OS(O)₂R^{b2A}, —OS(O)₂NR^{c2A}R^{d2A}, —NR^{c2A}C(O)R^{a2A}, —NR^{c2A}C(O)OR^{a2A}, —NR^{c2A}C(O)NR^{c2A}R^{d2A}, —NR^{c2A}S(O)₂R^{b2A}, —NR^{c2A}S(O)₂NR^{c2A}R^{d2A}, —NR^{c2A}OR^{a2A}, —NR^{c2A}S(O)R^{b2A}, —NR^{c2A}S(O)NR^{c2A}R^{d2A}, —S(O)R^{b2A}, —S(O)₂R^{b2A}, —S(O)NR^{c2A}R^{d2A}, —S(O)₂NR^{c2A}R^{d2A}, —C(═NR^{e2A})R^{a2A}, —C(═NR^{e2A})NR^{c2A}R^{d2A}, —NR^{c2A}C(═NR^{e2A})R^{a2A}, —NR^{c2A}C(═NR^{e2A})NR^{c2A}R^{d2A}, —NR^{c2A}S(O)(═NR^{e2A})R^{b2A}, —NR^{c2A}S(O)(═NR^{e2A})NR^{c2A}R^{d2A}, —OS(O)(═NR^{e2A})R^{b2A}, —S(O)(═NR^{e2A})R^{b2A}, —S(O)(═NR^{e2A})NR^{c2A}R^{d2A}, —C(O)NR^{c2A}S(O)₂R^{b2A}, —C(O)NR^{c2A}S(O)₂NR^{c2A}R^{d2A}, —S(O)₂NR^{c2A}C(O)R^{b2A}, —NR^{c2A}S(O)NR^{c2A}C(O)R^{b2A}, and —P(O)R^{f2A}R^{g2A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{2A} are each optionally substituted with 1, 2, 3, 4, 5, or 6 independently selected R^{G} substituents;
each R^{a2A}, R^{b2A}, R^{c2A}, and R^{d2A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a2A}, R^{b2A}, R^{c2A}, and R^{d2A} are each optionally substituted with 1, 2, 3, 4, 5, or 6 independently selected R^{G} substituents;
or, any R^{c2A} and R^{d2A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e2A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f2A} and R^{g2A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R³ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a3}, —C(O)OR^{a3}, —C(O)NR^{c3}R^{d3}, —S(O)₂R^{b3}, —S(O)₂NR^{c3}R^{d3}, —S(O)(═NR^{c3})R^{b3}, and —S(O)(=NR^{e3})NR^{c3}R^{d3}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R³ are each optionally substituted with 1, 2, 3, 4, 5, or 6 independently selected R^{G} substituents;
or, R³ and L³ together with the atoms to which they are attached form a 5-10 membered heterocycloalkyl group, wherein the 5-10 membered heterocycloalkyl group is optionally substituted with 1, 2, 3, 4, 5, or 6 independently selected R^{G} substituents;
each R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a3}, R^{b3}, R^{c3}, and R^{d3} are each optionally substituted with 1, 2, 3, 4, 5, or 6 independently selected R^{G} substituents;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁴ are each optionally substituted with 1, 2, 3, 4, 5, or 6 independently selected R^{G} substituents;
R⁵ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁵ are each optionally substituted with 1, 2, 3, 4, 5, or 6 independently selected R^{5A} substituents;
each R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R^{a}s, R^{b5}, R^{c}S, and R^{a5} are each optionally substituted with 1, 2, 3, 4, 5, or 6 independently selected R^{5A} substituents;
or, any R^{c5} and R^{d5} attached to the same N atom, together with the N atom to which they are attached, form a 4-14 membered heterocycloalkyl group, wherein the 4-14 membered heterocycloalkyl group is optionally substituted with 1, 2, 3, 4, 5, or 6 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{5A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5A}, —SR^{a5A}, —NR^{c5A}R^{d5A}, —NO₂, —C(O)R^{a5A}, —C(O)OR^{a5A}, —C(O)NR^{c5A}R^{d5A}, —C(O)NR^{c5A}(OR^{a5A}), —OC(O)R^{a5A}, —OC(O)NR^{c5A}R^{d5A}, —OC(O)OR^{a5A}, —OS(O)₂R^{b5A}, —OS(O)₂NR^{c5A}R^{d5A}, —NR^{c5A}C(O)R^{a5A}, —NR^{c5A}C(O)OR^{a5A}, —NR^{c5A}C(O)NR^{c5A}R^{d5A}, —NR^{c5A}S(O)₂R^{b5A}, —NR^{c5A}S(O)₂NR^{c5A}R^{d5A}, —NR^{c5A}OR^{a5A}, —NR^{c5A}S(O)R^{b5A}, —NR^{c5A}S(O)NR^{c5A}R^{d5A}, —S(O)R^{b5A}, —S(O)₂R^{b5A}, —S(O)NR^{c5A}R^{d5A}, —S(O)₂NR^{c5A}R^{d5A}, —C(═NR^{e5A})R^{a5A}, —C(═NR^{e5A})NR^{c5A}R^{d5A}, —NR^{c5A}C(═NR^{e5A})R^{a5A}, —NR^{c5A}C(═NR^{e5A})NR^{c5A}R^{d5A}, —NR^{c5A}S(O)(═NR^{e5A})R^{b5A}, —NR^{c5A}S(O)(═NR^{e5A})NR^{c5A}R^{d5A}, —OS(O)(═NR^{e5A})R^{b5A}, —S(O)(═NR^{e5A})R^{b5A}, —S(O)(═NR^{e5A})NR^{c5A}R^{d5A}, —C(O)NR^{c5A}S(O)₂R^{b5A}, —C(O)NR^{c5A}S(O)₂NR^{c5A}R^{d5A}, —S(O)₂NR^{c5A}C(O)R^{b5A}, —NR^{c5A}S(O)NR^{c5A}C(O)R^{b5A}, and —P(O)R^{f5A}R^{g5A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{5A} are each optionally substituted with 1, 2, 3, 4, 5, or 6 independently selected R^{G} substituents;
each R^{a5A}, R^{b5A}, R^{c5A}, and R^{d5A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a5A}, R^{b5A}, R^{c5A}, and R^{d5A} are each optionally substituted with 1, 2, 3, 4, 5, or 6 independently selected R^{G} substituents;
or, any R^{c5A} and R^{d5A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e5A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5A} and R^{g5A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁶ are each optionally substituted with 1, 2, 3, 4, 5, or 6 independently selected R^{6A} substituents;
each R^{6A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —C(O)NR^{c6A}(OR^{a6A}), —OC(O)R^{a6A}, —OC(O)NR^{c6A}R^{d6A}, —OC(O)OR^{a6A}, —OS(O)₂R^{b6A}, —OS(O)₂NR^{c6A}R^{d6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)₂NR^{c6A}R^{d6A}, —NR^{c6A}OR^{a6A}, —NR^{c6A}S(O)R^{b6A}, —NR^{c6A}S(O)NR^{c6A}R^{d6A}, —S(O)R^{b6A}, —S(O)₂R^{b6A}, —S(O)NR^{c6A}R^{d6A}, —S(O)₂NR^{c6A}R^{d6A}, —C(═NR^{e6A})R^{a6A}, —C(═NR^{e6A})NR^{c6A}R^{d6A}, —NR^{c6A}C(═NR^{e6A})R^{a6A}, —NR^{c6A}C(═NR^{e6A})NR^{c6A}R^{d6A}, —NR^{c6A}S(O)(═NR^{e6A})R^{b6A}, —NR^{c6A}S(O)(═NR^{e6A})NR^{c6A}R^{d6A}, —OS(O)(═NR^{e6A})R^{b6A}, —S(O)(═NR^{e6A})R^{b6A}, —S(O)(═NR^{e6A})NR^{c6A}R^{d6A}, —C(O)NR^{c6A}S(O)₂R^{b6A}, —C(O)NR^{c6A}S(O)₂NR^{c6A}R^{d6A}, —S(O)₂NR^{c6A}C(O)R^{b6A}, —NR^{c6A}S(O)NR^{c6A}C(O)R^{b6A}, and —P(O)R^{f6A}R^{g6A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{6A} are each optionally substituted with 1, 2, 3, 4, 5, or 6 independently selected R^{G} substituents;
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a6A}, R^{b6A}, R^{c6A}, and R^{d6A} are each optionally substituted with 1, 2, 3, 4, 5, or 6 independently selected R^{G} substituents;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e6A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f6A} and R^{g6A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}, —NR^{c7}C(O)R^{a7}, —NR^{c7}C(O)OR^{a7}, —NR^{c7}C(O)NR^{c7}R^{d7}, —NR^{c7}S(O)₂R^{b7}, —NR^{c7}S(O)₂NR^{c7}R^{a7}, —NR^{c7}OR^{a7}, —NR^{c7}S(O)R^{b7}, —NR^{c7}S(O)NR^{c7}R^{d7}, —S(O)R^{b7}, —S(O)₂R^{b7}, —S(O)NR^{c7}R^{d7}, —S(O)₂NR^{c7}R^{d7}, —C(═NR^{e7})R^{a7}, —C(═NR^{e7})NR^{c7}R^{d7}, —NR^{c7}C(═NR^{e7})R^{a7}, —NR^{c7}C(═NR^{e7})NR^{c7}R^{d7}, —NR^{c7}S(O)(═NR^{e7})R^{b7}, —NR^{c7}S(O)(═NR^{e7})NR^{c7}R^{d7}, —OS(O)(═NR^{e7})R^{b7}, —S(O)(═NR^{e7})R^{b7}, —S(O)(═NR^{e7})NR^{c7}R^{d7}, —C(O)NR^{c7}S(O)₂R^{b7}, —C(O)NR^{c7}S(O)₂NR^{c7}R^{a7}, —S(O)₂NR^{c7}C(O)R^{b7}, —NR^{c7}S(O)NR^{c7}C(O)R^{b7}, and —P(O)R^{f7}R^{g7}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁷ are each optionally substituted with 1, 2, 3, 4, 5, or 6 independently selected R^{7A} substituents;
or, R⁵ and R⁷ together with the atoms to which they are attached form a C₅₋₁₀ cycloalkyl or 5-10 membered heterocycloalkyl group, wherein the C₅₋₁₀ cycloalkyl and 5-10 membered heterocycloalkyl group is optionally substituted with 1, 2, 3, 4, 5, or 6 independently selected R^{7A} substituents;
or, R⁶ and R⁷ together with the atoms to which they are attached form a 5-10 membered heterocycloalkyl group, wherein the 5-10 membered heterocycloalkyl group is optionally substituted with 1, 2, 3, 4, 5, or 6 independently selected R^{7A} substituents;
each R^{a7}, R^{b7}, R^{c7}, and R^{d7} is independently selected from H, C₁₋₆ alkyl, C₁₋₆ haloalkyl, C₂₋₆ alkenyl, C₂₋₆ alkynyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R^{a7}, R^{b7}, R^{c7}, and R^{f7} are each optionally substituted with 1, 2, 3, 4, 5, or 6 independently selected R^{7A} substituents;
or, any R^{c7} and R^{d7} attached to the same N atom, together with the N atom to which they are attached, form a 4-14 membered heterocycloalkyl group, wherein the 4-14 membered heterocycloalkyl group is optionally substituted with 1, 2, 3, 4, 5, or 6 independently selected R^{7A} substituents;
each R^{c7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7A}, —SR^{a7A}, —NR^{c7A}R^{d7A}, —NO₂, —C(O)R^{a7A}, —C(O)OR^{a7A}, —C(O)NR^{c7A}R^{d7A}, —C(O)NR^{c7A}(OR^{a7A}), —OC(O)R^{a7A}, —OC(O)NR^{c7A}R^{d7A}, —OC(O)OR^{a7A}, —OS(O)₂R^{b7A}, —OS(O)₂NR^{c7A}R^{d7A}, —NR^{c7A}C(O)R^{a7A}, —NR^{c7A}C(O)OR^{a7A}, —NR^{c7A}C(O)NR^{c7A}R^{d7A}, —NR^{c7A}S(O)₂R^{b7A}, —NR^{c7A}S(O)₂NR^{c7A}R^{d7A}, —NR^{c7A}OR^{a7A}, —NR^{c7A}S(O)R^{b7A}, —NR^{c7A}S(O)NR^{c7A}R^{d7A}, —S(O)R^{b7A}, —S(O)₂R^{b7A}, —S(O)NR^{c7A}R^{d7A}, S(O)₂NR^{c7A}R^{d7A}, —C(═NR^{e7A})R^{a7A}, —C(═NR^{e7A})NR^{c7A}R^{d7A}, —NR^{c7A}C(═NR^{e7A})R^{a7A}, —NR^{c7A}C(═NR^{e7A})NR^{c7A}R^{d7A}, —NR^{c7A}S(O)(═NR^{e7A})R^{b7A}, —NR^{c7A}S(O)(═NR^{e7A})NR^{c7A}R^{d7A}, —OS(O)(═NR^{e7A})R^{b7A}, —S(O)(═NR^{e7A})R^{b7A}, —S(O)(═NR^{e7A})NR^{7A}R^{d7A}, —C(O)NR^{c7A}S(O)₂R^{b7A}, —C(O)NR^{7A}S(O)₂NR^{7A}R^{d7A}, —S(O)₂NR^{c7A}C(O)R^{b7A}, —NR^{c7A}S(O)NR^{c7A}C(O)R^{b7A}, and —P(O)R^{f7A}R^{g7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{7B} substituents;
each R^{a7A}, R^{b7A}, R^{c7A}, and R^{d7A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7A}, R^{b7A}, R^{c7A}, and R^{d7A} are each optionally substituted with 1, 2, 3, 4, 5, or 6 independently selected R^{7B} substituents;
or, any R^{c7A} and R^{d7A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{7B} substituents;
each R^{e7A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7A} and R^{g7A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7B} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7B}, —SR^{a7B}, —NR^{c7B}R^{d7B}, —NO₂, —C(O)R^{a7B}, —C(O)OR^{a7B}, —C(O)NR^{c7B}R^{d7B}, —C(O)NR^{c7B}(OR^{a7B}), —OC(O)R^{a7B}, —OC(O)NR^{c7B}R^{d7B}, —OC(O)OR^{a7B}, —OS(O)₂R^{b7B}, —OS(O)₂NR^{c7B}R^{d7B}, —NR^{c7B}C(O)R^{a7B}, —NR^{c7B}C(O)OR^{a7B}, —NR^{c7B}C(O)NR^{c7B}R^{d7B}, —NR^{c7B}S(O)₂R^{b7B}, —NR^{c7B}S(O)₂NR^{c7B}R^{d7B}, —NR^{c7B}OR^{a7B}, —NR^{c7B}S(O)R^{b7B}, —NR^{c7B}S(O)NR^{c7B}R^{d7B}, —S(O)R^{b7B}, —S(O)₂R^{b7B}, —S(O)NR^{c7B}R^{d7B}, —S(O)₂NR^{c7B}R^{d7B}, —C(═NR^{e7B})R^{a7B}, —C(═NR^{e7B})NR^{c7B}R^{d7B}, —NR^{c7B}C(═NR^{e7B})R^{a7B}, —NR^{c7B}C(═NR^{e7B})NR^{c7B}R^{d7B}, —NR^{c7B}S(O)(═NR^{e7B})R^{b7B}, —NR^{c7B}S(O)(═NR^{e7B})NR^{c7B}R^{d7B}, —OS(O)(═NR^{e7B})R^{b7B}, —S(O)(═NR^{e7B})R^{b7B}, —S(O)(═NR^{e7B})NR^{c7B}R^{d7B}, —C(O)NR^{c7B}S(O)₂R^{b7B}, —C(O)NR^{c7B}S(O)₂NR^{c7B}R^{d7B}, —S(O)₂NR^{c7B}C(O)R^{b7B}, —NR^{c7B}S(O)NR^{c7B}C(O)R^{b7B}, and —P(O)R^{f7B}R^{g7B}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7B} are each optionally substituted with 1, 2, 3, 4, 5, or 6 independently selected R^{G} substituents;
each R^{a7B}, R^{b7B}, R^{c7B}, and R^{d7B} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7B}, R^{b7B}, R^{c7B}, and R^{d7B} are each optionally substituted with 1, 2, 3, 4, 5, or 6 independently selected R^{G} substituents;
or, any R^{c7B} and R^{d7B} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e7B} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7B} and R^{g7B} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁸ is selected from H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a}s, —SR^{a}s, —NR^{c8}R^{d8}, —NO₂, —C(O)R^{a}s, —C(O)OR^{a}s, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(=NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R R^{g8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁸ are each optionally substituted with 1, 2, 3, 4, 5, or 6 independently selected R^{8A} substituents;
each R^{a8}, R^{b8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{b8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —NR^{c8A}S(O)₂NR^{c8A}R^{d8A}, —NR^{c8A}OR^{a8A}, —NR^{c8A}S(O)R^{b8A}, —NR^{c8A}S(O)NR^{c8A}R^{d8A}, —S(O)R^{b8A}, —S(O)₂R^{b8A}, —S(O)NR^{c8A}R^{d8A}_S(O)₂NR^{c8A}R^{d8A}, —C(═NR^{e8A})R^{a8A}, —C(═NR^{e8A})NR^{c8A}R^{d8A}, —NR^{c8A}C(═NR^{e8A})R^{a8A}, —NR^{c8A}C(═NR^{e8A})NR^{c8A}R^{d8A}, —NR^{c8A}S(O)(═NR^{e8A})R^{b8A}, —NR^{c8A}S(O)(═NR^{e8A})NR^{c8A}R^{d8A}, —OS(O)(═NR^{e8A})R^{b8A}, —S(O)(═NR^{e8A})R^{b8A}, —S(O)(═NR^{e8A})NR^{c8A}R^{d8A}, —C(O)NR^{c8A}S(O)₂R^{b8A}, —C(O)NR^{c8A}S(O)₂NR^{c8A}R^{d8A}, —S(O)₂NR^{c8A}C(O)R^{b8A}, —NR^{c8A}S(O)NR^{c8A}C(O)R^{b8A}, and —P(O)R^{f8A}R^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{b8A}, R^{c8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8A}, R^{b8A}, R^{c8A}, and R^{d8A} are each optionally substituted with 1, 2, 3, 4, 5, or 6 independently selected R^{G} substituents;
or, any R^{c8A} and R^{d8A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, X¹ is N or NR⁶.

In some embodiments, X¹ is N.

In some embodiments, X¹ is NR⁶.

In some embodiments, R⁶ is selected from H, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —C(O)R^{a6}, —C(O)OR^{a6}, —C(O)NR^{c6}R^{d6}, —S(O)R^{b6}, and —S(O)₂R^{b6}, wherein C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —C(O)R^{a6}, and —S(O)₂R^{b6}, wherein C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —C(O)R^{a6}, and —S(O)₂R^{b6}, wherein C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a6}, and —S(O)₂R^{b6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, —C(O)R^{a6}, and —S(O)₂R^{b6}, wherein the C₁₋₆ alkyl, C₂₋₆ alkenyl, C₂₋₆ alkynyl, and C₁₋₆ haloalkyl of R⁶ are each optionally substituted with 1, 2, 3, or 4 independently selected R^{6A} substituents.

In some embodiments, R⁶ is selected from H, C₁₋₆ alkyl, C₂₋₆ alkenyl, C₂₋₆ alkynyl, and C₁₋₆ haloalkyl, wherein the C₁₋₆ alkyl, C₂₋₆ alkenyl, C₂₋₆ alkynyl, and C₁₋₆ haloalkyl of R⁶ are each optionally substituted with 1, 2, 3, or 4 independently selected R^{6A} substituents.

In some embodiments, R⁶ is selected from H, C₁₋₆ alkyl, —C(O)R^{a6}, and —S(O)₂R^{b6}, wherein the C₁₋₆ alkyl of R⁶ is optionally substituted with 1, 2, 3, or 4 independently selected R^{6A} substituents.

In some embodiments, each Rae, R^{b6}, R^{c6}, and R^{d6} is independently selected from H, C₁₋₆ alkyl, C₂₋₆ alkenyl, C₂₋₆ alkynyl, and C₁₋₆ haloalkyl.

In some embodiments, each Rae, R^{b6}, R^{c6}, and R^{d6} is independently selected from H, C₁₋₆ alkyl, and C₁₋₆ haloalkyl.

In some embodiments, each Rae, R^{b6}, R^{c6}, and R^{d6} is independently selected from H and C₁₋₆ alkyl.

In some embodiments, each Rae, R^{b6}, R^{c6}, and R^{d6} is independently selected from H and C₁₋₃ alkyl.

In some embodiments, each R^{a6} and R^{b6} is independently selected from H, C₁₋₆ alkyl, C₂₋₆ alkenyl, C₂₋₆ alkynyl, and C₁₋₆ haloalkyl.

In some embodiments, each R^{a6} and R^{b6} is independently selected from H, C₁₋₆ alkyl, and C₁₋₆ haloalkyl.

In some embodiments, each R^{a6} and R^{b6} is independently selected from H and C₁₋₆ alkyl.

In some embodiments, each R^{a6} and R^{b6} is independently selected from H and C₁₋₃ alkyl.

In some embodiments, R⁶ is selected from H and C₁₋₆ alkyl, wherein the C₁₋₆ alkyl of R⁶ is optionally substituted with 1, 2, 3, or 4 independently selected R^{6A} substituents.

In some embodiments, R⁶ is selected from H, methyl, ethyl, methycarbonyl, and methylsulfonyl, wherein the methyl and ethyl of R⁶ is optionally substituted with 1 or 2 independently selected R^{6A} substituents.

In some embodiments, R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a6}, and —S(O)₂R^{b6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents; and
each R^{a6} and R^{b6} is independently selected from H and C₁₋₆ alkyl.

In some embodiments, R⁶ is selected from H, C₁₋₆ alkyl, C₂₋₆ alkenyl, C₂₋₆ alkynyl, C₁₋₆ haloalkyl, —C(O)R^{a6}, and —S(O)₂R^{b6}, wherein the C₁₋₆ alkyl, C₂₋₆ alkenyl, C₂₋₆ alkynyl, and C₁₋₆ haloalkyl of R⁶ are each optionally substituted with 1, 2, 3, or 4 independently selected R^{6A} substituents; and
each R^{a6} and R^{b6} is independently selected from H and C₁₋₆ alkyl.

In some embodiments, R⁶ is selected from H, C₁₋₆ alkyl, —C(O)R^{a6}, and —S(O)₂R^{b6}, wherein the C₁₋₆ alkyl of R⁶ is optionally substituted with 1, 2, 3, or 4 independently selected R^{6A} substituents; and
each R^{a6} and R^{b6} is independently selected from H and C₁₋₆ alkyl.

In some embodiments, R⁶ is selected from H, C₁₋₆ alkyl, —C(O)R^{a6}, and —S(O)₂R^{b6}, wherein the C₁₋₆ alkyl of R⁶ is optionally substituted with 1, 2, 3, or 4 independently selected R^{6A} substituents; and
each R^{a6} and R^{b6} is independently selected from H and C₁₋₃ alkyl.

In some embodiments, R⁶ is selected from H, methyl, ethyl, methycarbonyl, and methylsulfonyl, wherein the methyl and ethyl of R⁶ are optionally substituted with 1 or 2 independently selected R^{6A} substituents.

In some embodiments, R⁶ is selected from H, methyl, and ethyl, wherein the methyl and ethyl of R⁶ are optionally substituted with 1 or 2 independently selected R^{6A} substituents.

In some embodiments, each R^{6A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)R^{b6A}, —NR^{c6A}S(O)NR^{c6A}R^{d6A}, —S(O)R^{b6A}, —S(O)₂R^{b6A}, S(O)NR^{6A}R^{d6A}, and —S(O)₂NR^{6A}R^{d6A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{6A} are each optionally substituted with 1, 2, 3, or 4 independently selected R^{G} substituents.

In some embodiments, each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl.

In some embodiments, each R^{a6A}, R^{b6A}, R^{c6A}, and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, any R^{c6A} and R^{d6A} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents.

In some embodiments, each R^{a6A}, R^{b6A}, R^{c6A}, and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents.

In some embodiments, each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl; or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, each R^{6A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)R^{b6A}, —NR^{c6A}S(O)NR^{c6A}R^{d6A}, —S(O)R^{b6A}, —S(O)₂R^{b6A}, —S(O)NR^{6A}R^{d6A}, and —S(O)₂NR^{6A}R^{d6A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{6A} are each optionally substituted with 1, 2, 3, or 4 independently selected R^{G} substituents; and
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, each R^{6A} is independently selected from oxo, halo, C₁₋₆ alkyl, C₁₋₆ haloalkyl, 4-7 membered heterocycloalkyl, —CN, —OR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —S(O)R^{b6A}, —S(O)₂R^{b6A}, —S(O)NR^{c6A}R^{d6A}, and —S(O)₂NR^{c6A}R^{d6A}, wherein the C₁₋₆ alkyl, C₁₋₆ haloalkyl, and 4-7 membered heterocycloalkyl of R^{6A} are each optionally substituted with 1, 2, 3, or 4 independently selected R^{G} substituents.

In some embodiments, each R^{6A} is independently selected from oxo, halo, C₁₋₆ alkyl, C₁₋₆ haloalkyl, —CN, —OR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —S(O)R^{b6A}, —S(O)₂R^{b6A}, —S(O)NR^{c6A}R^{d6A}, and —S(O)₂NR^{c6A}R^{d6A}, wherein the C₁₋₆ alkyl and C₁₋₆ haloalkyl of R^{6A} are each optionally substituted with 1, 2, 3, or 4 independently selected R^{G} substituents.

In some embodiments, each R^{6A} is independently selected from oxo, halo, C₁₋₆ alkyl, C₁₋₆ haloalkyl, 4-7 membered heterocycloalkyl, —CN, —OR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —S(O)R^{b6A}, S(O)₂R^{b6A}, —S(O)NR^{c6A}R^{d6A}, and —S(O)₂NR^{c6A}R^{d6A}, wherein the C₁₋₆ alkyl, C₁₋₆ haloalkyl, and 4-7 membered heterocycloalkyl of R^{6A} are each optionally substituted with 1, 2, 3, or 4 independently selected R^{G} substituents; and each R^{a6A}, R^{b6A}, R^{c6A}, and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, each R^{6A} is independently selected from oxo, halo, C₁₋₆ alkyl, C₁₋₆ haloalkyl, —CN, —OR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —S(O)R^{b6A}, —S(O)₂R^{b6A}, —S(O)NR^{6A}R^{d6A}, and —S(O)₂NR^{c6A}R^{d6A}, wherein the C₁₋₆ alkyl and C₁₋₆ haloalkyl of R^{6A} are each optionally substituted with 1, 2, 3, or 4 independently selected R^{G} substituents; and
each R^{a6A}, R^{b6A}, R^{c6A}, and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, each R^{6A} is independently selected from CN, 4-7 membered heterocycloalkyl, and —C(O)NR^{c6A}R^{d6A}.

In some embodiments, each R^{6A} is independently selected from —C(O)NR^{c6A}R^{d6A}.

In some embodiments, each R^{6A} is independently selected from CN, 4-7 membered heterocycloalkyl, and —C(O)NR^{c6A}R^{d6A}; and
each R^{c6A} and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, each R^{6A} is independently selected from —C(O)NR^{6A}R^{d6A}; and each R^{c6A} and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

In some embodiments, each R^{6A} is independently selected from CN, azetidinyl, aminocarbonyl, methylaminocarbonyl, and morpholinylcarbonyl.

In some embodiments, each R^{6A} is independently selected from methylaminocarbonyl and morpholinylcarbonyl.

In some embodiments, X² is CR⁷.

In some embodiments, R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁷ are each optionally substituted with 1, 2, 3, or 4 independently selected R^{7A} substituents.

In some embodiments, each R^{a7}, R^{b7}, R^{c7}, and R¹⁷ is independently selected from H, C₁₋₆ alkyl, C₂₋₆ alkenyl, C₂₋₆ alkynyl, and C₁₋₆ haloalkyl.

In some embodiments, each R^{a7}, R^{b7}, R^{c7}, and R^{d7} is independently selected from H, C₁₋₆ alkyl, and C₁₋₆ haloalkyl.

In some embodiments, each R^{a7}, R^{b7}, R^{c7}, and R^{d7} is independently selected from H and C₁₋₆ alkyl.

In some embodiments, each R^{c7} and R^{d7} is independently selected from H, C₁₋₆ alkyl, C₂₋₆ alkenyl, C₂₋₆ alkynyl, and C₁₋₆ haloalkyl.

In some embodiments, each R^{c7} and R^{d7} is independently selected from H, C₁₋₆ alkyl, and C₁₋₆ haloalkyl.

In some embodiments, each R^{c7} and R^{d7} is independently selected from H and C₁₋₆ alkyl.

In some embodiments, each R^{c7} and R^{d7} is independently selected from H and C₁₋₃ alkyl.

In some embodiments, R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁷ are each optionally substituted with 1, 2, 3, or 4 independently selected R^{7A} substituents; and
each R^{c7} and R^{d7} is independently selected from H, C₁₋₆ alkyl, C₂₋₆ alkenyl, C₂₋₆ alkynyl, and C₁₋₆ haloalkyl.

In some embodiments, R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁷ are each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁷ are each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of Rare each optionally substituted with 1, 2, 3, or 4 independently selected R^{7A} substituents; and
each R^{c7} and R^{d7} is independently selected from H, C₁₋₆ alkyl, C₂₋₆ alkenyl, C₂₋₆ alkynyl, and C₁₋₆ haloalkyl.

In some embodiments, R⁷ is selected from H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁷ are each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁷ are each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of Rare each optionally substituted with 1, 2, 3, or 4 independently selected R^{7A} substituents; and
each R⁷ and R¹⁷ is independently selected from H, C₁₋₆ alkyl, C₂₋₆ alkenyl, C₂₋₆ alkynyl, and C₁₋₆ haloalkyl

In some embodiments, R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of Rare each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, and —C(O)NR^{c7}R^{d7}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl of R⁷ are each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, and —C(O)NR^{c7}R^{d7}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl of R⁷ are each optionally substituted with 1, 2, 3, or 4 independently selected R^{7A} substituents; and
each R^{c7} and R^{d7} is independently selected from H, C₁₋₆ alkyl, C₂₋₆ alkenyl, C₂₋₆ alkynyl, and C₁₋₆ haloalkyl.

In some embodiments, R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl of R⁷ are each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, phenyl, 8-10 membered heterocycloalkyl, 5-6 membered heteroaryl, and —C(O)NR^{c7}R^{d7}, wherein the C₁₋₆ alkyl, C₂₋₆ alkenyl, C₂₋₆ alkynyl, phenyl, 8-10 membered heterocycloalkyl, and 5-6 membered heteroaryl of R⁷ are each optionally substituted with 1, 2, 3, or 4 independently selected R^{7A} substituents.

In some embodiments, R⁷ is selected from H, halo, C₁₋₆ alkyl, C₂₋₆ alkenyl, C₂₋₆ alkynyl, phenyl, 8-10 membered heterocycloalkyl, 5-6 membered heteroaryl, and —C(O)NR^{c7}R^{d7}, wherein the C₁₋₆ alkyl, C₂₋₆ alkenyl, C₂₋₆ alkynyl, phenyl, 8-10 membered heterocycloalkyl, and 5-6 membered heteroaryl of R⁷ are each optionally substituted with 1, 2, 3, or 4 independently selected R^{7A} substituents; and each R⁷ and R¹⁷ is independently selected from H and C₁₋₆ alkyl.

In some embodiments, R⁷ is selected from H, halo, C₁₋₆ alkyl, phenyl, and 5-6 membered heteroaryl, wherein the C₁₋₆ alkyl, phenyl, and 5-6 membered heteroaryl of R⁷ are each optionally substituted with 1, 2, 3, or 4 independently selected R^{7A} substituents.

In some embodiments, each R⁷ is H, chloro, bromo, methyl, ethyl, ethenyl, ethynyl, propynyl, dimethylpropynyl, 5,6,7,8-tetrahydro-[1,2,4]triazolo[1,5-a]pyrazinyl, phenyl, pyrazolyl, pyridinyl, triazolyl, pyrimidinyl, and ethylaminocarbonyl, wherein the methyl, ethyl, ethenyl, ethynyl, propynyl, dimethylpropynyl, 5,6,7,8-tetrahydro-[1,2,4]triazolo[1,5-a]pyrazinyl, phenyl, pyrazolyl, pyridinyl, triazolyl, and pyrimidinyl of R⁷ are each optionally substituted with 1, 2, 3, or 4 independently selected R^{7A} substituents.

In some embodiments, each R⁷ is H, chloro, bromo, methyl, phenyl, pyrazolyl, and pyridinyl, wherein the methyl, phenyl, pyrazolyl, and pyridinyl of R⁷ are each optionally substituted with 1, 2, 3, or 4 independently selected R^{7A} substituents.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{7B} substituents.

In some embodiments, each R^{a7A}, R^{a7B}, R^{a7C}, and R^{a7D} is independently selected from H, C₁₋₆ alkyl, C₂₋₆ alkenyl, C₂₋₆ alkynyl, and C₁₋₆ haloalkyl.

In some embodiments, each R^{a7A}, R^{a7B}, R^{a7C}, and R^{a7D} is independently selected from H, C₁₋₆ alkyl, and C₁₋₆ haloalkyl.

In some embodiments, each R^{a7A}, R^{a7B}, R^{a7C}, and R^{a7D} is independently selected from H and C₁₋₆ alkyl.

In some embodiments, each R^{a7A}, R^{a7C}, and R^{a7D} is independently selected from H, C₁₋₆ alkyl, C₂₋₆ alkenyl, C₂₋₆ alkynyl, and C₁₋₆ haloalkyl.

In some embodiments, each R^{a7A}, R^{a7C}, and R^{a7D} is independently selected from H, C₁₋₆ alkyl, and C₁₋₆ haloalkyl.

In some embodiments, each R^{a7A}, R^{a7C}, and R^{a7D} is independently selected from H and C₁₋₆ alkyl.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{7B} substituents; and each R^{a7A}, R^{c7A}, and R^{d7A} is independently selected from H, C₁₋₆ alkyl, and C₁₋₆ haloalkyl.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{7B} substituents.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, CN, OR^{a7A}, —NR^{c7A}R^{d7A}, and C(O)NR^{7A}R^{d7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, or 4 independently selected R substituents.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, or 4 independently selected R^{7B} substituents; and each R^{a7A}, R^{c7A}, and R^{d7A} is independently selected from H, C₁₋₆ alkyl, and C₁₋₆ haloalkyl.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{7A} are each optionally substituted with 1, 2, 3, or 4 independently selected R^{7B} substituents.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{7A} are each optionally substituted with 1, 2, 3, or 4 independently selected R^{7B} substituents; and each R^{a7A}, R^{c7A}, and R^{d7A} is independently selected from H and C₁₋₆ alkyl.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, CN, OR^{a7A}, —NR^{c7A}R^{d7A}, and C(O)NR^{c7A}R^{d7A} wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl of R^{7A} are each optionally substituted with 1, 2, 3, or 4 independently selected R substituents.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, CN, OR^{a7A}, —NR^{c7A}R^{d7A}, and C(O)NR^{c7A}R^{d7A} wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl of R^{7A} are each optionally substituted with 1, 2, 3, or 4 independently selected R^{7B} substituents; and each R^{a7A}, R^{c7A}, and R^{d7A} is independently selected from H and C₁₋₆ alkyl.

In some embodiments, each R^{7A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl.

In some embodiments, each R^{7A} is independently selected from halo, C₁₋₆ alkyl, C₂₋₆ alkenyl, C₂₋₆ alkynyl, C₁₋₆ haloalkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alknyl, C₁₋₆ haloalkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents.

In some embodiments, each R^{7A} is independently selected from halo, C₁₋₆ alkyl, C₂₋₆ alkenyl, C₂₋₆ alkynyl, C₁₋₆ haloalkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alknyl, C₁₋₆ haloalkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents; and each R^{a7A}, R^{c7A}, and R^{d7A} is independently selected from H and C₁₋₆ alkyl.

In some embodiments, each R^{7A} is independently selected from halo, C₁₋₆ alkyl, C₂₋₆ alkenyl, C₂₋₆ alkynyl, C₁₋₆ haloalkyl, and 4-7 membered heterocycloalkyl.

In some embodiments, each R^{7A} is independently selected from C₁₋₆alkyl, C₁₋₆ haloalkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₁₋₆ haloalkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents.

In some embodiments, each R^{7A} is independently selected from C₁₋₆alkyl, C₁₋₆ haloalkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₁₋₆ haloalkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents; and each R^{a7A}, R^{c7A}, and R^{d7A} is independently selected from H and C₁₋₆ alkyl.

In some embodiments, each R^{7A} is independently selected from C₁₋₆alkyl, C₁₋₆ haloalkyl, and 4-7 membered heterocycloalkyl.

In some embodiments, each R^{7A} is independently selected from C₁₋₆alkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents.

In some embodiments, each R^{7A} is independently selected from C₁₋₆alkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents; and each R^{a7A}, R^{c7A}, and R^{d7A} is independently selected from H and C₁₋₃ alkyl.

In some embodiments, each R^{7A} is independently selected from C₁₋₆ alkyl and 4-7 membered heterocycloalkyl.

In some embodiments, each R^{7A} is independently selected from C₁₋₆ alkyl.

In some embodiments, each R^{7A} is independently selected from 4-7 membered heterocycloalkyl.

In some embodiments, each R^{7A} is independently selected from methyl, isopropyl, cyclopropyl, cyclobutyl, oxetanyl, azetidinyl, tetrahydrofuranyl, tetrahydropyranyl, piperidinyl, imidazolyl, pyrazolyl, pyridinyl, cyano, hydroxy, amino, and aminocarbonyl, wherein the methyl, isopropyl, cyclopropyl, oxetanyl, azetidinyl, tetrahydrofuranyl, tetrahydropyranyl, piperidinyl, imidazolyl, pyrazolyl, and pyridinyl of R^{7A} are each optionally substituted with 1, 2, 3, or 4 independently selected R^{7B} substituents.

In some embodiments, each R^{7B} is independently selected from C₁₋₆ alkyl, OR^{a7B}, NR^{c7B}R^{d7B}, C(O)R^{a7B}, and —C(O)NR^{c7B}R^{d7B}, wherein the C₁₋₆ alkyl is optionally substituted by R^{G}.

In some embodiments, each R^{7B} is independently selected from C₁₋₆ alkyl, OR^{a7B}, NR^{c7B}R^{d7B}, C(O)R^{a7B}, and —C(O)NR^{c7B}R^{d7B}, wherein the C₁₋₆ alkyl is optionally substituted by OH.

In some embodiments, each R^{a7B}, R^{b7B}, R^{c7B}, and R^{d7B} is independently selected from H, C₁₋₆ alkyl, C₂₋₆ alkenyl, C₂₋₆ alkynyl, and C₁₋₆ haloalkyl.

In some embodiments, each R^{a7B}, R^{b7B}, R^{c7B}, and R^{d7B} is independently selected from H, C₁₋₆ alkyl, and C₁₋₆ haloalkyl.

In some embodiments, each R^{a7B}, R^{b7B}, R^{c7B}, and R^{d7B} is independently selected from H and C₁₋₆ alkyl.

In some embodiments, each R^{a7B}, R^{b7B}, R^{c7B}, and R^{d7B} is independently selected from H and C₁₋₃ alkyl.

In some embodiments, each R^{a7B}, R^{c7B}, and R^{d7B} is independently selected from H, C₁₋₆ alkyl, C₂₋₆ alkenyl, C₂₋₆ alkynyl, and C₁₋₆ haloalkyl.

In some embodiments, each R^{a7B}, R^{c7B}, and R^{d7B} is independently selected from H, C₁₋₆ alkyl, and C₁₋₆ haloalkyl.

In some embodiments, each R^{a7B}, R^{c7B}, and R^{d7B} is independently selected from H and C₁₋₆ alkyl.

In some embodiments, each R^{a7B}, R^{c7B}, and R^{d7B} is independently selected from H and C₁₋₃ alkyl.

In some embodiments, each R^{7B} is independently selected from methyl, hydroxymethyl, hydroxy, amino, methylcarbonyl, and methylaminocarbonyl.

In some embodiments, each R^{7A} is methyl.

In some embodiments, each R^{7A} is morpholinyl.

In some embodiments, R⁷ is selected from H, chloro, bromo, methyl, hydroxymethyl, cyanomethyl, hydroxyethyl, cyanoethyl, pyridinylethyl, cyanoethenyl, pyridinylethenyl, ethynyl, cyclopropylethynyl, (hydroxycyclopropyl)ethynyl, (hydroxymethylcyclopropyl)ethynyl, (aminocyclopropyl)ethynyl, (hydroxy)(dimethyl)propynyl, (amino)(dimethyl)propynyl, (methyloxetanyl)ethynyl, tetrahydropyranylethynyl, (methylcarbonylpiperidinyl)ethynyl, (methylaminocarbonylpiperidinyl)ethynyl, pyridinylethynyl, (methylpyrazolyl)ethynyl, (methylimidazolyl)ethynyl, 5,6,7,8-tetrahydro-[1,2,4]triazolo[1,5-a]pyrazinyl, pyrimidinyl, isopropylpyrazolyl, tetrahydrofuranylpyrazolyl, methyltriazolyl, phenyl, aminocarbonylphenyl, ethylaminocarbonyl, methylpyrazolyl, pyridinyl, and morpholinylmethyl.

In some embodiments, R⁷ is selected from H, chloro, bromo, methyl,

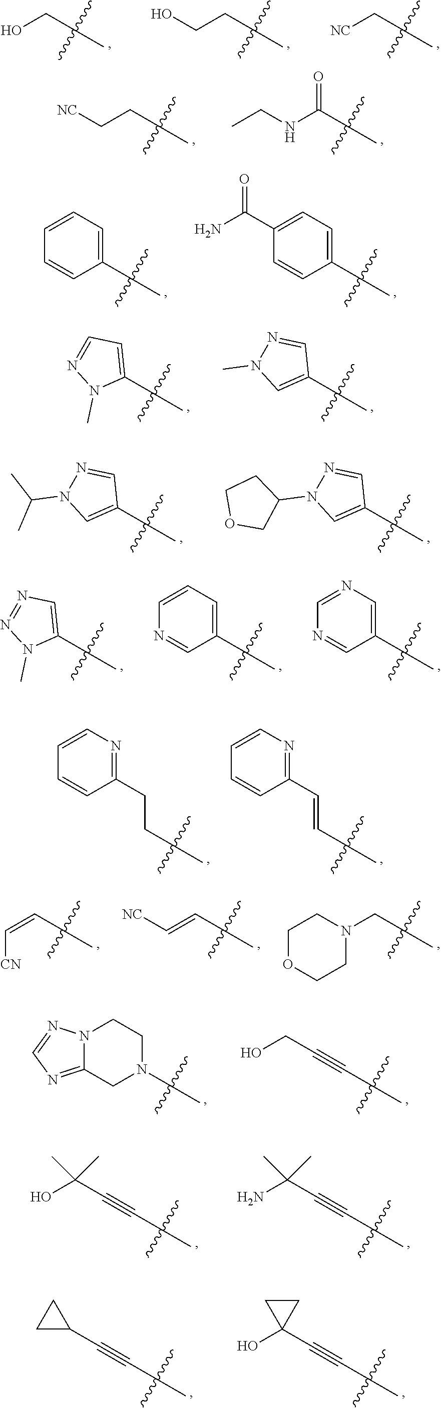

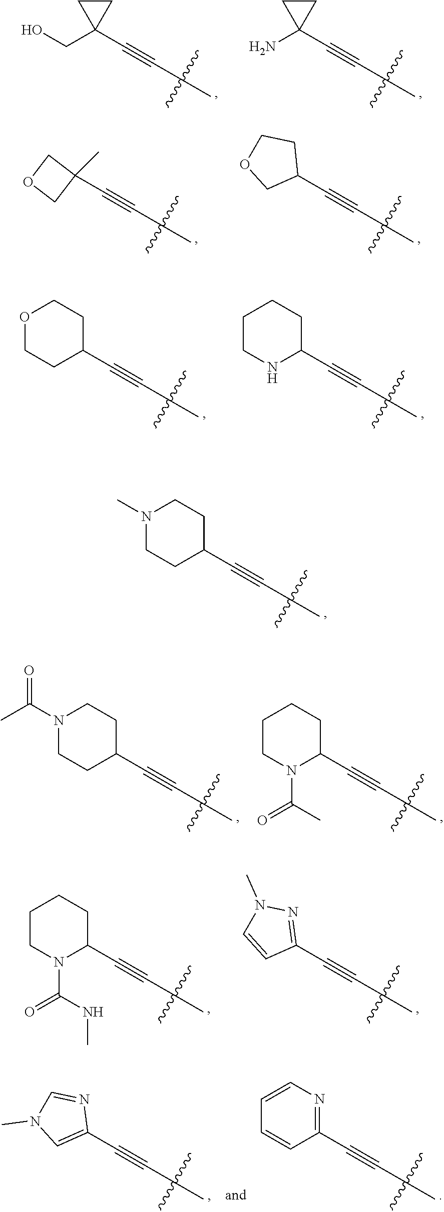

In some embodiments, R⁷ is selected from H, chloro, bromo, methyl, phenyl, methylpyrazolyl, pyridinyl, and morpholinylmethyl.

In some embodiments, R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents.

In some embodiments, R⁵ and R⁷ together with the atoms to which they are attached form a 5-10 membered heterocycloalkyl group, wherein the 5-10 membered heterocycloalkyl group is optionally substituted with 1, 2, 3, 4, 5, or 6 independently selected R^{7A} substituents.

In some embodiments, R⁶ and R⁷ together with the atoms to which they are attached form a 5-10 membered heterocycloalkyl group.

In some embodiments, R⁶ and R⁷ together with the atoms to which they are attached form a 5-7 membered heterocycloalkyl group.

In some embodiments, R⁶ and R⁷ together with the atoms to which they are attached form a 5-14 membered heterocycloalkyl group selected from

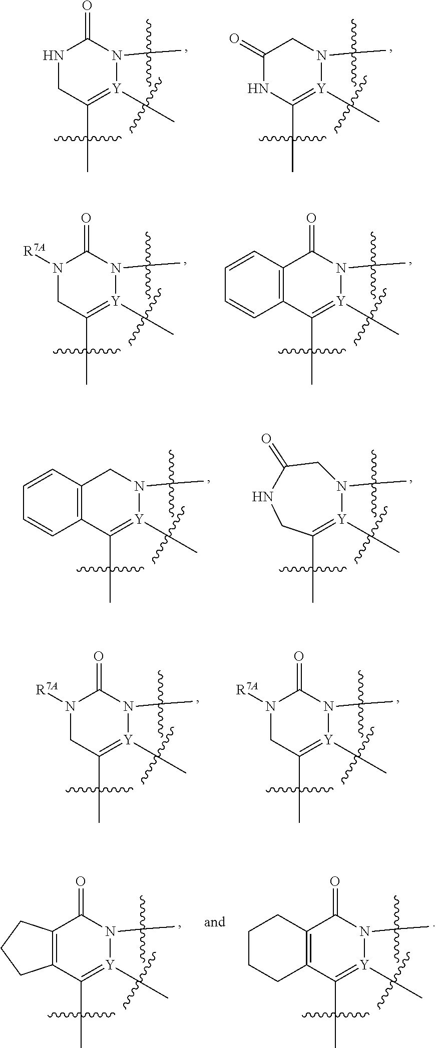

In some embodiments, R¹ and R⁷ together with the atoms to which they are attached form a 5-14 membered heterocycloalkyl group selected from

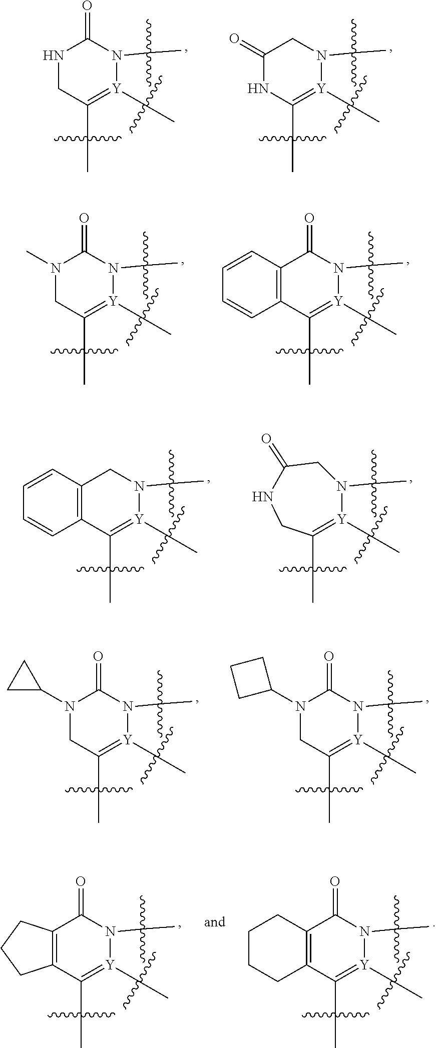

In some embodiments, X³ is CR⁸.

In some embodiments, R⁸ is selected from H, halo, C₁₋₆ alkyl, C₂₋₆ alkenyl, C₂₋₆ alkynyl, and C₁₋₆ haloalkyl.

In some embodiments, X³ is CH.

In some embodiments, Y is C.

In some embodiments, Y is N.

In some embodiments, Z is C.

In some embodiments, Z is N.

In some embodiments, Y is C and Z is C.

In some embodiments, Y is C and Z is N.

In some embodiments, R³ is selected from H, C₁₋₆ alkyl, C₁₋₆ haloalkyl, and —C(O)NR^{c3}R^{d3}.

In some embodiments, each R^{a3}, R^{b3}, R^{c3}, and R^{d3} is independently selected from H, C₁₋₆ alkyl, C₂₋₆ alkenyl, C₂₋₆ alkynyl, and C₁₋₆ haloalkyl.

In some embodiments, each R^{a3}, R^{b3}, R^{c3}, and R^{d3} is independently selected from H, C₁₋₆ alkyl, and C₁₋₆ haloalkyl.

In some embodiments, each R^{a3}, R^{b3}, R^{c3}, and R^{d3} is independently selected from H and C₁₋₆ alkyl.

In some embodiments, each R^{a3}, R^{b3}, R^{c3}, and R^{d3} is independently selected from H and C₁₋₃ alkyl.

In some embodiments, each R^{c3} and R^{d3} is independently selected from H, C₁₋₆ alkyl, C₂₋₆ alkenyl, C₂₋₆ alkynyl, and C₁₋₆ haloalkyl.

In some embodiments, each R^{c3} and R^{d3} is independently selected from H, C₁₋₆ alkyl, and C₁₋₆ haloalkyl.

In some embodiments, each R^{c3} and R^{d3} is independently selected from H and C₁₋₆ alkyl.

In some embodiments, each R^{c3} and R^{d3} is independently selected from H and C₁₋₃ alkyl.

In some embodiments, R³ is selected from H, C₁₋₆ alkyl, C₁₋₆ haloalkyl, and —C(O)NR^{c3}R^{d3}; and each R^{c3} and R^{d3} is independently selected from H and C₁₋₆ alkyl.

In some embodiments, R³ is selected from H, C₁₋₆ alkyl, and C₁₋₆ haloalkyl.

In some embodiments, R³ is selected from H, C₁₋₆ alkyl, and —C(O)NR^{c3}R^{d3}.

In some embodiments, R³ is selected from H, C₁₋₆ alkyl, and —C(O)NR^{c3}R^{d3}; and
each R^{c3} and R^{d3} is independently selected from H and C₁₋₃ alkyl.

In some embodiments, R³ is H or C₁₋₆ alkyl.

In some embodiments, R³ is H.

In some embodiments, R³ is C₁₋₆ alkyl.

In some embodiments, R³ is —C(O)NR^{c3}R^{d3}.

In some embodiments, R³ is —C(O)NR^{c3}R^{d3}; and each R^{c3} and R¹³ is independently selected from H and C₁₋₃ alkyl.

In some embodiments, R³ is selected from H, methyl, and aminomethylcarbonyl.

In some embodiments, R³ is H.

In some embodiments, R³ is methyl.

In some embodiments, R³ is aminomethylcarbonyl.

In some embodiments, R⁴ is selected from H, C₁₋₆ alkyl, and C₁₋₆ haloalkyl.

In some embodiments, R⁴ is selected from H and C₁₋₆ alkyl.

In some embodiments, R⁴ is selected from H and C₁₋₃ alkyl.

In some embodiments, R⁴ is H.

In some embodiments, R⁴ is C₁₋₆ alkyl.

In some embodiments, R⁴ is C₁₋₃ alkyl.

In some embodiments, R⁴ is methyl.

In some embodiments:
R³ is selected from H, C₁₋₆ alkyl, and —C(O)NR^{c3}R^{d3};
each R^{c3} and R¹³ is independently selected from H and C₁₋₃ alkyl; and
R⁴ is selected from H and C₁₋₆ alkyl.

In some embodiments:
R³ is selected from H, C₁₋₃ alkyl, and —C(O)NR^{c3}R^{d3};
each R^{c3} and R^{d3} is independently selected from H and C₁₋₃ alkyl; and
R⁴ is selected from H and C₁₋₃ alkyl.

In some embodiments, R³ is H or C₁₋₆ alkyl; and R⁴ is H.

In some embodiments, L¹ is —N(R^{L})C(O)— or —N(R^{L})—.

In some embodiments, L¹ is —N(R^{L})C(O)—.

In some embodiments, L¹ is —N(R^{L})—.

In some embodiments, L¹ is —NHC(O)—.

In some embodiments, L¹ is —NH—.

In some embodiments, L² is a bond.

In some embodiments, L³ is selected from C₁₋₆ alkylene, —O—, and —N(R^{L})—.

In some embodiments, L³ is selected from methylene, —O—, and —NH—.

In some embodiments, L³ is methylene.

In some embodiments, L³ is —O—.

In some embodiments, L³ is —NH—.

In some embodiments, L⁴ is a bond or —O—.

In some embodiments, L⁴ is a bond.

In some embodiments, L⁴ is —O—.

In some embodiments, Ring A is C₃₋₁₀ cycloalkyl, C₆₋₁₀ aryl, 4-10 membered heterocycloalkyl, or 5-10 membered heteroaryl.

In some embodiments, Ring A is C₆₋₁₀ aryl, 4-10 membered heterocycloalkyl, or 5-10 membered heteroaryl.

In some embodiments, Ring A is C₆₋₁₀ aryl.

In some embodiments, Ring A is phenyl.

In some embodiments, Ring A is 4-10 membered heterocycloalkyl.

In some embodiments, Ring A is 8-10 membered heterocycloalkyl.

In some embodiments, Ring A is 5-10 membered heteroaryl.

In some embodiments, Ring A is 8-10 membered heteroaryl.

In some embodiments, Ring A is phenyl, 8-10 membered heterocycloalkyl, or 8-10 membered heteroaryl.

In some embodiments, Ring A is phenyl, benzo[b]thiopheneyl, or indolinyl.

In some embodiments, n is 0, 1, 2, 3, or 4.

In some embodiments, n is 0, 1, 2, or 3.

In some embodiments, n is 0, 2, or 3.

In some embodiments, each R¹ is independently selected from oxo, halo, C₁₋₆ alkyl, C₂₋₆ alkenyl, C₂₋₆ alkynyl, C₁₋₆ haloalkyl, —CN, and —OR^{a1}, wherein the C₁₋₆ alkyl, C₂₋₆ alkenyl, C₂₋₆ alkynyl, and C₁₋₆ haloalkyl of R¹ are each optionally substituted with 1, 2, 3, 4, 5, or 6 independently selected R^{1A} substituents.

In some embodiments, each R¹ is independently selected from oxo, halo, C₁₋₆ alkyl, C₂₋₆ alkenyl, C₂₋₆ alkynyl, C₁₋₆ haloalkyl, —CN, and —OR^{a1}.

In some embodiments, each R^{a1}, R^{b1}, R^{c1}, and R^{d1} is independently selected from H, C₁₋₆ alkyl, C₂₋₆ alkenyl, C₂₋₆ alkynyl, and C₁₋₆ haloalkyl.

In some embodiments, each R^{a1}, R^{b1}, R^{c1}, and R^{d1} is independently selected from H, C₁₋₆ alkyl, and C₁₋₆ haloalkyl.

In some embodiments, each R^{a1}, R^{b1}, R^{c1}, and R^{d1} is independently selected from H and C₁₋₆ alkyl.

In some embodiments, each R^{a1}, R^{b1}, R^{c1}, and R^{d1} is H.

In some embodiments, each R¹ is independently selected from oxo, halo, C₁₋₆ alkyl, C₂₋₆ alkenyl, C₂₋₆ alkynyl, C₁₋₆ haloalkyl, —CN, and —OR^{a1}; and
each R^{a1} is independently selected from H, C₁₋₆ alkyl, C₂₋₆ alkenyl, C₂₋₆ alkynyl, and C₁₋₆ haloalkyl.

In some embodiments, each R¹ is independently selected from halo, C₁₋₆ alkyl, C₁₋₆ haloalkyl, and OR^{a1}; and
each R^{a1} is independently selected from H, C₁₋₆ alkyl, and C₁₋₆ haloalkyl.

In some embodiments, each R¹ is independently selected from fluoro, chloro, trifluoromethyl, and hydroxy.

In some embodiments, each R¹ is independently selected from fluoro, trifluoromethyl, and hydroxy.

In some embodiments, Ring A is selected from

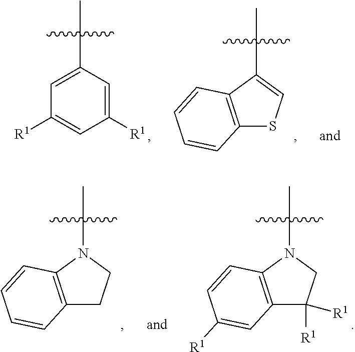

In some embodiments, Ring A is

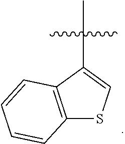 [SMILES: *c1csc2ccccc12]

In some embodiments, Ring A is

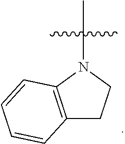 [SMILES: *N1CCc2ccccc21]

In some embodiments, Ring A is

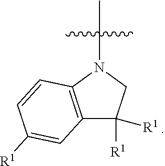

In some embodiments, Ring A is

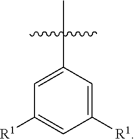

In some embodiments, Ring A is

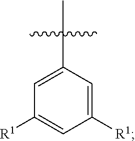

and
R¹ is independently selected from halo, C₁₋₆ alkyl, C₁₋₆ haloalkyl, and OR^{a1}; and
each R^{a1} is independently selected from H, C₁₋₆ alkyl, and C₁₋₆ haloalkyl.

In some embodiments, Ring A is

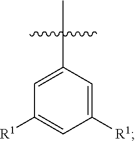

and
R¹ is independently selected from halo, C₁₋₆ alkyl, and C₁₋₆ haloalkyl.

In some embodiments, Ring A is

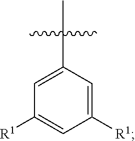

and
each R¹ is independently selected from fluoro, chloro, trifluoromethyl.

In some embodiments, Ring A is

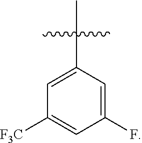 [SMILES: *c1cc(F)cc(C(F)(F)F)c1]

In some embodiments, Ring B is C₃₋₁₀ cycloalkyl, C₆₋₁₀ aryl, 4-10 membered heterocycloalkyl, or 5-10 membered heteroaryl.

In some embodiments, Ring B is C₃₋₇ cycloalkyl, phenyl, 4-7 membered heterocycloalkyl, or 5-6 membered heteroaryl.

In some embodiments, Ring B is C₆₋₁₀ aryl or 5-10 membered heteroaryl.

In some embodiments, Ring B is phenyl or 5-6 membered heteroaryl.

In some embodiments, Ring B is phenyl or pyridinyl.

In some embodiments, m is 0, 1, 2, or 3.

In some embodiments, m is 1 or 2.

In some embodiments, each R² is independently selected from halo, C₁₋₆ alkyl, C₂₋₆ alkenyl, C₂₋₆ alkynyl, and C₁₋₆ haloalkyl, wherein the C₁₋₆ alkyl, C₂₋₆ alkenyl, C₂₋₆ alkynyl, and C₁₋₆ haloalkyl of R² are each optionally substituted with 1, 2, 3, 4, 5, or 6 independently selected R^{2A} substituents.

In some embodiments, each R² is independently selected from halo, C₁₋₆ alkyl, C₂₋₆ alkenyl, C₂₋₆ alkynyl, and C₁₋₆ haloalkyl.

In some embodiments, each R² is independently selected from halo.

In some embodiments, each R² is independently chloro or fluoro.

In some embodiments, Ring B is

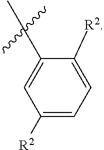

In some embodiments, Ring B is

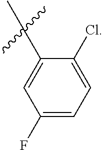 [SMILES: *c1cc(F)ccc1Cl]

In some embodiments:
X¹ is CR⁵, N, or NR⁶;
X² is CR⁷ or N;
X³ is CR⁸ or N;
Y is C or N;
Z is C or N;
n is 0, 1, 2, 3, or 4;
m is 0, 1, 2, 3, or 4;
L¹ is —NHC(O)— or —NH—;
L² is a bond;
L³ is selected from C₁₋₆ alkylene, —O—, and —NH—;
L⁴ is a bond or —O—;
each R¹ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a1}, —SR^{a1}, —NR^{c1}R^{d1}, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}C(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{c1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R are each optionally substituted with 1, 2, 3, 4, 5, or 6 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a1}, R^{b1}, R^{c1}, and R^{d1} are each optionally substituted with 1, 2, 3, 4, 5, or 6 independently selected R^{1A} substituents;
or, any R^{c1} and R^{d1} attached to the same N atom, together with the N atom to which they are attached, form a 4-7 membered heterocycloalkyl group, wherein the 4-10 membered heterocycloalkyl group is optionally substituted with 1, 2, 3, 4, 5, or 6 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy;
each R^{f1} and R^{g1} are independently selected from H, C₁₋₆ alkyl, C₁₋₆ alkoxy, C₁₋₆ haloalkyl, C₁₋₆ haloalkoxy;
each R^{1A} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R² is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)R^{a2}, —OC(O)NR^{c2}R^{d2}, —OC(O)OR^{a2}, —OS(O)₂R^{b2}, —OS(O)₂NR^{e2}R^{a2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —OS(O)(═NR^{e2})R^{b2}, —S(O)(═NR^{e2})R^{b2}, —S(O)(═NR^{e2})NR^{c2}R^{d2}, —C(O)NR^{c2}S(O)₂R^{b2}, —C(O)NR^{c2}S(O)₂NR^{c2}R^{d2}, —S(O)₂NR^{c2}C(O)R^{b2}, —NR^{c2}S(O)NR^{c2}C(O)R^{b2}, and —P(O)R^{f2}R^{g2}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R² are each optionally substituted with 1, 2, 3, 4, 5, or 6 independently selected R^{2A} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a2}, R^{b2}, R^{c2}, and R^{d2} are each optionally substituted with 1, 2, 3, 4, 5, or 6 independently selected R^{2A} substituents;
or, any R^{c2} and R^{d2} attached to the same N atom, together with the N atom to which they are attached, form a 4-7 membered heterocycloalkyl group, wherein the 4-10 membered heterocycloalkyl group is optionally substituted with 1, 2, 3, 4, 5, or 6 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy;
each R^{f2} and R^{g2} are independently selected from H, C₁₋₆ alkyl, C₁₋₆ alkoxy, C₁₋₆ haloalkyl, C₁₋₆ haloalkoxy;
each R^{2A} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R³ is selected from H, C₁₋₆ alkyl, C₁₋₆ haloalkyl, —C(O)R^{a3}, —C(O)OR^{a3}, —C(O)NR^{c3}R^{d3}, —S(O)₂R^{b3}, —S(O)₂NR^{c3}R^{d3}, —S(O)(═NR^{e3})R^{b3}, and —S(O)(═NR^{e3})NR^{c3}R^{d3}, wherein the C₁₋₆ alkyl, and C₁₋₆ haloalkyl of R³ are each optionally substituted with 1, 2, 3, 4, 5, or 6 independently selected R^{G} substituents;
or, R³ and L³ together with the atoms to which they are attached form a 5-10 membered heterocycloalkyl group, wherein the 5-10 membered heterocycloalkyl group is optionally substituted with 1, 2, 3, 4, 5, or 6 independently selected R^{G} substituents; each R^{a3}, R^{b3}, R^{c3}, and R^{d3} is independently selected from H, C₁₋₆ alkyl, and C₁₋₆ haloalkyl;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, C₁₋₆ alkyl, and C₁₋₆ haloalkyl;
R⁸ is selected from H, halo, CN, C₁₋₆ alkyl, C₁₋₆ haloalkyl, C₁₋₆ alkoxyl and C₁₋₆ haloalkoxyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
X¹ is N or NR⁶;
X² is CR⁷;
X³ is CR⁸;
Y is C or N;
Z is C or N;
n is 0, 1, 2, 3, or 4;
m is 0, 1, 2, 3, or 4;
Ring A is phenyl, 8-10 membered heterocycloalkyl, or 8-10 membered heteroaryl;
Ring B is phenyl or 5-6 membered heteroaryl;
Ring C is a 5-membered heteroaryl having 2 to 3 nitrogen atoms as ring members;
L¹ is —N(R^{L})C(O)— or —N(R^{L})—;
L² is a bond;
L³ is selected from C₁₋₆ alkylene, —O—, and —N(R^{L})—;
L⁴ is a bond or —O—;
each R^{L} is independently selected from H and C₁₋₆ alkyl;
each R¹ is independently selected from oxo, halo, C₁₋₆ alkyl, C₂₋₆ alkenyl, C₂₋₆ alkynyl, C₁₋₆ haloalkyl, —CN, and —OR^{a1};
each R^{a1} is independently selected from H, C₁₋₆ alkyl, C₂₋₆ alkenyl, C₂₋₆ alkynyl, and C₁₋₆ haloalkyl;
each R² is independently selected from halo, C₁₋₆ alkyl, C₂₋₆ alkenyl, C₂₋₆ alkynyl, and C₁₋₆ haloalkyl;
R³ is selected from H, C₁₋₆ alkyl, C₁₋₆ haloalkyl, and —C(O)NR^{c3}R^{d3};
each R^{c3} and R^{d3} is independently selected from H and C₁₋₆ alkyl
R⁴ is selected from H, C₁₋₆ alkyl, and C₁₋₆ haloalkyl;
R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a6}, —C(O)OR^{a6}, —C(O)NR^{c6}R^{d6}, —C(O)NR^{c6}(OR^{a6}), —S(O)R^{b6}, —S(O)₂R^{b6}, —S(O)NR^{c6}R^{d6}, —S(O)₂NR^{c6}R^{d6}, —C(═NR^{e6})R^{a6}, —C(═NR^{e6})NR^{c6}R^{d6}, —S(O)(=NR^{e6})R^{b6}, —S(O)(═NR^{e6})NR^{c6}R^{d6}, —C(O)NR^{c6}S(O)₂R^{b6}, —C(O)NR^{c6}S(O)₂NR^{c6}R^{d6}, and —S(O)₂NR^{c6}C(O)R^{b6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents;
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a6}, R^{b6}, R^{c6}, and R^{d6} are each optionally substituted with 1, 2, 3, 4, 5, or 6 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents;
each R^{6A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)R^{b6A}, —NR^{c6A}S(O)NR^{c6A}R^{d6A}, —S(O)R^{b6A}, —S(O)₂R^{b6A}, —S(O)NR^{c6A}R^{d6A}, and —S(O)₂NR^{c6A}R^{d6A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{6A} are each optionally substituted with 1, 2, 3, or 4 independently selected R^{G} substituents;
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6A}R^{b6A}, R^{c6A}, and R^{d6A} are each optionally substituted with 1, 2, 3, or 4 independently selected R^{G} substituents; or
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents;
R⁸ is selected from H, halo, C₁₋₆ alkyl, C₂₋₆ alkenyl, C₂₋₆ alkynyl, and C₁₋₆ haloalkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
X¹ is N or NR⁶;
X² is CR⁷;
X³ is CRB;
Y is C or N;
Z is C or N;
n is 0, 1, 2, 3, or 4;
m is 0, 1, 2, 3, or 4;
Ring A is phenyl, 8-10 membered heterocycloalkyl, or 8-10 membered heteroaryl;
Ring B is phenyl or 5-6 membered heteroaryl;
Ring C is a 5-membered heteroaryl having 2 to 3 nitrogen atoms as ring members;
L¹ is —N(R^{L})C(O)— or —N(R^{L})—;
L² is a bond;
L³ is selected from C₁₋₆ alkylene, —O—, and —N(R^{L})—;
L⁴ is a bond or —O—;
each R^{L} is independently selected from H and C₁₋₆ alkyl;
each R¹ is independently selected from oxo, halo, C₁₋₆ alkyl, C₂₋₆ alkenyl, C₂₋₆ alkynyl, C₁₋₆ haloalkyl, —CN, and —OR^{a1};
each R^{a1} is independently selected from H, C₁₋₆ alkyl, C₂₋₆ alkenyl, C₂₋₆ alkynyl, and C₁₋₆ haloalkyl;
each R² is independently selected from halo, C₁₋₆ alkyl, C₂₋₆ alkenyl, C₂₋₆ alkynyl, and C₁₋₆ haloalkyl;
R³ is selected from H, C₁₋₆ alkyl, and C₁₋₆ haloalkyl;
R⁴ is selected from H, C₁₋₆ alkyl, and C₁₋₆ haloalkyl;
R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents;
or, R⁶ and R⁷ together with the atoms to which they are attached form a 5-10 membered heterocycloalkyl group, wherein the 5-10 membered heterocycloalkyl group is optionally substituted with 1, 2, 3, 4, 5, or 6 independently selected R^{7A} substituents;
each R^{6A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)R^{b6A}, —NR^{c6A}S(O)NR^{c6A}R^{d6A}, —S(O)R^{b6A}, —S(O)₂R^{b6A}, —S(O)NR^{c6A}R^{d6A}, and —S(O)₂NR^{c6A}R^{d6A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{6A} are each optionally substituted with 1, 2, 3, or 4 independently selected R^{G} substituents;
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6A}, R^{b6A}, R^{c6A}, and R^{d6A} are each optionally substituted with 1, 2, 3, or 4 independently selected R^{G} substituents; or
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents;
R⁸ is selected from H, halo, C₁₋₆ alkyl, C₂₋₆ alkenyl, C₂₋₆ alkynyl, and C₁₋₆ haloalkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
X¹ is N or NR⁶;
X² is CR⁷;
X³ is CRB;
Y is C or N;
Z is C or N;
n is 0, 1, 2, 3, or 4;
m is 0, 1, 2, 3, or 4;
Ring A is phenyl, 8-10 membered heterocycloalkyl, or 8-10 membered heteroaryl;
Ring B is phenyl or 5-6 membered heteroaryl;
Ring C is a 5-membered heteroaryl having 2 to 3 nitrogen atoms as ring members;
L¹ is —N(R^{L})C(O)— or —N(R^{L})—;
L² is a bond;
L³ is selected from C₁₋₆ alkylene, —O—, and —N(R^{L})—;
L⁴ is a bond or —O—;
each R^{L} is independently selected from H and C₁₋₆ alkyl;
each R¹ is independently selected from oxo, halo, C₁₋₆ alkyl, C₂₋₆ alkenyl, C₂₋₆ alkynyl, C₁₋₆ haloalkyl, —CN, and —OR^{a1};
each R^{a1} is independently selected from H, C₁₋₆ alkyl, C₂₋₆ alkenyl, C₂₋₆ alkynyl, and C₁₋₆ haloalkyl;
each R² is independently selected from halo, C₁₋₆ alkyl, C₂₋₆ alkenyl, C₂₋₆ alkynyl, and C₁₋₆ haloalkyl;
R³ is selected from H, C₁₋₆ alkyl, C₁₋₆ haloalkyl, and —C(O)NR^{c3}R^{d3};
each R^{c3} and R^{d3} is independently selected from H and C₁₋₆ alkyl;
R⁴ is selected from H, C₁₋₆ alkyl, and C₁₋₆ haloalkyl;
R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a6}, and —S(O)₂R^{b6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents;
each R^{a6} and R^{b6} is independently selected from H and C₁₋₆ alkyl;
or, R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents;
each R^{6A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)R^{b6A}, —NR^{c6A}S(O)NR^{c6A}R^{d6A}, —S(O)R^{b6A}, —S(O)₂R^{b6A}, —S(O)NR^{c6A}R^{d6A}, and —S(O)₂NR^{c6A}R^{d6A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{6A} are each optionally substituted with 1, 2, 3, or 4 independently selected R^{G} substituents;
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6A}, R^{b6A}, R^{c6A}, and R^{d6A} are each optionally substituted with 1, 2, 3, or 4 independently selected R^{G} substituents; or
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents;
R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁷ are each optionally substituted with 1, 2, 3, or 4 independently selected R^{7A} substituents;
each R^{c7} and R^{d7} is independently selected from H, C₁₋₆ alkyl, C₂₋₆ alkenyl, C₂₋₆ alkynyl, and C₁₋₆ haloalkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, or 4 independently selected R^{7B} substituents;
each R^{a7A}, R^{c7A}, and R^{d7A} is independently selected from H, C₁₋₆ alkyl, and C₁₋₆ haloalkyl;
each R^{7B} is independently selected from C₁₋₆ alkyl, OR^{a7B}, NR^{c7B}R^{d7B}, C(O)R^{a7B}, and —C(O)NR^{c7B}R^{d7B}, wherein the C₁₋₆ alkyl is optionally substituted by OH;
each R^{a7B}, R^{c7B}, and R^{d7B} is independently selected from H and C₁₋₃ alkyl;
R⁸ is selected from H, halo, C₁₋₆ alkyl, C₂₋₆ alkenyl, C₂₋₆ alkynyl, and C₁₋₆ haloalkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
X¹ is N or NR⁶;
X² is CR⁷;
X³ is CR⁸;
Y is C or N;
Z is C or N;
n is 0, 1, 2, 3, or 4;
m is 0, 1, 2, 3, or 4;
Ring A is phenyl, 8-10 membered heterocycloalkyl, or 8-10 membered heteroaryl;
Ring B is phenyl or 5-6 membered heteroaryl;
Ring C is a 5-membered heteroaryl having 2 to 3 nitrogen atoms as ring members;
L¹ is —N(R^{L})C(O)— or —N(R^{L})—;
L² is a bond;
L³ is selected from C₁₋₆ alkylene, —O—, and —N(R^{L})—;
L⁴ is a bond or —O—;
each R^{L} is independently selected from H and C₁₋₆ alkyl;
each R¹ is independently selected from oxo, halo, C₁₋₆ alkyl, C₂₋₆ alkenyl, C₂₋₆ alkynyl, C₁₋₆ haloalkyl, —CN, and —OR^{a1};
each R^{a1} is independently selected from H, C₁₋₆ alkyl, C₂₋₆ alkenyl, C₂₋₆ alkynyl, and C₁₋₆ haloalkyl;
each R² is independently selected from halo, C₁₋₆ alkyl, C₂₋₆ alkenyl, C₂₋₆ alkynyl, and C₁₋₆ haloalkyl;
R³ is selected from H, C₁₋₆ alkyl, C₁₋₆ haloalkyl, and —C(O)NR^{c3}R^{d3};
each R^{c3} and R¹³ is independently selected from H and C₁₋₆ alkyl;
R⁴ is selected from H, C₁₋₆ alkyl, and C₁₋₆ haloalkyl;
R⁶ is selected from H, C₁₋₆ alkyl, —C(O)R^{a6}, and —S(O)₂R^{b6};
each R^{a6} and R^{b6} is independently selected from H and C₁₋₃ alkyl;
or, R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents;
each R^{6A} is independently selected from CN, 4-7 membered heterocycloalkyl, and —C(O)NR^{c6A}R^{d6A};
each R^{c6A} and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents;
R⁷ is selected from H, halo, C₁₋₆ alkyl, C₂₋₆ alkenyl, C₂₋₆ alkynyl, phenyl, 8-10 membered heterocycloalkyl, 5-6 membered heteroaryl, and —C(O)NR^{c7}R^{d7}, wherein the C₁₋₆ alkyl, C₂₋₆ alkenyl, C₂₋₆ alkynyl, phenyl, 8-10 membered heterocycloalkyl, and 5-6 membered heteroaryl of R⁷ are each optionally substituted with 1, 2, 3, or 4 independently selected R^{7A} substituents;
each R^{c7} and R^{d7} is independently selected from H and C₁₋₆ alkyl;
each R^{7A} is independently selected from C₁₋₆ alkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents;
each R^{a7A}, R^{c7A}, and R^{d7A} is independently selected from H and C₁₋₃ alkyl;
each R^{7B} is independently selected from C₁₋₆ alkyl, OR^{a7B}, NR^{c7B}R^{d7B}, C(O)R^{a7B}, and —C(O)NR^{c7B}R^{d7B}, wherein the C₁₋₆ alkyl is optionally substituted by OH;
each R^{a7B}, R^{c7B}, and R^{d7B} is independently selected from H and C₁₋₃ alkyl;
R⁸ is selected from H, halo, C₁₋₆ alkyl, C₂₋₆ alkenyl, C₂₋₆ alkynyl, and C₁₋₆ haloalkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, the compound of Formula I is a compound of Formula II:

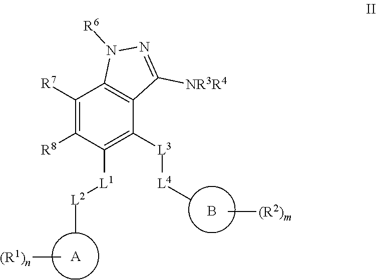

or a pharmaceutically acceptable salt thereof.

In some embodiments, the compound of Formula I is a compound of Formula IIa:

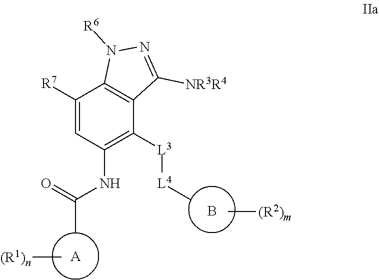

or a pharmaceutically acceptable salt thereof.

In some embodiments, the compound of Formula I is a compound of Formula III:

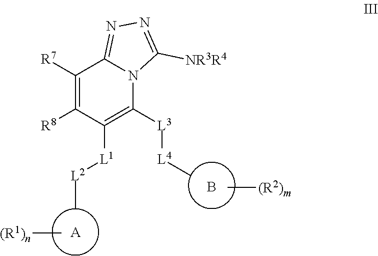

or a pharmaceutically acceptable salt thereof.

In some embodiments, the compound of Formula I is a compound of Formula IIIa:

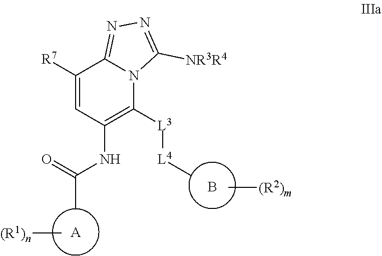

or a pharmaceutically acceptable salt thereof.

In some embodiments, the compound of Formula I is a compound of Formula IV:

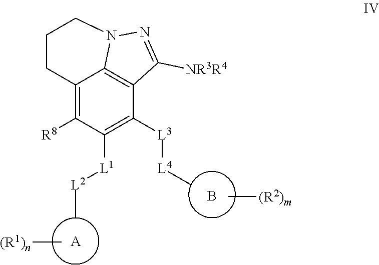

or a pharmaceutically acceptable salt thereof.

In some embodiments, the compound of Formula I is a compound of Formula IVa:

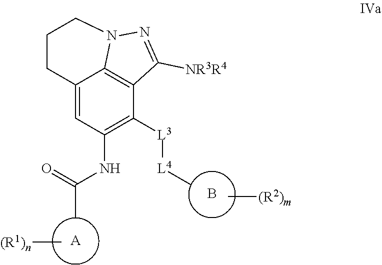

or a pharmaceutically acceptable salt thereof.

In some embodiments, the compound provided herein is selected from:
N-(3-amino-4-(2-chloro-5-fluorophenoxy)-1H-indazol-5-yl)-3-fluoro-5-(trifluoromethyl)benzamide;
N-(4-(2-chloro-5-fluorophenoxy)-3-(methylamino)-1H-indazol-5-yl)-3-fluoro-5-(trifluoromethyl)benzamide;
N-(3-amino-4-((5-chloropyridin-3-yl)oxy)-1H-indazol-5-yl)-3-fluoro-5-(trifluoromethyl)benzamide;
N-(3-amino-4-((2-chloro-5-fluorophenyl)amino)-1H-indazol-5-yl)-3-fluoro-5-(trifluoromethyl)benzamide;
N-(3-amino-4-(2-chloro-5-fluorobenzyl)-1H-indazol-5-yl)-3-fluoro-5-(trifluoromethyl)benzamide;
N-(3-amino-4-((2-chloro-5-fluorophenoxy)methyl)-1H-indazol-5-yl)-3-fluoro-5-(trifluoromethyl)benzamide;
N-(3-amino-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-ethyl-1H-indazol-5-yl)-3-fluoro-5-(trifluoromethyl)benzamide;
N-(3-amino-4-(2-chloro-5-fluorophenoxy)-1-(2-(methylamino)-2-oxoethyl)-1H-indazol-5-yl)-3-fluoro-5-(trifluoromethyl)benzamide;
N-(3-amino-4-(2-chloro-5-fluorophenoxy)-1-(2-morpholino-2-oxoethyl)-1H-indazol-5-yl)-3-fluoro-5-(trifluoromethyl)benzamide;
N-(3-amino-7-bromo-4-(2-chloro-5-fluorophenoxy)-1H-indazol-5-yl)-3-fluoro-5-(trifluoromethyl)benzamide;
N-(3-amino-7-chloro-4-(2-chloro-5-fluorophenoxy)-1H-indazol-5-yl)-3-fluoro-5-(trifluoromethyl)benzamide;
N-(3-amino-4-(2-chloro-5-fluorophenoxy)-7-methyl-1H-indazol-5-yl)-3-fluoro-5-(trifluoromethyl)benzamide;
N-(3-amino-4-(2-chloro-5-fluorophenoxy)-7-(1-methyl-1H-pyrazol-4-yl)-1H-indazol-5-yl)-3-fluoro-5-(trifluoromethyl)benzamide;
N-(3-amino-4-(2-chloro-5-fluorophenoxy)-7-(1-methyl-1H-pyrazol-5-yl)-1H-indazol-5-yl)-3-fluoro-5-(trifluoromethyl)benzamide;
N-(3-amino-4-(2-chloro-5-fluorophenoxy)-7-(pyridin-3-yl)-1H-indazol-5-yl)-3-fluoro-5-(trifluoromethyl)benzamide;
N-(3-amino-4-(2-chloro-5-fluorophenoxy)-7-phenyl-1H-indazol-5-yl)-3-fluoro-5-(trifluoromethyl)benzamide;
N-(3-amino-4-(2-chloro-5-fluorophenoxy)-1H-indazol-5-yl)benzo[b]thiophene-3-carboxamide;
N-(3-amino-4-(2-chloro-5-fluorophenoxy)-1H-indazol-5-yl)-5-fluoro-3-hydroxy-3-(trifluoromethyl)indoline-1-carboxamide;
N-(3-amino-5-(2-chloro-5-fluorophenoxy)-[1,2,4]triazolo[4,3-a]pyridin-6-yl)-3-fluoro-5-(trifluoromethyl)benzamide;
N-(3-amino-4-(2-chloro-5-fluorophenoxy)-7-(morpholinomethyl)-1H-indazol-5-yl)-3-fluoro-5-(trifluoromethyl)benzamide;
N-(2-amino-3-(2-chloro-5-fluorophenoxy)-7,8-dihydro-6H-pyrazolo[4,5,1-ij]quinolin-4-yl)-3-fluoro-5-(trifluoromethyl)benzamide;
N-(3-amino-4-(2-chloro-5-fluorophenoxy)-7-(hydroxymethyl)-1H-indazol-5-yl)-3-fluoro-5-(trifluoromethyl)benzamide;
N-(3-amino-7-chloro-4-(2-chloro-5-fluorophenoxy)-1-methyl-1H-indazol-5-yl)-3-fluoro-5-(trifluoromethyl)benzamide;
N-(3-amino-7-bromo-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-7-(1-isopropyl-1H-pyrazol-4-yl)-1-methyl-1H-indazol-5-yl)-3-fluoro-5-(trifluoromethyl)benzamide;
N-(3-amino-7-(4-carbamoylphenyl)-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-methyl-7-(1-(tetrahydrofuran-3-yl)-1H-pyrazol-4-yl)-1H-indazol-5-yl)-3-fluoro-5-(trifluoromethyl)benzamide;
N-(3-amino-4-(2-chloro-5-fluorophenoxy)-1-methyl-7-(pyrimidin-5-yl)-1H-indazol-5-yl)-3-fluoro-5-(trifluoromethyl)benzamide;
N-(3-amino-4-(2-chloro-5-fluorophenoxy)-1-methyl-7-(1-methyl-1H-1,2,3-triazol-5-yl)-1H-indazol-5-yl)-3-fluoro-5-(trifluoromethyl)benzamide;
N-(3-amino-4-(2-chloro-5-fluorophenoxy)-7-(cyanomethyl)-1-methyl-1H-indazol-5-yl)-3-fluoro-5-(trifluoromethyl)benzamide;
N-(6-amino-7-(2-chloro-5-fluorophenoxy)-2-oxo-2,3-dihydro-1H-pyrazolo[1,5,4-de]quinoxalin-8-yl)-3-fluoro-5-(trifluoromethyl)benzamide;
N-(3-amino-1-(2-amino-2-oxoethyl)-7-bromo-4-(2-chloro-5-fluorophenoxy)-1H-indazol-5-yl)-3-fluoro-5-(trifluoromethyl)benzamide;
N-(3-amino-7-chloro-4-(2-chloro-5-fluorophenoxy)-1-(2-cyanoethyl)-1H-indazol-5-yl)-3-fluoro-5-(trifluoromethyl)benzamide;
N-(3-(2-aminoacetamido)-7-chloro-4-(2-chloro-5-fluorophenoxy)-1-methyl-1H-indazol-5-yl)-3-fluoro-5-(trifluoromethyl)benzamide;
N-(2-amino-3-(2-chloro-5-fluorophenoxy)-8-oxo-6,7,8,9-tetrahydro-[1,4]diazepino[6,7,1-hi]indazol-4-yl)-3-fluoro-5-(trifluoromethyl)benzamide;
N-(3-amino-4-(2-chloro-5-fluorophenoxy)-1-methyl-7-(pyridin-2-ylethynyl)-1H-indazol-5-yl)-3-fluoro-5-(trifluoromethyl)benzamide;
N-(3-amino-4-(2-chloro-5-fluorophenoxy)-1-methyl-7-((tetrahydro-2H-pyran-4-yl)ethynyl)-1H-indazol-5-yl)-3-fluoro-5-(trifluoromethyl)benzamide;
N-(3-amino-4-(2-chloro-5-fluorophenoxy)-7-(3-hydroxy-3-methylbut-1-yn-1-yl)-1-methyl-1H-indazol-5-yl)-3-fluoro-5-(trifluoromethyl)benzamide;
N-(3-amino-7-(3-amino-3-methylbut-1-yn-1-yl)-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-methyl-7-((tetrahydrofuran-3-yl)ethynyl)-1H-indazol-5-yl)-3-fluoro-5-(trifluoromethyl)benzamide;
N-(3-amino-4-(2-chloro-5-fluorophenoxy)-7-(cyclopropylethynyl)-1-methyl-1H-indazol-5-yl)-3-fluoro-5-(trifluoromethyl)benzamide;
N-(3-amino-4-(2-chloro-5-fluorophenoxy)-7-((1-hydroxycyclopropyl)ethynyl)-1-methyl-1H-indazol-5-yl)-3-fluoro-5-(trifluoromethyl)benzamide;
N-(3-amino-4-(2-chloro-5-fluorophenoxy)-7-((1-(hydroxymethyl)cyclopropyl)ethynyl)-1-methyl-1H-indazol-5-yl)-3-fluoro-5-(trifluoromethyl)benzamide;
N-(3-amino-7-((1-aminocyclopropyl)ethynyl)-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-methyl-7-((3-methyloxetan-3-yl)ethynyl)-1H-indazol-5-yl)-3-fluoro-5-(trifluoromethyl)benzamide;
N-(3-amino-4-(2-chloro-5-fluorophenoxy)-7-ethynyl-1-methyl-1H-indazol-5-yl)-3-fluoro-5-(trifluoromethyl)benzamide;
N-(3-amino-4-(2-chloro-5-fluorophenoxy)-1-methyl-7-((1-methyl-1H-pyrazol-3-yl)ethynyl)-1H-indazol-5-yl)-3-fluoro-5-(trifluoromethyl)benzamide;
N-(3-amino-4-(2-chloro-5-fluorophenoxy)-7-(3-hydroxyprop-1-yn-1-yl)-1-methyl-1H-indazol-5-yl)-3-fluoro-5-(trifluoromethyl)benzamide;
N-(3-amino-4-(2-chloro-5-fluorophenoxy)-1-methyl-7-((1-methyl-1H-imidazol-4-yl)ethynyl)-1H-indazol-5-yl)-3-fluoro-5-(trifluoromethyl)benzamide;
N-(3-amino-4-(2-chloro-5-fluorophenoxy)-1-methyl-7-((1-methylpiperidin-4-yl)ethynyl)-1H-indazol-5-yl)-3-fluoro-5-(trifluoromethyl)benzamide;
N-(7-((1-acetylpiperidin-4-yl)ethynyl)-3-amino-4-(2-chloro-5-fluorophenoxy)-1-methyl-1H-indazol-5-yl)-3-fluoro-5-(trifluoromethyl)benzamide;
N-(7-chloro-4-(2-chloro-5-fluorophenoxy)-1-methyl-3-(methylamino)-1H-indazol-5-yl)-3-fluoro-5-(trifluoromethyl)benzamide;
N-(7-chloro-4-(2-chloro-5-fluorophenoxy)-3-(dimethylamino)-1-methyl-1H-indazol-5-yl)-3-fluoro-5-(trifluoromethyl)benzamide;
N-(3-amino-4-(2-chloro-5-fluorophenoxy)-7-(5,6-dihydro-[1,2,4]triazolo[1,5-a]pyrazin-7(8H)-yl)-1-methyl-1H-indazol-5-yl)-3-fluoro-5-(trifluoromethyl)benzamide;
N-(3-amino-1-(2-(azetidin-1-yl)ethyl)-7-chloro-4-(2-chloro-5-fluorophenoxy)-1H-indazol-5-yl)-3-fluoro-5-(trifluoromethyl)benzamide;
(E)-N-(3-amino-4-(2-chloro-5-fluorophenoxy)-7-(2-cyanovinyl)-1-methyl-1H-indazol-5-yl)-3-fluoro-5-(trifluoromethyl)benzamide;
(Z)—N-(3-amino-4-(2-chloro-5-fluorophenoxy)-7-(2-cyanovinyl)-1-methyl-1H-indazol-5-yl)-3-fluoro-5-(trifluoromethyl)benzamide;
N-(3-amino-4-(2-chloro-5-fluorophenoxy)-7-(2-cyanoethyl)-1-methyl-1H-indazol-5-yl)-3-fluoro-5-(trifluoromethyl)benzamide;
N-(3-amino-7-chloro-4-(2-chloro-5-fluorophenoxy)-1-methyl-1H-indazol-5-yl)-3-chloro-5-fluorobenzamide;
N-(3-amino-7-chloro-4-(2-chloro-5-fluorophenoxy)-1-methyl-1H-indazol-5-yl)-3-(trifluoromethyl)benzamide;
N-(3-amino-7-chloro-4-(2-chloro-5-fluorophenoxy)-1-methyl-1H-indazol-5-yl)-5-(trifluoromethyl)nicotinamide;
N-(3-amino-7-chloro-4-(2-chloro-5-fluorophenoxy)-1-methyl-1H-indazol-5-yl)benzo[b]thiophene-3-carboxamide;
N-(3-amino-7-chloro-4-(2-chloro-5-fluorophenoxy)-1-methyl-1H-indazol-5-yl)indoline-1-carboxamide;
3-Amino-4-(2-chloro-5-fluorophenoxy)-N-ethyl-5-(3-fluoro-5-(trifluoromethyl)benzamido)-1-methyl-1H-indazole-7-carboxamide;
N-(3-amino-4-(2-chloro-5-fluorophenoxy)-7-(2-hydroxyethyl)-1-methyl-1H-indazol-5-yl)-3-fluoro-5-(trifluoromethyl)benzamide;
N-(3-amino-4-(2-chloro-5-fluorophenoxy)-1-(2-hydroxyethyl)-7-(pyridin-2-ylethynyl)-1H-indazol-5-yl)-3-fluoro-5-(trifluoromethyl)benzamide;
N-(2-amino-3-(2-chloro-5-fluorophenoxy)-8-oxo-7,8-dihydro-6H-pyrazolo[4,5,1-ij]quinazolin-4-yl)-3-fluoro-5-(trifluoromethyl)benzamide;
N-(2-amino-3-(2-chloro-5-fluorophenoxy)-7-methyl-8-oxo-7,8-dihydro-6H-pyrazolo[4,5,1-ij]quinazolin-4-yl)-3-fluoro-5-(trifluoromethyl)benzamide;
N-(4-amino-3-(2-chloro-5-fluorophenoxy)-7H-pyrazolo[4,5,1-de]phenanthridin-2-yl)-3-fluoro-5-(trifluoromethyl)benzamide;
N-(2-amino-3-(2-chloro-5-fluorophenoxy)-7-cyclopropyl-8-oxo-7,8-dihydro-6H-pyrazolo[4,5,1-ij]quinazolin-4-yl)-3-fluoro-5-(trifluoromethyl)benzamide;
N-(2-amino-3-(2-chloro-5-fluorophenoxy)-7-cyclobutyl-8-oxo-7,8-dihydro-6H-pyrazolo[4,5,1-ij]quinazolin-4-yl)-3-fluoro-5-(trifluoromethyl)benzamide;
N-(4-amino-3-(2-chloro-5-fluorophenoxy)-7-oxo-7H-pyrazolo[4,5,1-de]phenanthridin-2-yl)-3-fluoro-5-(trifluoromethyl)benzamide;
N-(4-amino-3-(2-chloro-5-fluorophenoxy)-7-oxo-7,8,9,10-tetrahydrocyclopenta[c]pyrazolo[4,5,1-ij]quinolin-2-yl)-3-fluoro-5-(trifluoromethyl)benzamide;
N-(4-amino-3-(2-chloro-5-fluorophenoxy)-7-oxo-8,9,10,11-tetrahydro-7H-pyrazolo[4,5,1-de]phenanthridin-2-yl)-3-fluoro-5-(trifluoromethyl)benzamide;
(E)-N-(3-amino-4-(2-chloro-5-fluorophenoxy)-1-methyl-7-(2-(pyridin-2-yl)vinyl)-1H-indazol-5-yl)-3-fluoro-5-(trifluoromethyl)benzamide;
N-(3-amino-4-(2-chloro-5-fluorophenoxy)-1-methyl-7-(2-(pyridin-2-yl)ethyl)-1H-indazol-5-yl)-3-fluoro-5-(trifluoromethyl)benzamide;
N-(3-amino-4-(2-chloro-5-fluorophenoxy)-1-methyl-7-(piperidin-2-ylethynyl)-1H-indazol-5-yl)-3-fluoro-5-(trifluoromethyl)benzamide;
N-(7-((1-acetylpiperidin-2-yl)ethynyl)-3-amino-4-(2-chloro-5-fluorophenoxy)-1-methyl-1H-indazol-5-yl)-3-fluoro-5-(trifluoromethyl)benzamide;
2-((3-Amino-4-(2-chloro-5-fluorophenoxy)-5-(3-fluoro-5-(trifluoromethyl)benzamido)-1-methyl-1H-indazol-7-yl)ethynyl)-N-methylpiperidine-1-carboxamide;
N-(3-amino-7-bromo-4-(2-chloro-5-fluorophenoxy)-1-(methylsulfonyl)-1H-indazol-5-yl)-3-fluoro-5-(trifluoromethyl)benzamide; and
N-(1-acetyl-3-amino-7-bromo-4-(2-chloro-5-fluorophenoxy)-1H-indazol-5-yl)-3-fluoro-5-(trifluoromethyl)benzamide;
or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the terms “Cₙ₋ₘ” and “Cₘ₋ₙ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, from 2 to 6 carbon atoms, from 2 to 4 carbon atoms, from 2 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, and the like. In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-, 7-, 8-, 9-, or 10-membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-, 7-, 8-, 9-, or 10-membered monocyclic or bicyclic heteroaryl having 1, 2, 3, or 4 heteroatom ring members independently selected from N, O, and S. In some embodiments, the heteroaryl is a 5-6 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, and S. In some embodiments, the heteroaryl group contains 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 4 to 8 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, tetrahydrothiopheneyl, tetrahydrothiopheneyl 1,1-dioxide, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, 2-azaspiro[3.3]heptanyl, diazaspiro[3.3]heptanyl, azaspiro[3.5]nonanyl, 7-azaspiro[3.5]nonanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” or “alkylene linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, a “haloalkyl linking group” or “haloalkylene linking group” is a bivalent straight chain or branched haloalkyl linking group (“haloalkylene group”). Example haloalkylene groups include —CF₂—, —C₂F₄—, —CHF—, —CCl₂—, —CHCl—, —C₂Cl₄—, and the like.

As used herein, a “cycloalkyl linking group” or “cycloalkylene linking group” is a bivalent straight chain or branched cycloalkyl linking group (“cycloalkylene group”). Examples of “cycloalkyl linking groups” or “cycloalkylene groups” include cyclopropy-1,1,-diyl, cyclopropy-1,2-diyl, cyclobut-1,3,-diyl, cyclopent-1,3,-diyl, cyclopent-1,4,-diyl, cyclohex-1,2,-diyl, cyclohex-1,3,-diyl, cyclohex-1,4,-diyl, and the like.

As used herein, a “heterocycloalkyl linking group” or “heterocycloalkylene linking group” is a bivalent straight chain or branched heterocycloalkyl linking group (“heterocycloalkylene group”). Examples of “heterocycloalkyl linking groups” or “heterocycloalkylene groups” include azetidin-1,2-diyl, azetidin-1,3-diyl, pyrrolidin-1,2-diyl, pyrrolidin-1,3-diyl, pyrrolidin-2,3-diyl, piperidin-1,2-diyl, piperidin-1,3-diyl, piperidin-1,4-diyl, piperidin-2,3-diyl, piperidin-2,4-diyl, and the like.

As used herein, a “heteroaryl linking group” or “heteroarylene linking group” is a bivalent straight chain or branched heteroaryl linking group (“heteroarylene group”). Examples of “heteroaryl linking groups” or “heteroarylene groups” include pyrazol-1,3-diyl, imidazol-1,2,-diyl, pyridin-2,3-diyl, pyridin-2,4-diyl, pyridin-3,4-diyl, and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{G}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of a-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like.

The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

Compounds of the invention, including salts thereof, can be prepared using known organic synthesis techniques and according to various possible synthetic routes. Example synthetic methods for preparing compounds of the invention are provided in the Schemes below.

Compounds of Formula I (e.g., compounds of Formula I-6) can be prepared, for example, according to the procedures shown in Scheme I. Intermediate I-3 is prepared by a process comprising reacting compounds of Formula I-1 with I-2 promoted by a suitable reagent (e.g., NaH, LDA). Intermediate I-4 is prepared by a process comprising reacting intermediate I-3 under reductive conditions (e.g., Fe). Intermediate I-5 is prepared by a process comprising reacting intermediate I-4 with a suitable reagent (e.g., benzoyl chloride). Compounds of Formula I-6 can then be prepared by a process comprising reacting intermediate I-5 under suitable conditions (e.g., N₂H₄ monohydrate).

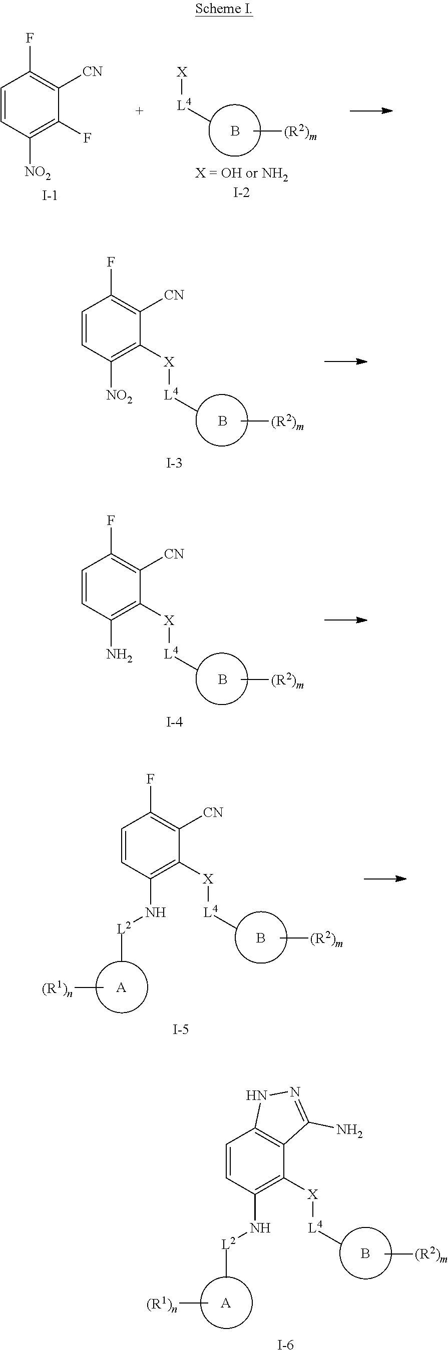

Compounds of Formula I (e.g., compounds of Formula II-6) can be prepared, for example, according to the procedures shown in Scheme II. Intermediate II-1 is prepared by a process comprising reacting compounds of Formula I-1 with suitable reagent (e.g., NH₄OH). Intermediate II-2 is prepared by a process comprising reacting intermediate II-1 under Sandmeyer reaction conditions. Intermediate II-3 is prepared by a process comprising reacting intermediate II-2 under reductive conditions (e.g., Fe). Intermediate II-4 is prepared by a process comprising reacting intermediate II-3 with a suitable reagent (e.g., benzoyl chloride). Intermediate II-5 is prepared by a process comprising reacting intermediate II-4 under suitable conditions (e.g., N₂H₄ monohydrate). Compounds of Formula II-6 are then prepared by a process comprising reacting intermediate II-5 under suitable conditions (e.g., transition metal-catalyzed cross-coupling reactions).

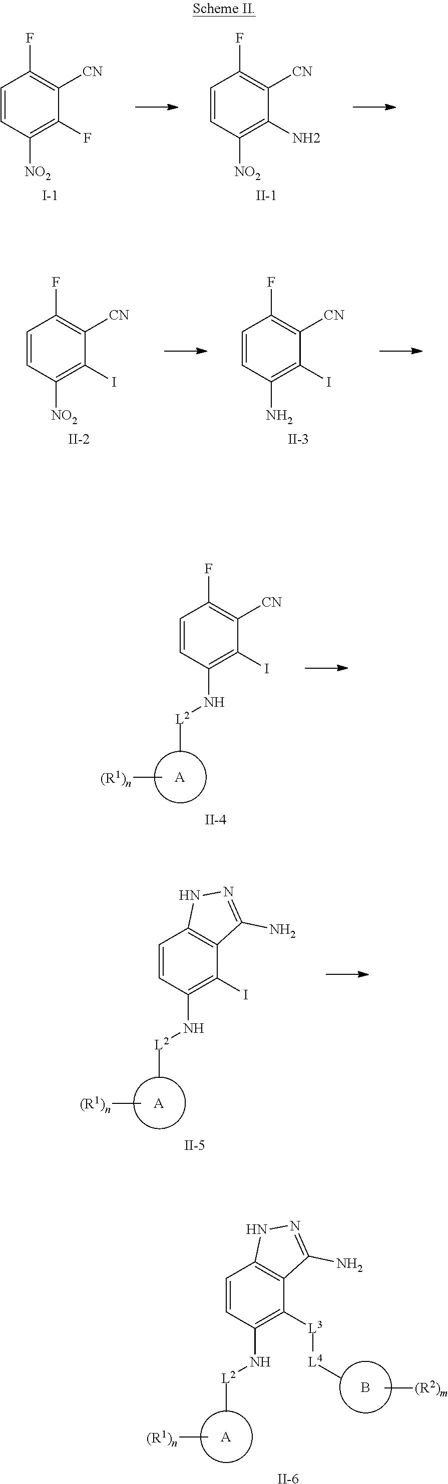

Compounds of Formula I (e.g., compounds of Formula III-3) can be prepared, for example, according to the procedures shown in Scheme III. Intermediate III-1 is prepared by a process comprising reacting compounds of Formula II-6 with suitable protecting group (e.g., isobenzofuran-1,3-dione). Intermediate III-2 is then prepared by a process comprising reacting intermediate III-1 with a suitable reagent (e.g., alkyl halide). Compounds of Formula III-3 are next prepared by a process comprising reacting Formula III-2 under suitable conditions (e.g., N₂H₄ monohydrate).

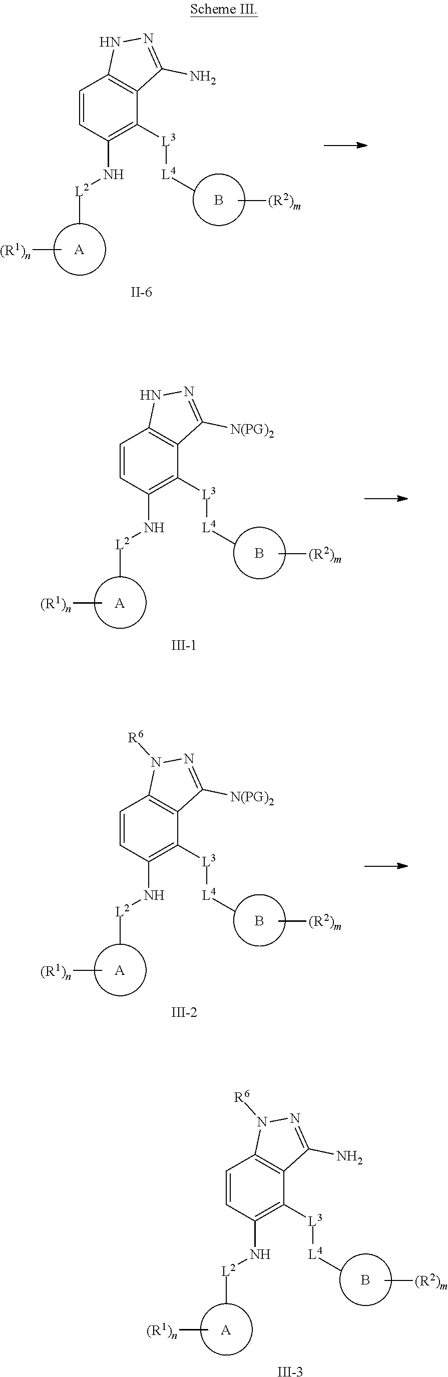

Compounds of Formula I (e.g., compounds of Formula IV-2) can be prepared, for example, according to the procedures shown in Scheme IV. Intermediate IV-1 is prepared by a process comprising reacting compounds of Formula III-1 with suitable reagent (e.g., NBS). Compounds of Formula IV-2 are then prepared by a process comprising reacting intermediate IV-1 under suitable conditions (e.g., transition metal-catalyzed cross-coupling reactions).

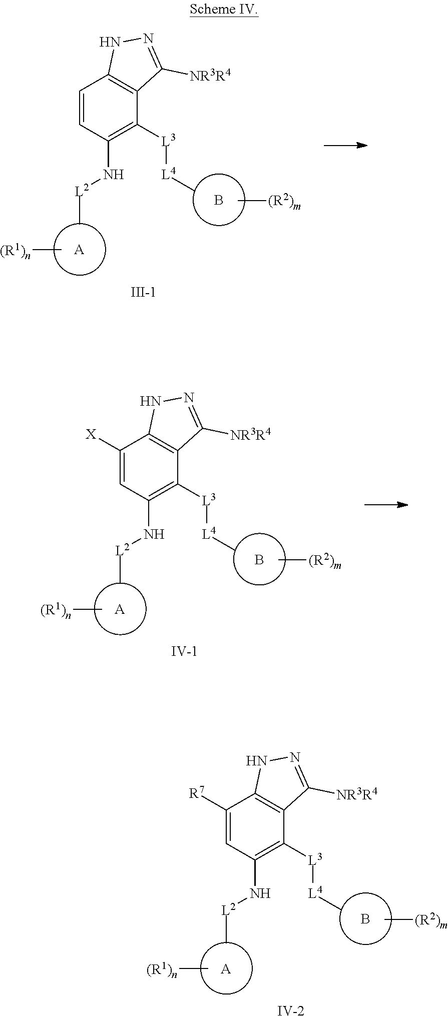

Compounds of Formula I (e.g., compounds of Formula V-9) can be prepared, for example, according to the procedures shown in Scheme V. Intermediate V-3 is prepared by a process comprising reacting compounds of Formula V-1 with V-2 promoted by a suitable reagent (e.g., NaH, LDA). Intermediate V-4 is then prepared by a process comprising reacting intermediate V-3 with a suitable reagent (e.g., tert-butyl hydrazinecarboxylate). Intermediate V-5 is prepared by a process comprising reacting intermediate V-4 under reductive conditions (e.g., Fe) followed by adding suitable protecting groups (“PG” of Scheme V). Intermediate V-6 is prepared by a deprotection of intermediate V-5 with a suitable reagent (e.g., TFA). Intermediate V-7 is prepared by a process comprising reacting compounds of V-6 with suitable reagent (e.g., di(1H-imidazol-1-yl)methanimine). Intermediate V-8 is prepared by a deprotection of intermediate V-7 with a suitable reagent. Next, compounds of Formula V-9 are prepared by a process comprising reacting intermediate V-8 with a suitable reagent (e.g., benzoyl chloride).

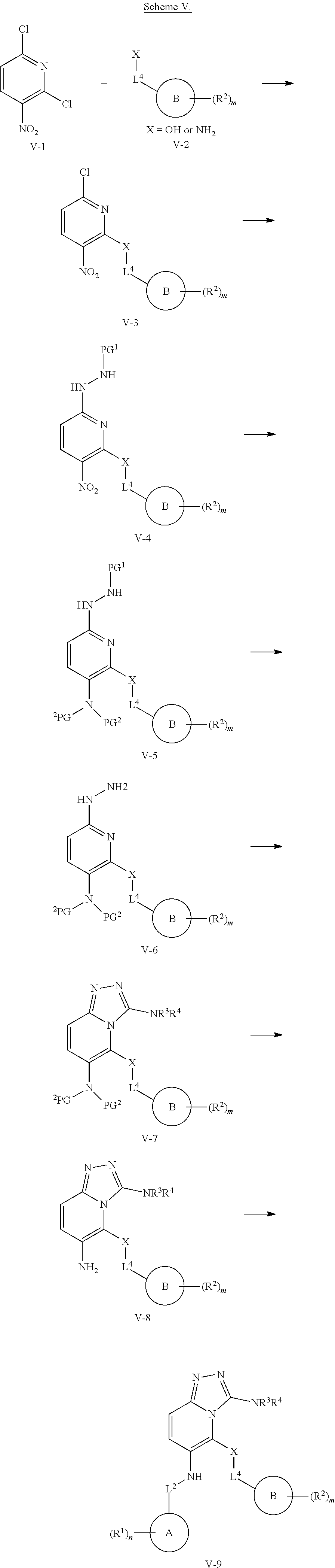

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

### Methods of Use

The present disclosure provides uses for compounds and compositions described herein. The compounds described herein can inhibit the activity of PI3Kα kinase. In some embodiments, provided compounds and compositions are for use in medicine (e.g., as therapy). In some embodiments, provided compounds and compositions are useful in treating a disease, disorder, or condition, wherein an underlying pathology is, wholly or partially, mediated by PI3Kα. In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with PI3Kα. In some embodiments, the present disclosure provides methods of administering provided compounds or compositions to a subject suffering from or susceptible to a disease, disorder, or condition, wherein an underlying pathology is, wholly or partially, mediated by PI3Kα. In some embodiments, an underlying pathology is of the disease, disorder, or condition provided herein is, wholly or partially, mediated by mutant PI3Kα. In some embodiments, the disease, disorder, or condition provided herein is associated with mutant PI3Kα.

In some embodiments, the compounds provided herein are useful as PI3Kα inhibitors. In some embodiments, the present disclosure provides methods of inhibiting PI3Kα in a subject comprising administering a provided compound or composition. In some embodiments, the present disclosure provides methods of inhibiting PI3Kα in a biological sample comprising contacting the sample with a provided compound or composition. In some embodiments, the compounds provided herein selectively inhibit PI3Kα over one or more other PI3K isoforms (e.g., Class 1 PI3K isoforms such as PI3Kβ, PI3Kδ, PI3Kγ, and the like). In some embodiments, the compounds provided herein selectively inhibit mutant PI3Kα over wild-type PI3Kα.

In some embodiments, the present disclosure provides methods of treating a disease, disorder or condition associated with PI3Kα in a subject in need thereof, comprising administering to the subject a compound, salt, or composition of the disclosure. In some embodiments, a disease, disorder or condition is associated with mutation of PI3Kα. In some embodiments, the present disclosure provides methods of treating a disease, disorder or condition, wherein an underlying pathology is, wholly or partially, mediated by PI3Kα, in a subject in need thereof, comprising administering to the subject a provided compound or composition.

In some embodiments, the present disclosure provides methods of treating a variety of PI3Kα-dependent diseases and disorders. In some embodiments, the disease of disorder is a cancer (e.g., breast cancer, brain cancer, prostate cancer, endometrial cancer, gastric cancer, leukemia, lymphoma, sarcoma, colorectal cancer, lung cancer, ovarian cancer, skin cancer, and head and neck cancer). In some embodiments, the disease or disorder associated with PI3Kα includes, but is not limited to, CLOVES syndrome (congenital lipomatous overgrowth, vascular malformations, epidermal naevi, scoliosis/skeletal and spinal syndrome), PIK3CA-related overgrowth syndrome (PROS), endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma. In some embodiments, the cancer is breast cancer.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a PI3Kα kinase with a compound described herein includes the administration of a compound described herein to an individual or patient, such as a human, having a PI3Kα kinase, as well as, for example, introducing a compound described herein into a sample containing a cellular or purified preparation containing the PI3Kα kinase.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapy

One or more additional therapeutic agents such as, for example, chemotherapeutics or other anti-cancer agents, anti-inflammatory agents, steroids, immunosuppressants, anesthetics (e.g., for use in combination with a surgical procedure), or other agents useful for treating diseases associated with PI3Kα can be used in combination with the compounds and salts provided herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, CDK4/6, MEKK, ERK, MAPK, mTOR, EGFR, HER2, HER3, HER4, INS—R, IGF-1R, IR—R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

For treating cancer and other proliferative diseases, compounds described herein can be used in combination with targeted therapies, including JAK kinase inhibitors (ruxolitinib, additional JAK1/2 and JAK1-selective, baricitinib or itacitinib), Pim kinase inhibitors (e.g., LGH447 and SGI-1776), PI3 kinase inhibitors including PI3K-delta selective and broad spectrum PI3K inhibitors (e.g., parsaclisib), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Axl, and Mer), angiogenesis inhibitors, interleukin receptor inhibitors, Cyclin Dependent kinase inhibitors (e.g., palbociclib, ribociclib, and abemaciclib), BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610), LSD1 inhibitors (e.g., GSK2979552, and INCB59872), estrogen receptor modulators (e.g., fulvestrant), androgen receptor modulators (e.g., enzalutamide), BCL2 inhibitors (e.g., venetoclax), hypoxia-inducible factor-2 alpha inhibitors (e.g., belzutifan), exportin-1 (XPO-1) inhibitors (e.g., selinexor), KRAS inhibitors (e.g., sotorasib), arginase inhibitors (e.g., INCB1158), indoleamine 2,3-dioxygenase inhibitors (e.g., epacadostat, NLG919 or BMS-986205), PARP inhibiors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

Example anti-inflammatory agents include, but are not limited to, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example steroids include, but are not limited to, corticosteroids such as cortisone, dexamethasone, hydrocortisone, methylprednisolone, prednisolone, and prednisone.

Example immunosuppressants include, but are not limited to, azathioprine, chlorambucil, cyclophosphamide, cyclosporine, daclizumab, infliximab, methotrexate, and tacrolimus.

Example anesthetics include, but are not limited, to local anesthetics (e.g., lidocaine, procain, ropivacaine) and general anesthetics (e.g., desflurane, enflurane, halothane, isoflurane, methoxyflurane, nitrous oxide, sevoflurane, mmobarbital, methohexital, thiamylal, thiopental, diazepam, lorazepam, midazolam, etomidate, ketamine, propofol, alfentanil, fentanyl, remifentanil, buprenorphine, butorphanol, hydromorphone levorphanol, meperidine, methadone, morphine, nalbuphine, oxymorphone, pentazocine).

In some embodiments, the additional therapeutic agent is administered simultaneously with a compound or salt provided herein. In some embodiments, the additional therapeutic agent is administered after administration of the compound or salt provided herein. In some embodiments, the additional therapeutic agent is administered prior to administration of the compound or salt provided herein. In some embodiments, the compound or salt provided herein is administered during a surgical procedure. In some embodiments, the compound or salt provided herein is administered in combination with an additional therapeutic agent during a surgical procedure.

As provided herein, the additional compounds, inhibitors, agents, etc. can be combined with the compounds provided herein in a single or continuous dosage form, or they can be administered simultaneously or sequentially as separate dosage forms.

### Pharmaceutical Formulations and Dosage Forms

When employed as pharmaceuticals, the compounds of the invention can be administered in the form of pharmaceutical compositions which refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

The liquid forms in which the compounds and compositions of the present invention can be incorporated for administration orally or by injection include aqueous solutions, suitably flavored syrups, aqueous or oil suspensions, and flavored emulsions with edible oils.

The compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are provided herein.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the PI3Kα enzyme in tissue samples, including human, and for identifying PI3Kα enzyme ligands by inhibition binding of a labeled compound. Accordingly, the present invention includes PI3Kα enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ^{77B}r, ¹²³I, ¹²⁴I, ¹²⁵I and ¹³¹I. The radionuclide that is incorporated in the instant radio-labeled compounds will depend on the specific application of that radio-labeled compound. For example, for in vitro FGFR enzyme labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ^{77B}r will generally be most useful.

One or more constituent atoms of the compounds presented herein can be replaced or substituted with isotopes of the atoms in natural or non-natural abundance. In some embodiments, one or more atoms are replaced or substituted by deuterium. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., the compound of any of Formulas I-IVa) can be perdeuterated.

In some embodiments, the compound provided herein (e.g., the compound of any of Formulas I-IVa), or a pharmaceutically acceptable salt thereof, comprises at least one deuterium atom.

In some embodiments, the compound provided herein (e.g., the compound of any of Formulas I-IVa), or a pharmaceutically acceptable salt thereof, comprises two or more deuterium atoms.

In some embodiments, the compound provided herein (e.g., the compound of any of Formulas I-IVa), or a pharmaceutically acceptable salt thereof, comprises three or more deuterium atoms.

In some embodiments, for a compound provided herein (e.g., the compound of any of Formulas I-IVa), or a pharmaceutically acceptable salt thereof, all of the hydrogen atoms are replaced by deuterium atoms (i.e., the compound is “perdeuterated”).

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the PI3Kα enzyme. Accordingly, the ability of a test compound to compete with the radio-labeled compound for binding to the PI3Kα enzyme directly correlates to its binding affinity.

### Kits

The present invention also includes pharmaceutical kits useful, for example, in the treatment or prevention of PI3Kα-associated diseases or disorders referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of PI3Kα as described below.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004). The compounds separated were typically subjected to analytical liquid chromatography mass spectrometry (LCMS) for purity check.

Some of the compounds prepared were also separated on a preparative scale by reverse-phase high performance liquid chromatography (RP-HPLC) with MS detector or flash chromatography (silica gel) as indicated in the Examples.

The following abbreviations may be used herein: AcOH (acetic acid); aq. (aqueous); br (broad); calc. (calculated); d (doublet); dd (doublet of doublets); DCM (dichloromethane); DIEA (N,N-diisopropylethylamine); DMF (N, N-dimethylformamide); Et (ethyl); EtOAc (ethyl acetate); g (gram(s)); h (hour(s)); HCl (hydrochloric acid); HPLC (high performance liquid chromatography); Hz (hertz); J (coupling constant); LCMS (liquid chromatography—mass spectrometry); LDA (lithium diisopropylamide); m (multiplet); M (molar); MS (Mass spectrometry); Me (methyl); MeCN (acetonitrile); MeOH (methanol); mg (milligram(s)); min. (minutes(s)); mL (milliliter(s)); mmol (millimole(s)); N (normal); NBS (N-bromosuccinimide); NCS (N-chlorosuccinimide); nM (nanomolar); NMR (nuclear magnetic resonance spectroscopy); Ph (phenyl); pM (picomolar); RP-HPLC (reverse phase high performance liquid chromatography); r.t. (room temperature), s (singlet); t (triplet or tertiary); tert (tertiary); tt (triplet of triplets); TFA (trifluoroacetic acid); THF (tetrahydrofuran); μg (microgram(s)); μL (microliter(s)); μM (micromolar); wt % (weight percent). Brine is saturated aqueous sodium chloride. In vacuo is under vacuum.

### Example 1. N-(3-Amino-4-(2-chloro-5-fluorophenoxy)-1H-indazol-5-yl)-3-fluoro-5-(trifluoromethyl)benzamide

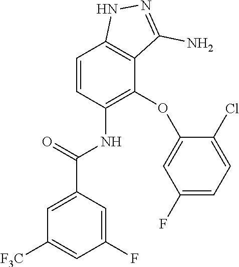 [SMILES: Nc1n[nH]c2ccc(NC(=O)c3cc(F)cc(C(F)(F)F)c3)c(Oc3cc(F)ccc3Cl)c12]

### Step 1. 2-(2-Chloro-5-fluorophenoxy)-6-fluoro-3-nitrobenzonitrile

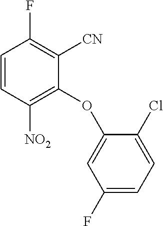 [SMILES: N#Cc1c(F)ccc([N+](=O)[O-])c1Oc1cc(F)ccc1Cl]

To a solution of 2-chloro-5-fluorophenol (159.2 mg, 1.09 mmol) in anhydrous tetrahydrofuran (3 mL) was added NaH (60% in mineral oil, 43.4 mg, 1.09 mmol) at 0° C. under nitrogen atmosphere. The resulting mixture was stirred at same temperature for 30 min before 2,6-difluoro-3-nitrobenzonitrile (200 mg, 1.09 mmol) was added in one portion. The reaction was then warmed to room temperature and stirred for 1 h before quenched with water; the mixture was extracted with ethyl acetate (2×20 mL). The combined organics were dried with anhydrous sodium sulfate, filtered, and concentrated under reduced pressure. The residue was purified by silica gel column chromatography, eluted with 20% ethyl acetate in hexanes to provide the desired product as a pale-yellow oil (270 mg, 80%).

### Step 2. 3-Amino-2-(2-chloro-5-fluorophenoxy)-6-fluorobenzonitrile

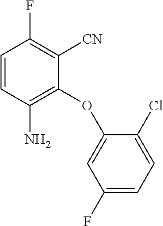 [SMILES: N#Cc1c(F)ccc(N)c1Oc1cc(F)ccc1Cl]

To a mixture of 2-(2-chloro-5-fluorophenoxy)-6-fluoro-3-nitrobenzonitrile (270 mg, 0.87 mmol) in MeOH (2 mL), THF (2 mL) and sat. NH₄Cl (1 mL) were added iron (230 mg, 4.1 mmol). The resulting mixture was stirred at 80° C. for 2 h. Upon cooling to room temperature, the mixture was concentrated under reduced pressure. The residue was extracted with ethyl acetate (2×20 mL). The combined organics were dried with anhydrous sodium sulfate, filtered, and concentrated under reduced pressure, which was then purified by silica gel column chromatography, eluted with 50% ethyl acetate in hexanes to provide the desired product as a colorless oil (170 mg, 70%).

### Step 3. N-(2-(2-Chloro-5-fluorophenoxy)-3-cyano-4-fluorophenyl)-3-fluoro-5-(trifluoromethyl)benzamide

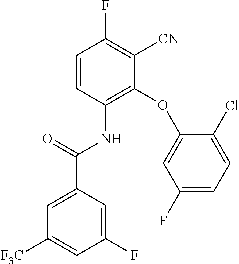 [SMILES: N#Cc1c(F)ccc(NC(=O)c2cc(F)cc(C(F)(F)F)c2)c1Oc1cc(F)ccc1Cl]

To a mixture of 3-amino-2-(2-chloro-5-fluorophenoxy)-6-fluorobenzonitrile (170 mg, 0.61 mmol) in pyridine (1 mL) were added 3-fluoro-5-(trifluoromethyl)benzoyl chloride (157 mg, 0.7 mmol) at 0° C. under nitrogen atmosphere. The resulting mixture was warmed to rt and stirred for 30 min before quenched with adding water. The mixture was extracted with ethyl acetate (2×20 mL). The combined organics were dried with anhydrous sodium sulfate, filtered, and concentrated under reduced pressure, which was then purified by silica gel column chromatography, eluted with 50% ethyl acetate in hexanes to provide the desired product as a colorless oil (229 mg, 80%).

### Step 4. N-(3-Amino-4-(2-chloro-5-fluorophenoxy)-1H-indazol-5-yl)-3-fluoro-5-(trifluoromethyl)benzamide

To a mixture of N-(2-(2-chloro-5-fluorophenoxy)-3-cyano-4-fluorophenyl)-3-fluoro-5-(trifluoromethyl)benzamide (229 mg, 0.49 mmol) in n-BuOH (1 mL) were added hydrazine monohydrate (0.2 mL) at rt. The resulting mixture was stirred at 110° C. for 1h. Upon cooling to room temperature, the mixture was concentrated under reduced pressure. The residue was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₁H₁₃ClF₅N₄O₂ (M+H)+ m/z=483.1; found 483.1.

### Example 2. N-(4-(2-Chloro-5-fluorophenoxy)-3-(methylamino)-1H-indazol-5-yl)-3-fluoro-5-(trifluoromethyl)benzamide

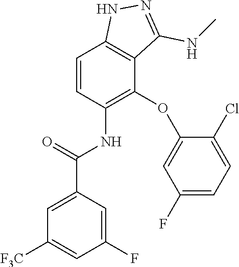 [SMILES: CNc1n[nH]c2ccc(NC(=O)c3cc(F)cc(C(F)(F)F)c3)c(Oc3cc(F)ccc3Cl)c12]

N-(3-Amino-4-(2-chloro-5-fluorophenoxy)-1H-indazol-5-yl)-3-fluoro-5-(trifluoromethyl)benzamide (Example 1; 20 mg, 0.04 mmol) was dissolved in MeOH (1 mL) then paraformaldehyde (6 mg, 0.2 mmol) and sodium methoxide (25% wt in MeOH, 0.12 mmol) were added. The resulting mixture was heated to 65° C. for 3.5 h, then removed from heat. NaBH₄ (4 mg, 0.1 mmol) was added and heating was continued for 2 h. The reaction was cooled to rt and purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₂H₁₅ClF₅N₄O₂ (M+H)+ m/z=497.1; found 497.1.

### Example 3. N-(3-Amino-4-((5-chloropyridin-3-yl)oxy)-1H-indazol-5-yl)-3-fluoro-5-(trifluoromethyl)benzamide

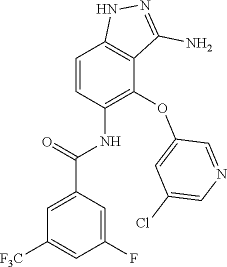 [SMILES: Nc1n[nH]c2ccc(NC(=O)c3cc(F)cc(C(F)(F)F)c3)c(Oc3cncc(Cl)c3)c12]

The title compound was prepared using similar procedures as described for Example 1, with 5-chloropyridin-3-ol replacing 2-chloro-5-fluorophenol in Step 1. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₀H₁₃ClF₄N₅O₂ (M+H)+ m/z=466.1; found 466.1.

### Example 4. N-(3-Amino-4-((2-chloro-5-fluorophenyl)amino)-1H-indazol-5-yl)-3-fluoro-5-(trifluoromethyl)benzamide

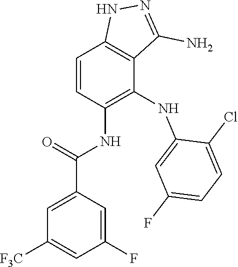 [SMILES: Nc1n[nH]c2ccc(NC(=O)c3cc(F)cc(C(F)(F)F)c3)c(Nc3cc(F)ccc3Cl)c12]

The title compound was prepared using similar procedures as described for Example 1, with 2-chloro-5-fluoroaniline replacing 2-chloro-5-fluorophenol and LDA replacing NaH in Step 1. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₁₄ClF₅N₅O (M+H)+ m/z=482.1; found 482.1.

### Example 5. N-(3-Amino-4-(2-chloro-5-fluorobenzyl)-1H-indazol-5-yl)-3-fluoro-5-(trifluoromethyl)benzamide

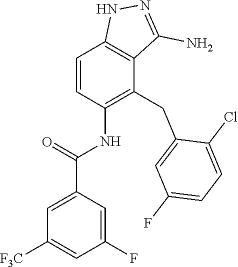 [SMILES: Nc1n[nH]c2ccc(NC(=O)c3cc(F)cc(C(F)(F)F)c3)c(Cc3cc(F)ccc3Cl)c12]

### Step 1. 2-Amino-6-fluoro-3-nitrobenzonitrile

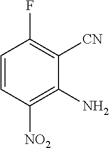 [SMILES: N#Cc1c(F)ccc([N+](=O)[O-])c1N]

To a solution of 2,6-difluoro-3-nitrobenzonitrile (7.6 g, 41.3 mmol) in anhydrous tetrahydrofuran (30 mL) was added ammonium hydroxide solution (30 mL) dropwise at 0° C. under nitrogen atmosphere. The resulting mixture was stirred at same temperature for 30 min before 30 mL of ice water was added. The crude product precipitated out and was collected by filtration followed by washing with water (2×30 mL) and dried at room temperature to give a yellow solid, which was used in next step without further purification.

### Step 2. 6-Fluoro-2-iodo-3-nitrobenzonitrile

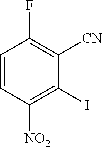 [SMILES: N#Cc1c(F)ccc([N+](=O)[O-])c1I]

2-Amino-6-fluoro-3-nitrobenzonitrile (3.0 g, 16.5 mmol) was suspended in 60 mL water while stirring. The mixture was cooled in an ice bath and 60 mL concentrated sulfuric acid was added slowly. A solution of sodium nitrite (1.2 g 17 mmol) in 60 mL water was added dropwise over 0.5 hour. The reaction mixture was stirred for an additional 0.25 hour at 0-5°. The mixture was poured into a well-stirred solution of potassium iodide (3.0 g 18 mmol) in 60 mL water. The resulting solid was collected by filtration, then stirred in 200 mL ethyl acetate. A solution of 200 mL 20% sodium hydrogen sulfite was added slowly. The organic layer was separated, washed with saturated sodium chloride, dried over magnesium sulfate and concentrated under reduced pressure to afford the crude product as brown solid, which was then purified by silica gel column chromatography, eluted with 20% ethyl acetate in hexanes to provide the desired product as a light brown solid (3.8 g, 80%).

### Step 3. 3-Amino-6-fluoro-2-iodobenzonitrile

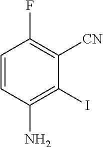 [SMILES: N#Cc1c(F)ccc(N)c1I]

To a mixture of 6-fluoro-2-iodo-3-nitrobenzonitrile (3.8 g, 13 mmol) in MeOH (200 mL), THF (200 mL) and sat. NH₄Cl (100 mL) were added iron (3.1 g, 55 mmol). The resulting mixture was stirred at 80° C. for 2 h. Upon cooling to room temperature, the mixture was concentrated under reduced pressure. The residue was extracted with ethyl acetate (2×400 mL). The combined organics were dried with anhydrous sodium sulfate, filtered, and concentrated under reduced pressure, which was then purified by silica gel column chromatography, eluted with 50% ethyl acetate in hexanes to provide the desired product as a colorless oil (2.7 g, 80%).

### Step 4. N-(3-Cyano-4-fluoro-2-iodophenyl)-3-fluoro-5-(trifluoromethyl)benzamide

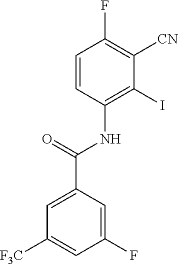 [SMILES: N#Cc1c(F)ccc(NC(=O)c2cc(F)cc(C(F)(F)F)c2)c1I]

To a mixture of 3-amino-6-fluoro-2-iodobenzonitrile (1.7 g, 6.1 mmol) in pyridine (10 mL) were added 3-fluoro-5-(trifluoromethyl)benzoyl chloride (1.6 g, 7.0 mmol) at 0° C. under nitrogen atmosphere. The resulting mixture was warmed to rt and stirred for 30 min before quenched with adding water. The mixture was extracted with ethyl acetate (2×200 mL). The combined organics were dried with anhydrous sodium sulfate, filtered, and concentrated under reduced pressure, which was then purified by silica gel column chromatography, eluted with 50% ethyl acetate in hexanes to provide the desired product as a colorless oil (2.3 g, 85%). LCMS calculated for C₁₅H₇F₅IN₂O (M+H)⁺ m/z=453.0; found 453.0.

### Step 5. N-(3-Amino-4-iodo-1H-indazol-5-yl)-3-fluoro-5-(trifluoromethyl)benzamide

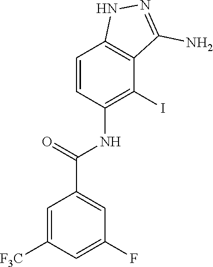 [SMILES: Nc1n[nH]c2ccc(NC(=O)c3cc(F)cc(C(F)(F)F)c3)c(I)c12]

To a mixture of N-(3-cyano-4-fluoro-2-iodophenyl)-3-fluoro-5-(trifluoromethyl)benzamide (560 mg, 1.0 mmol) in n-BuOH (5 mL) were added hydrazine monohydrate (1 mL) at rt. The resulting mixture was stirred at 110° C. for 1h. Upon cooling to room temperature, the mixture was concentrated under reduced pressure. The crude product precipitated out and was collected by filtration followed by washing with water (2×30 mL) and dried at room temperature to give a light brown solid, which was used in next step without further purification. LCMS calculated for C₁₅H₁₀F₄IN₄O (M+H)⁺ m/z=465.0; found 465.0.

### Step 6. N-(3-Amino-4-(2-chloro-5-fluorobenzyl)-1H-indazol-5-yl)-3-fluoro-5-(trifluoromethyl)benzamide

To the suspension of zinc powder (64 mg, 1.0 mmol) in tetrahydrofuran (1.5 mL) at 65° C. under nitrogen was added 1,2-dibromoethane (4 mg, 0.025 mmol) and trimethylsilyl chloride (5 mg, 0.045 mmol). The mixture was stirred at 65° C. for 30 minutes. A solution of 2-chloro-6-fluorobenzyl bromide (0.2 g, 0.9 mmol) in THF (3 mL) was added dropwise and the mixture was stirred with heating at 65° C. for 3 h. The mixture was cooled to ambient temperature and added N-(3-amino-4-iodo-1H-indazol-5-yl)-3-fluoro-5-(trifluoromethyl)benzamide (240 mg, 0.45 mmol), Sphos (20 mg, 0.045 mmol) and Pd(OAc)₂ (10 mg, 0.045 mmol). The mixture was heated to 70° C. for 1 h before quenched with sat. NH₄Cl solution. The mixture was extracted with ethyl acetate (2×200 mL). The combined organics were dried with anhydrous sodium sulfate, filtered, and concentrated under reduced pressure, which was then purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₁₅ClF₅N₄O (M+H)+ m/z=481.1; found 481.1.

### Example 6. N-(3-amino-4-((2-chloro-5-fluorophenoxy)methyl)-1H-indazol-5-yl)-3-fluoro-5-(trifluoromethyl)benzamide

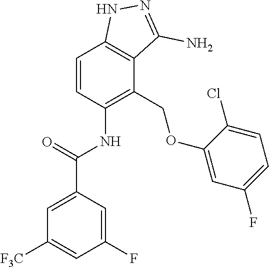 [SMILES: Nc1n[nH]c2ccc(NC(=O)c3cc(F)cc(C(F)(F)F)c3)c(COc3cc(F)ccc3Cl)c12]

### Step 1. N-(3-Cyano-4-fluoro-2-vinylphenyl)-3-fluoro-5-(trifluoromethyl)benzamide

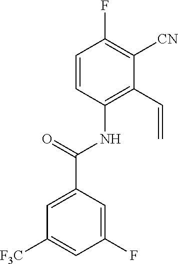 [SMILES: C=Cc1c(NC(=O)c2cc(F)cc(C(F)(F)F)c2)ccc(F)c1C#N]

The mixture of N-(3-cyano-4-fluoro-2-iodophenyl)-3-fluoro-5-(trifluoromethyl)benzamide (Example 5, Step 4, 300 mg, 0.66 mmol), 4,4,5,5-tetramethyl-2-vinyl-1,3,2-dioxaborolane (122 mg, 0.8 mmol), K₃PO₄ (169 mg, 0.8 mmol) and XPhos Pd G2 (39 mg, 0.05 mmol) in 1,4-dioxane (3 mL) and water (0.3 mL) was stirred at 80° C. for 7 h under nitrogen atmosphere. Upon cooling to room temperature, the mixture was concentrated under reduced pressure. The residue was purified by silica gel column chromatography, eluted with 20% ethyl acetate in hexanes to provide the desired product as a white solid (174 mg, 75%).

### Step 2. N-(3-Cyano-4-fluoro-2-formylphenyl)-3-fluoro-5-(trifluoromethyl)benzamide

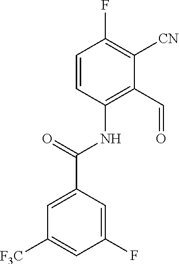 [SMILES: N#Cc1c(F)ccc(NC(=O)c2cc(F)cc(C(F)(F)F)c2)c1C=O]

To a mixture of N-(3-cyano-4-fluoro-2-vinylphenyl)-3-fluoro-5-(trifluoromethyl)benzamide (174 mg, 0.5 mmol) in 1,4-dioxane (3 mL) and water (1 mL) were added OSO₄ (4% wt water solution, 63 μL, 0.01 mmol) and sodium periodate (430 mg, 2.0 mmol). The resulting mixture was stirred at room temperature for 2 h before quenched with adding water. The mixture was extracted with ethyl acetate (2×30 mL). The combined organics were dried with anhydrous sodium sulfate, filtered, and concentrated under reduced pressure, which was then purified by silica gel column chromatography, eluted with 50% ethyl acetate in hexanes to provide the desired product as a colorless oil (153 mg, 88%). LCMS calculated for C₁₋₆H₈F₅N₂O₂ (M+H)+ m/z=355.0; found 355.0.

### Step 3. N-(2-(Bromomethyl)-3-cyano-4-fluorophenyl)-3-fluoro-5-(trifluoromethyl)benzamide

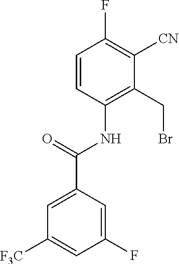 [SMILES: N#Cc1c(F)ccc(NC(=O)c2cc(F)cc(C(F)(F)F)c2)c1CBr]

To a mixture of N-(3-cyano-4-fluoro-2-formylphenyl)-3-fluoro-5-(trifluoromethyl)benzamide (153 mg, 0.44 mmol) in MeOH (4 mL) was added NaBH₄ (18.9 mg, 0.5 mmol) at 0° C. under nitrogen atmosphere. The resulting mixture was stirred at the same temperature for 30 min, and then quenched with sat. NH₄Cl. The mixture was extracted with ethyl acetate (2×10 mL). The combined organics were dried with anhydrous sodium sulfate, filtered, and concentrated under reduced pressure to afford the crude product. The crude product was then dissolved in DCM (2 mL), and PBr₃ (135 mg, 0.5 mmol) was added dropwise at 0° C. The reaction mixture was then warmed to rt and stirred at this temperature for 2 h before quenched with sat. NaHCO₃. The mixture was extracted with DCM (2×10 mL). The combined organics were dried with anhydrous sodium sulfate, filtered, and concentrated under reduced pressure to afford the crude product which is used in next step without future purification.

### Step 4. N-(2-((2-Chloro-5-fluorophenoxy)methyl)-3-cyano-4-fluorophenyl)-3-fluoro-5-(trifluoromethyl)benzamide

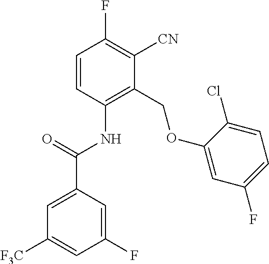 [SMILES: N#Cc1c(F)ccc(NC(=O)c2cc(F)cc(C(F)(F)F)c2)c1COc1cc(F)ccc1Cl]

N-(2-(Bromomethyl)-3-cyano-4-fluorophenyl)-3-fluoro-5-(trifluoromethyl)benzamide (50 mg, 0.11 mmol) was dissolved in DMF (1 mL) then 2-chloro-5-fluorophenol (29 mg, 0.2 mmol) and K₂CO₃ (27 mg, 0.2 mmol) were added. The resulting mixture was stirred at rt for 1 h, then diluted with water. The mixture was extracted with EtOAc (2×10 mL). The combined organics were dried with anhydrous sodium sulfate, filtered, and concentrated under reduced pressure to afford the crude product which is used in next step without future purification. LCMS calculated for C₂₂H₁₂ClF₆N₂O₂ (M+H)+ m/z=485.0; found 485.0.

### Step 5. N-(3-Amino-4-((2-chloro-5-fluorophenoxy)methyl)-1H-indazol-5-yl)-3-fluoro-5-(trifluoromethyl)benzamide

To a mixture of N-(2-((2-chloro-5-fluorophenoxy)methyl)-3-cyano-4-fluorophenyl)-3-fluoro-5-(trifluoromethyl)benzamide (30 mg, 0.06 mmol) in n-BuOH (1 mL) were added hydrazine monohydrate (0.2 mL) at rt. The resulting mixture was stirred at 110° C. for 1 h. Upon cooling to room temperature, the mixture was concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₂H₁₅ClF₅N₄O₂ (M+H)+ m/z=497.1; found 497.1.

### Example 7. N-(3-amino-4-(2-chloro-5-fluorophenoxy)-1-methyl-1H-indazol-5-yl)-3-fluoro-5-(trifluoromethyl)benzamide

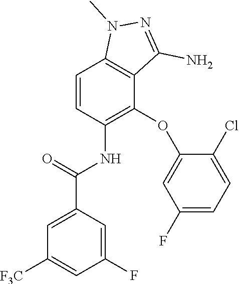 [SMILES: Cn1nc(N)c2c(Oc3cc(F)ccc3Cl)c(NC(=O)c3cc(F)cc(C(F)(F)F)c3)ccc21]

### Step 1. N-(4-(2-Chloro-5-fluorophenoxy)-3-(1,3-dioxoisoindolin-2-yl)-1H-indazol-5-yl)-3-fluoro-5-(trifluoromethyl)benzamide

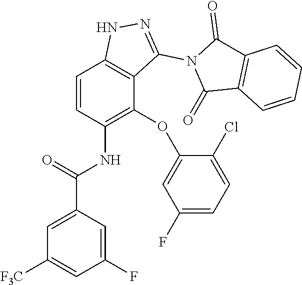 [SMILES: O=C(Nc1ccc2[nH]nc(N3C(=O)c4ccccc4C3=O)c2c1Oc1cc(F)ccc1Cl)c1cc(F)cc(C(F)(F)F)c1]

The mixture of N-(3-amino-4-(2-chloro-5-fluorophenoxy)-1H-indazol-5-yl)-3-fluoro-5-(trifluoromethyl)benzamide (Example 1; 3.0 g, 6.2 mmol) in AcOH (10 mL) was added isobenzofuran-1,3-dione (1.03 g, 7.0 mmol) at room temperature. The reaction was then heated at 120° C. under nitrogen atmosphere for 2 h. Upon cooling to room temperature, the reaction was concentrated under reduced pressure. The residue was taken in EtOAc (500 mL) and washed with sat. NaHCO₃, brine and the organics were dried with anhydrous sodium sulfate, filtered, and concentrated under reduced pressure. The crude product precipitated out and was collected by filtration followed by washing with EtOAc and dried at room temperature to give a light brown solid, which was used in next step without further purification. LCMS calculated for C₂₉H₁₅ClF₅N₄O₄ (M+H)⁺ m/z=613.0; found 613.0.

### Step 2. N-(4-(2-Chloro-5-fluorophenoxy)-3-(1,3-dioxoisoindolin-2-yl)-1-methyl-1H-indazol-5-yl)-3-fluoro-5-(trifluoromethyl)benzamide

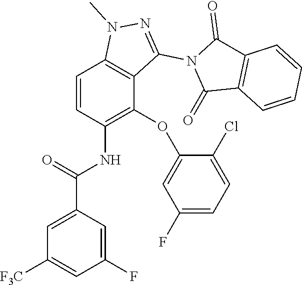 [SMILES: Cn1nc(N2C(=O)c3ccccc3C2=O)c2c(Oc3cc(F)ccc3Cl)c(NC(=O)c3cc(F)cc(C(F)(F)F)c3)ccc21]

N-(4-(2-chloro-5-fluorophenoxy)-3-(1,3-dioxoisoindolin-2-yl)-1H-indazol-5-yl)-3-fluoro-5-(trifluoromethyl)benzamide (50 mg, 0.08 mmol) was dissolved in DMF (1 mL) then iodomethane (14 mg, 0.1 mmol) and K₂CO₃ (27 mg, 0.2 mmol) were added. The resulting mixture was stirred at rt for 1 h, then diluted with water. The mixture was extracted with EtOAc (2×10 mL). The combined organics were dried with anhydrous sodium sulfate, filtered, and concentrated under reduced pressure to afford the crude product which is used in next step without future purification. LCMS calculated for C₃₀H₁₇ClF₅N₄O₄ (M+H)+ m/z=627.1; found 627.1.

### Step 3. N-(3-Amino-4-(2-chloro-5-fluorophenoxy)-1-methyl-1H-indazol-5-yl)-3-fluoro-5-(trifluoromethyl)benzamide

To a mixture of N-(4-(2-chloro-5-fluorophenoxy)-3-(1,3-dioxoisoindolin-2-yl)-1-methyl-1H-indazol-5-yl)-3-fluoro-5-(trifluoromethyl)benzamide (20 mg, 0.03 mmol) in MeOH (1 mL) were added hydrazine monohydrate (0.2 mL) at rt. The resulting mixture was stirred at rt for 1 h. The mixture was concentrated under reduced pressure. The residue was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₂H₁₅ClF₅N₄O₂ (M+H)+ m/z=497.1; found 497.1.

### Example 8. N-(3-Amino-4-(2-chloro-5-fluorophenoxy)-1-ethyl-1H-indazol-5-yl)-3-fluoro-5-(trifluoromethyl)benzamide

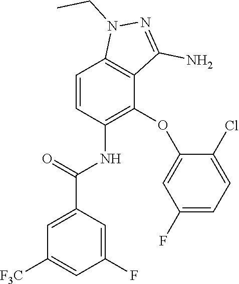 [SMILES: CCn1nc(N)c2c(Oc3cc(F)ccc3Cl)c(NC(=O)c3cc(F)cc(C(F)(F)F)c3)ccc21]

The title compound was prepared using similar procedures as described for Example 7, with iodoethane replacing iodomethane in Step 2. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₁₇ClF₅N₄O₂ (M+H)+ m/z=511.1; found 511.1.

### Example 9. N-(3-Amino-4-(2-chloro-5-fluorophenoxy)-1-(2-(methylamino)-2-oxoethyl)-1H-indazol-5-yl)-3-fluoro-5-(trifluoromethyl)benzamide

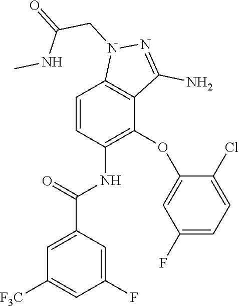 [SMILES: CNC(=O)Cn1nc(N)c2c(Oc3cc(F)ccc3Cl)c(NC(=O)c3cc(F)cc(C(F)(F)F)c3)ccc21]

### Step 1. 2-(4-(2-Chloro-5-fluorophenoxy)-3-(1,3-dioxoisoindolin-2-yl)-5-(3-fluoro-5-(trifluoromethyl)benzamido)-1H-indazol-1-yl)acetic acid

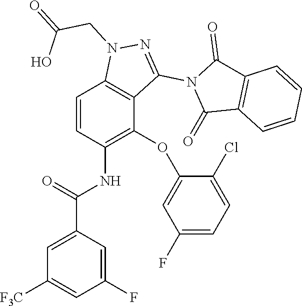 [SMILES: O=C(O)Cn1nc(N2C(=O)c3ccccc3C2=O)c2c(Oc3cc(F)ccc3Cl)c(NC(=O)c3cc(F)cc(C(F)(F)F)c3)ccc21]

N-(4-(2-chloro-5-fluorophenoxy)-3-(1,3-dioxoisoindolin-2-yl)-1H-indazol-5-yl)-3-fluoro-5-(trifluoromethyl)benzamide (Example 7, Step 1, 100 mg, 0.16 mmol) was dissolved in DMF (1 mL) then tert-butyl 2-bromoacetate (38 mg, 0.2 mmol) and K₂CO₃ (54 mg, 0.4 mmol) were added. The resulting mixture was stirred at rt for 1 h, then diluted with water. The mixture was extracted with EtOAc (2×10 mL). The combined organics were dried with anhydrous sodium sulfate, filtered, and concentrated under reduced pressure to afford the crude product which is dissolved in DCM (1 mL) and TFA (1 mL). The mixture is stirred at rt for 1 h before concentrated under reduced pressure. The residue was taken into EtOAc and washed with and washed with water, brine and the organics were dried with anhydrous sodium sulfate, filtered, and concentrated under reduced pressure to afford the crude product which is used in next step without future purification. LCMS calculated for C₃₁H₁₇ClF₅N₄O₆ (M+H)+ m/z=671.1; found 671.1.

### Step 2. N-(4-(2-Chloro-5-fluorophenoxy)-3-(1,3-dioxoisoindolin-2-yl)-1-(2-(methylamino)-2-oxoethyl)-1H-indazol-5-yl)-3-fluoro-5-(trifluoromethyl)benzamide

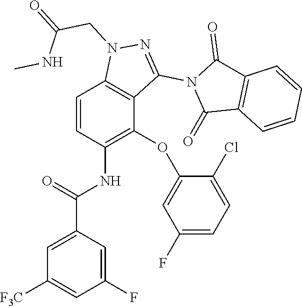 [SMILES: CNC(=O)Cn1nc(N2C(=O)c3ccccc3C2=O)c2c(Oc3cc(F)ccc3Cl)c(NC(=O)c3cc(F)cc(C(F)(F)F)c3)ccc21]

To a solution of 2-(4-(2-chloro-5-fluorophenoxy)-3-(1,3-dioxoisoindolin-2-yl)-5-(3-fluoro-5-(trifluoromethyl)benzamido)-1H-indazol-1-yl)acetic acid (20 mg, 0.03 mmol) in THF (1 mL) was added methylamine (2M THF solution, 30 μL, 0.06 mmol), N,N-Diisopropylethylamine (7 μL, 0.04 mmol) and propylphosphonic anhydride solution (50% THF solution, 25 mg, 0.04 mmol). The resulting mixture was stirred at room temperature for 2 h before quenched with adding water. The mixture was extracted with ethyl acetate (2×30 mL). The combined organics were dried with anhydrous sodium sulfate, filtered, and concentrated under reduced pressure to afford the crude product which is used in next step without future purification. LCMS calculated for C₃₂H₂₀ClF₅N₅O₅ (M+H)+ m/z=684.1; found 684.1.

### Step 3. N-(3-Amino-4-(2-chloro-5-fluorophenoxy)-1-(2-(methylamino)-2-oxoethyl)-1H-indazol-5-yl)-3-fluoro-5-(trifluoromethyl)benzamide

To a mixture of N-(4-(2-chloro-5-fluorophenoxy)-3-(1,3-dioxoisoindolin-2-yl)-1-(2-(methylamino)-2-oxoethyl)-1H-indazol-5-yl)-3-fluoro-5-(trifluoromethyl)benzamide (15 mg, 0.02 mmol) in MeOH (1 mL) were added hydrazine monohydrate (0.2 mL) at rt. The resulting mixture was stirred at rt for 1 h. The mixture was concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₄H₁₁ClF₅N₅O₃ (M+H)+ m/z=554.1; found 554.1.

### Example 10. N-(3-Amino-4-(2-chloro-5-fluorophenoxy)-1-(2-morpholino-2-oxoethyl)-1H-indazol-5-yl)-3-fluoro-5-(trifluoromethyl)benzamide

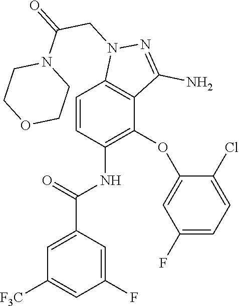 [SMILES: Nc1nn(CC(=O)N2CCOCC2)c2ccc(NC(=O)c3cc(F)cc(C(F)(F)F)c3)c(Oc3cc(F)ccc3Cl)c12]

The title compound was prepared using similar procedures as described for Example 9, with morpholine replacing methylamine in Step 2. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₂₂ClF₅N₅O₄ (M+H)+ m/z=610.1; found 610.2.

### Example 11. N-(3-Amino-7-bromo-4-(2-chloro-5-fluorophenoxy)-1H-indazol-5-yl)-3-fluoro-5-(trifluoromethyl)benzamide

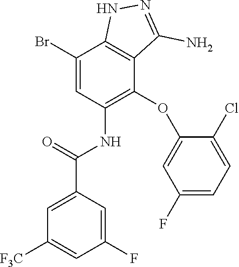 [SMILES: Nc1n[nH]c2c(Br)cc(NC(=O)c3cc(F)cc(C(F)(F)F)c3)c(Oc3cc(F)ccc3Cl)c12]

### Step 1. N-(7-Bromo-4-(2-chloro-5-fluorophenoxy)-3-(1,3-dioxoisoindolin-2-yl)-1H-indazol-5-yl)-3-fluoro-5-(trifluoromethyl)benzamide

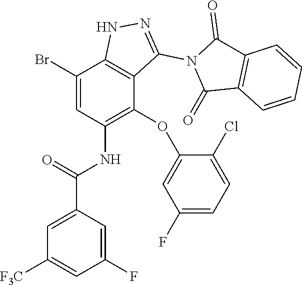 [SMILES: O=C(Nc1cc(Br)c2[nH]nc(N3C(=O)c4ccccc4C3=O)c2c1Oc1cc(F)ccc1Cl)c1cc(F)cc(C(F)(F)F)c1]

N-(4-(2-Chloro-5-fluorophenoxy)-3-(1,3-dioxoisoindolin-2-yl)-1H-indazol-5-yl)-3-fluoro-5-(trifluoromethyl)benzamide (Example 7, Step 1, 3.1 g, 5.06 mmol) was dissolved in MeCN (50 mL) then AcOH (303 mg, 5.06 mmol) and NBS (908 mg, 5.1 mmol) were added. The reaction was then heated at 100° C. under nitrogen atmosphere for 2 h. Upon cooling to room temperature, the reaction was concentrated under reduced pressure. The residue was taken in EtOAc (500 mL) and washed with sat. NaHCO₃, brine and the organics were dried with anhydrous sodium sulfate, filtered, and concentrated under reduced pressure. The crude product was then purified by silica gel column chromatography, eluted with 25% ethyl acetate in dichloromethane to provide the desired product as a purple solid (3.1 g, 88%). LCMS calculated for C₂₉H₁₄BrClF₅N₄O₄ (M+H)+ m/z=691.0; found 691.0.

### Step 2. N-(3-Amino-7-bromo-4-(2-chloro-5-fluorophenoxy)-1H-indazol-5-yl)-3-fluoro-5-(trifluoromethyl)benzamide

To a mixture of N-(7-bromo-4-(2-chloro-5-fluorophenoxy)-3-(1,3-dioxoisoindolin-2-yl)-1H-indazol-5-yl)-3-fluoro-5-(trifluoromethyl)benzamide (15 mg, 0.02 mmol) in MeOH (1 mL) were added hydrazine monohydrate (0.2 mL) at rt. The resulting mixture was stirred at rt for 1 h. The mixture was concentrated under reduced pressure. The residue was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₁H₁₂BrClF₅N₄O₂ (M+H)+ m/z=561.0; found 561.1.

### Example 12. N-(3-Amino-7-chloro-4-(2-chloro-5-fluorophenoxy)-1H-indazol-5-yl)-3-fluoro-5-(trifluoromethyl)benzamide

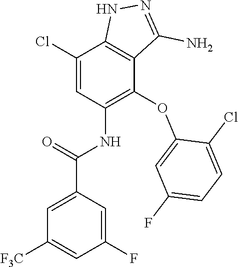 [SMILES: Nc1n[nH]c2c(Cl)cc(NC(=O)c3cc(F)cc(C(F)(F)F)c3)c(Oc3cc(F)ccc3Cl)c12]

The title compound was prepared using similar procedures as described for Example 11, with NCS replacing NBS in Step 1. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₁H₁₂Cl₂F₅N₄O₂(M+H)+ m/z=517.0; found 517.0.

### Example 13. N-(3-Amino-4-(2-chloro-5-fluorophenoxy)-7-methyl-1H-indazol-5-yl)-3-fluoro-5-(trifluoromethyl)benzamide

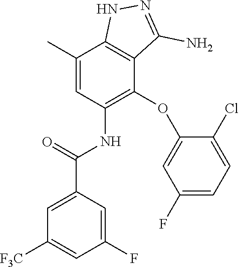 [SMILES: Cc1cc(NC(=O)c2cc(F)cc(C(F)(F)F)c2)c(Oc2cc(F)ccc2Cl)c2c(N)n[nH]c12]

### Step 1. N-(4-(2-Chloro-5-fluorophenoxy)-3-(1,3-dioxoisoindolin-2-yl)-7-methyl-1H-indazol-5-yl)-3-fluoro-5-(trifluoromethyl)benzamide

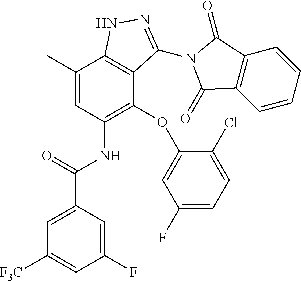 [SMILES: Cc1cc(NC(=O)c2cc(F)cc(C(F)(F)F)c2)c(Oc2cc(F)ccc2Cl)c2c(N3C(=O)c4ccccc4C3=O)n[nH]c12]

The mixture of N-(7-Bromo-4-(2-chloro-5-fluorophenoxy)-3-(1,3-dioxoisoindolin-2-yl)-1H-indazol-5-yl)-3-fluoro-5-(trifluoromethyl)benzamide (Example 11, Step 1, 30 mg, 0.04 mmol), methylboronic acid (6 mg, 0.1 mmol), K₃PO₄ (42 mg, 0.2 mmol) and PCy₃Pd G2 (7 mg, 0.01 mmol) in toluene (0.5 mL) and water (0.05 mL) was stirred at 100° C. for 4 h under nitrogen atmosphere. Upon cooling to room temperature, the mixture was diluted with water and extracted with EtOAc. The combined organics were dried with anhydrous sodium sulfate, filtered, and concentrated under reduced pressure to afford the crude product which is used in next step without future purification. LCMS calculated for C₃₀H₁₇ClF₅N₄O₄ (M+H)+ m/z=627.1; found 627.1.

### Step 2. N-(3-Amino-4-(2-chloro-5-fluorophenoxy)-7-methyl-1H-indazol-5-yl)-3-fluoro-5-(trifluoromethyl)benzamide

To a mixture of N-(4-(2-chloro-5-fluorophenoxy)-3-(1,3-dioxoisoindolin-2-yl)-7-methyl-1H-indazol-5-yl)-3-fluoro-5-(trifluoromethyl)benzamide (15 mg, 0.02 mmol) in MeOH (1 mL) were added hydrazine monohydrate (0.2 mL) at rt. The resulting mixture was stirred at rt for 1 h. The mixture was concentrated under reduced pressure. The residue was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₂H₁₅ClF₅N₄O₂ (M+H)+ m/z=497.1; found 497.1.

### Example 14. N-(3-Amino-4-(2-chloro-5-fluorophenoxy)-7-(1-methyl-1H-pyrazol-4-yl)-1H-indazol-5-yl)-3-fluoro-5-(trifluoromethyl)benzamide

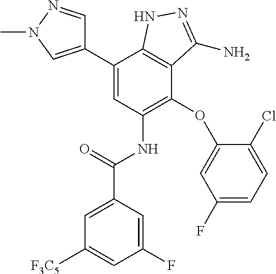

The titled compound was prepared using similar procedures as described for Example 13, with 1-methyl-4-(4,4,5,5-tetramethyl-1,3,2-dioxaborolan-2-yl)-1H-pyrazole replacing methylboronic acid and Xphos PdG2 replacing PCy₃Pd G2 in Step 1. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₁₇ClF₅N₆O₂(M+H)+ m/z=563.1; found 563.1.

### Example 15. N-(3-Amino-4-(2-chloro-5-fluorophenoxy)-7-(1-methyl-1H-pyrazol-5-yl)-1H-indazol-5-yl)-3-fluoro-5-(trifluoromethyl)benzamide

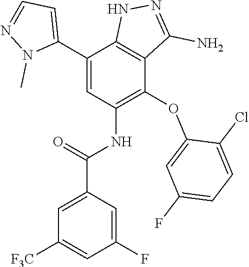 [SMILES: Cn1nccc1-c1cc(NC(=O)c2cc(F)cc(C(F)(F)F)c2)c(Oc2cc(F)ccc2Cl)c2c(N)n[nH]c12]

The title compound was prepared using similar procedures as described for Example 13, with 1-methyl-5-(4,4,5,5-tetramethyl-1,3,2-dioxaborolan-2-yl)-1H-pyrazole replacing methylboronic acid and Xphos PdG2 replacing PCy₃Pd G2 in Step 1. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₁₇ClF₅N₆O₂ (M+H)+ m/z=563.1; found 563.1.

### Example 16. N-(3-Amino-4-(2-chloro-5-fluorophenoxy)-7-(pyridin-3-yl)-1H-indazol-5-yl)-3-fluoro-5-(trifluoromethyl)benzamide

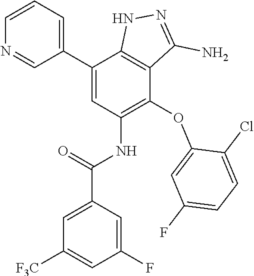 [SMILES: Nc1n[nH]c2c(-c3cccnc3)cc(NC(=O)c3cc(F)cc(C(F)(F)F)c3)c(Oc3cc(F)ccc3Cl)c12]

The title compound was prepared using similar procedures as described for Example 13, with pyridin-3-ylboronic acid replacing methylboronic acid and Xphos PdG2 replacing PCy₃Pd G2 in Step 1. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₁₆ClF₅N₅O₂ (M+H)+ m/z=560.1; found 560.1.

### Example 17. N-(3-Amino-4-(2-chloro-5-fluorophenoxy)-7-phenyl-1H-indazol-5-yl)-3-fluoro-5-(trifluoromethyl)benzamide

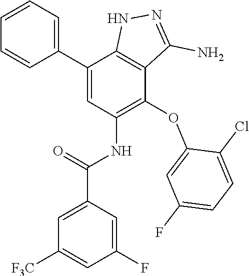 [SMILES: Nc1n[nH]c2c(-c3ccccc3)cc(NC(=O)c3cc(F)cc(C(F)(F)F)c3)c(Oc3cc(F)ccc3Cl)c12]

The title compound was prepared using similar procedures as described for Example 13, with phenylboronic acid replacing methylboronic acid and Xphos PdG2 replacing PCy₃Pd G2 in Step 1. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₇H₁₇ClF₅N₄O₂ (M+H)+ m/z=559.1; found 559.1.

### Example 18. N-(3-Amino-4-(2-chloro-5-fluorophenoxy)-1H-indazol-5-yl)benzo[b]thiophene-3-carboxamide

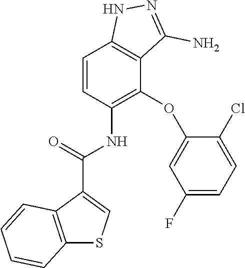 [SMILES: Nc1n[nH]c2ccc(NC(=O)c3csc4ccccc34)c(Oc3cc(F)ccc3Cl)c12]

The title compound was prepared using similar procedures as described for Example 1, with benzo[b]thiophene-3-carbonyl chloride replacing 3-fluoro-5-(trifluoromethyl)benzoyl chloride in Step 3. The final product was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₁₅ClFN₄O₂S (M+H)+ m/z=453.1; found 453.1.

### Example 19. N-(3-Amino-4-(2-chloro-5-fluorophenoxy)-1H-indazol-5-yl)-5-fluoro-3-hydroxy-3-(trifluoromethyl)indoline-1-carboxamide

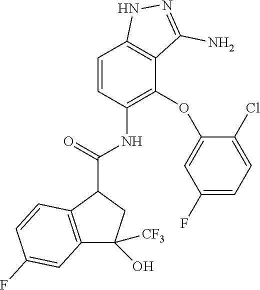 [SMILES: Nc1n[nH]c2ccc(NC(=O)C3CC(O)(C(F)(F)F)c4cc(F)ccc43)c(Oc3cc(F)ccc3Cl)c12]

### Step 1. 1-Benzyl-5-fluoroindoline-2,3-dione

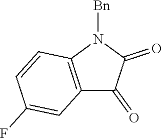 [SMILES: O=C1C(=O)N(Cc2ccccc2)c2ccc(F)cc21]

5-Fluoroindoline-2,3-dione (5.0 g, 30.28 mmol) was dissolved in DMF (100 mL) then BnBr (4.31 mL, 36.34 mmol) and K₂CO₃ (5.0 g, 36.34 mmol) were added. The resulting mixture was stirred at 80° C. for 3 h, then diluted with water. The mixture was extracted with EtOAc (2×300 mL). The combined organics were dried with anhydrous sodium sulfate, filtered, and concentrated under reduced pressure. The residue was purified by silica gel column chromatography, eluted with 10% ethyl acetate in hexanes to provide the desired product as a colorless oil (6.17 g, 80%). LCMS calculated for C₁₅H₁₁FNO₂ (M+H)+ m/z=256.1; found 256.0

### Step 2. 1-Benzyl-5-fluoro-3-(trifluoromethyl)-3-((trimethylsilyl)oxy)indoline

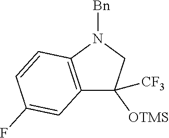

To a mixture of 1-benzyl-5-fluoroindoline-2,3-dione (6.17 g, 24.22 mmol) in THF (300 mL) was added TMSCF₃ (5.16 g, 36.26 mmol) at 0° C. under nitrogen atmosphere. The mixture was stirred at the same temperature for 10 min, followed by the addition of CsF (367 mg, 2.42 mmol). The resulting mixture was stirred at room temperature for 2 d and then quenched by adding water; the mixture was extracted with ethyl acetate (2×300 mL). The combined organics were washed with brine (3×100 mL), dried with anhydrous sodium sulfate. After filtered, the filtrate was concentrated under reduced pressure. The residue was dissolved in THF and BH₃·THF (1M THF solution, 30 mL) was added, the mixture was stirred at rt for 2 d. Aqueous HCl (3 M) was added dropwise and the mixture was subsequently neutralized with aqueous NaOH (2.5 M). Saturated aqueous NaCl (10 mL) was added and the mixture extracted with DCM (3×150 mL). The organic phase was further washed with water (2×15 mL), dried over Na₂SO₄, filtered and evaporated at reduced pressure. The residue was purified by silica gel column chromatography, eluted with 10% ethyl acetate in hexanes to provide the desired product as a colorless oil (5.9 g, 67%). LCMS calculated for C₁₋₆H₁₂F₄N (M-OTMS)⁺ m/z=294.1; found 294.1.

### Step 3. 5-Fluoro-3-(trifluoromethyl)-3-((trimethylsilyl)oxy)indoline

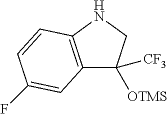

To a mixture of 1-benzyl-5-fluoro-3-(trifluoromethyl)-3-((trimethylsilyl)oxy)indoline (5.9 g, 15.4 mmol) and Pd/C (10% on C, 1.06 g, 1 mmol) under N₂ was added MeOH (200 mL). The resulting mixture was purged with H₂ for 10 min, and the reaction was stirred at rt under 1 atm H₂ for 12h. Upon completion, the reaction was filtrated though a pad of celite, concentrated under reduced pressure. The residue was purified by silica gel column chromatography, eluted with 20% ethyl acetate in hexanes to provide the desired product as a colorless oil (2.2 g, 50%). LCMS calculated for C₉H₆F₄N (M-OTMS)⁺ m/z=204.0; found 204.0.

### Step 4. N-(2-(2-Chloro-5-fluorophenoxy)-3-cyano-4-fluorophenyl)-5-fluoro-3-(trifluoromethyl)-3-((trimethylsilyl)oxy)indoline-1-carboxamide

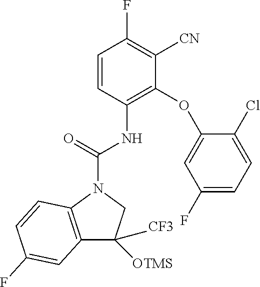

To a solution of triphosgene (31.7 mg, 0.11 mmol) in DCM (1 mL) under N₂ at 0° C. was added a solution of 3-amino-2-(2-chloro-5-fluorophenoxy)-6-fluorobenzonitrile (100 mg, 0.36 mmol) in DCM (1 mL). After stirring for 10 min triethylamine (49.7 μL, 0.36 mmol) was added. The reaction was stirred for a further 1 h and allowed to warm to room temperature over this time. The reaction was then added 5-fluoro-3-(trifluoromethyl)-3-((trimethylsilyl)oxy)indoline (104 mg, 0.36 mmol) and stirred for 4 h before quenched by added sat. NaHCO₃. The mixture was extracted with ethyl acetate (2×10 mL). The combined organics were washed with brine, dried with anhydrous sodium sulfate. After filtered, the filtrate was concentrated under reduced pressure. The residue was purified by silica gel column chromatography, eluted with 10% ethyl acetate in hexanes to provide the desired product as a yellow oil (92 mg, 43%). LCMS calculated for C₂₃H₁₁ClF₆N₃O₂ (M-OTMS)⁺ m/z=510.0; found 510.0.

### Step 5. N-(3-Amino-4-(2-chloro-5-fluorophenoxy)-1H-indazol-5-yl)-5-fluoro-3-(trifluoromethyl)-3-((trimethylsilyl)oxy)indoline-1-carboxamide

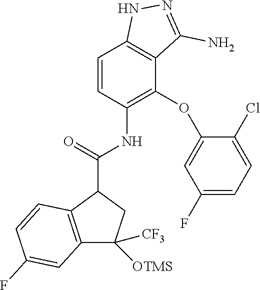

To a mixture of N-(2-(2-chloro-5-fluorophenoxy)-3-cyano-4-fluorophenyl)-5-fluoro-3-(trifluoromethyl)-3-((trimethylsilyl)oxy)indoline-1-carboxamide (92 mg, 0.15 mmol) in n-BuOH (1 mL) were added hydrazine monohydrate (0.2 mL) at rt. The resulting mixture was stirred at 100° C. for 1h. Upon cooling to room temperature, the mixture was concentrated under reduced pressure. The residue was purified by silica gel column chromatography, eluted with 4% MeOH in DCM to provide the desired product as a yellow oil (80 mg, 87%). LCMS calculated for C₂₃H₁₄ClF₅N₅O₂ (M-OTMS)⁺ m/z=522.1; found 522.1.

### Step 6. N-(3-Amino-4-(2-chloro-5-fluorophenoxy)-1H-indazol-5-yl)-5-fluoro-3-hydroxy-3-(trifluoromethyl)indoline-1-carboxamide

To a mixture of N-(3-amino-4-(2-chloro-5-fluorophenoxy)-1H-indazol-5-yl)-5-fluoro-3-(trifluoromethyl)-3-((trimethylsilyl)oxy)indoline-1-carboxamide (80 mg, 0.13 mmol) in THF (1 mL) were added tetrabutylammonium fluoride (1.0 M THF, 0.2 mL) at 0° C. The resulting mixture was warmed to rt and stirred for 1 h before quenched by adding sat. NH₄Cl. the mixture was concentrated under reduced pressure. The residue was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₁₆ClF₅N₅O₃ (M+H)+ m/z=540.1; found 540.1.

### Example 20. N-(3-Amino-5-(2-chloro-5-fluorophenoxy)-[1,2,4]triazolo[4,3-a]pyridin-6-yl)-3-fluoro-5-(trifluoromethyl)benzamide

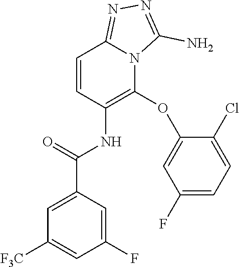 [SMILES: Nc1nnc2ccc(NC(=O)c3cc(F)cc(C(F)(F)F)c3)c(Oc3cc(F)ccc3Cl)n12]

### Step 1. 6-Chloro-2-(2-chloro-5-fluorophenoxy)-3-nitropyridine

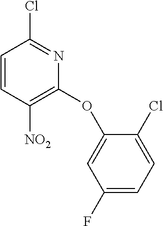 [SMILES: O=[N+]([O-])c1ccc(Cl)nc1Oc1cc(F)ccc1Cl]

To a solution of 2-chloro-5-fluorophenol (3.0 g, 20.7 mmol) in anhydrous tetrahydrofuran (100 mL) was added NaH (60% in mineral oil, 911 mg, 22.8 mmol) at 0° C. under nitrogen atmosphere. The resulting mixture was stirred at same temperature for 30 min before 2,6-dichloro-3-nitropyridine (4.0 g, 20.7 mmol) was added in one portion. The reaction was then warmed to room temperature and stirred for 1 h before quenched with water; the mixture was extracted with ethyl acetate (2×200 mL). The combined organics were dried with anhydrous sodium sulfate, filtered, and concentrated under reduced pressure. The residue was purified by silica gel column chromatography, eluted with 20% ethyl acetate in hexanes to provide the desired product as a pale-yellow solid (4.4 g, 70%).

### Step 2. tert-Butyl 2-(6-(2-chloro-5-fluorophenoxy)-5-nitropyridin-2-yl)hydrazine-1-carboxylate

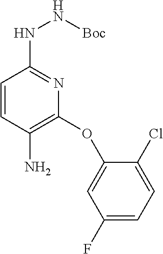 [SMILES: CC(C)(C)OC(=O)NNc1ccc(N)c(Oc2cc(F)ccc2Cl)n1]

To a mixture of 6-chloro-2-(2-chloro-5-fluorophenoxy)-3-nitropyridine (3.0 g, 9.9 mmol) in n-BuOH (50 mL) were added tert-butyl hydrazinecarboxylate (1.93 g, 14.85 mmol) and DIEA (2.19 mL, 11.9 mmol) at rt. The resulting mixture was stirred at 120° C. for 8h. Upon cooling to room temperature, the mixture was concentrated under reduced pressure. The residue was purified by silica gel column chromatography, eluted with 20% ethyl acetate in hexanes to provide the desired product as a pale-yellow oil (2.4 g, 60%). LCMS calculated for C₁₋₆H₁₇ClFN₄O₅(M+H)+ m/z=399.1; found 399.1.

### Step 3. tert-Butyl 2-(5-amino-6-(2-chloro-5-fluorophenoxy)pyridin-2-yl)hydrazine-1-carboxylate

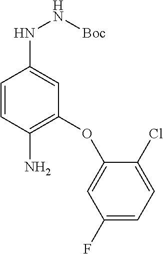 [SMILES: CC(C)(C)OC(=O)NNc1ccc(N)c(Oc2cc(F)ccc2Cl)c1]

To a mixture of tert-butyl 2-(6-(2-chloro-5-fluorophenoxy)-5-nitropyridin-2-yl)hydrazine-1-carboxylate (2.4 g, 5.94 mmol) in MeOH (30 mL), THF (30 mL) and sat. NH₄Cl (15 mL) were added iron (1.6 g, 29.7 mmol). The resulting mixture was stirred at 70° C. for 2 h. Upon cooling to room temperature, the mixture was concentrated under reduced pressure. The residue was extracted with DCM (2×200 mL). The combined organics were dried with anhydrous sodium sulfate, filtered, and concentrated under reduced pressure, which was used directly without purification in next step. LCMS calculated for C₁₋₆H₁₉ClFN₄O₃(M+H)+ m/z=369.1; found 369.1.

### Step 4. tert-Butyl 2-(6-(2-chloro-5-fluorophenoxy)-5-(1,3-dioxoisoindolin-2-yl)pyridin-2-yl)hydrazine-1-carboxylate

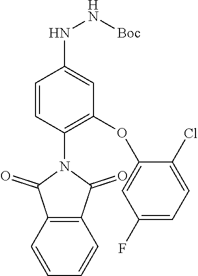 [SMILES: CC(C)(C)OC(=O)NNc1ccc(N2C(=O)c3ccccc3C2=O)c(Oc2cc(F)ccc2Cl)c1]

The mixture of tert-butyl 2-(5-amino-6-(2-chloro-5-fluorophenoxy)pyridin-2-yl)hydrazine-1-carboxylate (2.0 g, 5.4 mmol) in AcOH (30 mL) was added isobenzofuran-1,3-dione (809 mg, 5.6 mmol) at room temperature. The reaction was then heated at 100° C. under nitrogen atmosphere for 2 h. Upon cooling to room temperature, the reaction was concentrated under reduced pressure. The residue was taken in EtOAc (500 mL) and washed with sat. NaHCO₃, brine and the organics were dried with anhydrous sodium sulfate, filtered, and concentrated under reduced pressure. The residue was purified by silica gel column chromatography, eluted with 20% ethyl acetate in DCM to provide the desired product as a pale-yellow oil (1.7 g, 65%). LCMS calculated for C₂₄H₂₁ClFN₄O₅(M+H)+ m/z=499.1; found 499.1.

### Step 5. 2-(2-(2-Chloro-5-fluorophenoxy)-6-hydrazinylpyridin-3-yl)isoindoline-1,3-dione

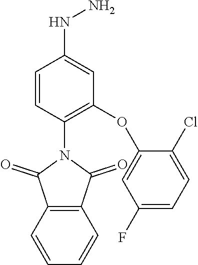 [SMILES: NNc1ccc(N2C(=O)c3ccccc3C2=O)c(Oc2cc(F)ccc2Cl)c1]

To a mixture of tert-butyl 2-(6-(2-chloro-5-fluorophenoxy)-5-(1,3-dioxoisoindolin-2-yl)pyridin-2-yl)hydrazine-1-carboxylate (1.7 g, 3.41 mmol) in DCM (10 mL) was added TFA (3 mL). The resulting mixture was stirred at rt for 2 h. Upon completion, the mixture was concentrated under reduced pressure. The residue was taken in DCM (200 mL) and washed with sat. NaHCO₃, brine and the organics were dried with anhydrous sodium sulfate, filtered, and concentrated under reduced pressure. The residue was used directly in next step without purification. LCMS calculated for C₁₉H₁₃ClFN₄O₃(M+H)+ m/z=399.1; found 399.1.

### Step 6. Ethyl (5-(2-chloro-5-fluorophenoxy)-6-(1,3-dioxoisoindolin-2-yl)-[1,2,4]triazolo[4,3-a]pyridin-3-yl)carbamate

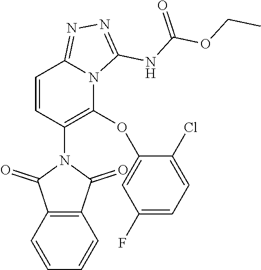 [SMILES: CCOC(=O)Nc1nnc2ccc(N3C(=O)c4ccccc4C3=O)c(Oc3cc(F)ccc3Cl)n12]

To a reaction mixture of 2-(2-(2-chloro-5-fluorophenoxy)-6-hydrazineylpyridin-3-yl)isoindoline-1,3-dione (1.0 g, 2.51 mmol) in 20 mL of THF was added O-ethyl carbonisothiocyanatidate (362 mg, 2.76 mmol) and the reaction was allowed to stir at room temperature for 20 min. The solvent was removed in vacuo. The residue was dissolved in 30 mL of methylene chloride and to it was added 2-chloro-1-methylpyridinium iodide (769 mg, 3.01 mmol) followed by triethylamine (0.5 mL, 3.76 mmol). The reaction mixture was stirred at room temperature for 2 h. The solvent was removed in vacuo and the residue was purified by silica gel chromatography to afford the title compound as a pale-yellow oil (689 mg, 55%). LCMS calculated for C₂₃H₁₆ClFN₅O₅(M+H)+ m/z=496.1; found 496.1.

### Step 7. Ethyl (6-amino-5-(2-chloro-5-fluorophenoxy)-[1,2,4]triazolo[4,3-a]pyridin-3-yl)carbamate

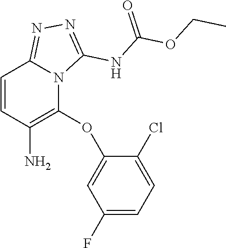 [SMILES: CCOC(=O)Nc1nnc2ccc(N)c(Oc3cc(F)ccc3Cl)n12]

To a mixture of ethyl (5-(2-chloro-5-fluorophenoxy)-6-(1,3-dioxoisoindolin-2-yl)-[1,2,4]triazolo[4,3-a]pyridin-3-yl)carbamate (689 mg, 1.38 mmol) in MeOH (30 mL) were added hydrazine monohydrate (2 mL) at rt. The resulting mixture was stirred at rt for 1 h. The mixture was concentrated under reduced pressure. The residue was used directly in next step without purification. LCMS calculated for C₁₅H₁₄ClFN₅O₃(M+H)+ m/z=366.1; found 366.1.

### Step 8. Ethyl (5-(2-chloro-5-fluorophenoxy)-6-(3-fluoro-5-(trifluoromethyl)benzamido)-[1,2,4]triazolo[4,3-a]pyridin-3-yl)carbamate

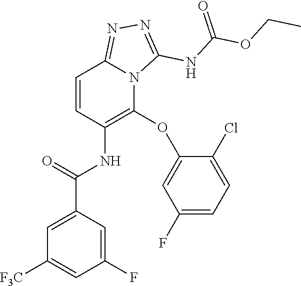 [SMILES: CCOC(=O)Nc1nnc2ccc(NC(=O)c3cc(F)cc(C(F)(F)F)c3)c(Oc3cc(F)ccc3Cl)n12]

To a mixture of ethyl (6-amino-5-(2-chloro-5-fluorophenoxy)-[1,2,4]triazolo[4,3-a]pyridin-3-yl)carbamate (200 mg, 0.54 mmol) in pyridine (2 mL) were added 3-fluoro-5-(trifluoromethyl)benzoyl chloride (134 mg, 0.6 mmol) at 0° C. under nitrogen atmosphere. The resulting mixture was warmed to rt and stirred for 30 min before quenched with adding water. The mixture was extracted with ethyl acetate (2×20 mL). The combined organics were dried with anhydrous sodium sulfate, filtered, and concentrated under reduced pressure and the residue was used directly in next step without purification. LCMS calculated for C₂₃H₁₆ClF₅N₅O₄ (M+H)+ m/z=556.1; found 556.1.

### Step 9. N-(3-Amino-5-(2-chloro-5-fluorophenoxy)-[1,2,4]triazolo[4,3-a]pyridin-6-yl)-3-fluoro-5-(trifluoromethyl)benzamide

To a mixture of ethyl (5-(2-chloro-5-fluorophenoxy)-6-(3-fluoro-5-(trifluoromethyl)benzamido)-[1,2,4]triazolo[4,3-a]pyridin-3-yl)carbamate (50 mg, 0.09 mmol) in dioxane (1 mL) were added sat. NaHCO₃ (0.2 mL) at rt. The resulting mixture was stirred at 120° C. for 1 h. The mixture was concentrated under reduced pressure. The residue was purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₀H₁₂ClF₅N₅O₂ (M+H)+ m/z=484.1; found 484.1.

### Example 21. N-(3-Amino-4-(2-chloro-5-fluorophenoxy)-7-(morpholinomethyl)-1H-indazol-5-yl)-3-fluoro-5-(trifluoromethyl)benzamide

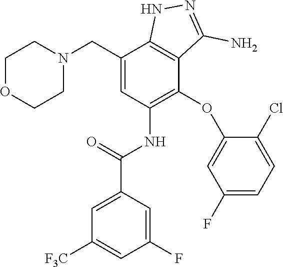 [SMILES: Nc1n[nH]c2c(CN3CCOCC3)cc(NC(=O)c3cc(F)cc(C(F)(F)F)c3)c(Oc3cc(F)ccc3Cl)c12]

### Step 1: N-(4-(2-chloro-5-fluorophenoxy)-3-(1,3-dioxoisoindolin-2-yl)-7-vinyl-1H-indazol-5-yl)-3-fluoro-5-(trifluoromethyl)benzamide

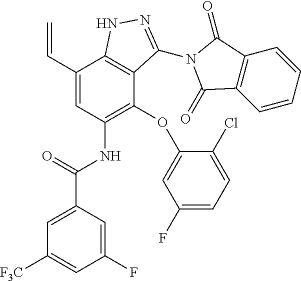 [SMILES: C=Cc1cc(NC(=O)c2cc(F)cc(C(F)(F)F)c2)c(Oc2cc(F)ccc2Cl)c2c(N3C(=O)c4ccccc4C3=O)n[nH]c12]

The mixture of N-(7-bromo-4-(2-chloro-5-fluorophenoxy)-3-(1,3-dioxoisoindolin-2-yl)-1H-indazol-5-yl)-3-fluoro-5-(trifluoromethyl)benzamide (Example 11, step 1, 300 mg, 0.4 mmol), potassium vinyltrifluoroborate (66 mg, 0.5 mmol), K₃PO₄ (420 mg, 2.0 mmol) and XphosPd G2 (70 mg, 0.1 mmol) in toluene (5 mL) and water (0.5 mL) was stirred at 100° C. for 4 h under nitrogen atmosphere. Upon cooling to room temperature, the mixture was diluted with water and extracted with DCM. The combined organics were dried with anhydrous sodium sulfate, filtered, and concentrated under reduced pressure to afford the crude product which was purified by silica gel chromatography to afford the title compound as a brown oil (102 mg, 40%). LCMS calculated for C₃₁H₁₇ClF₅N₄O₄ (M+H)+ m/z=639.1; found 639.1.

### Step 2: N-(4-(2-chloro-5-fluorophenoxy)-3-(1,3-dioxoisoindolin-2-yl)-7-formyl-1H-indazol-5-yl)-3-fluoro-5-(trifluoromethyl)benzamide

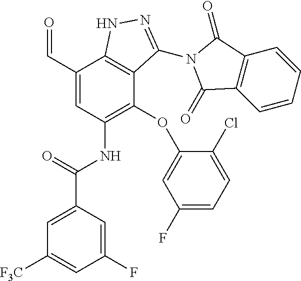 [SMILES: O=Cc1cc(NC(=O)c2cc(F)cc(C(F)(F)F)c2)c(Oc2cc(F)ccc2Cl)c2c(N3C(=O)c4ccccc4C3=O)n[nH]c12]

To a mixture of N-(4-(2-chloro-5-fluorophenoxy)-3-(1,3-dioxoisoindolin-2-yl)-7-vinyl-1H-indazol-5-yl)-3-fluoro-5-(trifluoromethyl)benzamide (102 mg, 0.16 mmol) in 1,4-dioxane (3 mL) and water (1 mL) was added OsO₄ (4% wt water solution, 63 μL, 0.01 mmol) and sodium periodate (172 mg, 0.8 mmol). The resulting mixture was stirred at room temperature for 2 h before quenched by adding water. The mixture was extracted with ethyl acetate (2×30 mL). The combined organics were dried with anhydrous sodium sulfate, filtered, and concentrated under reduced pressure, which was then purified by silica gel column chromatography, eluted with 50% ethyl acetate in DCM to provide the desired product as a colorless oil (73 mg, 72%). LCMS calculated for C₃₀H₁₅ClF₅N₄O₅ (M+H)⁺ m/z=641.1; found 641.1.

### Step 3: N-(4-(2-chloro-5-fluorophenoxy)-3-(1,3-dioxoisoindolin-2-yl)-7-(morpholinomethyl)-1H-indazol-5-yl)-3-fluoro-5-(trifluoromethyl)benzamide

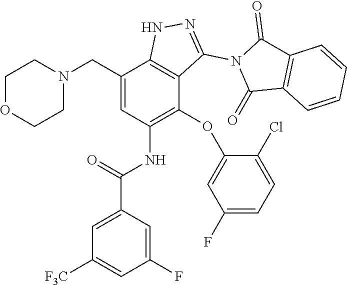 [SMILES: O=C(Nc1cc(CN2CCOCC2)c2[nH]nc(N3C(=O)c4ccccc4C3=O)c2c1Oc1cc(F)ccc1Cl)c1cc(F)cc(C(F)(F)F)c1]

To a mixture of N-(4-(2-chloro-5-fluorophenoxy)-3-(1,3-dioxoisoindolin-2-yl)-7-formyl-1H-indazol-5-yl)-3-fluoro-5-(trifluoromethyl)benzamide (10 mg, 0.015 mmol) in DCM (0.2 mL) was added morpholine (3 mg, 0.03 mmol) and sodium triacetoxyborohydride (4.7 mg, 0.022 mmol). The resulting mixture was stirred at room temperature for 2 h before quenched by adding water. The mixture was extracted with ethyl acetate (2×30 mL). The combined organics were dried with anhydrous sodium sulfate, filtered, and concentrated under reduced pressure to afford the crude product which was used directly in next step without further purification. LCMS calculated for C₃₄H₂₄ClF₅N₅O₅ (M+H)⁺ m/z=712.1; found 712.2.

### Step 4: N-(3-amino-4-(2-chloro-5-fluorophenoxy)-7-(morpholinomethyl)-1H-indazol-5-yl)-3-fluoro-5-(trifluoromethyl)benzamide

To a mixture of N-(4-(2-chloro-5-fluorophenoxy)-3-(1,3-dioxoisoindolin-2-yl)-7-(morpholinomethyl)-1H-indazol-5-yl)-3-fluoro-5-(trifluoromethyl)benzamide (7 mg, 0.01 mmol) in MeOH (1 mL) were added hydrazine monohydrate (0.2 mL) at rt. The resulting mixture was stirred at rt for 1 h. The mixture was concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₆H₂₂ClF₅N₅O₃ (M+H)+ m/z=582.1; found 582.2.

### Example 22. N-(2-amino-3-(2-chloro-5-fluorophenoxy)-7,8-dihydro-6H-pyrazolo[4,5,1-ij]quinolin-4-yl)-3-fluoro-5-(trifluoromethyl)benzamide

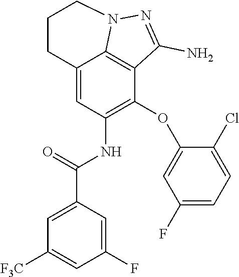 [SMILES: Nc1nn2c3c(cc(NC(=O)c4cc(F)cc(C(F)(F)F)c4)c(Oc4cc(F)ccc4Cl)c13)CCC2]

### Step 1: N-(4-(2-chloro-5-fluorophenoxy)-3-(1,3-dioxoisoindolin-2-yl)-7-vinyl-1H-indazol-5-yl)-3-fluoro-5-(trifluoromethyl)benzamide

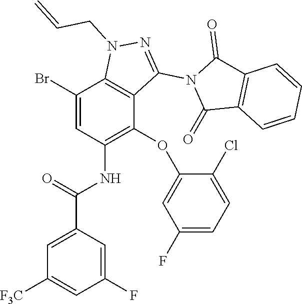 [SMILES: C=CCn1nc(N2C(=O)c3ccccc3C2=O)c2c(Oc3cc(F)ccc3Cl)c(NC(=O)c3cc(F)cc(C(F)(F)F)c3)cc(Br)c21]

N-(7-bromo-4-(2-chloro-5-fluorophenoxy)-3-(1,3-dioxoisoindolin-2-yl)-1H-indazol-5-yl)-3-fluoro-5-(trifluoromethyl)benzamide (Example 11, step 1, 100 mg, 0.13 mmol), was dissolved in DMF (1 mL) then 3-bromoprop-1-ene (23 mg, 0.2 mmol) and K₂CO₃ (54 mg, 0.4 mmol) were added. The resulting mixture was stirred at rt for 1 h, then diluted with water. The mixture was extracted with EtOAc (2×10 mL). The combined organics were dried with anhydrous sodium sulfate, filtered, and concentrated under reduced pressure to afford the crude product which was purified by silica gel chromatography to afford the title compound as a brown oil (66 mg, 70%). LCMS calculated for C₃₂H₁₈BrClF₅N₄O₄ (M+H)+ m/z=731.0; found 731.1.

### Step 2: N-(1-allyl-4-(2-chloro-5-fluorophenoxy)-3-(1,3-dioxoisoindolin-2-yl)-7-vinyl-1H-indazol-5-yl)-3-fluoro-5-(trifluoromethyl)benzamide

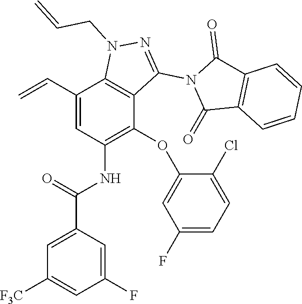 [SMILES: C=CCn1nc(N2C(=O)c3ccccc3C2=O)c2c(Oc3cc(F)ccc3Cl)c(NC(=O)c3cc(F)cc(C(F)(F)F)c3)cc(C=C)c21]

The mixture of N-(1-allyl-7-bromo-4-(2-chloro-5-fluorophenoxy)-3-(1,3-dioxoisoindolin-2-yl)-1H-indazol-5-yl)-3-fluoro-5-(trifluoromethyl)benzamide (66 mg, 0.088 mmol), potassium vinyltrifluoroborate (13 mg, 0.1 mmol), K₃PO₄ (42 mg, 0.2 mmol) and XphosPd G2 (7 mg, 0.01 mmol) in toluene (1 mL) and water (0.1 mL) was stirred at 100° C. for 4 h under nitrogen atmosphere. Upon cooling to room temperature, the mixture was diluted with water and extracted with DCM. The combined organics were dried with anhydrous sodium sulfate, filtered, and concentrated under reduced pressure to afford the crude product which was purified by silica gel chromatography to afford the title compound as a brown oil (30 mg, 50%). LCMS calculated for C₃₄H₂₁ClF₅N₄O₄ (M+H)+ m/z=679.1; found 679.1.

### Step 3: N-(3-(2-chloro-5-fluorophenoxy)-2-(1,3-dioxoisoindolin-2-yl)-8H-pyrazolo[4,5,1-ij]quinolin-4-yl)-3-fluoro-5-(trifluoromethyl)benzamide

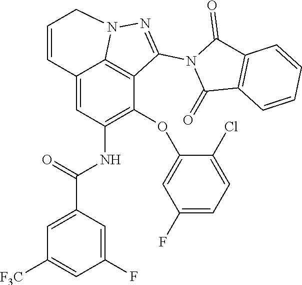 [SMILES: O=C(Nc1cc2c3c(c(N4C(=O)c5ccccc5C4=O)nn3CC=C2)c1Oc1cc(F)ccc1Cl)c1cc(F)cc(C(F)(F)F)c1]

The mixture of N-(1-allyl-4-(2-chloro-5-fluorophenoxy)-3-(1,3-dioxoisoindolin-2-yl)-7-vinyl-1H-indazol-5-yl)-3-fluoro-5-(trifluoromethyl)benzamide (30 mg, 0.044 mmol) and Grubbs catalyst G2 (4 mg, 0.004 mmol) in DCM (1 mL) was stirred at 40° C. for 12 h under nitrogen atmosphere. Upon completion, the mixture was purified by silica gel chromatography to afford the title compound as a brown oil (20 mg, 70%). LCMS calculated for C₃₂H₇ClF₅N₄O₄ (M+H)+ m/z=651.1; found 651.1.

### Step 4: N-(2-amino-3-(2-chloro-5-fluorophenoxy)-7,8-dihydro-6H-pyrazolo[4,5,1-ij]quinolin-4-yl)-3-fluoro-5-(trifluoromethyl)benzamide

The mixture of N-(3-(2-chloro-5-fluorophenoxy)-2-(1,3-dioxoisoindolin-2-yl)-8H-pyrazolo[4,5,1-ij]quinolin-4-yl)-3-fluoro-5-(trifluoromethyl)benzamide (20 mg, 0.03 mmol) and Pd/C (10% wt on carbon, 10 mg, 0.006 mmol) in MeOH (1 mL) was purged with hydrogen and stirred under 1 atm of hydrogen at rt for 6 h under nitrogen atmosphere. Upon completion, the mixture was added hydrazine monohydrate and stirred for 1 h before filtrated and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₄H₁₇ClF₅N₄O₂ (M+H)+ m/z=523.1; found 523.2.

### Example 23. N-(3-Amino-4-(2-chloro-5-fluorophenoxy)-7-(hydroxymethyl)-1H-indazol-5-yl)-3-fluoro-5-(trifluoromethyl)benzamide

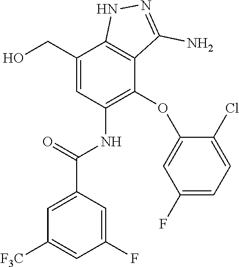 [SMILES: Nc1n[nH]c2c(CO)cc(NC(=O)c3cc(F)cc(C(F)(F)F)c3)c(Oc3cc(F)ccc3Cl)c12]

### Step 1: N-(4-(2-chloro-5-fluorophenoxy)-3-(1,3-dioxoisoindolin-2-yl)-7-(hydroxymethyl)-1H-indazol-5-yl)-3-fluoro-5-(trifluoromethyl)benzamide

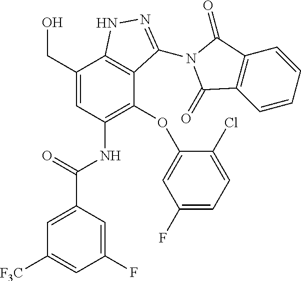 [SMILES: O=C(Nc1cc(CO)c2[nH]nc(N3C(=O)c4ccccc4C3=O)c2c1Oc1cc(F)ccc1Cl)c1cc(F)cc(C(F)(F)F)c1]

The mixture of N-(4-(2-chloro-5-fluorophenoxy)-3-(1,3-dioxoisoindolin-2-yl)-7-formyl-1H-indazol-5-yl)-3-fluoro-5-(trifluoromethyl)benzamide (Example 21, Step 2: 40 mg, 0.062 mmol) in DCM (1 mL) and MeOH (1 mL) was added NaBH₄ (4 mg, 0.1 mmol) under nitrogen atmosphere at 0° C. After completion, the reaction was diluted with water and extracted with DCM. The combined organics were dried with anhydrous sodium sulfate, filtered, and concentrated under reduced pressure to afford the crude product which was purified by silica gel chromatography to afford the title compound as a white solid (34 mg, 85%). LCMS calculated for C₃₀H₁₇ClF₅N₄O₅ (M+H)⁺ m/z=643.1; found 643.1.

### Step 2: N-(3-amino-4-(2-chloro-5-fluorophenoxy)-7-(hydroxymethyl)-1H-indazol-5-yl)-3-fluoro-5-(trifluoromethyl)benzamide

To a mixture of N-(4-(2-chloro-5-fluorophenoxy)-3-(1,3-dioxoisoindolin-2-yl)-7-(hydroxymethyl)-1H-indazol-5-yl)-3-fluoro-5-(trifluoromethyl)benzamide (34 mg, 0.052 mmol) in MeOH (1 mL) were added hydrazine monohydrate (0.2 mL) at rt. The resulting mixture was stirred at rt for 1 h. The mixture was concentrated under reduced pressure. The residue was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₂H₁₅ClF₅N₄O₃ (M+H)⁺ m/z=513.1; found 513.2; ¹H NMR (400 MHz, Methanol-d₄) δ 7.74 (s, 1H), 7.69-7.63 (m, 2H), 7.42 (s, 1H), 7.38 (dd, J=8.8, 5.7 Hz, 1H), 6.76 (m, 1H), 6.52 (dd, J=9.8, 2.9 Hz, 1H), 4.88 (s, 2H).

### Example 24. N-(3-Amino-7-chloro-4-(2-chloro-5-fluorophenoxy)-1-methyl-1H-indazol-5-yl)-3-fluoro-5-(trifluoromethyl)benzamide

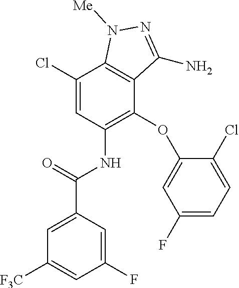 [SMILES: Cn1nc(N)c2c(Oc3cc(F)ccc3Cl)c(NC(=O)c3cc(F)cc(C(F)(F)F)c3)cc(Cl)c21]

### Step 1: N-(7-chloro-4-(2-chloro-5-fluorophenoxy)-3-(1,3-dioxoisoindolin-2-yl)-1H-indazol-5-yl)-3-fluoro-5-(trifluoromethyl)benzamide

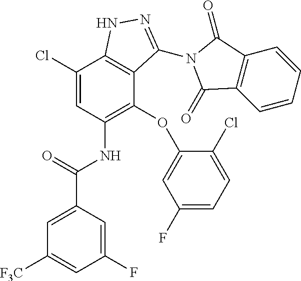 [SMILES: O=C(Nc1cc(Cl)c2[nH]nc(N3C(=O)c4ccccc4C3=O)c2c1Oc1cc(F)ccc1Cl)c1cc(F)cc(C(F)(F)F)c1]

To the mixture of N-(4-(2-chloro-5-fluorophenoxy)-3-(1,3-dioxoisoindolin-2-yl)-1H-indazol-5-yl)-3-fluoro-5-(trifluoromethyl)benzamide (Example 7, Step 1: 3 g, 4.9 mmol) in acetonitrile (30 mL) were added acetic acid (294 mg, 4.9 mmol) and N-Chlorosuccinimide (686 mg, 5.14 mmol). The reaction mixture was then stirred at 100° C. for 2 h under nitrogen atmosphere. Upon cooling to room temperature, the resulting mixture was concentrated under reduced pressure. The residue was purified by silica gel column chromatography, eluted with 50% ethyl acetate in petroleum ether to afford the desired product as a red solid (3 g, 94%). LCMS calculated for C₂₉H₁₄Cl₂F₅N₄O₄(M+H)⁺ m/z=647.0; found 647.1.

### Step 2: N-(7-chloro-4-(2-chloro-5-fluorophenoxy)-3-(1,3-dioxoisoindolin-2-yl)-1-methyl-1H-indazol-5-yl)-3-fluoro-5-(trifluoromethyl)benzamide

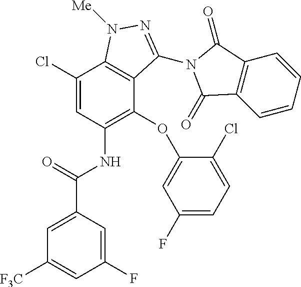 [SMILES: Cn1nc(N2C(=O)c3ccccc3C2=O)c2c(Oc3cc(F)ccc3Cl)c(NC(=O)c3cc(F)cc(C(F)(F)F)c3)cc(Cl)c21]

To the mixture of N-(7-chloro-4-(2-chloro-5-fluorophenoxy)-3-(1,3-dioxoisoindolin-2-yl)-1H-indazol-5-yl)-3-fluoro-5-(trifluoromethyl)benzamide (1.5 g, 2.32 mmol) and potassium carbonate (640 mg, 4.634 mmol) in N,N-Dimethylformamide (20 mL) was added iodomethane (493 mg, 3.48 mmol) dropwise at 0° C. The reaction was stirred for additional 1 h. The resulting mixture was diluted with water (200 mL). The aqueous layer was extracted with ethyl acetate (3×200 mL). The combined organic layer was washed with brine (2×200 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a yellow solid (700 mg, 45%). LCMS calculated for C₃₀H₁₆C₂F₅N₄O₄ (M+H)⁺ m/z=661.0; found 661.1.

### Step 3: N-(3-amino-7-chloro-4-(2-chloro-5-fluorophenoxy)-1-methyl-1H-indazol-5-yl)-3-fluoro-5-(trifluoromethyl)benzamide

To the mixture of N-(7-chloro-4-(2-chloro-5-fluorophenoxy)-3-(1,3-dioxoisoindolin-2-yl)-1-methyl-1H-indazol-5-yl)-3-fluoro-5-(trifluoromethyl)benzamide (670 mg, 1.0 mmol) in methanol (5 mL) was added hydrazine hydrate (98%, 1 mL) at room temperature. The reaction was stirred for additional 2 h. Upon cooling to 0° C., the mixture was neutralized to pH 7 with hydrochloric acid (2 M). The resulting mixture was extracted with ethyl acetate (3×100 mL), dried over anhydrous sodium sulfate. After filtration, the filtrate was concentrated under reduced pressure. The residue was purified by silica gel column chromatography, eluted with 40% ethyl acetate in dichloromethane to afford the desired product as a light-yellow solid (510 mg). LCMS calculated for C₂₂H₁₄C₁₂F₅N₄O₂(M+H)⁺ m/z=531.0; found 531.1; ¹H NMR (400 MHz, Chloroform-d) δ 8.30 (s, 1H), 7.79 (s, 1H), 7.66-7.59 (m, 2H), 7.53-7.47 (m, 1H), 7.43 (dd, J=8.8, 5.6 Hz, 1H), 6.83-6.74 (m, 1H), 6.46 (dd, J=9.2, 2.8 Hz, 1H), 4.21 (s, 3H).

### Example 25. N-(3-Amino-7-bromo-4-(2-chloro-5-fluorophenoxy)-1-methyl-1H-indazol-5-yl)-3-fluoro-5-(trifluoromethyl)benzamide

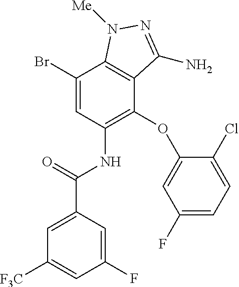 [SMILES: Cn1nc(N)c2c(Oc3cc(F)ccc3Cl)c(NC(=O)c3cc(F)cc(C(F)(F)F)c3)cc(Br)c21]

### Step 1: N-(7-bromo-4-(2-chloro-5-fluorophenoxy)-3-(1,3-dioxoisoindolin-2-yl)-1-methyl-1H-indazol-5-yl)-3-fluoro-5-(trifluoromethyl)benzamide

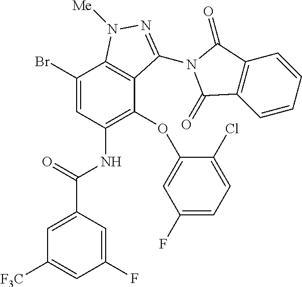 [SMILES: Cn1nc(N2C(=O)c3ccccc3C2=O)c2c(Oc3cc(F)ccc3Cl)c(NC(=O)c3cc(F)cc(C(F)(F)F)c3)cc(Br)c21]

To the mixture of N-(7-bromo-4-(2-chloro-5-fluorophenoxy)-3-(1,3-dioxoisoindolin-2-yl)-1H-indazol-5-yl)-3-fluoro-5-(trifluoromethyl)benzamide (Example 11, Step 1: 4.4 g, 6.36 mmol) and potassium carbonate (1.76 g, 12.7 mmol) in N,N-Dimethylformamide (50 mL) was added iodomethane (1.35 g, 9.54 mmol) dropwise at 0° C. The reaction was stirred at room temperature for 1 h. The resulting mixture was diluted with water (500 mL). The aqueous layer was extracted with ethyl acetate (3×300 mL). The combined organic layer was washed with brine (2×300 mL), dried over anhydrous sodium sulfate. After filtration, the filtrate was concentrated under reduced pressure. The residue was purified by silica gel column chromatography, eluted with 30% ethyl acetate in petroleum ether to afford the desired product as a yellow solid (2 g, 44%). LCMS calculated for C₃₀H₁₆BrClF₅N₄O₄ (M+H)⁺ m/z=705.0; found 705.1;

### Step 2: N-(3-amino-7-bromo-4-(2-chloro-5-fluorophenoxy)-1-methyl-1H-indazol-5-yl)-3-fluoro-5-(trifluoromethyl)benzamide

To the mixture of N-(7-bromo-4-(2-chloro-5-fluorophenoxy)-3-(1,3-dioxoisoindolin-2-yl)-1-methyl-1H-indazol-5-yl)-3-fluoro-5-(trifluoromethyl)benzamide (500 mg, 0.71 mmol) in methanol (30 mL) was added hydrazine hydrate (98%, 6 mL) at room temperature. The reaction was stirred for 2 h. Upon cooling to 0° C., the reaction was neutralized to pH 7 with hydrochloric acid (2 M). The resulting mixture was extracted with ethyl acetate (3×100 mL), dried over anhydrous sodium sulfate. After filtration, the filtrate was concentrated under reduced pressure. The residue was purified by silica gel column chromatography, eluted with 25% ethyl acetate in dichloromethane to afford the desired product as a light-yellow solid (400 mg). LCMS calculated for C₂₂H₁₄BrClF₅N₄O₂ (M+H)⁺ m/z=575.0; found 575.1; ¹H NMR (400 MHz, Methanol-d₄) δ 7.72 (s, 1H), 7.69-7.61 (m, 3H), 7.37 (dd, J=8.8, 5.6 Hz, 1H), 6.80-6.72 (m, 1H), 6.50 (dd, J=9.6, 2.8 Hz, 1H), 4.17 (s, 3H).

### Example 26. N-(3-Amino-4-(2-chloro-5-fluorophenoxy)-7-(1-isopropyl-1H-pyrazol-4-yl)-1-methyl-1H-indazol-5-yl)-3-fluoro-5-(trifluoromethyl)benzamide

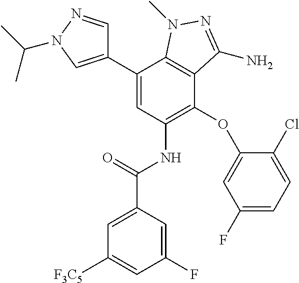

To a screw-cap vial equipped with a magnetic stir bar were placed N-[3-amino-7-bromo-4-(2-chloro-5-fluorophenoxy)-1-methylindazol-5-yl]-3-fluoro-5-(trifluoromethyl)benzamide (Example 25: 20 mg, 0.035 mmol), 1-isopropyl-4-(4,4,5,5-tetramethyl-1,3,2-dioxaborolan-2-yl)pyrazole (12 mg, 0.05 mmol), Pd(dppf)Cl₂·CH₂Cl₂ (3 mg, 0.004 mmol) and potassium carbonate (14 mg, 0.11 mmol). The vial was sealed with a Teflon-lined septum, evacuated and backfilled with nitrogen (this process was repeated a total of three times). Dioxane (2 mL) and water (0.4 mL) were added. The reaction was stirred at 80° C. for 1 h. Upon cooling to room temperature, the mixture was concentrated under reduced pressure. The residue was purified by silica gel column chromatography, eluted with 10% methanol in dichloromethane to afford crude product, which was purified by reversed-phase flash chromatography with the following conditions: column, C18 silica gel; mobile phase, acetonitrile in water (0.11% trifluoroacetic acid), 30% to 80% gradient in 20 min; detector, UV 254 nm. The collected fractions were lyophilized to afford the TFA salt of the desired product as an off-white solid. LCMS calculated for C₂₈H₂₃ClF₅N₆O₂(M+H)⁺ m/z=605.1; found 605.1. ¹H NMR (400 MHz, Chloroform-d) δ 8.17 (s, 1H), 7.79-7.61 (m, 2H), 7.64-7.57 (m, 2H), 7.54-7.47 (m, 1H), 7.47-7.42 (m, 1H), 6.84-6.78 (m, 1H), 6.52 (s, 1H), 5.04-4.61 (m, 3H), 3.63 (s, 3H), 1.61 (d, J=4.0 Hz, 6H).

### Example 27. N-(3-Amino-7-(4-carbamoylphenyl)-4-(2-chloro-5-fluorophenoxy)-1-methyl-1H-indazol-5-yl)-3-fluoro-5-(trifluoromethyl)benzamide

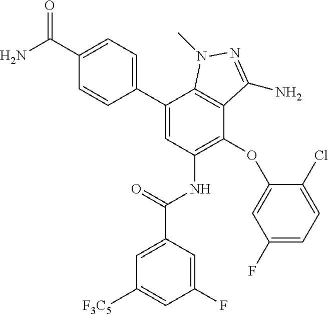

The title compound was prepared according to the procedure described in Example 26, using 4-(4,4,5,5-tetramethyl-1,3,2-dioxaborolan-2-yl)benzamide instead of 1-isopropyl-4-(4,4,5,5-tetramethyl-1,3,2-dioxaborolan-2-yl)pyrazole. LCMS calculated for C₂₉H₂₀ClF₅N₅O₃ (M+H)⁺ m/z=616.1; found 616.1; ¹H NMR (400 MHz, DMSO-d₆) δ 10.22 (s, 1H), 8.09 (s, 1H), 8.05-7.99 (m, 2H), 7.90 (d, J=8.4 Hz, 1H), 7.80-7.72 (m, 2H), 7.65-7.58 (m, 2H), 7.49 (dd, J=8.8, 5.6 Hz, 1H), 7.45 (s, 1H), 7.21 (s, 1H), 6.93-6.86 (m, 1H), 6.54 (dd, J=9.6, 2.8 Hz, 1H), 5.07 (s, 2H), 3.35 (s, 3H).

### Example 28. N-(3-Amino-4-(2-chloro-5-fluorophenoxy)-1-methyl-7-(1-(tetrahydrofuran-3-yl)-1H-pyrazol-4-yl)-1H-indazol-5-yl)-3-fluoro-5-(trifluoromethyl)benzamide

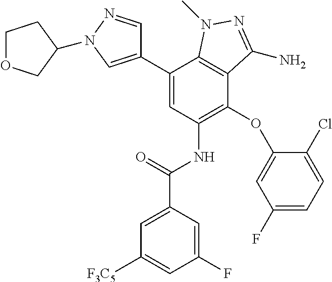

The title compound was prepared according to the procedure described in Example 26, using 1-(oxolan-3-yl)-4-(4,4,5,5-tetramethyl-1,3,2-dioxaborolan-2-yl)-1H-pyrazole instead of 1-isopropyl-4-(4,4,5,5-tetramethyl-1,3,2-dioxaborolan-2-yl)pyrazole. LCMS calculated for C₂₉H₂₃ClF₅N₆O₃ (M+H)⁺ m/z=633.1; found 633.1; ¹H NMR (400 MHz, DMSO-d₆) δ 10.18 (s, 1H), 8.11 (s, 1H), 7.90 (d, J=8.4 Hz, 1H), 7.81-7.71 (m, 3H), 7.49 (dd, J=8.8, 5.6 Hz, 1H), 7.16 (s, 1H), 6.92-6.85 (m, 1H), 6.47 (dd, J=9.6, 2.8 Hz, 1H), 5.15-5.05 (m, 1H), 4.06-3.93 (m, 3H), 3.89-3.82 (m, 1H), 3.54 (s, 3H), 2.48-2.33 (m, 2H).

### Example 29. N-(3-Amino-4-(2-chloro-5-fluorophenoxy)-1-methyl-7-(pyrimidin-5-yl)-1H-indazol-5-yl)-3-fluoro-5-(trifluoromethyl)benzamide

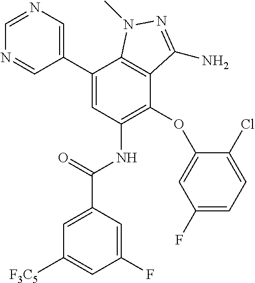

The title compound was prepared according to the procedure described in Example 26, using 5-(4,4,5,5-tetramethyl-1,3,2-dioxaborolan-2-yl)pyrimidine instead of 1-isopropyl-4-(4,4,5,5-tetramethyl-1,3,2-dioxaborolan-2-yl)pyrazole. LCMS calculated for C₂₆H₁₇ClF₅N₆O₂ (M+H)⁺ m/z=575.1; found 575.1; ¹H NMR (400 MHz, DMSO-d₆) δ 10.25 (s, 1H), 9.29 (s, 1H), 9.04 (s, 2H), 7.90 (d, J=8.4 Hz, 1H), 7.80-7.72 (m, 2H), 7.49 (dd, J=8.8, 5.6 Hz, 1H), 7.33 (s, 1H), 6.94-6.86 (m, 1H), 6.55 (dd, J=9.6, 2.8 Hz, 1H), 3.42 (s, 3H).

### Example 30. N-(3-Amino-4-(2-chloro-5-fluorophenoxy)-1-methyl-7-(1-methyl-1H-1,2,3-triazol-5-yl)-1H-indazol-5-yl)-3-fluoro-5-(trifluoromethyl)benzamide

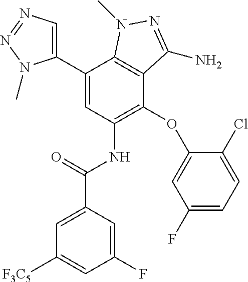

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (20 mg, 0.04 mmol), and XPhos Pd G3 (3 mg, 0.004 mmol). The vial was sealed with a Teflon-lined septum, evacuated and backfilled with nitrogen (this process was repeated a total of three times). 1-Methyl-5-(tributylstannyl)-1H-1,2,3-triazole (19.4 mg, 0.05 mmol) in dioxane (2 mL) was added. The resulting mixture was stirred at 100° C. for 16 h. Upon cooling to room temperature, the reaction mixture was concentrated under reduced pressure. The residue was purified by silica gel column chromatography, eluted with 10% methanol in dichloromethane to afford the crude product, which was then purified by reversed-phase flash chromatography with the following conditions (column: XBridge Prep Phenyl OBD Column 19*250 mm, 5 μm; mobile phase, acetonitrile in water (0.05% trifluoroacetic acid), Flow rate: 60 mL/min; Gradient: 29% B to 49% B in 10 min, 49% B to 49% B in 5 min; detector: UV 254/220 nm). Eluted fractions were collected and lyophilized to afford the TFA salt of the desired product as a light pink solid. LCMS calculated for C₂₅H₁₈ClF₅N₇O₂ (M+H)⁺ m/z=578.1; found 578.1; ¹H NMR (400 MHz, DMSO-d₆) δ 10.22 (s, 1H), 8.03 (s, 1H), 7.90 (d, J=8.4 Hz, 1H), 7.78-7.70 (m, 2H), 7.47 (dd, J=8.8, 5.6 Hz, 1H), 7.35 (s, 1H), 6.93-6.86 (m, 1H), 6.61 (dd, J=9.6, 2.8 Hz, 1H), 3.93 (s, 3H), 3.28 (s, 3H).

### Example 31. N-(3-Amino-4-(2-chloro-5-fluorophenoxy)-7-(cyanomethyl)-1-methyl-1H-indazol-5-yl)-3-fluoro-5-(trifluoromethyl)benzamide

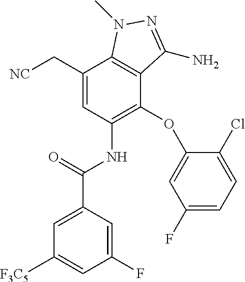

To a screw-cap vial equipped with a magnetic stir bar were added N-(3-amino-7-bromo-4-(2-chloro-5-fluorophenoxy)-1-methyl-1H-indazol-5-yl)-3-fluoro-5-(trifluoromethyl)benzamide (20 mg, 0.04 mmol), 4-(4,4,5,5-tetramethyl-1,3,2-dioxaborolan-2-yl)-1,2-oxazole (10 mg, 0.05 mmol), 1,1′-bis(diphenylphosphino)ferrocene-palladium(II)dichloride dichloromethane complex (5.7 mg, 0.007 mmol) and potassium fluoride (6 mg, 0.11 mmol). The vial was sealed with a Teflon-lined septum, evacuated and backfilled with nitrogen (this process was repeated a total of three times). Dimethyl sulfoxide (0.5 mL) and water (0.2 mL) were added. The reaction was stirred at 90° C. for 2 h. Upon cooling to room temperature, the resulting mixture was diluted with water (10 mL), and then extracted with ethyl acetate (3×10 mL). The combined organic layers were washed with brine (2×10 mL), dried over anhydrous sodium sulfate. After filtration, the filtrate was concentrated under reduced pressure. The residue was dissolved in methanol (0.5 mL) and water (0.2 mL). Potassium fluoride (6 mg, 0.11 mmol) was added at room temperature. The resulting mixture was stirred at 80° C. for additional 3 h. Upon cooling to room temperature, the resulting mixture was concentrated under reduced pressure. The residue was purified by Prep-TLC (8% methanol in dichloromethane) to afford the crude product which was further purified by reverse phase flash with the following conditions (Column: Xselect CSH C18 OBD Column 30*150 mm 5 m; Mobile phase: acetonitrile in water (0.1% 2,2,2-trifluoroacetic acid); Flow rate: 60 mL/min; Gradient: 38% B to 58% B in 8 min; detector: 254/220 nm). Fractions were collected and lyophilized to afford the TFA salt of the desired product as a white solid. LCMS calculated for C₂₄H₁₆ClF₅N₅O₂ (M+H)⁺ m/z=536.1; found 536.1. ¹H NMR (400 MHz, DMSO-d₆) δ 10.20 (s, 1H), 7.94-7.88 (m, 1H), 7.80-7.73 (m, 2H), 7.50-7.43 (m, 1H), 7.30 (s, 1H), 6.91-6.83 (m, 1H), 6.49-6.43 (m, 1H), 5.02 (s, 2H), 4.58 (s, 2H), 4.04 (s, 3H).

### Example 32. N-(6-Amino-7-(2-chloro-5-fluorophenoxy)-2-oxo-2,3-dihydro-1H-pyrazolo[1,5,4-de]quinoxalin-8-yl)-3-fluoro-5-(trifluoromethyl)benzamide

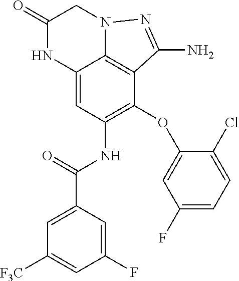 [SMILES: Nc1nn2c3c(cc(NC(=O)c4cc(F)cc(C(F)(F)F)c4)c(Oc4cc(F)ccc4Cl)c13)NC(=O)C2]

### Step 1: Tert-butyl 2-(7-bromo-4-(2-chloro-5-fluorophenoxy)-3-(1,3-dioxoisoindolin-2-yl)-5-(3-fluoro-5-(trifluoromethyl)benzamido)-1H-indazol-1-yl)acetate

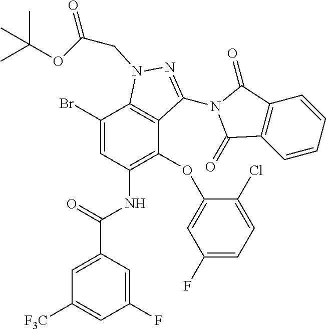 [SMILES: CC(C)(C)OC(=O)Cn1nc(N2C(=O)c3ccccc3C2=O)c2c(Oc3cc(F)ccc3Cl)c(NC(=O)c3cc(F)cc(C(F)(F)F)c3)cc(Br)c21]

To the mixture of N-[7-bromo-4-(2-chloro-5-fluorophenoxy)-3-(1,3-dioxoisoindol-2-yl)-1H-indazol-5-yl]-3-fluoro-5-(trifluoromethyl)benzamide (Example 11, Step 1: 300 mg, 0.43 mmol) and potassium carbonate (120 mg, 0.86 mmol) in N,N-Dimethylformamide (5 mL) was added tert-butyl 2-bromoacetate (85 mg, 0.43 mmol). The resulting mixture was stirred at room temperature for 1 h. The reaction was diluted with water (50 mL). The resulting mixture was extracted with ethyl acetate (3×50 mL). The combined organic layers were washed with brine (3×50 mL), dried over anhydrous sodium sulfate. After filtration, the filtrate was concentrated under reduced pressure. The residue was purified by silica gel column chromatography, eluted with 30% ethyl acetate in petroleum ether to afford the desired product as a light pink solid (90 mg, 26%). LCMS calculated for C₃₅H₂₄BrClF₅N₄O₆ (M+H)⁺ m/z=805.0; found 805.1.

### Step 2: 2-(7-Bromo-4-(2-chloro-5-fluorophenoxy)-3-(1,3-dioxoisoindolin-2-yl)-5-(3-fluoro-5-(trifluoromethyl)benzamido)-1H-indazol-1-yl)acetic acid

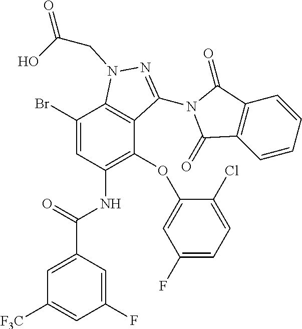 [SMILES: O=C(O)Cn1nc(N2C(=O)c3ccccc3C2=O)c2c(Oc3cc(F)ccc3Cl)c(NC(=O)c3cc(F)cc(C(F)(F)F)c3)cc(Br)c21]

To the mixture of tert-butyl 2-[7-bromo-4-(2-chloro-5-fluorophenoxy)-3-(1,3-dioxoisoindol-2-yl)-5-[3-fluoro-5-(trifluoromethyl)benzamido]indazol-1-yl]acetate (90 mg, 0.11 mmol) in dichloromethane (3 mL) was added trifluoroacetic acid (1 mL). The reaction was stirred at room temperature for 3 h. The mixture was concentrated under reduced pressure to afford the crude product as a yellow solid (90 mg). The crude product was used in the next step directly without further purification. LCMS calculated for C₃₁H₁₆BrClF₅N₄O₆ (M+H)⁺ m/z=749.0; found 749.0;

### Step 3: N-(1-(2-amino-2-oxoethyl)-7-bromo-4-(2-chloro-5-fluorophenoxy)-3-(1,3-dioxoisoindolin-2-yl)-1H-indazol-5-yl)-3-fluoro-5-(trifluoromethyl)benzamide

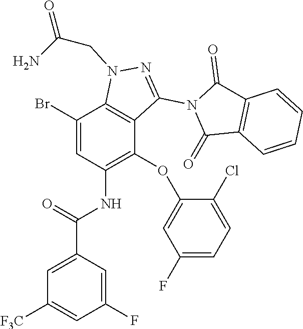 [SMILES: NC(=O)Cn1nc(N2C(=O)c3ccccc3C2=O)c2c(Oc3cc(F)ccc3Cl)c(NC(=O)c3cc(F)cc(C(F)(F)F)c3)cc(Br)c21]

To the mixture of [7-bromo-4-(2-chloro-5-fluorophenoxy)-3-(1,3-dioxoisoindol-2-yl)-5-[3-fluoro-5-(trifluoromethyl)benzamido]indazol-1-yl]acetic acid (90 mg, 0.12 mmol) in DMF (2 mL) was added 2-(7-azabenzotriazol-1-yl)-N,N,N′,N′-tetramethyluronium hexafluorophosphate (91 mg, 0.24 mmol). The mixture was stirred at room temperature for 15 min, followed by the addition of ammonium bicarbonate (19 mg, 0.24 mmol) and N,N-diisopropylethylamine (46 mg, 0.36 mmol). The reaction was stirred at room temperature for additional 1 h. The mixture was purified by reversed-phase flash chromatography with the following conditions: column, C18 silica gel; mobile phase, acetonitrile in water (10 mmol/L ammonium bicarbonate), 10% to 70% gradient in 20 min; detector, UV 254 nm. The collected fractions to afford the desired product as a white solid (45 mg, 52%). LCMS calculated for C₃₁H₁₇BrClF₅N₅O₅ (M+H)⁺ m/z=748.0; found 748.2;

### Step 4: 2-((7-(2-chloro-5-fluorophenoxy)-8-(3-fluoro-5-(trifluoromethyl)benzamido)-2-oxo-2,3-dihydro-1H-pyrazolo[1,5,4-de]quinoxalin-6-yl)carbamoyl)benzoic acid

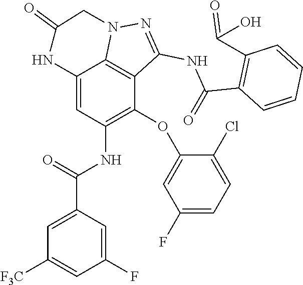 [SMILES: O=C1Cn2nc(NC(=O)c3ccccc3C(=O)O)c3c(Oc4cc(F)ccc4Cl)c(NC(=O)c4cc(F)cc(C(F)(F)F)c4)cc(c32)N1]

To a screw-cap vial equipped with a magnetic stir bar were added N-[7-bromo-1-(carbamoylmethyl)-4-(2-chloro-5-fluorophenoxy)-3-(1,3-dioxoisoindol-2-yl)indazol-5-yl]-3-fluoro-5-(trifluoromethyl)benzamide (45 mg, 0.06 mmol), cesium carbonate (39 mg, 0.12 mmol) and tBuXPhos Pd G3 (9.5 mg, 0.012 mmol). The vial was sealed with a Teflon-lined septum, evacuated and backfilled with nitrogen (this process was repeated a total of three times). 1,4-Dioxane (1 mL) was added. The reaction was stirred at 90° C. for 1 h. Upon cooling to room temperature, the mixture was purified by reversed-phase flash chromatography with the following conditions: column, C18 silica gel; mobile phase, acetonitrile in water (10 mmol/L ammonium bicarbonate), 10% to 50% gradient in 20 min; detector, UV 254 nm. The collected fractions to afford the desired product as a white solid (25 mg, 61%). LCMS calculated for C₃₁H₁₈ClF₅N₅O₆ (M+H)⁺ m/z=686.1; found 686.2;

### Step 5: N-(7-(2-chloro-5-fluorophenoxy)-6-(1,3-dioxoisoindolin-2-yl)-2-oxo-2,3-dihydro-1H-pyrazolo[1,5,4-de]quinoxalin-8-yl)-3-fluoro-5-(trifluoromethyl)benzamide

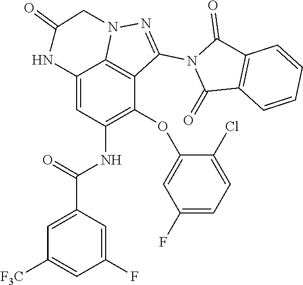 [SMILES: O=C1Cn2nc(N3C(=O)c4ccccc4C3=O)c3c(Oc4cc(F)ccc4Cl)c(NC(=O)c4cc(F)cc(C(F)(F)F)c4)cc(c32)N1]

The mixture of 2-((7-(2-chloro-5-fluorophenoxy)-8-(3-fluoro-5-(trifluoromethyl)benzamido)-2-oxo-2,3-dihydro-1H-pyrazolo[1,5,4-de]quinoxalin-6-yl)carbamoyl)benzoic acid, 2-(7-azabenzotriazol-1-yl)-N,N,N′,N′-tetramethyluronium hexafluorophosphate (21 mg, 0.055 mmol) and N,N-diisopropylethylamine (9.4 mg, 0.07 mmol) in DMF (1 mL) was stirred at room temperature for 30 min. The reaction mixture was diluted with ethyl acetate (50 mL). The resulting mixture was washed with brine (3×30 mL) and dried over anhydrous sodium sulfate. After filtration, the filtrate was concentrated under reduced pressure. The residue was purified by silica gel column chromatography, eluted with 8% methanol in dichloromethane to afford the desired product as a white solid (12 mg, 24%). LCMS calculated for C₃₁H₁₆ClF₅N₅O₅ (M+H)⁺ m/z=668.1; found 668.2;

### Step 6: N-(6-amino-7-(2-chloro-5-fluorophenoxy)-2-oxo-2,3-dihydro-1H-pyrazolo[1,5,4-de]quinoxalin-8-yl)-3-fluoro-5-(trifluoromethyl)benzamide

To the mixture of N-(7-(2-chloro-5-fluorophenoxy)-6-(1,3-dioxoisoindolin-2-yl)-2-oxo-2,3-dihydro-1H-pyrazolo[11,5,4-de]quinoxalin-8-yl)-3-fluoro-5-(trifluoromethyl)benzamide (12 mg, 0.02 mmol) in methanol (0.5 mL) was added hydrazine hydrate (98%, 0.1 mL) at room temperature. The reaction was stirred for 30 min. The resulting mixture was concentrated under reduced pressure. The residue was purified by Prep-HPLC with the following conditions (Column: YMC-Actus Triart C18 ExRS 30*150 mm, 5 μm; Mobile Phase: acetonitrile in water (10 mmol/L ammonium bicarbonate); Flow rate: 60 mL/min; Gradient: 34% B to 51.5% B in 10 min, 51.5% B to 51.5% B in 5 min; Detector: 254/220 nm). Eluted fractions were collected and lyophilized to afford the desired product as a white solid. LCMS calculated for C₂₃H₁₄ClF₅N₅O₃ (M+H)⁺ m/z=538.1; found 538.1; ¹H NMR (400 MHz, DMSO-d₆) δ 11.07 (s, 1H), 10.14 (s, 1H), 7.94-7.87 (m, 1H), 7.86-7.78 (m, 2H), 7.47 (dd, J=8.8, 6.0 Hz, 1H), 6.91-6.83 (m, 1H), 6.63 (s, 1H), 6.51-6.45 (m, 1H), 5.06 (s, 2H), 4.91 (s, 2H).

### Example 33. N-(3-Amino-1-(2-amino-2-oxoethyl)-7-bromo-4-(2-chloro-5-fluorophenoxy)-1H-indazol-5-yl)-3-fluoro-5-(trifluoromethyl)benzamide

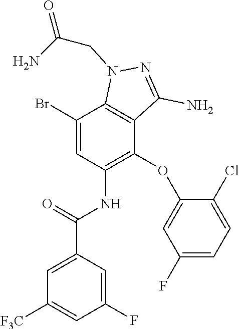 [SMILES: NC(=O)Cn1nc(N)c2c(Oc3cc(F)ccc3Cl)c(NC(=O)c3cc(F)cc(C(F)(F)F)c3)cc(Br)c21]

To the mixture of N-(1-(2-amino-2-oxoethyl)-7-bromo-4-(2-chloro-5-fluorophenoxy)-3-(1,3-dioxoisoindolin-2-yl)-1H-indazol-5-yl)-3-fluoro-5-(trifluoromethyl)benzamide (Example 32, Step 3: 25 mg, 0.03 mmol) in methanol (1 mL) was added hydrazine hydrate (98%, 0.2 mL) at room temperature. The reaction was stirred for 30 min. The resulting mixture was purified by Prep-HPLC with the following conditions (Column: Xselect CSH C18 OBD Column 30*150 mm 5 m; Mobile Phase: acetonitrile in water (0.05% trifluoroacetic acid); Flow rate: 60 mL/min; Gradient: 34% B to 54% B in 8 min, 54% B to 54% B in 5 min; Detector: 254/220 nm). Eluted fractions were collected and lyophilized to afford the TFA salt of the desired product as a white solid. LCMS calculated for C₂₃H₁₅BrClF₅N₅O₃ (M+H)⁺ m/z=618.0; found 618.0; ¹H NMR (400 MHz, DMSO-d₆) δ 10.22 (s, 1H), 7.94-7.87 (m, 1H), 7.79-7.71 (m, 2H), 7.59 (s, 1H), 7.50-7.39 (m, 2H), 7.23 (s, 1H), 6.92-6.84 (m, 1H), 6.56-6.49 (m, 1H), 5.16 (s, 2H), 5.06 (s, 2H).

### Example 34. N-(3-Amino-7-chloro-4-(2-chloro-5-fluorophenoxy)-1-(2-cyanoethyl)-1H-indazol-5-yl)-3-fluoro-5-(trifluoromethyl)benzamide

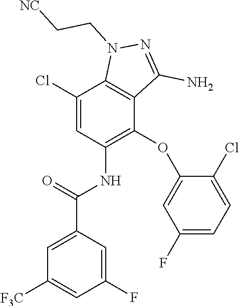 [SMILES: N#CCCn1nc(N)c2c(Oc3cc(F)ccc3Cl)c(NC(=O)c3cc(F)cc(C(F)(F)F)c3)cc(Cl)c21]

### Step 1: N-(7-chloro-4-(2-chloro-5-fluorophenoxy)-1-(2-cyanoethyl)-3-(1,3-dioxoisoindolin-2-yl)-1H-indazol-5-yl)-3-fluoro-5-(trifluoromethyl)benzamide

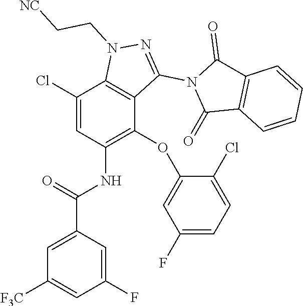 [SMILES: N#CCCn1nc(N2C(=O)c3ccccc3C2=O)c2c(Oc3cc(F)ccc3Cl)c(NC(=O)c3cc(F)cc(C(F)(F)F)c3)cc(Cl)c21]

To the mixture of N-[7-chloro-4-(2-chloro-5-fluorophenoxy)-3-(1,3-dioxoisoindol-2-yl)-1H-indazol-5-yl]-3-fluoro-5-(trifluoromethyl)benzamide (Example 24, Step 1: 20 mg, 0.03 mmol) and 3-bromopropanenitrile (8.3 mg, 0.06 mmol) in N,N-Dimethylformamide (1 mL) were added cesium carbonate (13 mg, 0.09 mmol) and potassium iodide (10 mg, 0.06 mmol) at room temperature. The resulting mixture was stirred at 60° C. for 16 h. Upon cooling to room temperature. The resulting mixture was purified by reversed-phase flash chromatography with the following conditions: column, C18 silica gel; mobile phase, acetonitrile in water (0.1% aqueous ammonia), 30% to 95% gradient in 20 min; detector, UV 254 nm. The collected fractions were lyophilized to afford the desired product as a white solid (10 mg, 46%). LCMS calculated for C₃₂H₁₅Cl₂F₅N₅O₄ (M−H⁻ m/z=698.0; found 698.1.

### Step 2: N-(3-amino-7-chloro-4-(2-chloro-5-fluorophenoxy)-1-(2-cyanoethyl)-1H-indazol-5-yl)-3-fluoro-5-(trifluoromethyl)benzamide

To the mixture of N-[7-chloro-4-(2-chloro-5-fluorophenoxy)-1-(2-cyanoethyl)-3-(1,3-dioxoisoindol-2-yl)indazol-5-yl]-3-fluoro-5-(trifluoromethyl)benzamide (10 mg, 0.014 mmol) in methanol (0.5 mL) was added hydrazine hydrate (98%, 0.2 mL) at room temperature. The resulting mixture was stirred at room temperature for 1 h, and then was concentrated under reduced pressure. The residue was purified by Prep-HPLC with the following conditions (Column: Xselect CSH C18 OBD Column 30*150 mm 5 m; Mobile Phase: acetonitrile in water (0.1% 2,2,2-trifluoroacetic acid); Flow rate: 60 mL/min; Gradient: 42% B to 62% B in 8 min; detector, UV 254/220 nm). Eluted fractions were collected and lyophilized to afford the TFA salt of the desired product as a white solid. LCMS calculated for C₂₄H₁₅Cl₂F₅N₅O₂ (M+H)⁺ m/z=570.1; found 570.1; ¹H NMR (400 MHz, DMSO-d₆) δ 10.23 (s, 1H), 7.95-7.89 (m, 1H), 7.78-7.71 (m, 2H), 7.53-7.43 (m, 2H), 6.93-6.84 (m, 1H), 6.56-6.49 (m, 1H), 5.32 (s, 2H), 4.74 (t, J=6.4 Hz, 2H), 3.06 (t, J=6.4 Hz, 2H).

### Example 35. N-(3-(2-Aminoacetamido)-7-chloro-4-(2-chloro-5-fluorophenoxy)-1-methyl-1H-indazol-5-yl)-3-fluoro-5-(trifluoromethyl)benzamide

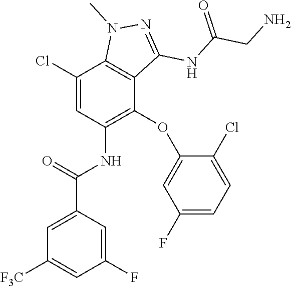 [SMILES: Cn1nc(NC(=O)CN)c2c(Oc3cc(F)ccc3Cl)c(NC(=O)c3cc(F)cc(C(F)(F)F)c3)cc(Cl)c21]

### Step 1: Tert-butyl (2-((7-chloro-4-(2-chloro-5-fluorophenoxy)-5-(3-fluoro-5-(trifluoromethyl)benzamido)-1-methyl-1H-indazol-3-yl)amino)-2-oxoethyl)carbamate

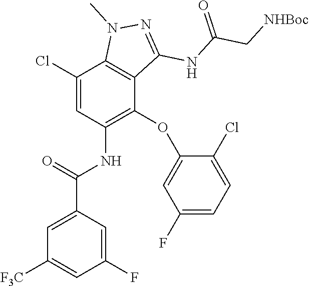 [SMILES: Cn1nc(NC(=O)CNC(=O)OC(C)(C)C)c2c(Oc3cc(F)ccc3Cl)c(NC(=O)c3cc(F)cc(C(F)(F)F)c3)cc(Cl)c21]

To the mixture of [(tert-butoxycarbonyl)amino]acetic acid (20 mg, 0.11 mmol) in N,N-Dimethylformamide (1 mL) was added 2-(7-azabenzotriazol-1-yl)-N,N,N′,N′-tetramethyluronium hexafluorophosphate (54 mg, 0.14 mmol) at room temperature. The mixture was stirred for 15 min, followed by the addition of N-(3-amino-7-chloro-4-(2-chloro-5-fluorophenoxy)-1-methyl-1H-indazol-5-yl)-3-fluoro-5-(trifluoromethyl)benzamide (Example 24: 30 mg, 0.056 mmol) and N,N-diisopropylethylamine (22 mg, 0.17 mmol). The reaction mixture was stirred at 60° C. for additional 16 h. Upon cooling to room temperature, the resulting mixture was diluted with water (20 mL). The resulting mixture was extracted with ethyl acetate (3×20 mL). The combined organic layers were washed with brine (3×20 mL), dried over anhydrous sodium sulfate. After filtration, the filtrate was concentrated under reduced pressure. The residue was purified by silica gel column chromatography, eluted with 80% ethyl acetate in petroleum ether to afford the desired product as a yellow solid (20 mg, 51%). LCMS calculated for C₂₉H₂₅Cl₂F₅N₅O₅(M+H)⁺ m/z=688.1; found 688.2.

### Step 2: N-(3-(2-aminoacetamido)-7-chloro-4-(2-chloro-5-fluorophenoxy)-1-methyl-1H-indazol-5-yl)-3-fluoro-5-(trifluoromethyl)benzamide

To the mixture of tert-butyl (2-((7-chloro-4-(2-chloro-5-fluorophenoxy)-5-(3-fluoro-5-(trifluoromethyl)benzamido)-1-methyl-1H-indazol-3-yl)amino)-2-oxoethyl)carbamate (20 mg, 0.03 mmol) in dichloromethane (0.5 mL) was added trifluoroacetic acid (0.1 mL) at room temperature. The reaction was stirred for 1 h. The resulting mixture was concentrated under reduced pressure. The residue was purified by Prep-HPLC with the following conditions (Column: Sunfire prep C18 column, 30*150 mm, 5 m; Mobile Phase: methanol in water (0.05% trifluoroacetic acid); Flow rate: 60 mL/min; Gradient: 40% B to 60% B in 10 min, 60% B to 60% B in 5 min; Detector: 254/220 nm). Eluted fractions were collected and lyophilized to afford the TFA salt of the desired product as a white solid. LCMS calculated for C₂₄H₁₇Cl₂F₅N₅O₃(M+H)⁺ m/z=588.1; found 588.1; ¹H NMR (400 MHz, DMSO-d₆) δ 10.37 (s, 1H), 10.31 (s, 1H), 8.15-8.05 (m, 2H), 7.96-7.90 (m, 1H), 7.79-7.72 (m, 2H), 7.67 (s, 1H), 7.50-7.42 (m, 1H), 6.90-6.81 (m, 1H), 6.40-6.33 (m, 1H), 4.34 (s, 3H), 3.54 (s, 2H).

### Example 36. N-(2-Amino-3-(2-chloro-5-fluorophenoxy)-8-oxo-6,7,8,9-tetrahydro-[1,4]diazepino[6,7,1-hi]indazol-4-yl)-3-fluoro-5-(trifluoromethyl)benzamide

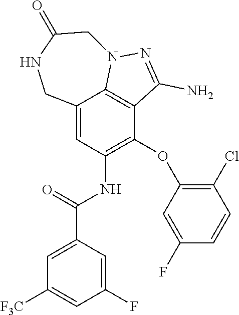 [SMILES: Nc1nn2c3c(cc(NC(=O)c4cc(F)cc(C(F)(F)F)c4)c(Oc4cc(F)ccc4Cl)c13)CNC(=O)C2]

### Step 1: N-(7-bromo-4-(2-chloro-5-fluorophenoxy)-3-(1,3-dioxoisoindolin-2-yl)-1-(tetrahydro-2H-pyran-2-yl)-1H-indazol-5-yl)-3-fluoro-5-(trifluoromethyl)benzamide

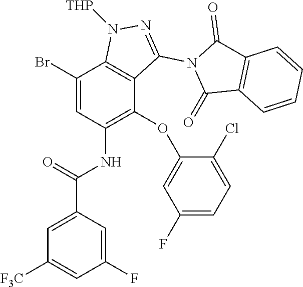

To the mixture of N-(7-bromo-4-(2-chloro-5-fluorophenoxy)-3-(1,3-dioxoisoindolin-2-yl)-1H-indazol-5-yl)-3-fluoro-5-(trifluoromethyl)benzamide (Example 11, Step 1: 2 g, 2.9 mmol) in dichloromethane (20 mL) were added p-toluenesulfonic acid (50 mg, 0.29 mmol) and 3,4-dihydro-2H-pyran (486 mg, 5.8 mmol) at room temperature. The reaction was stirred for 16 h. The resulting mixture was concentrated under reduced pressure. The residue was purified by silica gel column chromatography, eluted with 20% ethyl acetate in petroleum ether to afford the desired product as a pink solid (1.3 g, 58%). LCMS calculated for C₃₄H₂₀BrClF₅N₄O₅ (M−H)⁻ m/z=773.0; found 773.1.

### Step 2: N-(4-(2-chloro-5-fluorophenoxy)-3-(1,3-dioxoisoindolin-2-yl)-7-(hydroxymethyl)-1-(tetrahydro-2H-pyran-2-yl)-1H-indazol-5-yl)-3-fluoro-5-(trifluoromethyl)benzamide

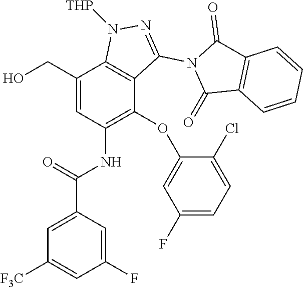

To a screw-cap vial equipped with a magnetic stir bar were placed N-(7-bromo-4-(2-chloro-5-fluorophenoxy)-3-(1,3-dioxoisoindolin-2-yl)-1-(tetrahydro-2H-pyran-2-yl)-1H-indazol-5-yl)-3-fluoro-5-(trifluoromethyl)benzamide (300 mg, 0.39 mmol), XPhos Pd G3 (65 mg, 0.08 mmol) and (tributylstannyl)methanol (248 mg, 0.77 mmol). The vial was sealed with a Teflon-lined septum, evacuated and backfilled with nitrogen (this process was repeated a total of three times). 1,4-Dioxane (5 mL) was added. The resulting mixture was stirred at 80° C. for 3 h. Upon cooling to room temperature, the resulting mixture was concentrated under reduced pressure. The residue was purified by silica gel column chromatography, eluted with 50% ethyl acetate in petroleum ether to afford the desired product as a white solid (230 mg, 82%). LCMS calculated for C₃₅H₂₅ClF₅N₄O₆ (M+H)⁺ m/z=727.1; found 727.2.

### Step 3: N-(7-(azidomethyl)-4-(2-chloro-5-fluorophenoxy)-3-(1,3-dioxoisoindolin-2-yl)-1-(tetrahydro-2H-pyran-2-yl)-1H-indazol-5-yl)-3-fluoro-5-(trifluoromethyl)benzamide

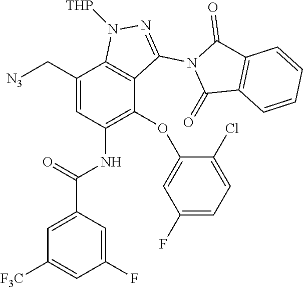

To the mixture of N-(4-(2-chloro-5-fluorophenoxy)-3-(1,3-dioxoisoindolin-2-yl)-7-(hydroxymethyl)-1-(tetrahydro-2H-pyran-2-yl)-1H-indazol-5-yl)-3-fluoro-5-(trifluoromethyl)benzamide (230 mg, 0.32 mmol) in tetrahydrofuran (5 mL) were added diphenyl azidophosphate (103 uL, 0.47 mmol) and 1,8-diazabicyclo[5.4.0]undec-7-ene (95 uL, 0.63 mmol) at room temperature. The resulting mixture was stirred for 4 h. The reaction was quenched with water. The resulting mixture was extracted with ethyl acetate (3×20 mL), dried over anhydrous sodium sulfate. After filtration, the filtrate was concentrated under reduced pressure. The residue was purified by silica gel column chromatography, eluted with 30% ethyl acetate in petroleum ether to afford the desired product as a white solid (210 mg, 88%). LCMS calculated for C₃₅H₂₂ClF₅N₇O₅ (M−H)⁻ m/z=750.1; found 750.2.

### Step 4: N-(7-(aminomethyl)-4-(2-chloro-5-fluorophenoxy)-3-(1,3-dioxoisoindolin-2-yl)-1-(tetrahydro-2H-pyran-2-yl)-1H-indazol-5-yl)-3-fluoro-5-(trifluoromethyl)benzamide

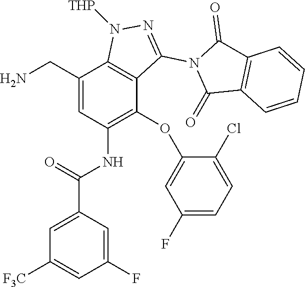

To the mixture of N-(7-(azidomethyl)-4-(2-chloro-5-fluorophenoxy)-3-(1,3-dioxoisoindolin-2-yl)-1-(tetrahydro-2H-pyran-2-yl)-1H-indazol-5-yl)-3-fluoro-5-(trifluoromethyl)benzamide (210 mg, 0.28 mmol) in tetrahydrofuran (3 mL) and water (3 mL) was added triphenylphosphine (146 mg, 0.56 mmol) at room temperature. The reaction was stirred at room temperature for 16 h. The resulting mixture was concentrated under reduced pressure. The residue was purified by silica gel column chromatography, eluted with 7% methanol in dichloromethane to afford the desired product as a yellow solid (130 mg, 64%). LCMS calculated for C₃₅H₂₆ClF₅N₅O₅ (M+H)⁺ m/z=726.2; found 726.2.

### Step 5: N-(4-(2-chloro-5-fluorophenoxy)-7-((2-chloroacetamido)methyl)-3-(1,3-dioxoisoindolin-2-yl)-1-(tetrahydro-2H-pyran-2-yl)-1H-indazol-5-yl)-3-fluoro-5-(trifluoromethyl)benzamide

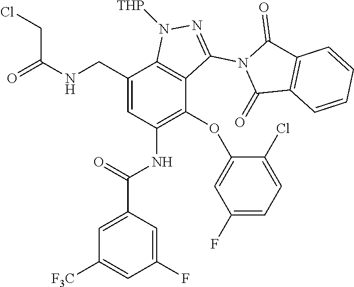

To the mixture of N-(7-(aminomethyl)-4-(2-chloro-5-fluorophenoxy)-3-(1,3-dioxoisoindolin-2-yl)-1-(tetrahydro-2H-pyran-2-yl)-1H-indazol-5-yl)-3-fluoro-5-(trifluoromethyl)benzamide (60 mg, 0.08 mmol) and triethylamine (23 uL, 0.17 mmol) in dichloromethane (1 mL) was added 2-chloroacetyl chloride (11 mg, 0.1 mmol) at room temperature. The reaction was stirred at room temperature for 1 h. The mixture was quenched with water at room temperature. The resulting mixture was extracted with dichloromethane (3×20 mL), dried over anhydrous sodium sulfate. After filtration, the filtrate was concentrated under reduced pressure. The residue was purified by silica gel column chromatography, eluted with 20% ethyl acetate in dichloromethane to afford the desired product as a white solid (55 mg, 83%). LCMS calculated for C₃₇H₂₅Cl₂F₅N₅O₆(M−H)⁻ m/z=800.1; found 800.0.

### Step 6: N-(4-(2-chloro-5-fluorophenoxy)-7-((2-chloroacetamido)methyl)-3-(1,3-dioxoisoindolin-2-yl)-1H-indazol-5-yl)-3-fluoro-5-(trifluoromethyl)benzamide

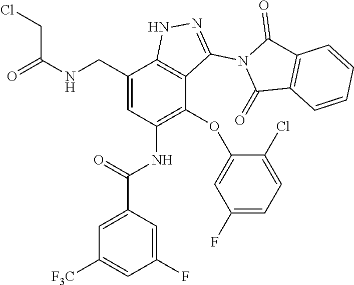 [SMILES: O=C(CCl)NCc1cc(NC(=O)c2cc(F)cc(C(F)(F)F)c2)c(Oc2cc(F)ccc2Cl)c2c(N3C(=O)c4ccccc4C3=O)n[nH]c12]

To the mixture of N-(4-(2-chloro-5-fluorophenoxy)-7-((2-chloroacetamido)methyl)-3-(1,3-dioxoisoindolin-2-yl)-1-(tetrahydro-2H-pyran-2-yl)-1H-indazol-5-yl)-3-fluoro-5-(trifluoromethyl)benzamide (55 mg, 0.07 mmol) in dichloromethane (0.6 mL) was added trifluoroacetic acid (0.2 mL). The resulting mixture was stirred for 4 h at room temperature. The resulting mixture was concentrated under reduced pressure to afford crude product as a white solid. The crude product was used in the next step directly without further purification. LCMS calculated for C₃₂H₁₉Cl₂F₅N₅O₅(M+H)⁺ m/z=718.1; found 718.1.

### Step 7: N-(3-(2-chloro-5-fluorophenoxy)-2-(1,3-dioxoisoindolin-2-yl)-8-oxo-6,7,8,9-tetrahydro-[1,4]diazepino[6,7,1-hi]indazol-4-yl)-3-fluoro-5-(trifluoromethyl)benzamide

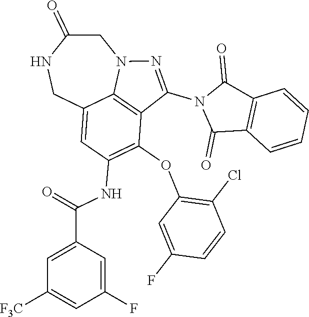 [SMILES: O=C1Cn2nc(N3C(=O)c4ccccc4C3=O)c3c(Oc4cc(F)ccc4Cl)c(NC(=O)c4cc(F)cc(C(F)(F)F)c4)cc(c32)CN1]

To the mixture of N-(4-(2-chloro-5-fluorophenoxy)-7-((2-chloroacetamido)methyl)-3-(1,3-dioxoisoindolin-2-yl)-1H-indazol-5-yl)-3-fluoro-5-(trifluoromethyl)benzamide (60 mg, 0.08 mmol) in N,N-Dimethylformamide (2 mL) was added potassium carbonate (34 mg, 0.25 mmol). The reaction was stirred for additional 4 h. The resulting mixture was diluted with water (20 mL). The resulting mixture was extracted with ethyl acetate (3×20 mL). The combined organic layers were washed with brine (2×30 mL), dried over anhydrous sodium sulfate. After filtration, the filtrate was concentrated under reduced pressure. The residue was purified by silica gel column chromatography, eluted with 7% methanol in dichloromethane to afford the desired product as a yellow solid (30 mg, 53%). LCMS calculated for C₃₂H₁₈ClF₅N₅O₅ (M+H)⁺ m/z=682.1; found 682.2.

### Step 8: N-(2-amino-3-(2-chloro-5-fluorophenoxy)-8-oxo-6,7,8,9-tetrahydro-[1,4]diazepino[6,7,1-hi]indazol-4-yl)-3-fluoro-5-(trifluoromethyl)benzamide

To the mixture of N-(3-(2-chloro-5-fluorophenoxy)-2-(1,3-dioxoisoindolin-2-yl)-8-oxo-6,7,8,9-tetrahydro-[1,4]diazepino[6,7,1-hi]indazol-4-yl)-3-fluoro-5-(trifluoromethyl)benzamide (30 mg, 0.04 mmol) in methanol (0.5 mL) was added hydrazine hydrate (98%, 0.1 mL) at room temperature. The reaction was stirred for 20 min. The mixture was purified by Prep-HPLC with the following conditions (Column: Sunfire prep C18 column, 30*150 mm; Mobile phase, acetonitrile in water (0.1% formic acid); Flow rate: 60 mL/min; Gradient: 30% B to 50% B in 10 min, 50% B to 50% B in 5 min; Detector, UV 254 nm). Eluted fractions were collected and lyophilized to afford the desired product as a white solid. LCMS calculated for C₂₄H₁₆ClF₅N₅O₃ (M+H)⁺ m/z=552.1; found 552.1; ¹H NMR (400 MHz, DMSO-d₆) δ 10.16 (s, 1H), 8.53-8.48 (m, 1H), 7.94-7.89 (m, 1H), 7.83-7.74 (m, 2H), 7.50-7.43 (m, 1H), 7.16 (s, 1H), 6.92-6.82 (m, 1H), 6.51-6.44 (m, 1H), 5.05 (s, 2H), 5.01 (s, 2H), 4.58 (d, J=6.0 Hz, 2H).

### Example 37. N-(3-Amino-4-(2-chloro-5-fluorophenoxy)-1-methyl-7-(pyridin-2-ylethynyl)-1H-indazol-5-yl)-3-fluoro-5-(trifluoromethyl)benzamide

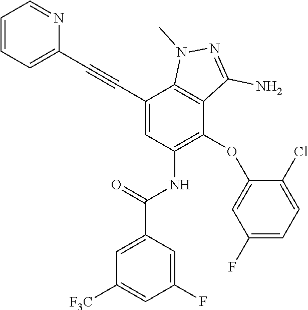 [SMILES: Cn1nc(N)c2c(Oc3cc(F)ccc3Cl)c(NC(=O)c3cc(F)cc(C(F)(F)F)c3)cc(C#Cc3ccccn3)c21]

To a screw-cap vial equipped with a magnetic stir bar were placed N-[3-amino-7-bromo-4-(2-chloro-5-fluorophenoxy)-1-methylindazol-5-yl]-3-fluoro-5-(trifluoromethyl)benzamide (Example 25: 30 mg, 0.05 mmol), Pd(PPh₃)₂Cl₂ (18 mg, 0.03 mmol) and cuprous iodide (2 mg, 0.01 mmol). The vial was sealed with a Teflon-lined septum, evacuated and backfilled with nitrogen (this process was repeated a total of three times). 2-Ethynylpyridine (27 mg, 0.26 mmol) and triethylamine (0.75 mL) in N,N-Dimethylformamide (0.75 mL) were added. The reaction was stirred at 100° C. for 2 h. Upon cooling to room temperature, the resulting mixture was diluted with water (20 mL). The resulting mixture was extracted with ethyl acetate (3×20 mL). The combined organic layers were washed with brine (1×50 mL), dried over anhydrous sodium sulfate. After filtration, the filtrate was concentrated under reduced pressure. The residue was purified by silica gel column chromatography, eluted with 10% methanol in dichloromethane to afford crude product, which was further purified by reversed-phase flash chromatography with the following conditions: column, C18 silica gel; mobile phase, acetonitrile in water (0.1% trifluoroacetic acid), 30% to 95% gradient in 30 min; detector, UV 254 nm. The collected fractions were lyophilized to afford the TFA salt of the desired product as a brown yellow solid. LCMS calculated for C₂₉H₁₈ClF₅N₅O₂(M+H)⁺ m/z=598.1; found 598.1; ¹H NMR (400 MHz, Chloroform-d) δ 8.80-8.74 (m, 1H), 8.45 (s, 1H), 7.98-7.89 (m, 1H), 7.85-7.80 (m, 1H), 7.73-7.67 (m, 1H), 7.66-7.60 (m, 2H), 7.53-7.38 (m, 3H), 6.82-6.73 (m, 1H), 6.54-6.46 (m, 1H), 4.31 (s, 3H).

### Example 38. N-(3-Amino-4-(2-chloro-5-fluorophenoxy)-1-methyl-7-((tetrahydro-2H-pyran-4-yl)ethynyl)-1H-indazol-5-yl)-3-fluoro-5-(trifluoromethyl)benzamide

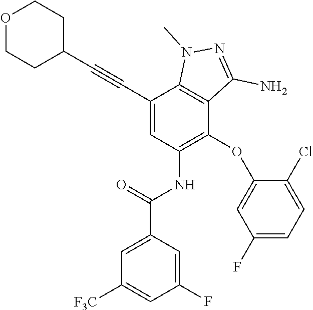 [SMILES: Cn1nc(N)c2c(Oc3cc(F)ccc3Cl)c(NC(=O)c3cc(F)cc(C(F)(F)F)c3)cc(C#CC3CCOCC3)c21]

The title compound was prepared according to the procedure described in Example 37, using 4-ethynyloxane instead of 2-ethynylpyridine. LCMS calculated for C₂₉H₂₃ClF₅N₄O₃ (M+H)⁺ m/z=605.1; found 605.1; ¹H NMR (400 MHz, DMSO-d₆) δ 10.12 (s, 1H), 7.90 (d, J=8.6 Hz, 1H), 7.78-7.70 (m, 2H), 7.46 (dd, J=8.8, 5.6 Hz, 1H), 7.37 (s, 1H), 6.90-6.83 (m, 1H), 6.49 (dd, J=9.6, 2.8 Hz, 1H), 5.02 (s, 2H), 4.10 (s, 3H), 3.89-3.79 (m, 2H), 3.53-3.42 (m, 2H), 3.08-2.96 (m, 1H), 1.96-1.84 (m, 2H), 1.75-1.61 (m, 2H).

### Example 39. N-(3-Amino-4-(2-chloro-5-fluorophenoxy)-7-(3-hydroxy-3-methylbut-1-yn-1-yl)-1-methyl-1H-indazol-5-yl)-3-fluoro-5-(trifluoromethyl)benzamide

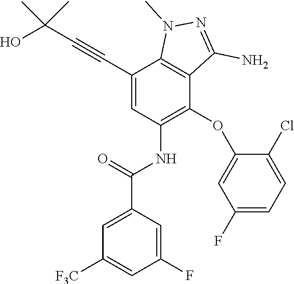 [SMILES: Cn1nc(N)c2c(Oc3cc(F)ccc3Cl)c(NC(=O)c3cc(F)cc(C(F)(F)F)c3)cc(C#CC(C)(C)O)c21]

The title compound was prepared according to the procedure described in Example 37, using 2-methylbut-3-yn-2-ol instead of 2-ethynylpyridine. LCMS calculated for C₂₇H₂₁ClF₅N₄O₃ (M+H)⁺ m/z=579.1; found 579.1; ¹H NMR (400 MHz, DMSO-d₆) δ 10.12 (s, 1H), 7.90 (d, J=8.4 Hz, 1H), 7.78-7.70 (m, 2H), 7.47 (dd, J=8.8, 5.6 Hz, 1H), 7.36 (s, 1H), 6.92-6.83 (m, 1H), 6.49 (dd, J=9.6, 2.8 Hz, 1H), 5.58 (s, 1H), 5.06 (s, 2H), 4.11 (s, 3H), 1.52 (s, 6H).

### Example 40. N-(3-Amino-7-(3-amino-3-methylbut-1-yn-1-yl)-4-(2-chloro-5-fluorophenoxy)-1-methyl-1H-indazol-5-yl)-3-fluoro-5-(trifluoromethyl)benzamide

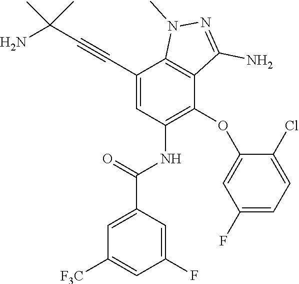 [SMILES: Cn1nc(N)c2c(Oc3cc(F)ccc3Cl)c(NC(=O)c3cc(F)cc(C(F)(F)F)c3)cc(C#CC(C)(C)N)c21]

The title compound was prepared according to the procedure described in Example 37, using 2-methylbut-3-yn-2-amine instead of 2-ethynylpyridine. LCMS calculated for C₂₇H₂₀ClF₅N₅O₂ (M−H)⁻ m/z=576.1; found 576.0; ¹H NMR (400 MHz, DMSO-d₆) δ 10.16 (s, 1H), 8.64 (s, 2H), 7.91 (d, J=8.4 Hz, 1H), 7.76-7.69 (m, 2H), 7.53-7.42 (m, 2H), 6.92-6.84 (m, 1H), 6.51 (dd, J=9.6, 2.8 Hz, 1H), 5.14 (s, 2H), 4.09 (s, 3H), 1.69 (s, 6H).

### Example 41. N-(3-Amino-4-(2-chloro-5-fluorophenoxy)-1-methyl-7-((tetrahydrofuran-3-yl)ethynyl)-1H-indazol-5-yl)-3-fluoro-5-(trifluoromethyl)benzamide

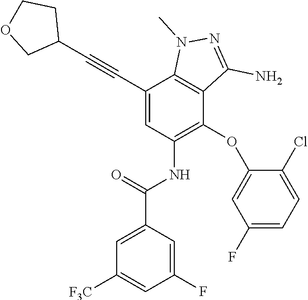 [SMILES: Cn1nc(N)c2c(Oc3cc(F)ccc3Cl)c(NC(=O)c3cc(F)cc(C(F)(F)F)c3)cc(C#CC3CCOC3)c21]

The title compound was prepared according to the procedure described in Example 37, using 3-ethynyltetrahydrofuran instead of 2-ethynylpyridine. LCMS calculated for C₂₈H₂₁ClF₅N₄O₃ (M+H)⁺ m/z=591.1; found 591.1; ¹H NMR (400 MHz, DMSO-d₆) δ 10.12 (s, 1H), 7.90 (d, J=8.8 Hz, 1H), 7.77-7.70 (m, 2H), 7.46 (dd, J=8.8, 5.6 Hz, 1H), 7.37 (s, 1H), 6.90-6.84 (m, 1H), 6.49 (dd, J=9.6, 2.8 Hz, 1H), 5.06 (s, 2H), 4.08 (s, 3H), 3.99 (t, J=7.6 Hz, 1H), 3.91-3.75 (m, 2H), 3.73-3.67 (m, 1H), 3.43-3.36 (m, 1H), 2.34-2.23 (m, 1H), 2.08-1.97 (m, 1H).

### Example 42. N-(3-Amino-4-(2-chloro-5-fluorophenoxy)-7-(cyclopropylethynyl)-1-methyl-1H-indazol-5-yl)-3-fluoro-5-(trifluoromethyl)benzamide

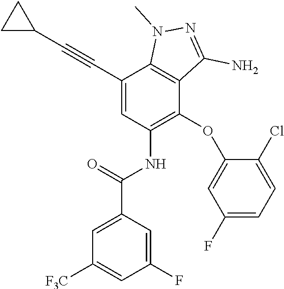 [SMILES: Cn1nc(N)c2c(Oc3cc(F)ccc3Cl)c(NC(=O)c3cc(F)cc(C(F)(F)F)c3)cc(C#CC3CC3)c21]

The title compound was prepared according to the procedure described in Example 37, using ethynylcyclopropane instead of 2-ethynylpyridine. LCMS calculated for C₂₇H₁₉ClF₅N₄O₂ (M+H)⁺ m/z=561.1; found 561.1; H NMR (400 MHz, DMSO-d₆) δ 10.11 (s, 1H), 7.94-7.86 (m, 1H), 7.77-7.70 (m, 2H), 7.45 (dd, J=8.8, 5.6 Hz, 1H), 7.32 (s, 1H), 6.91-6.82 (m, 1H), 6.47 (dd, J=9.6, 2.8 Hz, 1H), 5.00 (s, 2H), 4.06 (s, 3H), 1.71-1.61 (m, 1H), 1.01-0.92 (m, 2H), 0.86-0.78 (m, 2H).

### Example 43. N-(3-Amino-4-(2-chloro-5-fluorophenoxy)-7-((1-hydroxycyclopropyl)ethynyl)-1-methyl-1H-indazol-5-yl)-3-fluoro-5-(trifluoromethyl)benzamide

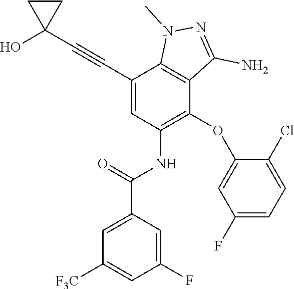 [SMILES: Cn1nc(N)c2c(Oc3cc(F)ccc3Cl)c(NC(=O)c3cc(F)cc(C(F)(F)F)c3)cc(C#CC3(O)CC3)c21]

The title compound was prepared according to the procedure described in Example 37, using 1-ethynylcyclopropan-1-ol instead of 2-ethynylpyridine. LCMS calculated for C₂₇H₁₉ClF₅N₄O₃ (M+H)⁺ m/z=577.1; found 577.1; ¹H NMR (400 MHz, Methanol-d₄) δ 7.70 (s, 1H), 7.67-7.62 (m, 2H), 7.43 (s, 1H), 7.36 (dd, J=8.8, 5.6 Hz, 1H), 6.79-6.73 (m, 1H), 6.53-6.48 (dd, J=9.6, 2.8 Hz, 1H), 4.17 (s, 3H), 1.17-1.05 (m, 4H).

### Example 44. N-(3-Amino-4-(2-chloro-5-fluorophenoxy)-7-((1-(hydroxymethyl)cyclopropyl)ethynyl)-1-methyl-1H-indazol-5-yl)-3-fluoro-5-(trifluoromethyl)benzamide

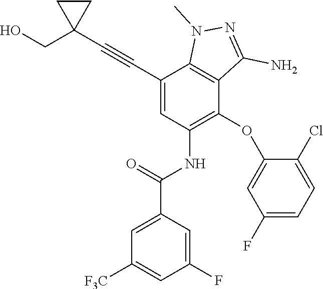 [SMILES: Cn1nc(N)c2c(Oc3cc(F)ccc3Cl)c(NC(=O)c3cc(F)cc(C(F)(F)F)c3)cc(C#CC3(CO)CC3)c21]

The title compound was prepared according to the procedure described in Example 37, using (1-ethynylcyclopropyl)methanol instead of 2-ethynylpyridine. LCMS calculated for C₂₈H₂₁ClF₅N₄O₃ (M+H)⁺ m/z=591.1; found 591.1; ¹H NMR (400 MHz, DMSO-d₆) δ 10.12 (s, 1H), 7.90 (d, J=8.4 Hz, 1H), 7.77-7.71 (m, 2H), 7.46 (dd, J=8.8, 5.6 Hz, 1H), 7.33 (s, 1H), 6.90-6.83 (m, 1H), 6.47 (dd, J=9.6, 2.8 Hz, 1H), 5.08 (s, 2H), 4.09 (s, 3H), 3.49 (s, 2H), 1.00-0.94 (m, 2H), 0.94-0.88 (m, 2H).

### Example 45. N-(3-Amino-7-((1-aminocyclopropyl)ethynyl)-4-(2-chloro-5-fluorophenoxy)-1-methyl-1H-indazol-5-yl)-3-fluoro-5-(trifluoromethyl)benzamide

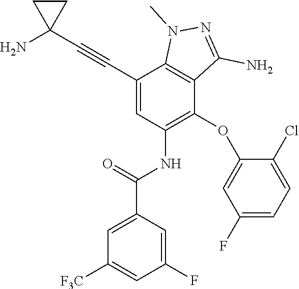 [SMILES: Cn1nc(N)c2c(Oc3cc(F)ccc3Cl)c(NC(=O)c3cc(F)cc(C(F)(F)F)c3)cc(C#CC3(N)CC3)c21]

The title compound was prepared according to the procedure described in Example 37, using 1-ethynylcyclopropan-1-amine instead of 2-ethynylpyridine. LCMS calculated for C₂₇H₂₀ClF₅N₅O₂ (M+H)⁺ m/z=576.1; found 576.1; ¹H NMR (400 MHz, DMSO-d₆) δ 10.15 (s, 1H), 8.75 (s, 2H), 7.94-7.90 (m, 1H), 7.76-7.69 (m, 2H), 7.51-7.41 (m, 2H), 6.91-6.84 (m, 1H), 6.50 (dd, J=9.6, 2.8 Hz, 1H), 5.14 (s, 2H), 4.07 (s, 3H), 1.43-1.38 (m, 4H).

### Example 46. N-(3-Amino-4-(2-chloro-5-fluorophenoxy)-1-methyl-7-((3-methyloxetan-3-yl)ethynyl)-1H-indazol-5-yl)-3-fluoro-5-(trifluoromethyl)benzamide

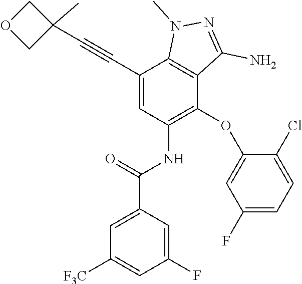 [SMILES: Cn1nc(N)c2c(Oc3cc(F)ccc3Cl)c(NC(=O)c3cc(F)cc(C(F)(F)F)c3)cc(C#CC3(C)COC3)c21]

The title compound was prepared according to the procedure described in Example 37, using 3-ethynyl-3-methyloxetane instead of 2-ethynylpyridine. LCMS calculated for C₂₈H₂₁ClF₅N₄O₃ (M+H)⁺ m/z=591.1; found 591.1; H NMR (400 MHz, DMSO-d₆) δ 10.13 (s, 1H), 7.90 (d, J=8.4 Hz, 1H), 7.78-7.70 (m, 2H), 7.46 (dd, J=8.8, 5.6 Hz, 1H), 7.41 (s, 1H), 6.91-6.84 (m, 1H), 6.50 (dd, J=9.6, 2.8 Hz, 1H), 4.81 (d, J=5.6 Hz, 2H), 4.49 (d, J=5.6 Hz, 2H), 4.11 (s, 3H), 1.69 (s, 3H).

### Example 47. N-(3-Amino-4-(2-chloro-5-fluorophenoxy)-7-ethynyl-1-methyl-1H-indazol-5-yl)-3-fluoro-5-(trifluoromethyl)benzamide

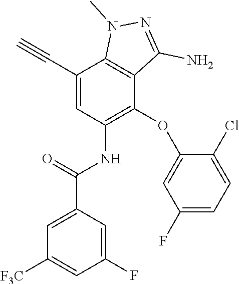 [SMILES: C#Cc1cc(NC(=O)c2cc(F)cc(C(F)(F)F)c2)c(Oc2cc(F)ccc2Cl)c2c(N)nn(C)c12]

### Step 1: N-(3-amino-4-(2-chloro-5-fluorophenoxy)-1-methyl-7-((trimethylsilyl)ethynyl)-1H-indazol-5-yl)-3-fluoro-5-(trifluoromethyl)benzamide

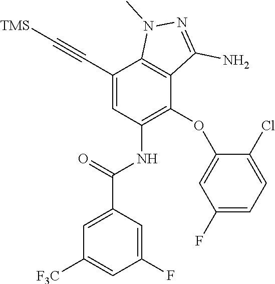 [SMILES: Cn1nc(N)c2c(Oc3cc(F)ccc3Cl)c(NC(=O)c3cc(F)cc(C(F)(F)F)c3)cc(C#C[Si](C)(C)C)c21]

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 200 mg, 0.35 mmol), Pd(PPh₃)₄ (32 mg, 0.03 mmol) and cuprous iodide (2.7 mg, 0.014 mmol). The vial was sealed with a Teflon-lined septum, evacuated and backfilled with nitrogen (this process was repeated a total of three times). Trimethylsilylacetylene (68 mg, 0.7 mmol) and triethylamine (2 mL) in N,N-Dimethylformamide (2 mL) were added. The reaction was stirred at 100° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a yellow solid (200 mg, 97%). LCMS calculated for C₂₇H₂₃ClF₅N₄O₂Si (M+H)⁺ m/z=593.1; found 593.1.

### Step 2: N-(3-amino-4-(2-chloro-5-fluorophenoxy)-7-ethynyl-1-methyl-1H-indazol-5-yl)-3-fluoro-5-(trifluoromethyl)benzamide

The mixture of N-(3-amino-4-(2-chloro-5-fluorophenoxy)-1-methyl-7-((trimethylsilyl)ethynyl)-1H-indazol-5-yl)-3-fluoro-5-(trifluoromethyl)benzamide (100 mg, 0.17 mmol) and potassium carbonate (23 mg, 0.17 mmol) in methanol (5 mL) was stirred for 1 h at room temperature. The resulting mixture was concentrated under reduced pressure. The residue was purified by silica gel column chromatography, eluted with 50% ethyl acetate in petroleum ether to afford crude product which was then purified by Prep-HPLC with the following conditions (Column: XBridge Prep Phenyl OBD Column 19*250 mm; Mobile Phase: acetonitrile in water (0.05% trifluoroacetic acid); Flow rate: 60 mL/min; Gradient: 37% B to 55% B in 10 min; Detector: 254/220 nm). Eluted fractions were collected and lyophilized to afford the TFA salt of the desired product as a light-yellow solid. LCMS calculated for C₂₄H₁₅ClF₅N₄O₂ (M+H)⁺ m/z=521.1; found 521.2; ¹H NMR (400 MHz, DMSO-d₆) δ 10.13 (s, 1H), 7.93-7.87 (m, 1H), 7.77-7.70 (m, 2H), 7.48-7.42 (m, 2H), 6.92-6.83 (m, 1H), 6.53 (dd, J=9.6, 2.8 Hz, 1H), 5.12 (s, 2H), 4.63 (s, 1H), 4.09 (s, 3H).

### Example 48. N-(3-Amino-4-(2-chloro-5-fluorophenoxy)-1-methyl-7-((1-methyl-1H-pyrazol-3-yl)ethynyl)-1H-indazol-5-yl)-3-fluoro-5-(trifluoromethyl)benzamide

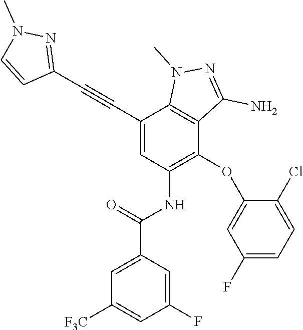 [SMILES: Cn1ccc(C#Cc2cc(NC(=O)c3cc(F)cc(C(F)(F)F)c3)c(Oc3cc(F)ccc3Cl)c3c(N)nn(C)c23)n1]

To a screw-cap vial equipped with a magnetic stir bar were placed N-(3-amino-4-(2-chloro-5-fluorophenoxy)-7-ethynyl-1-methyl-1H-indazol-5-yl)-3-fluoro-5-(trifluoromethyl)benzamide (Example 47, Step 1: 20 mg, 0.1 mmol), Pd(PPh₃)₂Cl₂ (13.5 mg, 0.02 mmol) and cuprous iodide (3.7 mg, 0.02 mmol). The vial was sealed with a Teflon-lined septum, evacuated and backfilled with nitrogen (this process was repeated a total of three times). 3-Iodo-1-methylpyrazole (20 mg, 0.1 mmol) and triethylamine (0.5 mL) in N,N-Dimethylformamide (0.5 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 50% ethyl acetate in petroleum ether to afford crude product, which was then purified by reversed-phase flash chromatography with the following conditions (Column: Xselect CSH C18 OBD Column 30*150 mm; Mobile Phase: acetonitrile in water (0.05% 2,2,2-trifluoroacetic acid); Flow rate: 60 mL/min; 41% B to 61% B in 10 min; Detector: 254/220 nm). Eluted fractions were collected and lyophilized to afford the TFA salt of the desired product as a white solid. LCMS calculated for C₂₈H₁₉ClF₅N₆O₂(M+H)⁺ m/z=601.1; found 601.1; ¹H NMR (400 MHz, DMSO-d₆) δ 10.16 (s, 1H), 7.94-7.88 (m, 1H), 7.83-7.80 (m, 1H), 7.77-7.70 (m, 2H), 7.52-7.42 (m, 2H), 6.91-6.84 (m, 1H), 6.60 (d, J=2.4 Hz, 1H), 6.55 (dd, J=9.6, 2.8 Hz, 1H), 5.13 (s, 2H), 4.14 (s, 3H), 3.90 (s, 3H).

### Example 49. N-(3-Amino-4-(2-chloro-5-fluorophenoxy)-7-(3-hydroxyprop-1-yn-1-yl)-1-methyl-1H-indazol-5-yl)-3-fluoro-5-(trifluoromethyl)benzamide

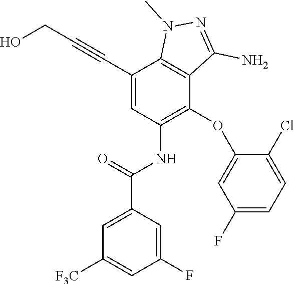 [SMILES: Cn1nc(N)c2c(Oc3cc(F)ccc3Cl)c(NC(=O)c3cc(F)cc(C(F)(F)F)c3)cc(C#CCO)c21]

### Step 1: N-(3-amino-4-(2-chloro-5-fluorophenoxy)-1-methyl-7-(3-((tetrahydro-2H-pyran-2-yl)oxy)prop-1-yn-1-yl)-1H-indazol-5-yl)-3-fluoro-5-(trifluoromethyl)benzamide

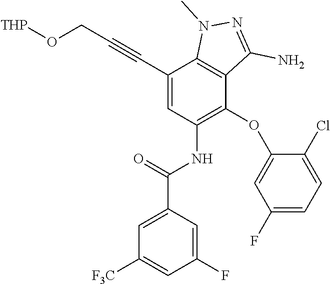

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 50 mg, 0.09 mmol), Pd(PPh₃)₂Cl₂ (12 mg, 0.02 mmol) and cuprous iodide (3.3 mg, 0.017 mmol). The vial was sealed with a Teflon-lined septum, evacuated and backfilled with nitrogen (this process was repeated a total of three times). 2-(Prop-2-yn-1-yloxy)oxane (24 mg, 0.17 mmol) and triethylamine (0.5 mL) in N,N-Dimethylformamide (0.5 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a brown oil (20 mg, 36%). LCMS calculated for C₃₀H₂₅ClF₅N₄O₄ (M+H)+m/z=635.1; found 635.2;

### Step 2: N-(3-amino-4-(2-chloro-5-fluorophenoxy)-7-(3-hydroxyprop-1-yn-1-yl)-1-methyl-1H-indazol-5-yl)-3-fluoro-5-(trifluoromethyl)benzamide

The mixture of N-(3-amino-4-(2-chloro-5-fluorophenoxy)-1-methyl-7-(3-((tetrahydro-2H-pyran-2-yl)oxy)prop-1-yn-1-yl)-1H-indazol-5-yl)-3-fluoro-5-(trifluoromethyl)benzamide (20 mg, 0.03 mmol) in trifluoroacetic acid (1 mL) was stirred at 50° C. for 1 h. Upon cooling to room temperature, the resulting mixture was concentrated under reduced pressure. The residue was purified by Prep-HPLC with the following conditions (Column: Sunfire prep C18 column, 30*150 mm; Mobile Phase: acetonitrile in water (0.05% trifluoroacetic acid); Flow rate: 60 mL/min; Gradient: 37% B to 55% B in 10 min; Detector: 254/220 nm). Eluted fractions were collected and lyophilized to afford the TFA salt of the desired product as a white solid. LCMS calculated for C₂₅H₁₇ClF₅N₄O₃ (M+H)⁺ m/z=551.1; found 551.1; ¹H NMR (400 MHz, DMSO-d₆) δ 10.13 (s, 1H), 7.93-7.87 (m, 1H), 7.76-7.70 (m, 2H), 7.46 (dd, J=8.8, 5.6 Hz, 1H), 7.38 (s, 1H), 6.91-6.83 (m, 1H), 6.51 (dd, J=9.6, 2.8 Hz, 1H), 5.42 (s, 1H), 5.08 (s, 2H), 4.40 (s, 2H), 4.09 (s, 3H).

### Example 50. N-(3-Amino-4-(2-chloro-5-fluorophenoxy)-1-methyl-7-((1-methyl-1H-imidazol-4-yl)ethynyl)-1H-indazol-5-yl)-3-fluoro-5-(trifluoromethyl)benzamide

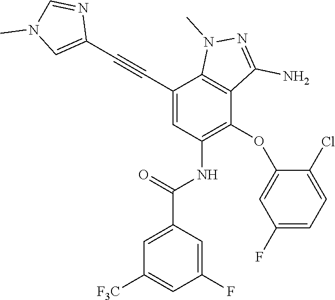 [SMILES: Cn1cnc(C#Cc2cc(NC(=O)c3cc(F)cc(C(F)(F)F)c3)c(Oc3cc(F)ccc3Cl)c3c(N)nn(C)c23)c1]

### Step 1: 1-Methyl-4-((trimethylsilyl)ethynyl)-1H-imidazole

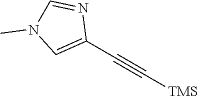

To a screw-cap vial equipped with a magnetic stir bar were placed 4-iodo-1-methylimidazole (1 g, 4.8 mmol), Pd(PPh₃)₄ (444 mg, 0.4 mmol) and cuprous iodide (37 mg, 0.2 mmol). The vial was sealed with a Teflon-lined septum, evacuated and backfilled with nitrogen (this process was repeated a total of three times). Trimethylsilylacetylene (944 mg, 9.6 mmol) and triethylamine (0.5 mL) in N,N-Dimethylformamide (10 mL) were added. The reaction was stirred at 100° C. for 2 h. Upon cooling to room temperature, the resulting mixture was diluted with water (100 mL). The resulting mixture was extracted with ethyl acetate (3×100 mL). The combined organic layers were washed with brine (100 mL), dried over anhydrous sodium sulfate. After filtration, the filtrate was concentrated under reduced pressure. The residue was purified by silica gel column chromatography, eluted with 25% ethyl acetate in petroleum ether to afford the desired product (780 mg, 91%) as a brown oil. LCMS calculated for C₉H₁₅N₂Si (M+H)⁺ m/z=179.1; found 179.2;

### Step 2: 4-Ethynyl-1-methyl-1H-imidazole

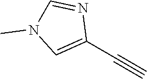

The mixture of 1-methyl-4-[2-(trimethylsilyl)ethynyl]imidazole (300 mg, 1.68 mmol) and potassium carbonate (465 mg, 3.36 mmol) in methanol (10 mL) was stirred at room temperature for 1 h. The resulting mixture was concentrated under reduced pressure. The resulting mixture was extracted with ethyl acetate (3×50 mL). The combined organic layers were washed with brine (50 mL), dried over anhydrous sodium sulfate. After filtration, the filtrate was concentrated under reduced pressure, which was then purified by reversed-phase flash chromatography with the following conditions: (Column, C18 silica gel; Mobile phase: acetonitrile in water (10 mmol/L ammonium bicarbonate); 10% to 50% gradient in 30 min; Detector: 254 nm), eluted fractions were collected to afford the desired product as a light-yellow solid (72 mg, 40%). LCMS calculated for C₆H₇N₂ (M+H)⁺ m/z=107.1; found 107.2; ¹H NMR (300 MHz, Chloroform-d) δ 7.41 (d, J=1.2 Hz, 1H), 7.12 (d, J=1.2 Hz, 1H), 3.70 (s, 3H), 3.07 (s, 1H).

### Step 3: N-(3-amino-4-(2-chloro-5-fluorophenoxy)-1-methyl-7-((1-methyl-1H-imidazol-4-yl)ethynyl)-1H-indazol-5-yl)-3-fluoro-5-(trifluoromethyl)benzamide

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 20 mg, 0.035 mmol), Pd(PPh₃)₂Cl₂ (4.9 mg, 0.007 mmol) and cuprous iodide (1.3 mg, 0.007 mmol). The vial was sealed with a Teflon-lined septum, evacuated and backfilled with nitrogen (this process was repeated a total of three times). 4-Ethynyl-1-methyl-1H-imidazole (7.4 mg, 0.07 mmol) and triethylamine (0.5 mL) in N,N-Dimethylformamide (0.5 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 50% ethyl acetate in petroleum ether to afford crude product, which was then purified by reversed-phase flash chromatography with the following conditions (Column: Xselect CSH C18 OBD Column 30*150 mm; Mobile Phase: acetonitrile in water (0.05% 2,2,2-trifluoroacetic acid); Flow rate: 60 mL/min; 21% B to 41% B in 10 min; detector, 254/220 nm). Eluted fractions were collected and lyophilized to afford the TFA salt of the desired product as a yellow solid. LCMS calculated for C₂₈H₁₉ClF₅N₆O₂(M+H)⁺ m/z=601.1; found 601.3; ¹H NMR (400 MHz, DMSO-d₆) δ 10.17 (s, 1H), 8.26 (s, 1H), 7.94-7.88 (m, 1H), 7.85 (s, 1H), 7.78-7.70 (m, 2H), 7.53-7.43 (m, 2H), 6.93-6.85 (m, 1H), 6.55 (dd, J=9.6, 2.8 Hz, 1H), 4.13 (s, 3H), 3.77 (s, 3H).

### Example 51. N-(3-Amino-4-(2-chloro-5-fluorophenoxy)-1-methyl-7-((1-methylpiperidin-4-yl)ethynyl)-1H-indazol-5-yl)-3-fluoro-5-(trifluoromethyl)benzamide

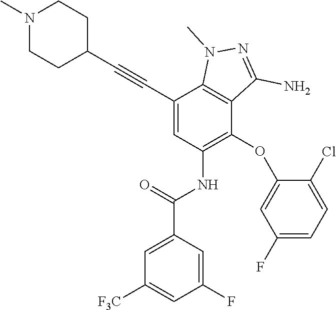 [SMILES: CN1CCC(C#Cc2cc(NC(=O)c3cc(F)cc(C(F)(F)F)c3)c(Oc3cc(F)ccc3Cl)c3c(N)nn(C)c23)CC1]

### Step 1: 4-Ethynylpiperidine

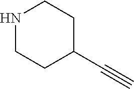

To the mixture of tert-butyl 4-ethynylpiperidine-1-carboxylate (1 g, 4.78 mmol) in dichloromethane (15 mL) was added trifluoroacetic acid (1 mL) at room temperature. The reaction was stirred for additional 2 h. The mixture was concentrated under reduced pressure. The crude product was used in the next step directly without further purification. LCMS calculated for C₇H₁₂N (M+H)⁺ m/z=110.1; found 110.0.

### Step 2: 4-Ethynyl-1-methylpiperidine

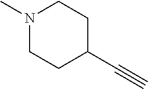

To the mixture of 4-ethynylpiperidine (100 mg, 0.92 mmol) and formaldehyde (37% in water, 111 mg, 1.37 mmol) in methanol (2 mL) was added acetic acid (165 mg, 2.75 mmol) at room temperature. The reaction was stirred for 1 h. To the above mixture was added sodium cyanoborohydride (173 mg, 2.75 mmol). The reaction mixture was stirred for additional 1 h. The resulting mixture was concentrated under reduced pressure. The residue was purified by reversed-phase flash chromatography with the following conditions (Column: C18 silica gel; Mobile phase: acetonitrile in water (10 mmol/L ammonium bicarbonate), 5% to 20% gradient in 10 min; Detector: 220 nm), eluted fractions were collected and lyophilized to afford the desired product as a white solid (30 mg). LCMS calculated for C₈H₁₄N (M+H)⁺ m/z=124.1; found 124.3.

### Step 3: N-(3-amino-4-(2-chloro-5-fluorophenoxy)-1-methyl-7-((1-methylpiperidin-4-yl)ethynyl)-1H-indazol-5-yl)-3-fluoro-5-(trifluoromethyl)benzamide

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 20 mg, 0.035 mmol), Pd(PPh₃)₂Cl₂ (4.9 mg, 0.007 mmol) and cuprous iodide (1.3 mg, 0.007 mmol). The vial was sealed with a Teflon-lined septum, evacuated and backfilled with nitrogen (this process was repeated a total of three times). 4-Ethynyl-1-methylpiperidine (8.6 mg, 0.07 mmol) and triethylamine (0.7 mL) in N,N-Dimethylformamide (0.7 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 10% methanol in dichloromethane to afford crude product, which was then purified by reversed-phase flash chromatography with the following conditions (column, C18 silica gel; Mobile Phase: acetonitrile in water (0.1% 2,2,2-trifluoroacetic acid); 40% to 80% gradient in 30 min; Detector: 254/220 nm). Eluted fractions were collected and lyophilized to afford the TFA salt of the desired product as an off-white solid. LCMS calculated for C₃₀H₂₆ClF₅N₅O₂ (M+H)⁺ m/z=618.2; found 618.2; ¹H NMR (400 MHz, DMSO-d₆) δ 10.08 (s, 1H), 9.32 (s, 1H), 7.85 (d, J=8.4 Hz, 1H), 7.71-7.63 (m, 2H), 7.43-7.36 (m, 1H), 7.30 (s, 1H), 6.85-6.77 (m, 1H), 6.45 (dd, J=9.6, 2.8 Hz, 1H), 5.01 (s, 2H), 4.02 (s, 3H), 3.01-2.81 (m, 4H), 2.80-2.70 (m, 2H), 2.63-2.54 (m, 1H), 2.21-2.13 (m, 1H), 1.99-1.91 (m, 2H), 1.82-1.71 (m, 1H).

### Example 52. N-(7-((1-Acetylpiperidin-4-yl)ethynyl)-3-amino-4-(2-chloro-5-fluorophenoxy)-1-methyl-1H-indazol-5-yl)-3-fluoro-5-(trifluoromethyl)benzamide

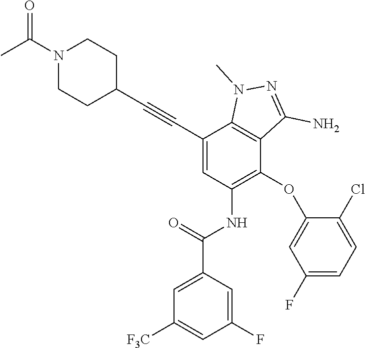 [SMILES: CC(=O)N1CCC(C#Cc2cc(NC(=O)c3cc(F)cc(C(F)(F)F)c3)c(Oc3cc(F)ccc3Cl)c3c(N)nn(C)c23)CC1]

### Step 1: 1-(4-Ethynylpiperidin-1-yl)ethan-1-one

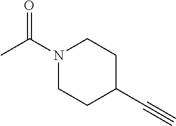

To the mixture of 4-ethynylpiperidine (Example 51, Step 1: 100 mg, 0.92 mmol) and triethylamine (278 mg, 2.75 mmol) in dichloromethane (2 mL) was added acetic anhydride (121 mg, 1.2 mmol) at room temperature. The reaction was stirred for 2 h. The resulting mixture was concentrated under reduced pressure. The residue was purified by silica gel column chromatography, eluted with 10% methanol in dichloromethane to afford the desired product as a yellow solid (50 mg, 36%). LCMS calculated for C₉H₁₄NO (M+H)⁺ m/z=152.1; found 152.2;

### Step 2: N-(7-((I-acetylpiperidin-4-yl)ethynyl)-3-amino-4-(2-chloro-5-fluorophenoxy)-1-methyl-1H-indazol-5-yl)-3-fluoro-5-(trifluoromethyl)benzamide

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 20 mg, 0.035 mmol), Pd(PPh₃)₂Cl₂ (4.9 mg, 0.007 mmol) and cuprous iodide (1.3 mg, 0.007 mmol). The vial was sealed with a Teflon-lined septum, evacuated and backfilled with nitrogen (this process was repeated a total of three times). 1-(4-Ethynylpiperidin-1-yl)ethan-1-one (10.51 mg, 0.07 mmol) and triethylamine (0.7 mL) in N,N-Dimethylformamide (0.7 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 10% methanol in dichloromethane to afford crude product, which was then purified by reversed-phase flash chromatography with the following conditions (column, Xselect CSH C18 OBD, 30*150 mm, 5 m; Mobile Phase: acetonitrile in water (0.05% 2,2,2-trifluoroacetic acid); Flow rate: 60 mL/min; 40% to 60% gradient in 8 min; Detector: 254/220 nm). Eluted fractions were collected and lyophilized to afford the TFA salt of the desired product as a white solid. LCMS calculated for C₃₁H₂₆ClF₅N₅O₃ (M+H)⁺ m/z=646.2; found 646.2; H NMR (400 MHz, DMSO-d₆) δ 10.12 (s, 1H), 7.90 (d, J=8.4 Hz, 1H), 7.77-7.70 (m, 2H), 7.46 (dd, J=8.8, 5.6 Hz, 1H), 7.38 (s, 1H), 6.91-6.83 (m, 1H), 6.49 (dd, J=9.6, 2.8 Hz, 1H), 4.10 (s, 3H), 3.98-3.90 (m, 1H), 3.75-3.66 (m, 1H), 3.35-3.25 (m, 1H), 3.18-3.08 (m, 1H), 3.08-2.99 (m, 1H), 2.01 (s, 3H), 1.99-1.84 (m, 2H), 1.72-1.61 (m, 1H), 1.60-1.48 (m, 1H).

### Examples 53-54. N-(7-chloro-4-(2-chloro-5-fluorophenoxy)-1-methyl-3-(methylamino)-1H-indazol-5-yl)-3-fluoro-5-(trifluoromethyl)benzamide & N-(7-chloro-4-(2-chloro-5-fluorophenoxy)-3-(dimethylamino)-1-methyl-1H-indazol-5-yl)-3-fluoro-5-(trifluoromethyl)benzamide

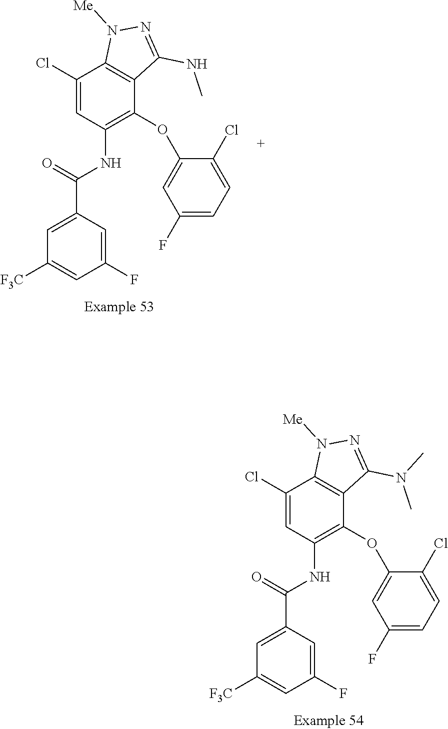 [SMILES: CNc1nn(C)c2c(Cl)cc(NC(=O)c3cc(F)cc(C(F)(F)F)c3)c(Oc3cc(F)ccc3Cl)c12 ; CN(C)c1nn(C)c2c(Cl)cc(NC(=O)c3cc(F)cc(C(F)(F)F)c3)c(Oc3cc(F)ccc3Cl)c12]

To the mixture of N-(3-amino-7-chloro-4-(2-chloro-5-fluorophenoxy)-1-methyl-1H-indazol-5-yl)-3-fluoro-5-(trifluoromethyl)benzamide (Example 24: 60 mg, 0.11 mmol) and formaldehyde (27 mg, 0.34 mmol, 37% in water) in methanol (2 mL) was added trifluoroacetic acid (20 mg, 0.34 mmol) dropwise at room temperature. The reaction was stirred for 1 h. To the above mixture was added sodium cyanoborohydride (21 mg, 0.34 mmol) at 0° C. and the mixture was stirred for additional 1 h. The resulting mixture was concentrated under reduced pressure. The residue was purified by silica gel column chromatography, eluted with 10% methanol in dichloromethane to afford crude product, which was then purified by Prep-HPLC with the following conditions (Column: XBridge Prep Phenyl OBD Column 19*250 mm; Mobile Phase: acetonitrile in water (0.05% 2,2,2-trifluoroacetic acid); Flow rate: 60 mL/min; 41% B to 61% B in 10 min; detector, 254/220 nm).

Eluted fractions were collected and lyophilized to afford the TFA salt of N-(7-chloro-4-(2-chloro-5-fluorophenoxy)-1-methyl-3-(methylamino)-1H-indazol-5-yl)-3-fluoro-5-(trifluoromethyl)benzamide as a white solid. LCMS calculated for C₂₃H₁₆Cl₂F₅N₄O₂(M+H)⁺ m/z=545.1; found 545.0; ¹H NMR (400 MHz, DMSO-d₆) δ 10.09 (s, 1H), 7.93-7.87 (m, 1H), 7.73-7.66 (m, 2H), 7.44-7.37 (m, 2H), 6.89-6.81 (m, 1H), 6.57-6.50 (m, 1H), 5.30 (q, J=5.2 Hz, 1H), 4.10 (s, 3H), 2.79 (d, J=5.2 Hz, 3H).

Eluted fractions were collected and lyophilized to afford the TFA salt of N-(7-chloro-4-(2-chloro-5-fluorophenoxy)-3-(dimethylamino)-1-methyl-1H-indazol-5-yl)-3-fluoro-5-(trifluoromethyl)benzamide as a pink solid. LCMS calculated for C₂₄H₁₈Cl₂F₅N₄O₂(M+H)⁺ m/z=559.1; found 559.1; ¹H NMR (400 MHz, DMSO-d₆) δ 10.27 (s, 1H), 7.95-7.88 (m, 1H), 7.79-7.71 (m, 2H), 7.57 (s, 1H), 7.46 (dd, J=8.8, 5.6 Hz, 1H), 6.87-6.80 (m, 1H), 6.30 (dd, J=9.6, 2.8 Hz, 1H), 4.20 (s, 3H), 2.76 (s, 6H).

### Example 55. N-(3-Amino-4-(2-chloro-5-fluorophenoxy)-7-(5,6-dihydro-[1,2,4]triazolo[1,5-a]pyrazin-7(8H)-yl)-1-methyl-1H-indazol-5-yl)-3-fluoro-5-(trifluoromethyl)benzamide

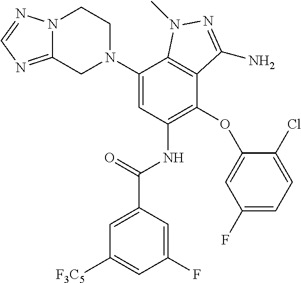

### Step 1: N-(4-(2-chloro-5-fluorophenoxy)-7-(5,6-dihydro-[1,2,4]triazolo[1,5-a]pyrazin-7(8H)-yl)-3-(1,3-dioxoisoindolin-2-yl)-1-methyl-1H-indazol-5-yl)-3-fluoro-5-(trifluoromethyl)benzamide

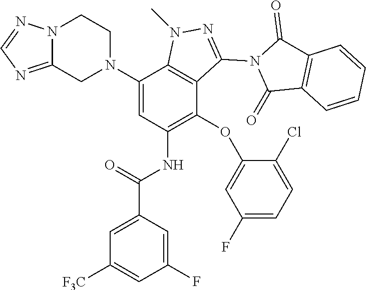 [SMILES: Cn1nc(N2C(=O)c3ccccc3C2=O)c2c(Oc3cc(F)ccc3Cl)c(NC(=O)c3cc(F)cc(C(F)(F)F)c3)cc(N3CCn4ncnc4C3)c21]

To a screw-cap vial equipped with a magnetic stir bar were placed N-(7-bromo-4-(2-chloro-5-fluorophenoxy)-3-(1,3-dioxoisoindolin-2-yl)-1-methyl-1H-indazol-5-yl)-3-fluoro-5-(trifluoromethyl)benzamide (Example 25, Step 1: 60 mg, 0.08 mmol), RuPhos Pd G2 (13 mg, 0.02 mmol) and cesium carbonate (83 mg, 0.25 mmol). The vial was sealed with a Teflon-lined septum, evacuated and backfilled with nitrogen (this process was repeated a total of three times). 5,6,7,8-Tetrahydro-[1,2,4]triazolo[1,5-a]pyrazine (21 mg, 0.17 mmol) in dioxane (2 mL) were added. The reaction was stirred at 100° C. for 1 h. Upon cooling to room temperature, the resulting mixture was concentrated under reduced pressure. The residue was purified by Prep-TLC (10% methanol in dichloromethane) to afford the desired product as a yellow solid (30 mg, 47%). LCMS calculated for C₃₅H₂₃ClF₅N₈O₄ (M+H)⁺ m/z=749.1; found 749.2;

### Step 2: N-(3-amino-4-(2-chloro-5-fluorophenoxy)-7-(5,6-dihydro-[1,2,4]triazolo[1,5-a]pyrazin-7(8H)-yl)-1-methyl-1H-indazol-5-yl)-3-fluoro-5-(trifluoromethyl)benzamide

The mixture of N-(4-(2-chloro-5-fluorophenoxy)-7-(5,6-dihydro-[1,2,4]triazolo[1,5-a]pyrazin-7(8H)-yl)-3-(1,3-dioxoisoindolin-2-yl)-1-methyl-1H-indazol-5-yl)-3-fluoro-5-(trifluoromethyl)benzamide (30 mg, 0.04 mmol) and hydrazine hydrate (98%, 0.5 mL) in methanol (2.5 mL) was stirred at room temperature for 1 h. The resulting mixture was concentrated under reduced pressure and then purified by Prep-HPLC with the following conditions (Column: Xselect CSH C18 OBD Column 30*150 mm 5 m; Mobile Phase: acetonitrile in water (0.05% trifluoroacetic acid); Flow rate: 60 mL/min; Gradient: 31% B to 51% B in 8 min; Detector: 254/220 nm). Eluted fractions were collected and lyophilized to afford the TFA salt of the desired product as a white solid. LCMS calculated for C₂₇H₂₁ClF₅N₈O₂ (M+H)⁺ m/z=619.1; found 619.1; ¹H NMR (400 MHz, DMSO-d₆) δ 10.17 (s, 1H), 8.01 (s, 1H), 7.93-7.86 (m, 1H), 7.82-7.73 (m, 2H), 7.46 (dd, J=8.8, 5.6 Hz, 1H), 7.13 (s, 1H), 6.92-6.83 (m, 1H), 6.47 (dd, J=9.6, 2.8 Hz, 1H), 4.50-4.24 (m, 6H), 3.96 (s, 3H).

### Example 56. N-(3-Amino-1-(2-(azetidin-1-yl)ethyl)-7-chloro-4-(2-chloro-5-fluorophenoxy)-1H-indazol-5-yl)-3-fluoro-5-(trifluoromethyl)benzamide

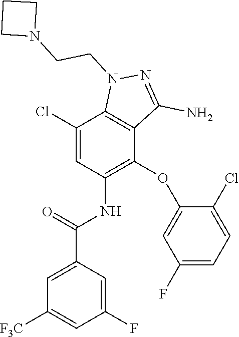 [SMILES: Nc1nn(CCN2CCC2)c2c(Cl)cc(NC(=O)c3cc(F)cc(C(F)(F)F)c3)c(Oc3cc(F)ccc3Cl)c12]

### Step 1: N-(1-(2-(azetidin-1-yl)ethyl)-7-chloro-4-(2-chloro-5-fluorophenoxy)-3-(1,3-dioxoisoindolin-2-yl)-1H-indazol-5-yl)-3-fluoro-5-(trifluoromethyl)benzamide

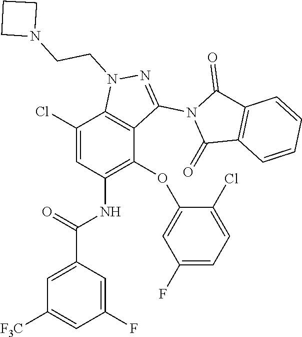 [SMILES: O=C(Nc1cc(Cl)c2c(c(N3C(=O)c4ccccc4C3=O)nn2CCN2CCC2)c1Oc1cc(F)ccc1Cl)c1cc(F)cc(C(F)(F)F)c1]

To the mixture of N-[7-chloro-4-(2-chloro-5-fluorophenoxy)-3-(1,3-dioxoisoindol-2-yl)-1H-indazol-5-yl]-3-fluoro-5-(trifluoromethyl)benzamide (Example 24, Step 1: 20 mg, 0.03 mmol) and potassium carbonate (13 mg, 0.09 mmol) in N,N-Dimethylformamide (1 mL) was added 1-(2-chloroethyl)azetidine hydrochloride (5.8 mg, 0.04 mmol) at room temperature. The reaction was stirred at 50° C. for 16 h. Upon cooling to room temperature, the resulting mixture was diluted with water (20 mL). The resulting mixture was extracted with ethyl acetate (3×20 mL). The combined organic layers were washed with brine (30 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a yellow solid. LCMS calculated for C₃₄H₂₃Cl₂F₅N₅O₄(M+H)⁺ m/z=730.1; found 730.1.

### Step 2: N-(3-amino-1-(2-(azetidin-1-yl)ethyl)-7-chloro-4-(2-chloro-5-fluorophenoxy)-1H-indazol-5-yl)-3-fluoro-5-(trifluoromethyl)benzamide

The mixture of N-(1-(2-(azetidin-1-yl)ethyl)-7-chloro-4-(2-chloro-5-fluorophenoxy)-3-(1,3-dioxoisoindolin-2-yl)-1H-indazol-5-yl)-3-fluoro-5-(trifluoromethyl)benzamide (30 mg, 0.04 mmol) and hydrazine hydrate (98%, 0.2 mL) in methanol (1 mL) was stirred at room temperature for 1 h. The resulting mixture was concentrated under reduced pressure. The residue was purified by Prep-HPLC with the following conditions (Column: XBridge Prep Phenyl OBD Column 19*250 mm; Mobile Phase: acetonitrile in water (0.1% ammonium hydroxide+10 mmol/L ammonium bicarbonate); Flow rate: 60 mL/min; Gradient: 39% B to 57% B in 10 min; Detector: 254/220 nm). Eluted fractions were collected and lyophilized to afford the desired product as a white solid. LCMS calculated for C₂₆H₂₁Cl₂F₅N₅O₂(M+H)⁺ m/z=600.1; found 600.2. ¹H NMR (400 MHz, DMSO-d₆) δ 10.21 (s, 1H), 7.94-7.88 (m, 1H), 7.77-7.70 (m, 2H), 7.49-7.43 (m, 2H), 7.91-6.84 (m, 1H), 6.50 (dd, J=9.6, 2.8 Hz, 1H), 5.16 (s, 2H), 4.38 (t, J=6.8 Hz, 2H), 3.10 (t, J=7.2 Hz, 4H), 2.75 (t, J=6.8 Hz, 2H), 1.98-1.88 (m, 2H).

### Examples 57-58. (E)-N-(3-amino-4-(2-chloro-5-fluorophenoxy)-7-(2-cyanovinyl)-1-methyl-1H-indazol-5-yl)-3-fluoro-5-(trifluoromethyl)benzamide & (Z)—N-(3-amino-4-(2-chloro-5-fluorophenoxy)-7-(2-cyanovinyl)-1-methyl-1H-indazol-5-yl)-3-fluoro-5-(trifluoromethyl)benzamide

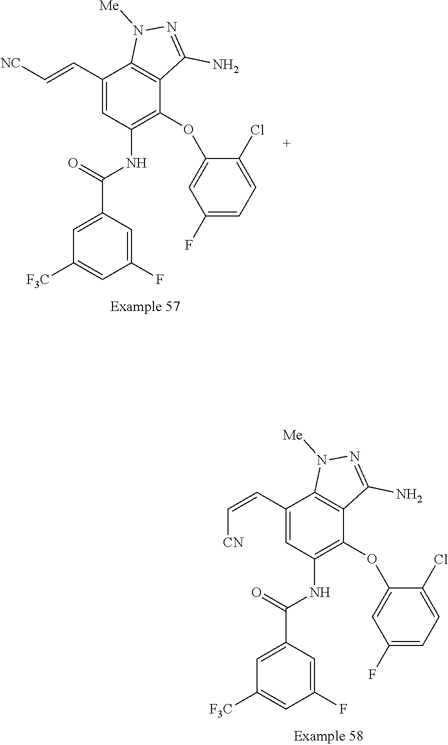 [SMILES: Cn1nc(N)c2c(Oc3cc(F)ccc3Cl)c(NC(=O)c3cc(F)cc(C(F)(F)F)c3)cc(/C=C/C#N)c21 ; Cn1nc(N)c2c(Oc3cc(F)ccc3Cl)c(NC(=O)c3cc(F)cc(C(F)(F)F)c3)cc(/C=C\C#N)c21]

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 20 mg, 0.04 mmol), Pd(OAc)₂ (0.78 mg, 0.004 mmol) and tri(o-tolyl)phosphine (2.11 mg, 0.007 mmol). The vial was sealed with a Teflon-lined septum, evacuated and backfilled with nitrogen (this process was repeated a total of three times). Acrylonitrile (2.4 mg, 0.05 mmol) and triethylamine (0.1 mL) in N,N-Dimethylformamide (1 mL) were added. The reaction was stirred at 135° C. for 2 h. Upon cooling to room temperature, the resulting mixture was diluted with water (20 mL). The resulting mixture was extracted with ethyl acetate (3×20 mL). The combined organic layers were washed with brine (20 mL), dried over anhydrous sodium sulfate. After filtration, the filtrate was concentrated under reduced pressure. The residue was purified by silica gel column chromatography, eluted with 50% ethyl acetate in petroleum ether to afford crude product, which was then purified by Prep-HPLC with the following conditions (column: YMC-Actus Triart C18 ExRS 30*150 mm, 5 m; Mobile Phase: acetonitrile in water (10 mmol/L ammonium bicarbonate); Flow rate: 60 mL/min mL/min; Gradient: 46% B to 63% B in 10 min; Wave Length: 254 nm/220 nm).

Eluted fractions were collected and lyophilized to afford (E)-N-(3-amino-4-(2-chloro-5-fluorophenoxy)-7-(2-cyanovinyl)-1-methyl-1H-indazol-5-yl)-3-fluoro-5-(trifluoromethyl)benzamide a yellow solid. LCMS calculated for C₂₅H₁₆ClF₅N₅O₂(M+H)+m/z=548.1; found 548.1. ¹H NMR (400 MHz, DMSO-d₆) δ 10.19 (s, 1H), 8.22 (d, J=16.4 Hz, 1H), 7.94-7.89 (m, 1H), 7.79-7.71 (m, 2H), 7.68 (s, 1H), 7.45 (dd, J=8.8, 5.6 Hz, 1H), 6.92-6.84 (m, 1H), 6.53 (dd, J=9.6, 2.8 Hz, 1H), 6.36 (d, J=16.4 Hz, 1H), 5.13 (s, 2H), 4.02 (s, 3H).

Eluted fractions were collected and lyophilized to afford (Z)—N-(3-amino-4-(2-chloro-5-fluorophenoxy)-7-(2-cyanovinyl)-1-methyl-1H-indazol-5-yl)-3-fluoro-5-(trifluoromethyl)benzamide as a yellow solid. C₂₅H₁₆ClF₅N₅O₂(M+H)⁺ m/z=548.1; found 548.0. ¹H NMR (400 MHz, DMSO-d₆) δ 10.22 (s, 1H), 8.05 (d, J=11.6 Hz, 1H), 7.94-7.88 (m, 1H), 7.79-7.78 (m, 2H), 7.62 (s, 1H), 7.45 (dd, J=8.8, 5.6 Hz, 1H), 6.92-6.84 (m, 1H), 6.54 (dd, J=9.6, 2.8 Hz, 1H), 6.05 (d, J=11.6 Hz, 1H), 5.14 (s, 2H), 3.94 (s, 3H).

### Example 59. N-(3-Amino-4-(2-chloro-5-fluorophenoxy)-7-(2-cyanoethyl)-1-methyl-1H-indazol-5-yl)-3-fluoro-5-(trifluoromethyl)benzamide

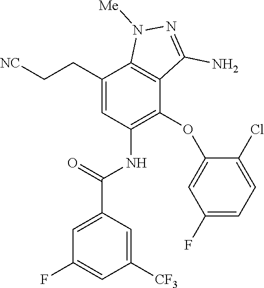 [SMILES: Cn1nc(N)c2c(Oc3cc(F)ccc3Cl)c(NC(=O)c3cc(F)cc(C(F)(F)F)c3)cc(CCC#N)c21]

To the mixture of (E)-N-(3-amino-4-(2-chloro-5-fluorophenoxy)-7-(2-cyanovinyl)-1-methyl-1H-indazol-5-yl)-3-fluoro-5-(trifluoromethyl)benzamide (Example 57: 20 mg, 0.04 mmol) in ethyl acetate (3 mL) was added Pd/C (10%, 5.4 mg, 0.004 mmol) at room temperature. The flask was evacuated and flushed with nitrogen, followed by flushing with hydrogen (this process was repeated a total of three times). The reaction was stirred at room temperature for 2 h. The resulting mixture was filtered, the filter cake was washed with ethyl acetate (30 mL), and the filtrate was concentrated under reduced pressure. The residue was purified by silica gel column chromatography, eluted with 50% ethyl acetate in dichloromethane to afford the crude product, which was then purified by reversed-phase flash chromatography with the following conditions (Column, C18 silica gel; Mobile phase, acetonitrile in water (0.1% 2,2,2-trifluoroacetic acid); 30% to 70% gradient in 20 min; Detector: 254/220 nm). Eluted fractions were collected and lyophilized to afford the TFA salt of the desired product as a white solid. LCMS calculated for C₂₅H₁₈ClF₅N₅O₂(M+H)⁺ m/z=550.1; found 550.1; ¹H NMR (400 MHz, DMSO-d₆) δ 10.18 (s, 1H), 7.90 (d, J=8.4 Hz, 1H), 7.82-7.74 (m, 2H), 7.46 (dd, J=8.8, 5.6 Hz, 1H), 7.18 (s, 1H), 6.90-6.83 (m, 1H), 6.41 (dd, J=9.6, 2.8 Hz, 1H), 4.01 (s, 3H), 3.36 (t, J=7.2 Hz, 2H), 2.96 (t, J=7.2 Hz, 2H).

### Example 60. N-(3-Amino-7-chloro-4-(2-chloro-5-fluorophenoxy)-1-methyl-1H-indazol-5-yl)-3-chloro-5-fluorobenzamide

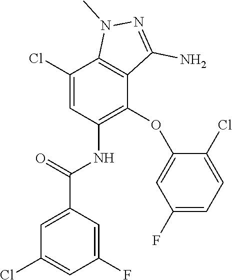 [SMILES: Cn1nc(N)c2c(Oc3cc(F)ccc3Cl)c(NC(=O)c3cc(F)cc(Cl)c3)cc(Cl)c21]

### Step 1: Tert-butyl (2-(2-chloro-5-fluorophenoxy)-3-cyano-4-fluorophenyl)carbamate

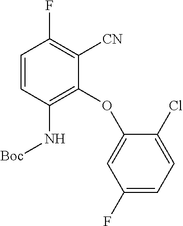 [SMILES: CC(C)(C)OC(=O)Nc1ccc(F)c(C#N)c1Oc1cc(F)ccc1Cl]

To the mixture of 3-amino-2-(2-chloro-5-fluorophenoxy)-6-fluorobenzonitrile (Example 1, Step 2: 5 g, 17.8 mmol), and 4-dimethylaminopyridine (217 mg, 1.8 mmol) in tetrahydrofuran (100 mL) was added di-tert-butyl dicarbonate (9.72 g, 44.5 mmol) dropwise at room temperature. The reaction was stirred for additional 6 h. The resulting mixture was diluted with water (200 mL). The aqueous solution was extracted with ethyl acetate (3×200 mL). The combined organic layers were washed with brine (300 mL), dried over anhydrous sodium sulfate. After filtration, the filtrate was concentrated under reduced pressure. The residue was purified by silica gel column chromatography, eluted with 40% ethyl acetate in petroleum ether to afford the desired product as a white solid (6.5 g, 95%). LCMS calculated for C₁₈H₁₆ClF₂N₂O₃ (M+H)⁺ m/z=381.1; found 381.0.

### Step 2: Tert-butyl (3-amino-4-(2-chloro-5-fluorophenoxy)-1H-indazol-5-yl)carbamate

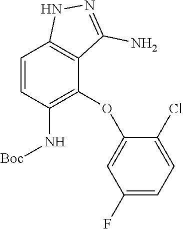 [SMILES: CC(C)(C)OC(=O)Nc1ccc2[nH]nc(N)c2c1Oc1cc(F)ccc1Cl]

To the mixture of tert-butyl (2-(2-chloro-5-fluorophenoxy)-3-cyano-4-fluorophenyl)carbamate (3 g, 7.9 mmol) in 1-butanol (30 mL) was added hydrazine hydrate (98%, 3 mL) at room temperature. The reaction was stirred at 110° C. for 2 h. Upon cooling to room temperature. The resulting mixture was concentrated under reduced pressure. The residue was purified by silica gel column chromatography, eluted with 50% ethyl acetate in petroleum ether to afford the desired product as a colorless oil (3 g, 97%). LCMS calculated for C₁₈H₁₉ClFN₄O₃(M+H)⁺ m/z=393.1; found 393.3.

### Step 3: Tert-butyl (4-(2-chloro-5-fluorophenoxy)-3-(1,3-dioxoisoindolin-2-yl)-1H-indazol-5-yl)carbamate

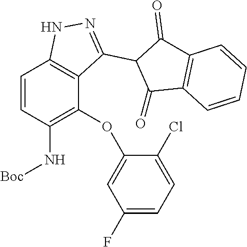 [SMILES: CC(C)(C)OC(=O)Nc1ccc2[nH]nc(C3C(=O)c4ccccc4C3=O)c2c1Oc1cc(F)ccc1Cl]

The mixture of tert-butyl N-[3-amino-4-(2-chloro-5-fluorophenoxy)-1H-indazol-5-yl]carbamate (3 g, 7.6 mmol) and phthalic anhydride (1.7 g, 11.5 mmol) in dioxane (30 mL) was stirred at 120° C. for 16 h. Upon cooling to room temperature. The resulting mixture was concentrated under reduced pressure. The residue was purified by silica gel column chromatography, eluted with 50% ethyl acetate in petroleum ether to afford the desired product as a white solid. LCMS calculated for C₂₆H₁₉ClFN₄O₅(M−H)⁻ m/z=521.1; found 521.3.

### Step 4: Tert-butyl (7-chloro-4-(2-chloro-5-fluorophenoxy)-3-(1,3-dioxoisoindolin-2-yl)-1H-indazol-5-yl)carbamate

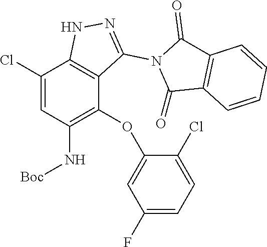 [SMILES: CC(C)(C)OC(=O)Nc1cc(Cl)c2[nH]nc(N3C(=O)c4ccccc4C3=O)c2c1Oc1cc(F)ccc1Cl]

To the mixture of tert-butyl (4-(2-chloro-5-fluorophenoxy)-3-(1,3-dioxoisoindolin-2-yl)-1H-indazol-5-yl)carbamate (600 mg, 1.15 mmol) and AcOH (69 mg, 1.15 mmol) in acetonitrile (10 mL) was added N-chlorosuccinimide (184 mg, 1.38 mmol) in portions at 0° C. The reaction was stirred at 60° C. for 2 h. Upon cooling to room temperature, the resulting mixture was diluted with water (50 mL). The aqueous solution was extracted with ethyl acetate (3×50 mL). The combined organic layers were washed with sat. sodium thiosulfate (2×50 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a purple solid (300 mg, 47%). LCMS calculated for C₂₆H₁₈Cl₂FN₄O₅(M−H)⁻ m/z=555.1; found 555.1.

### Step 5: Tert-butyl (7-chloro-4-(2-chloro-5-fluorophenoxy)-3-(1,3-dioxoisoindolin-2-yl)-1-methyl-1H-indazol-5-yl)carbamate

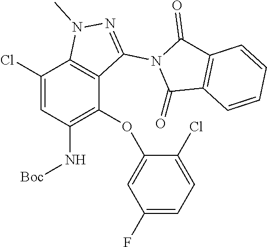 [SMILES: Cn1nc(N2C(=O)c3ccccc3C2=O)c2c(Oc3cc(F)ccc3Cl)c(NC(=O)OC(C)(C)C)cc(Cl)c21]

To the mixture of tert-butyl N-[7-chloro-4-(2-chloro-5-fluorophenoxy)-3-(1,3-dioxoisoindol-2-yl)-1H-indazol-5-yl]carbamate (300 mg, 0.54 mmol) and potassium carbonate (223 mg, 1.61 mmol) in N,N-dimethylformamide (5 mL) was added iodomethane (92 mg, 0.65 mmol) dropwise at 0° C. The reaction was stirred at room temperature for 2 h. The resulting mixture was diluted with water (50 mL). The aqueous solution was extracted with ethyl acetate (3×50 mL). The combined organic layers were washed with brine (50 mL), dried over anhydrous sodium sulfate. After filtration, the filtrate was concentrated under reduced pressure. The residue was purified by silica gel column chromatography, eluted with 20% ethyl acetate in petroleum ether to afford the desired product as a purple solid (170 mg, 55%). LCMS calculated for C₂₇H₂₀Cl₂FN₄O₅(M−H)⁻ m/z=569.1; found 569.0.

### Step 6: 2-(5-Amino-7-chloro-4-(2-chloro-5-fluorophenoxy)-1-methyl-1H-indazol-3-yl)isoindoline-1,3-dione

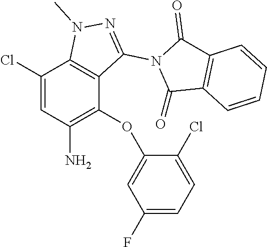 [SMILES: Cn1nc(N2C(=O)c3ccccc3C2=O)c2c(Oc3cc(F)ccc3Cl)c(N)cc(Cl)c21]

The mixture of tert-butyl N-[7-chloro-4-(2-chloro-5-fluorophenoxy)-3-(1,3-dioxoisoindol-2-yl)-1-methylindazol-5-yl]carbamate (170 mg, 0.298 mmol) and hydrochloric acid in 1,4-dioxane (4 M, 1 mL) in dichloromethane (2 mL) was stirred at room temperature for 2 h. The reaction was concentrated under reduced pressure. The resulting mixture was diluted with water (20 mL). The mixture was basified to pH 8 with saturated aq. sodium bicarbonate. The aqueous solution was extracted with dichloromethane (3×20 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a purple solid (120 mg, 85%). LCMS calculated for C₂₂H₁₄Cl₂FN₄O₃(M+H)⁺ m/z=471.0; found 471.1.

### Step 7: 3-Chloro-N-(7-chloro-4-(2-chloro-5-fluorophenoxy)-3-(1,3-dioxoisoindolin-2-yl)-1-methyl-1H-indazol-5-yl)-5-fluorobenzamide

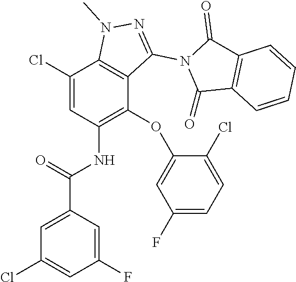 [SMILES: Cn1nc(N2C(=O)c3ccccc3C2=O)c2c(Oc3cc(F)ccc3Cl)c(NC(=O)c3cc(F)cc(Cl)c3)cc(Cl)c21]

To the mixture of 2-[5-amino-7-chloro-4-(2-chloro-5-fluorophenoxy)-1-methylindazol-3-yl]isoindole-1,3-dione (30 mg, 0.064 mmol) in pyridine (1 mL) was added 3-chloro-5-fluorobenzoyl chloride (14.7 mg, 0.08 mmol) at 0° C. The reaction was stirred at room temperature for 1 h. The resulting mixture was diluted with water (10 mL). The aqueous solution was extracted with ethyl acetate (3×10 mL). The resulting mixture was concentrated under reduced pressure to afford desired product. The crude product was used in the next step directly without further purification. LCMS calculated for C₂₉H₁₄Cl₃F₂N₄O₄(M−H)⁻ m/z=625.0; found 625.2.

### Step 8: N-(3-amino-7-chloro-4-(2-chloro-5-fluorophenoxy)-1-methyl-1H-indazol-5-yl)-3-chloro-5-fluorobenzamide

To the mixture of 3-chloro-N-(7-chloro-4-(2-chloro-5-fluorophenoxy)-3-(1,3-dioxoisoindolin-2-yl)-1-methyl-1H-indazol-5-yl)-5-fluorobenzamide (30 mg, 0.05 mmol) in methanol (1 mL) was added hydrazine hydrate (98%, 0.2 mL) at room temperature. The reaction was stirred for 1 h. The resulting mixture was concentrated under reduced pressure. The residue was purified by Prep-HPLC with the following conditions (Column: Sunfire prep C18 column, 30*150 mm; Mobile Phase: acetonitrile in water (0.05% trifluoroacetic acid); Flow rate: 60 mL/min; Gradient: 45% B to 65% B in 10 min; Detector: 254/220 nm). Eluted fractions were collected and lyophilized to afford the TFA salt of the desired product as a white solid. LCMS calculated for C₂₁H₁₄Cl₃F₂N₄O₂(M+H)⁺ m/z=497.0; found 497.1; ¹H NMR (400 MHz, DMSO-d₆) δ 10.07 (s, 1H), 7.68-7.61 (m, 1H), 7.54-7.46 (m, 2H), 7.45-7.38 (m, 2H), 6.95-6.86 (m, 1H), 6.52-6.45 (m, 1H), 5.09 (s, 2H), 4.07 (s, 3H).

### Example 61. N-(3-Amino-7-chloro-4-(2-chloro-5-fluorophenoxy)-1-methyl-1H-indazol-5-yl)-3-(trifluoromethyl)benzamide

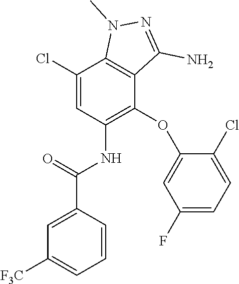 [SMILES: Cn1nc(N)c2c(Oc3cc(F)ccc3Cl)c(NC(=O)c3cccc(C(F)(F)F)c3)cc(Cl)c21]

### Step 1: N-(7-chloro-4-(2-chloro-5-fluorophenoxy)-3-(1,3-dioxoisoindolin-2-yl)-1-methyl-1H-indazol-5-yl)-3-(trifluoromethyl)benzamide

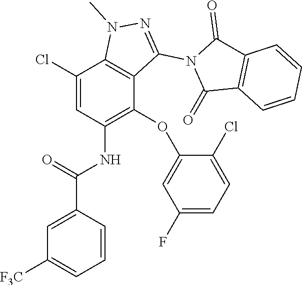 [SMILES: Cn1nc(N2C(=O)c3ccccc3C2=O)c2c(Oc3cc(F)ccc3Cl)c(NC(=O)c3cccc(C(F)(F)F)c3)cc(Cl)c21]

To the mixture of 2-[5-amino-7-chloro-4-(2-chloro-5-fluorophenoxy)-1-methylindazol-3-yl]isoindole-1,3-dione (Example 60, Step 6: 20 mg, 0.04 mmol) in pyridine (1 mL) was added 3-(trifluoromethyl)benzoyl chloride (9.74 mg, 0.05 mmol) at 0° C. The reaction was stirred for 1 h. The resulting mixture was diluted with water (10 mL). The aqueous layer was extracted with ethyl acetate (3×10 mL). The combined organic layers were washed with brine (10 mL), dried over anhydrous sodium sulfate. After filtration, the filtrate was concentrated under reduced pressure. The crude product was used in the next step directly without further purification. LCMS calculated for C₃₀H₁₇Cl₂F₄N₄O₄(M+H)⁺ m/z=643.1; found 643.1.

### Step 2: N-(3-amino-7-chloro-4-(2-chloro-5-fluorophenoxy)-1-methyl-1H-indazol-5-yl)-3-(trifluoromethyl)benzamide

To the mixture of N-[7-chloro-4-(2-chloro-5-fluorophenoxy)-3-(1,3-dioxoisoindol-2-yl)-1-methylindazol-5-yl]-3-(trifluoromethyl)benzamide (20 mg, 0.03 mmol) in methanol (1 mL) was added hydrazine hydrate (98%, 0.2 mL) at room temperature. The reaction was stirred for 1 h. The resulting mixture was concentrated under reduced pressure. The residue was purified by Prep-HPLC with the following conditions (Column: Sunfire prep C18 column, 30*150 mm, 5 m; Mobile Phase: acetonitrile in water (0.05% trifluoroacetic acid); Flow rate: 60 mL/min mL/min; Gradient: 48% B to 68% B in 10 min; Wave Length: 254 nm/220 nm nm). Eluted fractions were collected and lyophilized to afford the TFA salt of the desired product as a white solid. LCMS calculated for C₂₂H₁₅Cl₂F₄N₄O₂(M+H)⁺ m/z=513.1; found 513.0; ¹H NMR (400 MHz, DMSO-d₆) δ 10.13 (s, 1H), 7.97-7.87 (m, 3H), 7.72-7.64 (m, 1H), 7.49-7.42 (m, 2H), 6.91-6.82 (m, 1H), 6.50 (dd, J=9.6, 2.8 Hz, 1H), 5.09 (s, 2H), 4.07 (s, 3H).

### Example 62. N-(3-Amino-7-chloro-4-(2-chloro-5-fluorophenoxy)-1-methyl-1H-indazol-5-yl)-5-(trifluoromethyl)nicotinamide

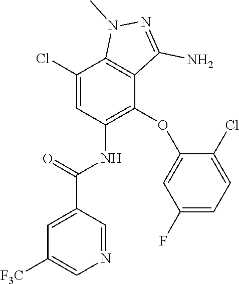 [SMILES: Cn1nc(N)c2c(Oc3cc(F)ccc3Cl)c(NC(=O)c3cncc(C(F)(F)F)c3)cc(Cl)c21]

The title compound was prepared according to the procedure described in Example 61, using 5-(trifluoromethyl)nicotinoyl chloride instead of 3-(trifluoromethyl)benzoyl chloride. LCMS calculated for C₂₁H₁₄Cl₂F₄N₅O₂(M+H)⁺ m/z=514.0; found 514.0. ¹H NMR (400 MHz, DMSO-d₆) δ 10.30 (s, 1H), 9.14-9.11 (m, 1H), 9.05-9.02 (m, 1H), 8.28-8.25 (m, 1H), 7.50-7.43 (m, 2H), 6.92-6.85 (m, 1H), 6.53 (dd, J=9.6, 2.8 Hz, 1H), 5.10 (s, 2H), 4.08 (s, 3H).

### Example 63. N-(3-Amino-7-chloro-4-(2-chloro-5-fluorophenoxy)-1-methyl-1H-indazol-5-yl)benzo[b]thiophene-3-carboxamide

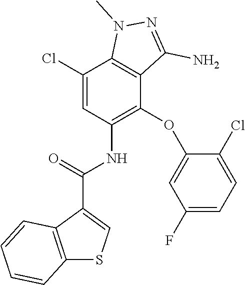 [SMILES: Cn1nc(N)c2c(Oc3cc(F)ccc3Cl)c(NC(=O)c3csc4ccccc34)cc(Cl)c21]

The title compound was prepared according to the procedure described in Example 61, using benzo[b]thiophene-3-carbonyl chloride instead of 3-(trifluoromethyl)benzoyl chloride. LCMS calculated for C₂₃H₁₆Cl₂FN₄O₂S (M+H) m/z=501.1; found 501.2. ¹H NMR (400 MHz, DMSO-d₆) δ 9.95 (s, 1H), 8.25-8.19 (m, 1H), 8.16 (s, 1H), 8.05-7.99 (m, 1H), 7.53-7.45 (m, 2H), 7.44-7.37 (m, 2H), 6.90-6.83 (m, 1H), 6.55-6.47 (m, 1H), 5.03 (s, 2H), 4.08 (s, 3H).

### Example 64. N-(3-Amino-7-chloro-4-(2-chloro-5-fluorophenoxy)-1-methyl-1H-indazol-5-yl)indoline-1-carboxamide

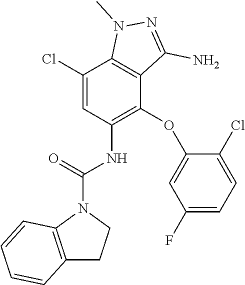 [SMILES: Cn1nc(N)c2c(Oc3cc(F)ccc3Cl)c(NC(=O)N3CCc4ccccc43)cc(Cl)c21]

### Step 1: N-(7-chloro-4-(2-chloro-5-fluorophenoxy)-3-(1,3-dioxoisoindolin-2-yl)-1-methyl-1H-indazol-5-yl)indoline-1-carboxamide

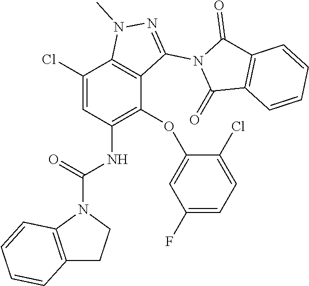 [SMILES: Cn1nc(N2C(=O)c3ccccc3C2=O)c2c(Oc3cc(F)ccc3Cl)c(NC(=O)N3CCc4ccccc43)cc(Cl)c21]

To a stirred mixture of 2-[5-amino-7-chloro-4-(2-chloro-5-fluorophenoxy)-1-methylindazol-3-yl]isoindole-1,3-dione (Example 60, Step 6: 30 mg, 0.06 mmol) and pyridine (25 mg, 0.32 mmol) in DCM (1 mL) was added 4-nitrophenyl carbonochloridate (12.8 mg, 0.06 mmol) dropwise at room temperature. The reaction was stirred for additional 4 h. The resulting mixture was concentrated under reduce pressure. The residue was taken in tetrahydrofuran (1 mL). To the above mixture was added indoline (8.3 mg, 0.07 mmol) at room temperature. The reaction was stirred for 16 h. The resulting mixture was diluted with water (10 mL). The aqueous solution was extracted with ethyl acetate (3×10 mL). The combined organic layers were washed with brine (10 mL), dried over anhydrous sodium sulfate. After filtration, the filtrate was concentrated under reduced pressure. The crude product was used in the next step directly without further purification. LCMS calculated for C₃₁H₂₁Cl₂FN₅O₄(M+H)⁺ m/z=616.1; found 616.2.

### Step 2: N-(3-Amino-7-chloro-4-(2-chloro-5-fluorophenoxy)-1-methyl-1H-indazol-5-yl)indoline-1-carboxamide

To the mixture of N-[7-chloro-4-(2-chloro-5-fluorophenoxy)-3-(1,3-dioxoisoindol-2-yl)-1-methylindazol-5-yl]-2,3-dihydroindole-1-carboxamide (30 mg, 0.05 mmol) in methanol (1 mL) was added hydrazine hydrate (98%, 0.2 mL) at room temperature. The reaction was stirred for 1 h. The resulting mixture was concentrated under reduced pressure. The residue was purified by Prep-HPLC with the following conditions (Column: XBridge Prep Phenyl OBD Column 19*250 mm, 5 m; Mobile Phase: acetonitrile in water (10 mmol/L ammonium bicarbonate+0.1% ammonium hydroxide); Flow rate: 60 mL/min mL/min; Gradient: 39% B to 57% B in 10 min; Wave Length: 254 nm/220 nm.). Eluted fractions were collected and lyophilized to afford the desired product as a white solid. LCMS calculated for C₂₃H₁₉Cl₂FN₅O₂ (M+H)⁺ m/z=486.1; found 486.2. ¹H NMR (400 MHz, DMSO-d₆) δ 8.15 (s, 1H), 7.72-7.66 (m, 1H), 7.53 (dd, J=8.8, 5.6 Hz, 1H), 7.40 (s, 1H), 7.17-7.11 (m, 1H), 7.10-7.02 (m, 1H), 6.94-6.81 (m, 2H), 6.52 (dd, J=9.6, 2.8 Hz, 1H), 5.04 (s, 2H), 4.06 (s, 3H), 3.69 (t, J=8.4 Hz, 2H), 3.04 (t, J=8.4 Hz, 2H).

### Example 65. 3-Amino-4-(2-chloro-5-fluorophenoxy)-N-ethyl-5-(3-fluoro-5-(trifluoromethyl)benzamido)-1-methyl-1H-indazole-7-carboxamide

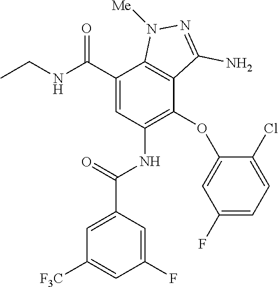 [SMILES: CCNC(=O)c1cc(NC(=O)c2cc(F)cc(C(F)(F)F)c2)c(Oc2cc(F)ccc2Cl)c2c(N)nn(C)c12]

Step 1: methyl 4-(2-chloro-5-fluorophenoxy)-3-(1,3-dioxoisoindolin-2-yl)-5-(3-fluoro-5-(trifluoromethyl)benzamido)-1-methyl-1H-indazole-7-carboxylate

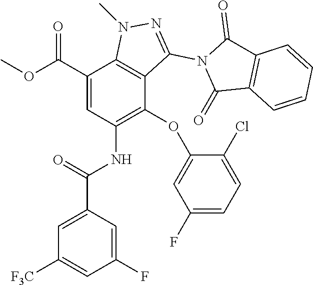 [SMILES: COC(=O)c1cc(NC(=O)c2cc(F)cc(C(F)(F)F)c2)c(Oc2cc(F)ccc2Cl)c2c(N3C(=O)c4ccccc4C3=O)nn(C)c12]

To the mixture of N-(7-bromo-4-(2-chloro-5-fluorophenoxy)-3-(1,3-dioxoisoindolin-2-yl)-1-methyl-1H-indazol-5-yl)-3-fluoro-5-(trifluoromethyl)benzamide (Example 25, Step 1: 100 mg, 0.14 mmol) in methanol (3 mL) were added XantPhos (16.4 mg, 0.03 mmol), Pd(OAc)₂ (3.2 mg, 0.014 mmol) and triethylamine (143 mg, 1.42 mmol) in a pressure tank. The tank was evacuated and flushed three times with nitrogen, followed by flushing with carbon monoxide. The mixture was stirred at 70° C. for 16 h under an atmosphere of carbon monoxide (balloon). Upon cooling to room temperature, the mixture was filtered. The filtrate was concentrated under reduced pressure. The residue was purified by silica gel column chromatography, eluted with 10% methanol in dichloromethane to afford the desired product as a white solid (80 mg, 82%). LCMS calculated for C₃₂H₁₉ClF₅N₄O₆ (M+H)⁺ m/z=685.1; found 685.1;

### Step 2: 3-(2-carboxybenzamido)-4-(2-chloro-5-fluorophenoxy)-5-(3-fluoro-5-(trifluoromethyl)benzamido)-1-methyl-1H-indazole-7-carboxylic acid

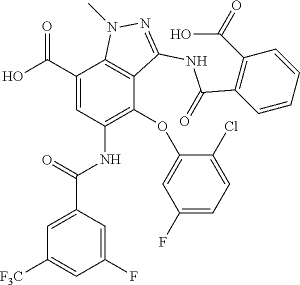 [SMILES: Cn1nc(NC(=O)c2ccccc2C(=O)O)c2c(Oc3cc(F)ccc3Cl)c(NC(=O)c3cc(F)cc(C(F)(F)F)c3)cc(C(=O)O)c21]

To the mixture of methyl 4-(2-chloro-5-fluorophenoxy)-3-(1,3-dioxoisoindolin-2-yl)-5-(3-fluoro-5-(trifluoromethyl)benzamido)-1-methyl-1H-indazole-7-carboxylate (80 mg, 0.12 mmol) in tetrahydrofuran (0.6 mL), methanol (0.2 mL) and water (0.2 mL) was added lithium hydroxide monohydrate (5.6 mg, 0.23 mmol) at room temperature. The reaction was stirred for additional 3 h. The mixture was concentrated under reduced pressure. Water (1 mL) was added, and the mixture was neutralized to pH 5 with aq. hydrochloric acid (1 M). The precipitated solids were collected by filtration and washed with water (3×5 mL), and then was dried under vacuum. The crude product was used in the next step directly without further purification. LCMS calculated for C₃₁H₁₉ClF₅N₄O₇ (M+H)⁺ m/z=689.1; found 689.1;

### Step 3: N′-(4-(2-chloro-5-fluorophenoxy)-7-(ethylcarbamoyl)-5-(3-fluoro-5-(trifluoromethyl)benzamido)-1-methyl-1H-indazol-3-yl)-N²-ethylphthalamide

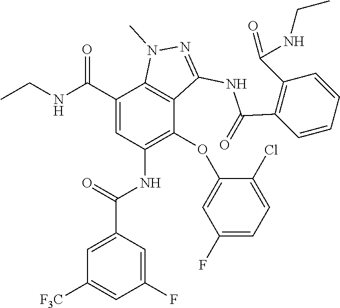 [SMILES: CCNC(=O)c1ccccc1C(=O)Nc1nn(C)c2c(C(=O)NCC)cc(NC(=O)c3cc(F)cc(C(F)(F)F)c3)c(Oc3cc(F)ccc3Cl)c12]

The mixture of 3-(2-carboxybenzamido)-4-(2-chloro-5-fluorophenoxy)-5-(3-fluoro-5-(trifluoromethyl)benzamido)-1-methyl-1H-indazole-7-carboxylic acid (20 mg, 0.03 mmol) and 2-(7-Azabenzotriazol-1-yl)-N,N,N′,N′-tetramethyluronium hexafluorophosphate (33 mg, 0.09 mmol) in N,N-dimethylformamide (2 mL) was stirred at room temperature for 0.5 h. To the above mixture was added N,N-diisopropylethylamine (18.8 mg, 0.15 mmol) and ethylamine (65% in water, 4.5 mg, 0.06 mmol). The reaction was stirred for additional 1 h. The resulting mixture was diluted with water (20 mL). The aqueous solution was extracted with ethyl acetate (3×20 mL). The combined organic layers were washed with brine (50 mL), dried over anhydrous sodium sulfate. After filtration, the filtrate was concentrated under reduced pressure. The crude product was used in the next step directly without further purification. LCMS calculated for C₃₅H₂₉ClF₅N₆O₅ (M+H)⁺ m/z=743.2; found 743.2;

### Step 4: 3-Amino-4-(2-chloro-5-fluorophenoxy)-N-ethyl-5-(3-fluoro-5-(trifluoromethyl)benzamido)-1-methyl-1H-indazole-7-carboxamide

To the mixture of N′-(4-(2-chloro-5-fluorophenoxy)-7-(ethylcarbamoyl)-5-(3-fluoro-5-(trifluoromethyl)benzamido)-1-methyl-1H-indazol-3-yl)-N²-ethylphthalamide (17 mg, 0.02 mmol) in methanol (3 mL) was added hydrazine hydrate (98%, 0.3 mL) at room temperature.

The reaction was stirred at room temperature for 1 h. The resulting mixture was concentrated under reduced pressure. The residue was purified by silica gel column chromatography, eluted with 10% methanol in dichloromethane to afford crude product, which was then purified by Prep-HPLC with the following conditions (Column: Sunfire prep C18 column, 30*150 mm; Mobile Phase: acetonitrile in water (0.05% trifluoroacetic acid); Flow rate: 60 mL/min; Gradient: 35% B to 55% B in 10 min; Detector: 254/220 nm). Eluted fractions were collected and lyophilized to afford the TFA salt of the desired product as an off-white solid. LCMS calculated for C₂₅H₂₀ClF₅N₅O₃ (M+H)⁺ m/z=568.1; found 568.1. ¹H NMR (400 MHz, DMSO-d₆) δ 10.19 (s, 1H), 8.70 (t, J=5.6 Hz, 1H), 7.95-7.87 (m, 1H), 7.79-7.73 (m, 2H), 7.48 (dd, J=8.8, 5.6 Hz, 1H), 7.35 (s, 1H), 6.93-6.83 (m, 1H), 6.46 (dd, J=9.6, 2.8 Hz, 1H), 4.99 (s, 2H), 3.77 (s, 3H), 3.37-3.26 (m, 2H), 1.16 (t, J=7.2 Hz, 3H).

### Example 66. N-(3-Amino-4-(2-chloro-5-fluorophenoxy)-7-(2-hydroxyethyl)-1-methyl-1H-indazol-5-yl)-3-fluoro-5-(trifluoromethyl)benzamide

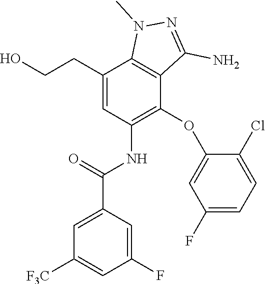 [SMILES: Cn1nc(N)c2c(Oc3cc(F)ccc3Cl)c(NC(=O)c3cc(F)cc(C(F)(F)F)c3)cc(CCO)c21]

### Step 1: N-(4-(2-chloro-5-fluorophenoxy)-3-(1,3-dioxoisoindolin-2-yl)-1-methyl-7-vinyl-1H-indazol-5-yl)-3-fluoro-5-(trifluoromethyl)benzamide

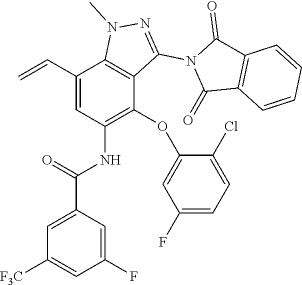 [SMILES: C=Cc1cc(NC(=O)c2cc(F)cc(C(F)(F)F)c2)c(Oc2cc(F)ccc2Cl)c2c(N3C(=O)c4ccccc4C3=O)nn(C)c12]

To a screw-cap vial equipped with a magnetic stir bar were placed N-[7-bromo-4-(2-chloro-5-fluorophenoxy)-3-(1,3-dioxoisoindol-2-yl)-1-methylindazol-5-yl]-3-fluoro-5-(trifluoromethyl)benzamide (Example 11, Step 1: 50 mg, 0.071 mmol), 2-ethenyl-4,4,5,5-tetramethyl-1,3,2-dioxaborolane (16.4 mg, 0.11 mmol), XPhos Pd G3 (11.99 mg, 0.01 mmol) and potassium carbonate (29 mg, 0.2 mmol). The vial was sealed with a Teflon-lined septum, evacuated and backfilled with nitrogen (this process was repeated a total of three times). Toluene (1 mL) and water (0.2 mL) were added. The reaction was stirred at 80° C. for 2 h. Upon cooling to room temperature, the resulting mixture was concentrated under reduced pressure. The residue was purified by silica gel column chromatography, eluted with 50% ethyl acetate in petroleum ether to afford the desired product as a yellow solid (30 mg, 65%). LCMS calculated for C₃₂H₁₇ClF₅N₄O₄ (M−H)⁻ m/z=651.1; found 651.1.

### Step 2: N-(4-(2-chloro-5-fluorophenoxy)-3-(1,3-dioxoisoindolin-2-yl)-7-(2-hydroxyethyl)-1-methyl-1H-indazol-5-yl)-3-fluoro-5-(trifluoromethyl)benzamide

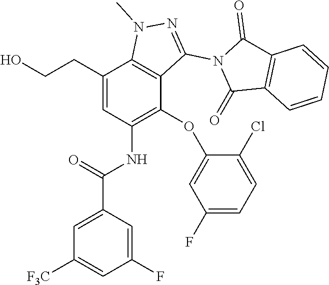 [SMILES: Cn1nc(N2C(=O)c3ccccc3C2=O)c2c(Oc3cc(F)ccc3Cl)c(NC(=O)c3cc(F)cc(C(F)(F)F)c3)cc(CCO)c21]

To a screw-cap vial equipped with a magnetic stir bar was placed N-(4-(2-chloro-5-fluorophenoxy)-3-(1,3-dioxoisoindolin-2-yl)-1-methyl-7-vinyl-1H-indazol-5-yl)-3-fluoro-5-(trifluoromethyl)benzamide (30 mg, 0.05 mmol). The vial was sealed with a Teflon-lined septum, evacuated and backfilled with nitrogen (this process was repeated a total of three times). Tetrahydrofuran (1 mL) was added, followed by the addition of borane-tetrahydrofuran complex (1 M, 0.25 mL, 0.25 mmol) at 0° C. The reaction was stirred at room temperature for 2 h. To the above solution was added sodium perborate tetrahydrate (38 mg, 0.46 mmol) in water (0.2 mL). The resulting mixture was then stirred for 16 h, and then was concentrated under reduced pressure. The residue was purified by Prep-TLC (50% ethyl acetate in petroleum ether) to afford the desired product as a yellow solid (25 mg, 81%). LCMS calculated for C₃₂H₂₁ClF₅N₄O₅ (M+H)⁺ m/z=671.1; found 671.3.

### Step 3: N-(3-amino-4-(2-chloro-5-fluorophenoxy)-7-(2-hydroxyethyl)-1-methyl-1H-indazol-5-yl)-3-fluoro-5-(trifluoromethyl)benzamide

To the mixture of N-(4-(2-chloro-5-fluorophenoxy)-3-(1,3-dioxoisoindolin-2-yl)-7-(2-hydroxyethyl)-1-methyl-1H-indazol-5-yl)-3-fluoro-5-(trifluoromethyl)benzamide (25 mg, 0.04 mmol) in methanol (2 mL) was added hydrazine hydrate (98%, 0.2 mL) at room temperature. The reaction was stirred for additional 1 h. The resulting mixture was concentrated under reduced pressure, the residue was purified by Prep-HPLC with the following conditions (Column: XBridge Prep Phenyl OBD Column 19*250 mm; Mobile Phase: acetonitrile in water (0.05% 2,2,2-trifluoroacetic acid); Flow rate: 60 mL/min; Gradient: 47% B to 67% B in 10 min; Wave Length: 254/220 nm). Eluted fractions were collected and lyophilized to afford the TFA salt of the desired product as a white solid. LCMS calculated for C₂₄H₁₉ClF₅N₄O₃ (M+H)⁺ m/z=541.1; found 541.1; ¹H NMR (400 MHz, DMSO-d₆) δ 10.14 (s, 1H), 7.92-7.86 (m, 1H), 7.81-7.73 (m, 2H), 7.46 (dd, J=8.8, 5.6 Hz, 1H), 7.11 (s, 1H), 6.90-6.83 (m, 1H), 6.41 (dd, J=9.6, 2.8 Hz, 1H), 4.87 (s, 2H), 4.00 (s, 3H), 3.74 (t, J=6.8 Hz, 2H), 3.14 (d, J=6.8 Hz, 2H).

### Example 67. N-(3-Amino-4-(2-chloro-5-fluorophenoxy)-1-(2-hydroxyethyl)-7-(pyridin-2-ylethynyl)-1H-indazol-5-yl)-3-fluoro-5-(trifluoromethyl)benzamide

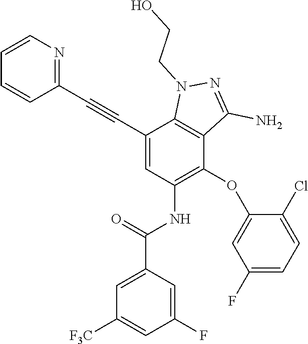 [SMILES: Nc1nn(CCO)c2c(C#Cc3ccccn3)cc(NC(=O)c3cc(F)cc(C(F)(F)F)c3)c(Oc3cc(F)ccc3Cl)c12]

### Step 1: N-(7-bromo-4-(2-chloro-5-fluorophenoxy)-3-(1,3-dioxoisoindolin-2-yl)-1-(2-((tetrahydro-2H-pyran-2-yl)oxy)ethyl)-1H-indazol-5-yl)-3-fluoro-5-(trifluoromethyl)benzamide

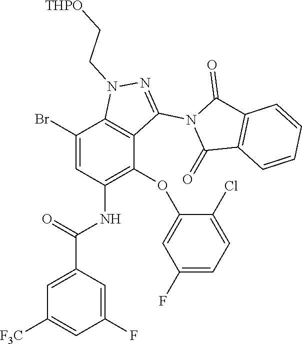

To the mixture of N-[7-bromo-4-(2-chloro-5-fluorophenoxy)-3-(1,3-dioxoisoindol-2-yl)-1H-indazol-5-yl]-3-fluoro-5-(trifluoromethyl)benzamide (Example 11, Step 1: 140 mg, 0.14 mmol) and potassium carbonate (39.44 mg, 0.284 mmol) in dimethylformamide (2 mL) was added 2-(2-bromoethoxy)oxane (59 mg, 0.28 mmol) at room temperature. The reaction was stirred at 50° C. for 12 h. The resulting mixture was diluted with water (50 mL). The aqueous solution was extracted with ethyl acetate (3×50 mL). The combined organic layers were washed with brine (50 mL), dried over anhydrous sodium sulfate. After filtration, the filtrate was concentrated under reduced pressure. The residue was purified by silica gel column chromatography, eluted with 35% ethyl acetate in petroleum ether to afford the desired product as a light-yellow oil (70 mg, 57%). LCMS calculated for C₃₆H₂₄BrClF₅N₄O₆ (M−H)⁻ m/z=817.0; found 816.9.

### Step 2: N-(3-amino-7-bromo-4-(2-chloro-5-fluorophenoxy)-1-(2-((tetrahydro-2H-pyran-2-yl)oxy)ethyl)-1H-indazol-5-yl)-3-fluoro-5-(trifluoromethyl)benzamide

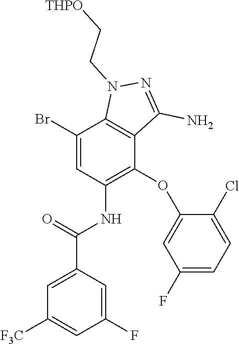

To the mixture of N-[7-bromo-4-(2-chloro-5-fluorophenoxy)-3-(1,3-dioxoisoindol-2-yl)-1-[2-(oxan-2-yloxy)ethyl]indazol-5-yl]-3-fluoro-5-(trifluoromethyl)benzamide (70 mg, 0.08 mmol) in methanol (2 mL) was added hydrazine hydrate (98%, 0.2 mL) at room temperature. The reaction was stirred for additional 30 min. The resulting mixture was concentrated under reduced pressure. The residue was purified by silica gel column chromatography, eluted with 50% ethyl acetate in petroleum ether to afford the desired product as a light-yellow solid (50 mg, 86%). LCMS calculated for C₂₈H₂₄BrClF₅N₄O₄ (M+H)⁺ m/z=689.1; found 689.1;

### Step 3: N-(3-amino-4-(2-chloro-5-fluorophenoxy)-7-(pyridin-2-ylethynyl)-1-(2-((tetrahydro-2H-pyran-2-yl)oxy)ethyl)-1H-indazol-5-yl)-3-fluoro-5-(trifluoromethyl)benzamide

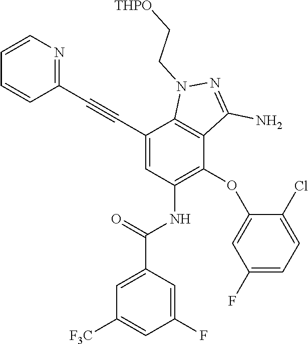

To a screw-cap vial equipped with a magnetic stir bar were placed N-[3-amino-7-bromo-4-(2-chloro-5-fluorophenoxy)-1-[2-(oxan-2-yloxy)ethyl]indazol-5-yl]-3-fluoro-5-(trifluoromethyl)benzamide (50 mg, 0.07 mmol), Pd(PPh₃)₂Cl₂ (25 mg, 0.04 mmol) and cuprous iodide (3 mg, 0.01 mmol). The vial was sealed with a Teflon-lined septum, evacuated and backfilled with nitrogen (this process was repeated a total of three times). 2-Ethynylpyridine (36 mg, 0.35 mmol) and triethylamine (0.5 mL) in N,N-Dimethylformamide (0.5 mL) were added. The reaction was stirred at 100° C. for 2 h. The resulting mixture was concentrated under reduced pressure. The residue was purified by reversed-phase flash chromatography with the following conditions (column, C18 silica gel; mobile phase, acetonitrile in water (0.1% formic acid), 5% to 90% gradient in 20 min; detector, UV 254 nm), eluted fractions were collected and lyophilized to afford the desired product as a brown solid (47 mg, 92%). LCMS calculated for C₃₅H₂₈ClF₅N₅O₄ (M+H)⁺ m/z=712.2; found 712.3;

### Step 4: N-(3-amino-4-(2-chloro-5-fluorophenoxy)-1-(2-hydroxyethyl)-7-(pyridin-2-ylethynyl)-1H-indazol-5-yl)-3-fluoro-5-(trifluoromethyl)benzamide

To the mixture of N-[3-amino-4-(2-chloro-5-fluorophenoxy)-1-[2-(oxan-2-yloxy)ethyl]-7-[2-(pyridin-2-yl)ethynyl]indazol-5-yl]-3-fluoro-5-(trifluoromethyl)benzamide (40 mg, 0.06 mmol) in dichloromethane (0.75 mL) was added 2,2,2-trifluoroacetic acid (0.25 mL) at room temperature. The reaction was stirred for additional 2 h. The resulting mixture was concentrated under reduced pressure. The residue was purified by silica gel column chromatography, eluted with 10% methanol in dichloromethane to afford crude product, which was then purified by Prep-HPLC with the following conditions (Column: Sunfire prep C18 column, 30*150 mm, 5 m; Mobile Phase: acetonitrile in water (0.05% trifluoroacetic acid); Flow rate: 60 mL/min mL/min; Gradient: 22% B to 42% B in 10 min; Wave Length: 254 nm/220 nm; RT1(min): 10.75). Eluted fractions were collected and lyophilized to afford the TFA salt of the desired product as an orange solid. LCMS calculated for C₃₀H₂₀ClF₅N₅O₃ (M+H)⁺ m/z=628.1; found 628.2. ¹H NMR (400 MHz, DMSO-d₆) δ 10.19 (s, 1H), 8.67-8.64 (m, 1H), 7.94-7.88 (m, 2H), 7.78-7.70 (m, 3H), 7.60 (s, 1H), 7.51-7.42 (m, 2H), 6.92-6.85 (m, 1H), 6.58 (dd, J=9.6, 2.8 Hz, 1H), 4.67 (t, J=6.0 Hz, 2H), 3.86 (t, J=6.0 Hz, 2H).

### Example 68. N-(2-Amino-3-(2-chloro-5-fluorophenoxy)-8-oxo-7,8-dihydro-6H-pyrazolo[4,5,1-ij]quinazolin-4-yl)-3-fluoro-5-(trifluoromethyl)benzamide

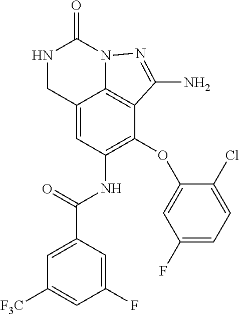 [SMILES: Nc1nn2c3c(cc(NC(=O)c4cc(F)cc(C(F)(F)F)c4)c(Oc4cc(F)ccc4Cl)c13)CNC2=O]

### Step 1: N-(4-(2-chloro-5-fluorophenoxy)-3-(4,5,6,7-tetrachloro-1,3-dioxoisoindolin-2-yl)-1H-indazol-5-yl)-3-fluoro-5-(trifluoromethyl)benzamide

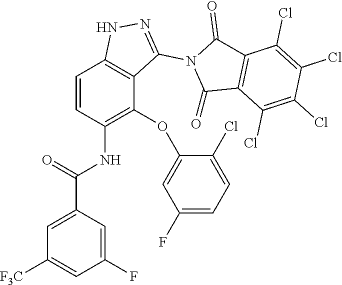 [SMILES: O=C(Nc1ccc2[nH]nc(N3C(=O)c4c(Cl)c(Cl)c(Cl)c(Cl)c4C3=O)c2c1Oc1cc(F)ccc1Cl)c1cc(F)cc(C(F)(F)F)c1]

To the mixture of N-[3-amino-4-(2-chloro-5-fluorophenoxy)-1H-indazol-5-yl]-3-fluoro-5-(trifluoromethyl)benzamide (Example 1: 4.5 g, 9.3 mmol) in acetic acid (90 mL) was added tetrachlorophthalic anhydride (4 g, 14 mmol) in portions at room temperature. The reaction was stirred at 120° C. for 2 h. Upon cooling to room temperature, the resulting mixture was concentrated under reduced pressure. The residue was purified by silica gel column chromatography, eluted with 50% ethyl acetate in petroleum ether to afford the desired product as a brown yellow solid (5.8 g, 83%). LCMS calculated for C₂₉H₉Cl₅F₅N₄O₄ (M−H)⁻ m/z=746.9; found 746.9.

### Step 2: N-(7-Bromo-4-(2-chloro-5-fluorophenoxy)-3-(4,5,6,7-tetrachloro-1,3-dioxoisoindolin-2-yl)-1H-indazol-5-yl)-3-fluoro-5-(trifluoromethyl)benzamide

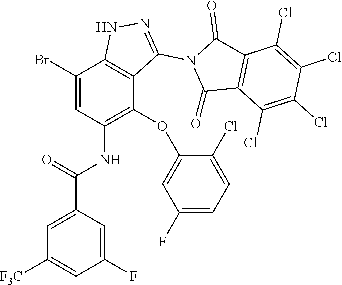 [SMILES: O=C(Nc1cc(Br)c2[nH]nc(N3C(=O)c4c(Cl)c(Cl)c(Cl)c(Cl)c4C3=O)c2c1Oc1cc(F)ccc1Cl)c1cc(F)cc(C(F)(F)F)c1]

To the mixture of N-[4-(2-chloro-5-fluorophenoxy)-3-(4,5,6,7-tetrachloro-1,3-dioxoisoindol-2-yl)-1H-indazol-5-yl]-3-fluoro-5-(trifluoromethyl)benzamide (5.8 g, 7.73 mmol) and acetic acid (464 mg, 7.73 mmol) in acetonitrile (100 mL) was added N-bromosuccinimide (1.44 g, 8.1 mmol) in portions at room temperature. The reaction was stirred at 100° C. for 3 h. Upon cooling to room temperature, the resulting mixture was diluted with water (100 mL). The aqueous solution was extracted with ethyl acetate (3×150 mL). The combined organic layers were washed with sat. sodium thiosulfate (2×100 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a yellow solid (4.3 g, 67%). LCMS calculated for C₂₉H₈BrCl₅F₅N₄O₄(M−H)⁻ m/z=824.8; found 824.8.

### Step 3: N-(7-Bromo-4-(2-chloro-5-fluorophenoxy)-3-(4,5,6,7-tetrachloro-1,3-dioxoisoindolin-2-yl)-1-(tetrahydro-2H-pyran-2-yl)-1H-indazol-5-yl)-3-fluoro-5-(trifluoromethyl)benzamide

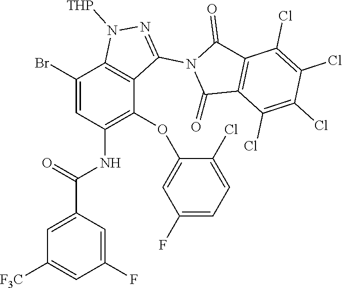

To the mixture of N-[7-bromo-4-(2-chloro-5-fluorophenoxy)-3-(4,5,6,7-tetrachloro-1,3-dioxoisoindol-2-yl)-1H-indazol-5-yl]-3-fluoro-5-(trifluoromethyl)benzamide (4 g, 4.8 mmol) and 4-methylbenzenesulfonic acid (83 mg, 0.48 mmol) in dichloromethane (40 mL) was added dihydropyran (811 mg, 9.6 mmol) dropwise at room temperature. The reaction was stirred for additional 16 h. The mixture was concentrated under reduced pressure. The residue was purified by silica gel column chromatography, eluted with 30% ethyl acetate in petroleum ether to afford the desired product as a light-yellow solid (3.5 g, 79%). LCMS calculated for C₃₄H₁₆BrCl₅F₅N₄O₅(M−H)⁻ m/z=908.9; found 908.9.

### Step 4: N-(4-(2-chloro-5-fluorophenoxy)-3-(4,5,6,7-tetrachloro-1,3-dioxoisoindolin-2-yl)-1-(tetrahydro-2H-pyran-2-yl)-7-vinyl-1H-indazol-5-yl)-3-fluoro-5-(trifluoromethyl)benzamide

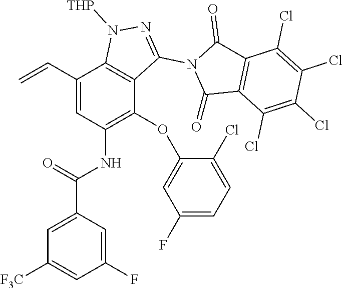

To a screw-cap vial equipped with a magnetic stir bar were placed N-[7-bromo-4-(2-chloro-5-fluorophenoxy)-1-(oxan-2-yl)-3-(4,5,6,7-tetrachloro-1,3-dioxoisoindol-2-yl)indazol-5-yl]-3-fluoro-5-(trifluoromethyl)benzamide (2 g, 2.2 mmol), 2-ethenyl-4,4,5,5-tetramethyl-1,3,2-dioxaborolane (674 mg, 4.4 mmol), XPhos Pd G3 (370 mg, 0.44 mmol) and potassium carbonate (907 mg, 6.57 mmol). The vial was sealed with a Teflon-lined septum, evacuated and backfilled with nitrogen (this process was repeated a total of three times). Toluene (40 mL) and water (8 mL) were added. The reaction was stirred at 85° C. for 3 h. The resulting mixture was concentrated under reduced pressure. The residue was purified by silica gel column chromatography, eluted with 30% ethyl acetate in petroleum ether to afford the desired product as a light orange solid (650 mg, 34%). LCMS calculated for C₃₆H₁₉C₁₅F₅N₄O₅ (M−H)⁻ m/z=857.0; found 857.0.

### Step 5: N-(4-(2-chloro-5-fluorophenoxy)-7-formyl-3-(4,5,6,7-tetrachloro-1,3-dioxoisoindolin-2-yl)-1-(tetrahydro-2H-pyran-2-yl)-1H-indazol-5-yl)-3-fluoro-5-(trifluoromethyl)benzamide

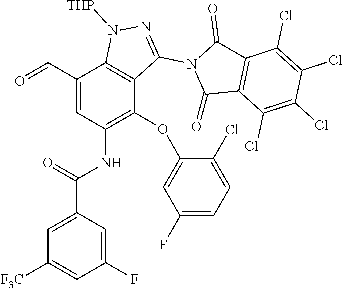

To the mixture of N-[4-(2-chloro-5-fluorophenoxy)-7-ethenyl-1-(oxan-2-yl)-3-(4,5,6,7-tetrachloro-1,3-dioxoisoindol-2-yl)indazol-5-yl]-3-fluoro-5-(trifluoromethyl)benzamide (300 mg, 0.35 mmol) and 2,6-dimethylpyridine (74.7 mg, 0.7 mmol) in tetrahydrofuran (5 mL) and water (1 mL) were added sodium periodate (298 mg, 1.4 mmol) and potassium osmate(VI) dihydrate (26 mg, 0.07 mmol) in portions at room temperature. The reaction was stirred for 16 h. The resulting mixture was concentrated under reduced pressure. The residue was purified by silica gel column chromatography, eluted with 30% ethyl acetate in petroleum ether to afford the desired product as a light-yellow solid (300 mg, 50%). LCMS calculated for C₃₅H₁₇Cl₅F₅N₄O₆(M−H)⁻ m/z=859.0; found 858.9.

### Step 6: N-(3-amino-4-(2-chloro-5-fluorophenoxy)-7-(((2,4-dimethoxybenzyl)amino)methyl)-1-(tetrahydro-2H-pyran-2-yl)-1H-indazol-5-yl)-3-fluoro-5-(trifluoromethyl)benzamide

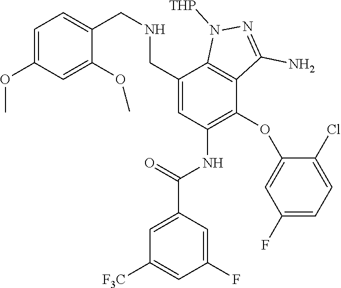

To the mixture of N-[4-(2-chloro-5-fluorophenoxy)-7-formyl-1-(oxan-2-yl)-3-(4,5,6,7-tetrachloro-1,3-dioxoisoindol-2-yl)indazol-5-yl]-3-fluoro-5-(trifluoromethyl)benzamide (90 mg, 0.1 mmol) and 1-(2,4-dimethoxyphenyl)methanamine (52.3 mg, 0.3 mmol) in methanol (3 mL) was added acetic acid (13 mg, 0.2 mmol) at room temperature. The mixture was stirred for 1 h, followed by the addition of sodium cyanoborohydride (20 mg, 0.3 mmol). The reaction was stirred for additional 16 h. The resulting mixture was concentrated under reduced pressure. The residue was purified by reversed-phase flash chromatography with the following conditions: column, C18 silica gel; mobile phase, acetonitrile in water (0.1% formic acid), 10% to 50% gradient in 20 min; detector, UV 254 nm, eluted fractions were collected and lyophilized to afford the desired product as a white solid (25 mg, 32%). LCMS calculated for C₃₆H₃₄ClF₅N₅O₅ (M+H)⁺ m/z=746.2; found 746.3.

### Step 7: N-(3-amino-7-(aminomethyl)-4-(2-chloro-5-fluorophenoxy)-1H-indazol-5-yl)-3-fluoro-5-(trifluoromethyl)benzamide

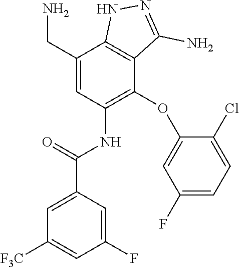 [SMILES: NCc1cc(NC(=O)c2cc(F)cc(C(F)(F)F)c2)c(Oc2cc(F)ccc2Cl)c2c(N)n[nH]c12]

The mixture of N-[3-amino-4-(2-chloro-5-fluorophenoxy)-7-({[(2,4-dimethoxyphenyl)methyl]amino}methyl)-1-(oxan-2-yl)indazol-5-yl]-3-fluoro-5-(trifluoromethyl)benzamide (25 mg, 0.03 mmol) and 2,2,2-trifluoroacetic acid (1 mL) was stirred at 110° C. for 1 h. Upon cooling to room temperature, the resulting mixture was concentrated under reduced pressure. The residue was purified by reversed-phase flash chromatography with the following conditions: column, C18 silica gel; mobile phase, acetonitrile in water (10 mmol/L ammonium bicarbonate), 10% to 80% gradient in 20 min; detector, UV 254 nm, eluted fractions were collected and lyophilized to afford the desired product as a white solid. LCMS calculated for C₂₂H₁₆ClF₅N₅O₂ (M+H)⁺ m/z=512.1; found 512.1.

### Step 8: N-(2-amino-3-(2-chloro-5-fluorophenoxy)-8-oxo-7,8-dihydro-6H-pyrazolo[4,5,1-ij]quinazolin-4-yl)-3-fluoro-5-(trifluoromethyl)benzamide

To the mixture of N-[3-amino-7-(aminomethyl)-4-(2-chloro-5-fluorophenoxy)-1H-indazol-5-yl]-3-fluoro-5-(trifluoromethyl)benzamide (5 mg, 0.01 mmol) in acetonitrile (0.1 mL) was added 1,1′-carbonyldiimidazole (1.8 mg, 0.01 mmol), followed by 1,8-diazabicyclo[5.4.0]undec-7-ene (0.3 mg, 0.002 mmol) at room temperature. The reaction was stirred for 1 h. The resulting mixture was concentrated under reduced pressure. The residue purified by Prep-HPLC with the following conditions (Column: Sunfire prep C18 column, 30*150 mm, 5 m; Mobile Phase: acetonitrile in water (0.1% formic acid); Flow rate: 60 mL/min mL/min; Gradient: isocratic 32% B to 50% B in 10 min; Wave Length: 254 nm/220 nm nm). Eluted fractions were collected and lyophilized to afford the desired product as a white solid. LCMS calculated for C₂₃H₁₄ClF₅N₅O₃ (M+H)⁺ m/z=538.1; found 538.1.

### Example 69. N-(2-Amino-3-(2-chloro-5-fluorophenoxy)-7-methyl-8-oxo-7,8-dihydro-6H-pyrazolo[4,5,1-ij]quinazolin-4-yl)-3-fluoro-5-(trifluoromethyl)benzamide

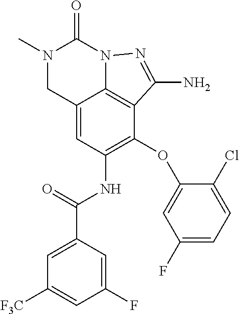 [SMILES: CN1Cc2cc(NC(=O)c3cc(F)cc(C(F)(F)F)c3)c(Oc3cc(F)ccc3Cl)c3c(N)nn(c23)C1=O]

### Step 1: N-(3-amino-4-(2-chloro-5-fluorophenoxy)-7-((methylamino)methyl)-1-(tetrahydro-2H-pyran-2-yl)-1H-indazol-5-yl)-3-fluoro-5-(trifluoromethyl)benzamide

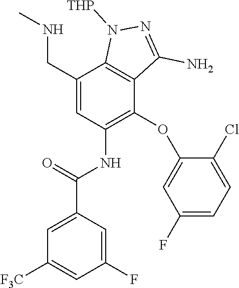

To the mixture of N-[4-(2-chloro-5-fluorophenoxy)-7-formyl-1-(oxan-2-yl)-3-(4,5,6,7-tetrachloro-1,3-dioxoisoindol-2-yl)indazol-5-yl]-3-fluoro-5-(trifluoromethyl)benzamide (Example 68, Step 5: 90 mg, 0.1 mmol) and methanamine (2 M in THF, 104 μL, 0.2 mmol) in methanol (2 mL) was added acetic acid (6.3 mg, 0.1 mmol) at room temperature. The reaction was stirred for additional 16 h. To the above mixture was added sodium cyanoborohydride (13 mg, 0.2 mmol) at room temperature. The reaction was stirred for another 5 h. The resulting mixture was concentrated under reduced pressure. The residue was purified by reversed-phase flash chromatography with the following conditions (column, C18 silica gel; mobile phase, acetonitrile in water (10 mmol/L ammonium bicarbonate), 5% to 95% gradient in 25 min; detector, UV 254 nm). Eluted fractions were collected and lyophilized to afford the desired product as a white solid (20 mg, 31%). LCMS calculated for C₂₈H₂₆ClF₅N₅O₃ (M+H)⁺ m/z=610.2; found 609.9.

### Step 2: N-(3-amino-4-(2-chloro-5-fluorophenoxy)-7-((methylamino)methyl)-1H-indazol-5-yl)-3-fluoro-5-(trifluoromethyl)benzamide

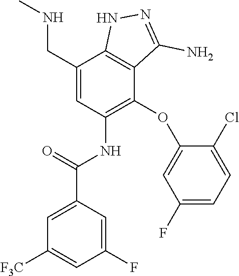 [SMILES: CNCc1cc(NC(=O)c2cc(F)cc(C(F)(F)F)c2)c(Oc2cc(F)ccc2Cl)c2c(N)n[nH]c12]

To the mixture of N-[3-amino-4-(2-chloro-5-fluorophenoxy)-7-[(methylamino)methyl]-1-(oxan-2-yl)indazol-5-yl]-3-fluoro-5-(trifluoromethyl)benzamide (20 mg, 0.033 mmol) in dichloromethane (0.5 mL) was added 2,2,2-trifluoroacetic acid (0.2 mL) at room temperature. The reaction was stirred for 1 h. The resulting mixture was concentrated under reduced pressure. The residue was neutralized to pH 9 with saturated aqueous sodium bicarbonate solution. The aqueous solution was extracted with dichloromethane (3×10 mL), dried over anhydrous sodium sulfate. After filtration, the filtrate was concentrated under reduced pressure. The residue was purified by Prep-TLC (10% methanol in dichloromethane) to afford the desired product as a white solid. LCMS calculated for C₂₃H₁₈ClF₅N₅O₂ (M+H)⁺ m/z=526.1; found 526.1.

### Step 3: N-(2-amino-3-(2-chloro-5-fluorophenoxy)-7-methyl-8-oxo-7,8-dihydro-6H-pyrazolo[4,5,1-ij]quinazolin-4-yl)-3-fluoro-5-(trifluoromethyl)benzamide

To the mixture of N-[3-amino-4-(2-chloro-5-fluorophenoxy)-7-[(methylamino)methyl]-1H-indazol-5-yl]-3-fluoro-5-(trifluoromethyl)benzamide (10 mg, 0.02 mmol) in acetonitrile (0.5 mL) was added 1,1′-carbonyldiimidazole (6.2 mg, 0.04 mmol), followed by 1,8-diazabicyclo[5.4.0]undec-7-ene (0.9 mg, 0.006 mmol) at room temperature. The reaction was stirred for 2 h. The resulting mixture was concentrated under reduced pressure. The crude product was purified by Prep-HPLC with the following conditions (Column: Sunfire prep C18 column, 30*150 mm, 5 m; Mobile Phase: acetonitrile in water (0.1% formic acid); Flow rate: 60 mL/min; Gradient: 35% B to 55% B in 10 min; Wave Length: 254/220 nm). Eluted fractions were collected and lyophilized to afford the desired product as a white solid. LCMS calculated for C₂₄H₁₆ClF₅N₅O₃ (M+H)⁺ m/z=552.1; found 552.1. ¹H NMR (400 MHz, Chloroform-d) δ 8.24 (s, 1H), 8.01 (s, 1H), 7.71-7.62 (m, 2H), 7.55-7.48 (m, 1H), 7.45 (dd, J=8.8, 5.6 Hz, 1H), 6.85-6.77 (m, 1H), 6.52-6.44 (m, 1H), 4.91 (s, 2H), 3.22 (s, 3H).

### Example 70. N-(4-Amino-3-(2-chloro-5-fluorophenoxy)-7H-pyrazolo[4,5,1-de]phenanthridin-2-yl)-3-fluoro-5-(trifluoromethyl)benzamide

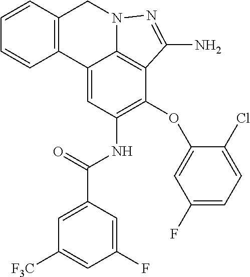 [SMILES: Nc1nn2c3c(cc(NC(=O)c4cc(F)cc(C(F)(F)F)c4)c(Oc4cc(F)ccc4Cl)c13)-c1ccccc1C2]

### Step 1: N-(4-(2-chloro-5-fluorophenoxy)-3-(1,3-dioxoisoindolin-2-yl)-7-(2-(hydroxymethyl)phenyl)-1-(tetrahydro-2H-pyran-2-yl)-1H-indazol-5-yl)-3-fluoro-5-(trifluoromethyl)benzamide

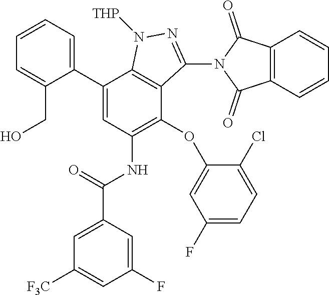

To a screw-cap vial equipped with a magnetic stir bar were placed N-[7-bromo-4-(2-chloro-5-fluorophenoxy)-3-(1,3-dioxoisoindol-2-yl)-1-(oxan-2-yl)indazol-5-yl]-3-fluoro-5-(trifluoromethyl)benzamide (Example 36, Step 1: 150 mg, 0.19 mmol), [2-(4,4,5,5-tetramethyl-1,3,2-dioxaborolan-2-yl)phenyl]methanol (59 mg, 0.25 mmol), Pd(dppf)Cl₂CH₂Cl₂ (16 mg, 0.019 mmol) and potassium carbonate (53 mg, 0.38 mmol). The vial was sealed with a Teflon-lined septum, evacuated and backfilled with nitrogen (this process was repeated a total of three times). 1,4-Dioxane (2 mL) and water (0.4 mL) were added. The reaction was stirred at 80° C. for 2 h. Upon cooling to room temperature, the resulting mixture was concentrated under reduced pressure. The residue was purified by silica gel column chromatography, eluted with 50% ethyl acetate in petroleum ether to afford the desired product as a white solid (100 mg, 64%). LCMS calculated for C₄₁H₂₇ClF₅N₄O₆ (M−H)⁻ m/z=801.2; found 801.1.

### Step 2: N-(4-(2-Chloro-5-fluorophenoxy)-3-(1,3-dioxoisoindolin-2-yl)-7-(2-(hydroxymethyl)phenyl)-1H-indazol-5-yl)-3-fluoro-5-(trifluoromethyl)benzamide

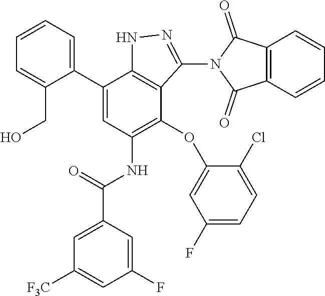 [SMILES: O=C(Nc1cc(-c2ccccc2CO)c2[nH]nc(N3C(=O)c4ccccc4C3=O)c2c1Oc1cc(F)ccc1Cl)c1cc(F)cc(C(F)(F)F)c1]

To the mixture of N-{3-amino-7-[2-(bromomethyl)phenyl]-4-(2-chloro-5-fluorophenoxy)-1-(oxan-2-yl)indazol-5-yl}-3-fluoro-5-(trifluoromethyl)benzamide (100 mg, 0.14 mmol) in dichloroethane (1 mL) was added hydrogen chloride (4M in 1,4-dioxane, 0.5 mL) at room temperature. The reaction was stirred for 2 h. The resulting mixture was concentrated under reduced pressure. The crude product was used in the next step directly without further purification. LCMS calculated for C₃₆H₂₁ClF₅N₄O₅ (M+H)⁺ m/z=719.1; found 719.2.

### Step 3: N-(3-(2-chloro-5-fluorophenoxy)-4-(1,3-dioxoisoindolin-2-yl)-7H-pyrazolo[4,5,1-de]phenanthridin-2-yl)-3-fluoro-5-(trifluoromethyl)benzamide

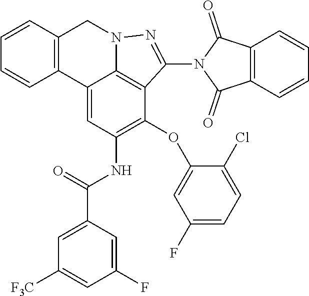 [SMILES: O=C(Nc1cc2c3c(c(N4C(=O)c5ccccc5C4=O)nn3Cc3ccccc3-2)c1Oc1cc(F)ccc1Cl)c1cc(F)cc(C(F)(F)F)c1]

To the mixture of N-[4-(2-chloro-5-fluorophenoxy)-3-(1,3-dioxoisoindol-2-yl)-7-[2-(hydroxymethyl)phenyl]-1H-indazol-5-yl]-3-fluoro-5-(trifluoromethyl)benzamide (70 mg, 0.1 mmol) and triphenylphosphane (41 mg, 0.16 mmol) in tetrahydrofuran (0.5 mL) was added diisopropyl azodiformate (30 mg, 0.15 mmol) at room temperature. The reaction was stirred for 2 h. The resulting mixture was concentrated under reduced pressure. The residue was purified by silica gel column chromatography, eluted with 25% ethyl acetate in petroleum ether to afford the desired product as a white solid (30 mg). LCMS calculated for C₃₆H₁₇ClF₅N₄O₄ (M−H)⁻ m/z=699.1; found 699.1.

### Step 4: N-(4-amino-3-(2-chloro-5-fluorophenoxy)-7H-pyrazolo[4,5,1-de]phenanthridin-2-yl)-3-fluoro-5-(trifluoromethyl)benzamide

To the mixture of N-(3-(2-chloro-5-fluorophenoxy)-4-(1,3-dioxoisoindolin-2-yl)-7H-pyrazolo[4,5,1-de]phenanthridin-2-yl)-3-fluoro-5-(trifluoromethyl)benzamide (30 mg, 0.04 mmol) in methanol (1 mL) was added hydrazine hydrate (98%, 0.1 mL) at room temperature. The reaction was stirred for 1 h. The residue was purified by reversed-phase flash chromatography with the following conditions: column, C18 silica gel; mobile phase, methanol in water (0.1% formic acid), 10% to 50% gradient in 10 min; detector, UV 254 nm. Eluted fractions were collected and lyophilized to afford the desired product as a yellow solid. LCMS calculated for C₂₅H₁₇ClF₅N₄O₂ (M+H)⁺ m/z=571.1; found 571.1. ¹H NMR (400 MHz, DMSO-d₆) δ 10.19 (s, 1H), 8.05-7.98 (m, 1H), 7.95-7.89 (m, 1H), 7.87-7.78 (m, 2H), 7.71 (s, 1H), 7.47 (dd, J=8.8, 5.6 Hz, 1H), 7.44-7.35 (m, 3H), 6.94-6.84 (m, 1H), 6.59 (dd, J=9.6, 2.8 Hz, 1H), 5.53 (s, 2H), 5.09 (s, 2H).

### Example 71. N-(2-Amino-3-(2-chloro-5-fluorophenoxy)-7-cyclopropyl-8-oxo-7,8-dihydro-6H-pyrazolo[4,5,1-ij]quinazolin-4-yl)-3-fluoro-5-(trifluoromethyl)benzamide

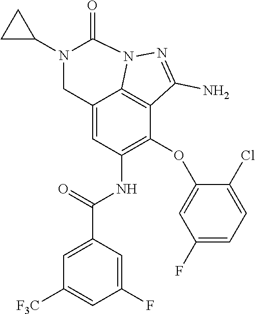 [SMILES: Nc1nn2c3c(cc(NC(=O)c4cc(F)cc(C(F)(F)F)c4)c(Oc4cc(F)ccc4Cl)c13)CN(C1CC1)C2=O]

### Step 1: N-(4-(2-chloro-5-fluorophenoxy)-3-(1,3-dioxoisoindolin-2-yl)-7-formyl-1-(tetrahydro-2H-pyran-2-yl)-1H-indazol-5-yl)-3-fluoro-5-(trifluoromethyl)benzamide

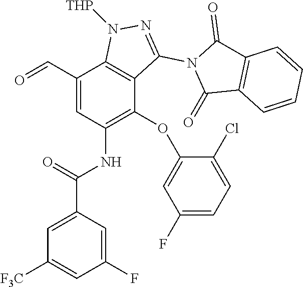

To the mixture of N-[4-(2-chloro-5-fluorophenoxy)-3-(1,3-dioxoisoindol-2-yl)-7-(hydroxymethyl)-1-(oxan-2-yl)indazol-5-yl]-3-fluoro-5-(trifluoromethyl)benzamide (Example 36, Step 2: 100 mg, 0.14 mmol) in dichloromethane (5 mL) was added manganese dioxide (60 mg, 0.7 mmol) at room temperature. The reaction was stirred at 50° C. for 6 h. Upon cooling to room temperature, the resulting mixture was filtered, the filter cake was washed with dichloromethane (3×20 mL). The filtrate was concentrated under reduced pressure. The residue was purified by silica gel column chromatography, eluted with 30% ethyl acetate in petroleum ether to afford the desired product as a white solid (80 mg, 80%). LCMS calculated for C₃₅H₂₁ClF₅N₄O₆ (M−H)⁻ m/z=723.1; found 723.2.

### Step 2: N-(3-amino-4-(2-chloro-5-fluorophenoxy)-7-((cyclopropylamino)methyl)-1-(tetrahydro-2H-pyran-2-yl)-1H-indazol-5-yl)-3-fluoro-5-(trifluoromethyl)benzamide

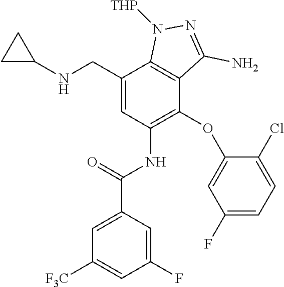

To the mixture of N-[4-(2-chloro-5-fluorophenoxy)-3-(1,3-dioxoisoindol-2-yl)-7-formyl-1-(oxan-2-yl)indazol-5-yl]-3-fluoro-5-(trifluoromethyl)benzamide (40 mg, 0.06 mmol) and cyclopropanamine (6.3 mg, 0.11 mmol) in methanol (1 mL) was added acetic acid (3 mg, 0.05 mmol) at room temperature. The reaction was stirred at 50° C. for 1 h. Upon cooling to room temperature, to the above mixture was added sodium cyanoborohydride (7 mg, 0.11 mmol) at room temperature. The resulting mixture was stirred for additional 1 h. To the above mixture was added hydrazine hydrate (98%, 0.1 mL) at room temperature. The reaction mixture was stirred for additional 3 h. The resulting mixture was concentrated under reduced pressure. The residue was purified by silica gel column chromatography, eluted with 10% methanol in dichloromethane to afford the desired product as an off-white solid (25 mg, 59%). LCMS calculated for C₃₀H₂₈ClF₅N₅O₃ (M+H)⁺ m/z=636.2; found 636.2.

### Step 3: N-(3-amino-4-(2-chloro-5-fluorophenoxy)-7-((cyclopropylamino)methyl)-1H-indazol-5-yl)-3-fluoro-5-(trifluoromethyl)benzamide

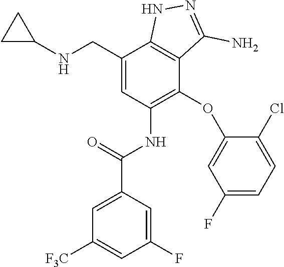 [SMILES: Nc1n[nH]c2c(CNC3CC3)cc(NC(=O)c3cc(F)cc(C(F)(F)F)c3)c(Oc3cc(F)ccc3Cl)c12]

To the mixture of N-[3-amino-4-(2-chloro-5-fluorophenoxy)-7-[(cyclopropylamino)methyl]-1-(oxan-2-yl)indazol-5-yl]-3-fluoro-5-(trifluoromethyl)benzamide (25 mg, 0.04 mmol) in dichloromethane (3 mL) was added 2,2,2-trifluoroacetic acid (0.3 mL) at room temperature. The reaction was stirred for 2 h. The resulting mixture was concentrated under reduced pressure. The residue was purified by reversed-phase flash chromatography with the following conditions: column, C18 silica gel; mobile phase, acetonitrile in water (10 mmol/L ammonium bicarbonate), 10% to 95% gradient in 20 min; detector, UV 254 nm, eluted fractions were collected and lyophilized to the desired product as a white solid (12 mg, 55%). LCMS calculated for C₂₅H₂₀ClF₅N₅O₂ (M+H)⁺ m/z=552.1; found 552.2.

### Step 4: N-(2-amino-3-(2-chloro-5-fluorophenoxy)-7-cyclopropyl-8-oxo-7,8-dihydro-6H-pyrazolo[4,5,1-ij]quinazolin-4-yl)-3-fluoro-5-(trifluoromethyl)benzamide

To the mixture of N-[3-amino-4-(2-chloro-5-fluorophenoxy)-7-[(cyclopropylamino)methyl]-1H-indazol-5-yl]-3-fluoro-5-(trifluoromethyl)benzamide (12 mg, 0.02 mmol) in acetonitrile (0.5 mL) was added 1,1′-carbonyldiimidazole (5.29 mg, 0.03 mmol), followed by 1,8-diazabicyclo[5.4.0]undec-7-ene (0.66 mg, 0.004 mmol) at room temperature. The reaction was stirred for 1 h. The resulting mixture was concentrated under reduced pressure. The crude product was purified by Prep-HPLC with the following conditions (Column: Xselect CSH C18 OBD Column 30*150 mm 5 m; Mobile Phase: acetonitrile in water (0.1% formic acid); Flow rate: 60 mL/min; Gradient: 37% B to 57% B in 8 min; Wave Length: 254/220 nm). Eluted fractions were collected and lyophilized to afford the desired product as a white solid. LCMS calculated for C₂₆H₁₈ClF₅N₅O₃ (M+H)⁺ m/z=578.1; found 578.2. ¹H NMR (400 MHz, DMSO-d₆) δ 10.20 (s, 1H), 7.95-7.88 (m, 1H), 7.79-7.72 (m, 2H), 7.43 (dd, J=8.8, 5.6 Hz, 1H), 7.31 (s, 1H), 6.91-6.83 (m, 1H), 6.54 (dd, J=9.6, 2.8 Hz, 1H), 5.74 (s, 2H), 4.86 (s, 2H), 2.76-2.67 (m, 1H), 0.88-0.73 (m, 4H).

### Example 72. N-(2-Amino-3-(2-chloro-5-fluorophenoxy)-7-cyclobutyl-8-oxo-7,8-dihydro-6H-pyrazolo[4,5,1-ij]quinazolin-4-yl)-3-fluoro-5-(trifluoromethyl)benzamide

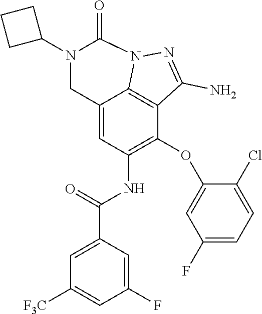 [SMILES: Nc1nn2c3c(cc(NC(=O)c4cc(F)cc(C(F)(F)F)c4)c(Oc4cc(F)ccc4Cl)c13)CN(C1CCC1)C2=O]

### Step 1: N-(3-amino-4-(2-chloro-5-fluorophenoxy)-7-((cyclobutylamino)methyl)-1-(tetrahydro-2H-pyran-2-yl)-1H-indazol-5-yl)-3-fluoro-5-(trifluoromethyl)benzamide

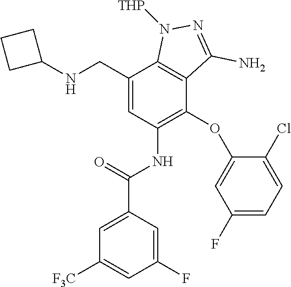

To the mixture of N-[4-(2-chloro-5-fluorophenoxy)-3-(1,3-dioxoisoindol-2-yl)-7-formyl-1-(oxan-2-yl)indazol-5-yl]-3-fluoro-5-(trifluoromethyl)benzamide (Example 71, Step 1: 40 mg, 0.055 mmol) and cyclobutylamine (7.85 mg, 0.11 mmol) in methanol (1 mL) was added acetic acid (3 mg, 0.05 mmol) at room temperature. The reaction was stirred at 50° C. for 1 h. Upon cooling to room temperature, to the above mixture was added sodium cyanoborohydride (7 mg, 0.11 mmol) at room temperature. The resulting mixture was stirred for additional 1 h. To the above mixture was added hydrazine hydrate (98%, 0.1 mL) at room temperature. The reaction mixture was stirred for additional 3 h. The resulting mixture was concentrated under reduced pressure. The residue was purified by silica gel column chromatography, eluted with 10% methanol in dichloromethane to afford the desired product as a white solid (28 mg, 78%). LCMS calculated for C₃₁H₃₀ClF₅N₅O₃ (M+H)⁺ m/z=650.2; found 650.4.

### Step 2: N-(3-amino-4-(2-chloro-5-fluorophenoxy)-7-((cyclobutylamino)methyl)-1H-indazol-5-yl)-3-fluoro-5-(trifluoromethyl)benzamide

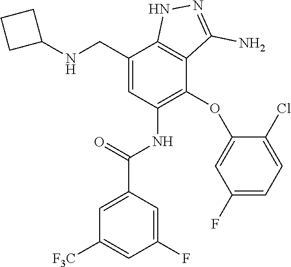 [SMILES: Nc1n[nH]c2c(CNC3CCC3)cc(NC(=O)c3cc(F)cc(C(F)(F)F)c3)c(Oc3cc(F)ccc3Cl)c12]

To the mixture of N-[3-amino-4-(2-chloro-5-fluorophenoxy)-7-[(cyclobutylamino)methyl]-1-(oxan-2-yl)indazol-5-yl]-3-fluoro-5-(trifluoromethyl)benzamide (27 mg, 0.04 mmol) in dichloromethane (3 mL) was added 2,2,2-trifluoroacetic acid (0.3 mL) at room temperature. The reaction was stirred for 2 h. The resulting mixture was concentrated under reduced pressure. The residue was purified by reversed-phase flash chromatography with the following conditions: column, C18 silica gel; mobile phase, acetonitrile in water (10 mmol/L ammonium bicarbonate), 10% to 90% gradient in 20 min; detector, UV 254 nm, eluted fractions were collected and lyophilized to the desired product as a light-yellow solid (18 mg, 77%). LCMS calculated for C₂₆H₂₂ClF₅N₅O₂(M+H)⁺ m/z=566.1; found 566.3.

### Step 3: N-(2-amino-3-(2-chloro-5-fluorophenoxy)-7-cyclobutyl-8-oxo-7,8-dihydro-6H-pyrazolo[4,5,1-ij]quinazolin-4-yl)-3-fluoro-5-(trifluoromethyl)benzamide

To the mixture of N-[3-amino-4-(2-chloro-5-fluorophenoxy)-7-[(cyclobutylamino)methyl]-1H-indazol-5-yl]-3-fluoro-5-(trifluoromethyl)benzamide (13 mg, 0.02 mmol) in acetonitrile (1.5 mL) was added 1,1′-carbonyldiimidazole (7.5 mg, 0.05 mmol), followed by 1,8-diazabicyclo[5.4.0]undec-7-ene (1 mg, 0.007 mmol) at room temperature. The reaction was stirred for additional 2 h. The resulting mixture was concentrated under reduced pressure. The crude product was purified by Prep-HPLC with the following conditions (Column: X select CSH F-Phenyl OBD column 30*250 mm, 5 m; Mobile Phase: acetonitrile in water (0.1% formic acid; Flow rate: 60 mL/min; Gradient: 34% B to 52% B in 10 min; Wave Length: 254 nm/220 nm). Eluted fractions were collected and lyophilized to afford the desired product as a white solid. LCMS calculated for C₂₇H₂₀ClF₅N₅O₃ (M+H)⁺ m/z=592.1; found 592.1. ¹H NMR (400 MHz, DMSO-d₆) δ 10.21 (s, 1H), 7.95-7.88 (m, 1H), 7.80-7.72 (m, 2H), 7.43 (dd, J=8.8, 5.6 Hz, 1H), 7.34 (s, 1H), 6.93-6.83 (m, 1H), 6.56 (dd, J=9.6, 2.8 Hz, 1H), 5.75 (s, 2H), 4.91 (s, 2H), 4.88-4.76 (m, 1H), 2.42-2.28 (m, 1H), 2.16-2.05 (m, 3H), 1.74-1.64 (m, 2H).

### Example 73. N-(4-Amino-3-(2-chloro-5-fluorophenoxy)-7-oxo-7H-pyrazolo[4,5,1-de]phenanthridin-2-yl)-3-fluoro-5-(trifluoromethyl)benzamide

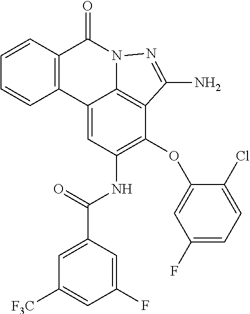 [SMILES: Nc1nn2c(=O)c3ccccc3c3cc(NC(=O)c4cc(F)cc(C(F)(F)F)c4)c(Oc4cc(F)ccc4Cl)c1c32]

### Step 1: N-(3-amino-7-bromo-4-(2-chloro-5-fluorophenoxy)-1-(tetrahydro-2H-pyran-2-yl)-1H-indazol-5-yl)-3-fluoro-5-(trifluoromethyl)benzamide

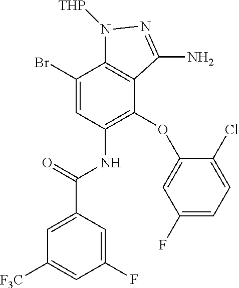

To the mixture of N-(7-bromo-4-(2-chloro-5-fluorophenoxy)-3-(1,3-dioxoisoindolin-2-yl)-1-(tetrahydro-2H-pyran-2-yl)-1H-indazol-5-yl)-3-fluoro-5-(trifluoromethyl)benzamide (Example 36, Step 1: 1.3 g, 1.67 mmol) in methanol (10 mL) was added hydrazine hydrate (98%, 0.3 mL) at room temperature. The reaction was stirred for additional 1 h. The resulting mixture was concentrated under reduced pressure. The residue was purified by silica gel column chromatography, eluted with 20% ethyl acetate in petroleum ether to afford the desired product as a yellow solid (900 mg, 83%). LCMS calculated for C₂₆H₂₀BrClF₅N₄O₃ (M+H)⁺ m/z=645.0; found 645.1.

### Step 2: Methyl 2-(3-amino-4-(2-chloro-5-fluorophenoxy)-5-(3-fluoro-5-(trifluoromethyl)benzamido)-1-(tetrahydro-2H-pyran-2-yl)-1H-indazol-7-yl)benzoate

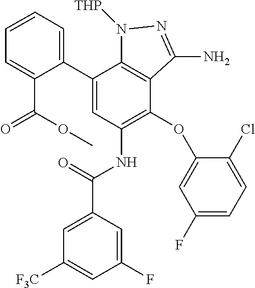

To a screw-cap vial equipped with a magnetic stir bar were placed N-[3-amino-7-bromo-4-(2-chloro-5-fluorophenoxy)-1-(oxan-2-yl)indazol-5-yl]-3-fluoro-5-(trifluoromethyl)benzamide (100 mg, 0.16 mmol), 2-(methoxycarbonyl)phenylboronic acid (42 mg, 0.23 mmol), Pd(dppf)Cl₂CH₂Cl₂ (13 mg, 0.02 mmol) and potassium carbonate (64 mg, 0.46 mmol). The vial was sealed with a Teflon-lined septum, evacuated and backfilled with nitrogen (this process was repeated a total of three times). Dioxane (1 mL) and water (0.2 mL) were added. The reaction was stirred at 80° C. for 2 h. Upon cooling to room temperature, the mixture was concentrated under reduced pressure. The residue was purified by silica gel column chromatography, eluted with 50% ethyl acetate in petroleum ether to afford the desired product as a white solid (50 mg, 46%). LCMS calculated for C₃₄H₂₇ClF₅N₄O₅ (M+H)⁺ m/z=701.2; found 701.2.

### Step 3: Methyl 2-(3-amino-4-(2-chloro-5-fluorophenoxy)-5-(3-fluoro-5-(trifluoromethyl)benzamido)-1H-indazol-7-yl)benzoate

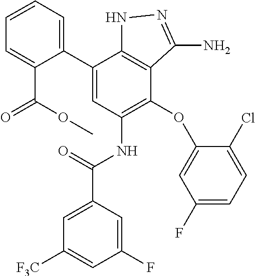 [SMILES: COC(=O)c1ccccc1-c1cc(NC(=O)c2cc(F)cc(C(F)(F)F)c2)c(Oc2cc(F)ccc2Cl)c2c(N)n[nH]c12]

To the mixture of methyl 2-[3-amino-4-(2-chloro-5-fluorophenoxy)-5-[3-fluoro-5-(trifluoromethyl)benzamido]-1-(oxan-2-yl)indazol-7-yl]benzoate (50 mg, 0.07 mmol) and triethylsilane (1 mL) in dichloromethane (0.2 mL) was added 2,2,2-trifluoroacetic acid (0.1 mL) at 0° C. The reaction was stirred at room temperature for 2 h. The resulting mixture was concentrated under reduced pressure. The crude product was used in the next step directly without further purification. LCMS calculated for C₂₉H₁₉ClF₅N₄O₄ (M+H)⁺ m/z=617.1; found 617.1.

### Step 4: N-(4-amino-3-(2-chloro-5-fluorophenoxy)-7-oxo-7H-pyrazolo[4,5,1-de]phenanthridin-2-yl)-3-fluoro-5-(trifluoromethyl)benzamide

To the mixture of methyl 2-[3-amino-4-(2-chloro-5-fluorophenoxy)-5-[3-fluoro-5-(trifluoromethyl)benzamido]-1H-indazol-7-yl]benzoate (20 mg, 0.03 mmol) in methanol (0.5 mL) was added sodium methoxide (3.5 mg, 0.06 mmol) at room temperature. The reaction was stirred for additional 16 h. The resulting mixture was concentrated under reduced pressure. The residue was purified by reversed-phase flash chromatography with the following conditions (column, C18 silica gel; mobile phase, acetonitrile in water (0.1% formic acid), 10% to 50% gradient in 10 min; detector, UV 254 nm). Eluted fractions were collected and lyophilized to afford the desired product as a white solid as a white solid. LCMS calculated for C₂₈H₁₅ClF₅N₄O₃ (M+H)⁺ m/z=585.1; found 585.1. ¹H NMR (400 MHz, DMSO-d₆) δ 10.40 (s, 1H), 8.58-8.52 (m, 2H), 8.52-8.46 (m, 1H), 7.98-7.88 (m, 2H), 7.83-7.72 (m, 3H), 7.42 (dd, J=8.8, 5.6 Hz, 1H), 6.94-6.86 (m, 1H), 6.78 (dd, J=9.6, 2.8 Hz, 1H), 6.62 (s, 2H).

### Example 74. N-(4-Amino-3-(2-chloro-5-fluorophenoxy)-7-oxo-7,8,9,10-tetrahydrocyclopenta[c]pyrazolo[4,5,1-ij]quinolin-2-yl)-3-fluoro-5-(trifluoromethyl)benzamide

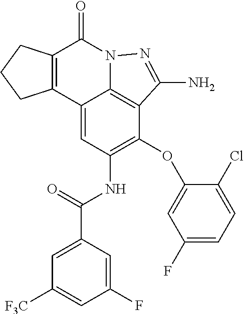 [SMILES: Nc1nn2c(=O)c3c(c4cc(NC(=O)c5cc(F)cc(C(F)(F)F)c5)c(Oc5cc(F)ccc5Cl)c1c42)CCC3]

### Step 1: Methyl 2-(((trifluoromethyl)sulfonyl)oxy)cyclopent-1-ene-1-carboxylate

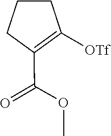 [SMILES: COC(=O)C1=C(OS(=O)(=O)C(F)(F)F)CCC1]

To the mixture of methyl 2-oxocyclopentane-1-carboxylate (1 g, 7 mmol) in diethyl ether (14 mL) was added sodium hydride (60% in mineral oil, 1.41 g, 35.2 mmol) in portions at 0° C. The reaction was stirred for 0.5 h at 0° C., followed by the addition of triflic anhydride (3.97 g, 14 mmol) dropwise at 0° C. The reaction mixture was stirred at the same temperature for 1 h. The resulting mixture was quenched with water at 0° C. The aqueous solution was extracted with dichloromethane (3×50 mL). The combined organic layers were washed with brine (30 mL), dried over anhydrous sodium sulfate. After filtration, the filtrate was concentrated under reduced pressure. The residue was purified by silica gel column chromatography, eluted with 10% ethyl acetate in petroleum ether to afford the desired product as a light-yellow oil (820 mg, 40%). LCMS calculated for C₈H₁₀F₃O₅S (M+H)⁺ m/z=275.0; found 275.1.

### Step 2: Methyl 2-(4,4,5,5-tetramethyl-1,3,2-dioxaborolan-2-yl)cyclopent-1-ene-1-carboxylate

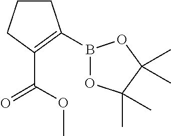 [SMILES: COC(=O)C1=C(B2OC(C)(C)C(C)(C)O2)CCC1]

To a screw-cap vial equipped with a magnetic stir bar were placed methyl 2-(((trifluoromethyl)sulfonyl)oxy)cyclopent-1-ene-1-carboxylate (720 mg, 2.6 mmol), bis(pinacolato)diboron (733 mg, 2.9 mmol), Pd(Ph₃P)₂Cl₂ (55.3 mg, 0.08 mmol), triphenylphosphine (41 mg, 0.16 mmol) and potassium carbonate (541 mg, 3.9 mmol). The vial was sealed with a Teflon-lined septum, evacuated and backfilled with nitrogen (this process was repeated a total of three times). 1,4-Dioxane (7 mL) was added. The reaction was stirred at 80° C. for 16 h. Upon cooling to room temperature, the mixture was concentrated under reduced pressure. The residue was purified by silica gel column chromatography, eluted with 10% ethyl acetate in petroleum ether to afford the desired product as a white solid (500 mg, 75%). LCMS calculated for C₁₃H₂₂BO₄ (M+H)⁺ m/z=253.2; found 253.0.

### Step 3: Methyl 2-(3-amino-4-(2-chloro-5-fluorophenoxy)-5-(3-fluoro-5-(trifluoromethyl)benzamido)-1-(tetrahydro-2H-pyran-2-yl)-1H-indazol-7-yl)cyclopent-1-ene-1-carboxylate

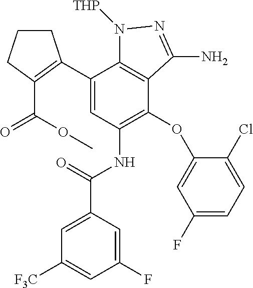

To a screw-cap vial equipped with a magnetic stir bar were placed N-[3-amino-7-bromo-4-(2-chloro-5-fluorophenoxy)-1-(oxan-2-yl)indazol-5-yl]-3-fluoro-5-(trifluoromethyl)benzamide (Example 73, Step 1: 100 mg, 0.16 mmol), methyl 2-(4,4,5,5-tetramethyl-1,3,2-dioxaborolan-2-yl)cyclopent-1-ene-1-carboxylate (78 mg, 0.31 mmol), Pd(dppf)Cl₂ (11 mg, 0.015 mmol) and potassium carbonate (43 mg, 0.31 mmol). The vial was sealed with a Teflon-lined septum, evacuated and backfilled with nitrogen (this process was repeated a total of three times). 1,4-Dioxane (1 mL) and water (0.2 mL) were added. The reaction was stirred at 80° C. for 16 h. Upon cooling to room temperature, the resulting mixture was concentrated under reduced pressure. The residue was purified by silica gel column chromatography, eluted with 50% ethyl acetate in petroleum ether to afford the desired product as a yellow solid (30 mg, 23%). LCMS calculated for C₃₃H₂₉ClF₅N₄O₅ (M+H)⁺ m/z=691.2; found 691.2.

### Step 4: N-(4-amino-3-(2-chloro-5-fluorophenoxy)-7-oxo-7,8,9,10-tetrahydrocyclopenta[c]pyrazolo[4,5,1-ij]quinolin-2-yl)-3-fluoro-5-(trifluoromethyl)benzamide

To the mixture of methyl 2-[3-amino-4-(2-chloro-5-fluorophenoxy)-5-[3-fluoro-5-(trifluoromethyl)benzamido]-1-(oxan-2-yl)indazol-7-yl]cyclopent-1-ene-1-carboxylate (25 mg, 0.04 mmol) and triethylsilane (21 mg, 0.18 mmol) in dichloromethane (1 mL) was added 2,2,2-trifluoroacetic acid (0.1 mL) at room temperature. The reaction was stirred for 16 h. The resulting mixture was concentrated under reduced pressure. The residue was purified by silica gel column chromatography, eluted with 50% ethyl acetate in petroleum ether to afford crude product, which was further purified by reversed-phase flash chromatography with the following conditions: (Column, C18 silica gel; mobile phase, acetonitrile in water (0.1% formic acid), 10% to 95% gradient in 20 min; detector, UV 254 nm). Eluted fractions were collected and lyophilized to afford the desired product as a yellow solid. LCMS calculated for C₂₇H₁₇ClF₅N₄O₃ (M+H)⁺ m/z=575.1; found 575.0. ¹H NMR (400 MHz, DMSO-d₆) δ 10.32 (s, 1H), 7.97-7.90 (m, 1H), 7.87 (s, 1H), 7.78-7.71 (m, 2H), 7.41 (dd, J=8.8, 5.6 Hz, 1H), 6.93-6.84 (m, 1H), 6.72 (dd, J=9.6, 2.8 Hz, 1H), 6.55 (s, 2H), 3.15 (t, J=7.6 Hz, 2H), 2.87 (t, J=7.6 Hz, 2H), 2.25-2.13 (m, 2H).

### Example 75. N-(4-Amino-3-(2-chloro-5-fluorophenoxy)-7-oxo-8,9,10,11-tetrahydro-7H-pyrazolo[4,5,1-de]phenanthridin-2-yl)-3-fluoro-5-(trifluoromethyl)benzamide

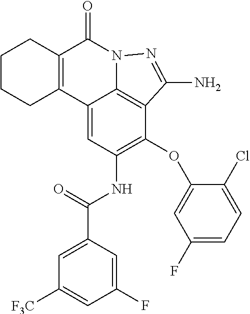 [SMILES: Nc1nn2c(=O)c3c(c4cc(NC(=O)c5cc(F)cc(C(F)(F)F)c5)c(Oc5cc(F)ccc5Cl)c1c42)CCCC3]

### Step 1: Ethyl 2-(3-amino-4-(2-chloro-5-fluorophenoxy)-5-(3-fluoro-5-(trifluoromethyl)benzamido)-1-(tetrahydro-2H-pyran-2-yl)-1H-indazol-7-yl)cyclohex-1-ene-1-carboxylate

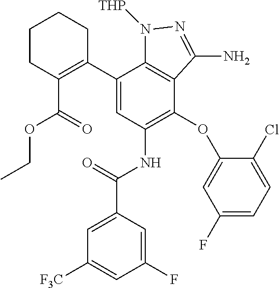

To a screw-cap vial equipped with a magnetic stir bar were placed N-[3-amino-7-bromo-4-(2-chloro-5-fluorophenoxy)-1-(oxan-2-yl)indazol-5-yl]-3-fluoro-5-(trifluoromethyl)benzamide (Example 73, Step 1: 200 mg, 0.31 mmol), ethyl 2-(4,4,5,5-tetramethyl-1,3,2-dioxaborolan-2-yl)cyclohex-1-ene-1-carboxylate (173 mg, 0.62 mmol), Pd(dppf)Cl₂ (23 mg, 0.03 mmol) and potassium carbonate (86 mg, 0.62 mmol). The vial was sealed with a Teflon-lined septum, evacuated and backfilled with nitrogen (this process was repeated a total of three times). 1,4-Dioxane (2 mL) and water (0.4 mL) were added. The reaction was stirred at 80° C. for 16 h. Upon cooling to room temperature, the resulting mixture was concentrated under reduced pressure. The residue was purified by reversed-phase flash chromatography with the following conditions: column, C18 silica gel; mobile phase, acetonitrile in water (0.1% formic acid), 10% to 95% gradient in 30 min; detector, UV 254 nm), eluted fractions were collected and lyophilized to afford the desired product as a light-yellow solid (20 mg, 9%). LCMS calculated for C₃₅H₃₃ClF₅N₄O₅ (M+H)⁺ m/z=719.2; found 719.2.

### Step 2: N-(4-amino-3-(2-chloro-5-fluorophenoxy)-7-oxo-8,9,10,11-tetrahydro-7H-pyrazolo[4,5,1-de]phenanthridin-2-yl)-3-fluoro-5-(trifluoromethyl)benzamide

To the mixture of methyl 2-[3-amino-4-(2-chloro-5-fluorophenoxy)-5-[3-fluoro-5-(trifluoromethyl)benzamido]-1-(oxan-2-yl)indazol-7-yl]cyclohex-1-ene-1-carboxylate (20 mg, 0.03 mmol) and triethylsilane (16.5 mg, 0.14 mmol) in dichloromethane (1 mL) was added 2,2,2-trifluoroacetic acid (0.1 mL) at room temperature. The reaction was stirred for 16 h. The resulting mixture was concentrated under reduced pressure. The residue was purified by reversed-phase flash chromatography with the following conditions: column, C18 silica gel; mobile phase, acetonitrile in water (0.1% formic acid), 10% to 90% gradient in 20 min; detector, UV 254 nm). Eluted fractions were collected and lyophilized to afford the desired product as a yellow solid. LCMS calculated for C₂₈H₁₉ClF₅N₄O₃ (M+H)⁺ m/z=589.1; found 589.1. ¹H NMR (400 MHz, DMSO-d₆) δ 10.33 (s, 1H), 7.96-7.89 (m, 2H), 7.78-7.71 (m, 2H), 7.41 (dd, J=8.8, 5.6 Hz, 1H), 6.93-6.86 (m, 1H), 6.71 (dd, J=9.6, 2.8 Hz, 1H), 6.55 (s, 2H), 2.95-2.87 (m, 2H), 2.64-2.57 (m, 2H), 1.85-1.76 (m, 4H).

### Example 76. (E)-N-(3-Amino-4-(2-chloro-5-fluorophenoxy)-1-methyl-7-(2-(pyridin-2-yl)vinyl)-1H-indazol-5-yl)-3-fluoro-5-(trifluoromethyl)benzamide

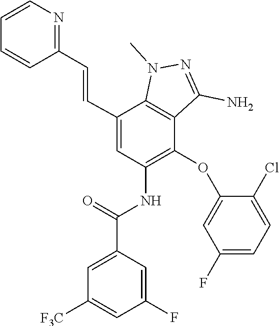 [SMILES: Cn1nc(N)c2c(Oc3cc(F)ccc3Cl)c(NC(=O)c3cc(F)cc(C(F)(F)F)c3)cc(/C=C/c3ccccn3)c21]

### Step 1: (E)-N-(4-(2-chloro-5-fluorophenoxy)-3-(1,3-dioxoisoindolin-2-yl)-1-methyl-7-(2-(pyridin-2-yl)vinyl)-1H-indazol-5-yl)-3-fluoro-5-(trifluoromethyl)benzamide

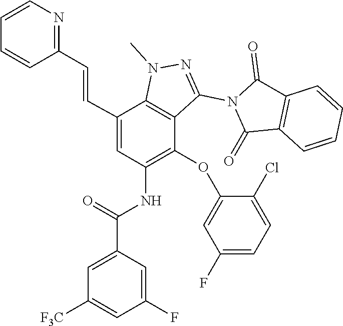 [SMILES: Cn1nc(N2C(=O)c3ccccc3C2=O)c2c(Oc3cc(F)ccc3Cl)c(NC(=O)c3cc(F)cc(C(F)(F)F)c3)cc(/C=C/c3ccccn3)c21]

To a screw-cap vial equipped with a magnetic stir bar were placed N-(7-bromo-4-(2-chloro-5-fluorophenoxy)-3-(1,3-dioxoisoindolin-2-yl)-1-methyl-1H-indazol-5-yl)-3-fluoro-5-(trifluoromethyl)benzamide (Example 25, Step 1: 80 mg, 0.1 mmol), Pd(OAc)₂ (2.54 mg, 0.01 mmol) and tri(2-methylphenyl)phosphine (7 mg, 0.02 mmol). The vial was sealed with a Teflon-lined septum, evacuated and backfilled with nitrogen (this process was repeated a total of three times). 2-Vinylpyridine (15.5 mg, 0.15 mmol) and triethylamine (0.1 mL) in N,N-Dimethylformamide (1 mL) were added. The reaction was stirred at 135° C. for 3 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 50% ethyl acetate in petroleum ether to afford the desired product as a yellow solid (70 mg, 84.6%). LCMS calculated for C₃₇H₂₂ClF₅N₅O₄ (M+H)⁺ m/z=730.1; found 730.1.

### Step 2: (E)-N-(3-amino-4-(2-chloro-5-fluorophenoxy)-1-methyl-7-(2-(pyridin-2-yl)vinyl)-1H-indazol-5-yl)-3-fluoro-5-(trifluoromethyl)benzamide

To the mixture of (E)-N-(4-(2-chloro-5-fluorophenoxy)-3-(1,3-dioxoisoindolin-2-yl)-1-methyl-7-(2-(pyridin-2-yl)vinyl)-1H-indazol-5-yl)-3-fluoro-5-(trifluoromethyl)benzamide (20 mg, 0.03 mmol) and hydrazine hydrate (98%, 0.1 mL) in methanol (0.5 mL) was stirred at room temperature for 16 h. The resulting mixture was concentrated under reduced pressure.

The residue was purified by reversed-phase flash chromatography with the following conditions (Column: C18 silica gel; Mobile phase, acetonitrile in water (0.1% trifluoroacetic acid), 10% to 50% gradient in 20 min; Detector, 254 nm), eluted fractions were lyophilized to afford the TFA salt of the desired product as a red solid. LCMS calculated for C₂₉H₂₀ClF₅N₅O₂ (M+H)⁺ m/z=600.1; found 600.1. ¹H NMR (400 MHz, DMSO-d₆) δ 10.23 (s, 1H), 8.73-8.67 (m, 1H), 8.36 (d, J=15.6 Hz, 1H), 8.08-8.00 (m, 1H), 7.95-7.89 (m, 1H), 7.88-7.75 (m, 3H), 7.68 (s, 1H), 7.52-7.53 (m, 2H), 7.26 (d, J=15.6 Hz, 1H), 6.92-6.84 (m, 1H), 6.53 (dd, J=9.6, 2.8 Hz, 1H), 5.02 (s, 2H), 4.08 (s, 3H).

### Example 77. N-(3-Amino-4-(2-chloro-5-fluorophenoxy)-1-methyl-7-(2-(pyridin-2-yl)ethyl)-1H-indazol-5-yl)-3-fluoro-5-(trifluoromethyl)benzamide

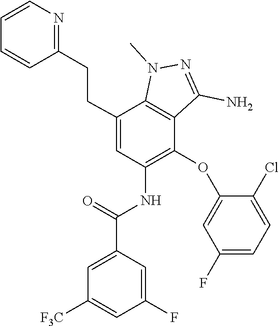 [SMILES: Cn1nc(N)c2c(Oc3cc(F)ccc3Cl)c(NC(=O)c3cc(F)cc(C(F)(F)F)c3)cc(CCc3ccccn3)c21]

### Step 1: N-(4-(2-chloro-5-fluorophenoxy)-3-(1,3-dioxoisoindolin-2-yl)-1-methyl-7-(2-(pyridin-2-yl)ethyl)-1H-indazol-5-yl)-3-fluoro-5-(trifluoromethyl)benzamide

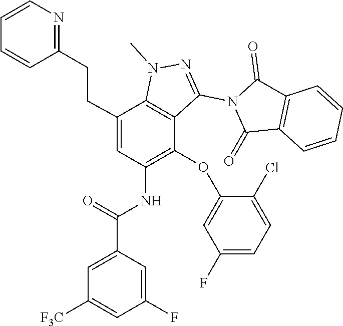 [SMILES: Cn1nc(N2C(=O)c3ccccc3C2=O)c2c(Oc3cc(F)ccc3Cl)c(NC(=O)c3cc(F)cc(C(F)(F)F)c3)cc(CCc3ccccn3)c21]

To the mixture of N-[4-(2-chloro-5-fluorophenoxy)-3-(1,3-dioxoisoindol-2-yl)-1-methyl-7-[(E)-2-(pyridin-2-yl)ethenyl]indazol-5-yl]-3-fluoro-5-(trifluoromethyl)benzamide (Example 76, Step 1: 40 mg, 0.05 mmol) in methanol (2 mL) was added Pd/C (10%, 4 mg) at room temperature. The flask was evacuated and flushed three times with nitrogen, followed by flushing with hydrogen. The mixture was stirred at room temperature for 16 h under an atmosphere of hydrogen (balloon). The resulting mixture was filtered, the filter cake was washed with methanol (30 mL), and the filtrate was concentrated under reduced pressure. The crude product was used in the next step directly without further purification. LCMS calculated for C₃₇H₂₄ClF₅N₅O₄ (M+H)⁺ m/z=732.1; found 732.1.

### Step 2: N-(3-amino-4-(2-chloro-5-fluorophenoxy)-1-methyl-7-(2-(pyridin-2-yl)ethyl)-1H-indazol-5-yl)-3-fluoro-5-(trifluoromethyl)benzamide

To the mixture of N-(4-(2-chloro-5-fluorophenoxy)-3-(1,3-dioxoisoindolin-2-yl)-1-methyl-7-(2-(pyridin-2-yl)ethyl)-1H-indazol-5-yl)-3-fluoro-5-(trifluoromethyl)benzamide (15 mg, 0.02 mmol) in methanol (1 mL) was added hydrazine hydrate (98%, 0.1 mL) at room temperature. The reaction was stirred for 2 h. The resulting mixture was concentrated under reduced pressure. The residue was purified by reversed-phase flash chromatography with the following conditions: (Column, C18 silica gel; Mobile phase: acetonitrile in water (0.1% trifluoroacetic acid), 10% to 50% gradient in 20 min; Detector: 254 nm), eluted fractions were lyophilized to afford the TFA salt of the desired product as a white solid. LCMS calculated for C₂₉H₂₂ClF₅N₅O₂ (M+H)⁺ m/z=602.1; found 602.1. ¹H NMR (400 MHz, DMSO-d₆) δ 10.14 (s, 1H), 8.77-8.71 (m, 1H), 8.29-8.20 (m, 1H), 7.94-7.87 (m, 1H), 7.84-7.73 (m, 3H), 7.73-7.66 (m, 1H), 7.48 (dd, J=8.8, 5.6 Hz, 1H), 7.07 (s, 1H), 6.91-6.82 (m, 1H), 6.40 (dd, J=9.6, 2.8 Hz, 1H), 6.06 (s, 3H), 3.51-3.43 (m, 2H), 3.37-3.29 (m, 2H).

### Example 78. N-(3-Amino-4-(2-chloro-5-fluorophenoxy)-1-methyl-7-(piperidin-2-ylethynyl)-1H-indazol-5-yl)-3-fluoro-5-(trifluoromethyl)benzamide

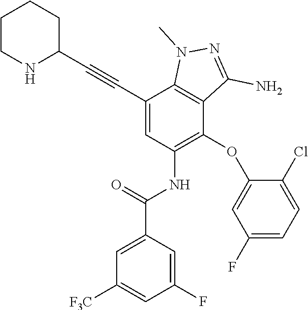 [SMILES: Cn1nc(N)c2c(Oc3cc(F)ccc3Cl)c(NC(=O)c3cc(F)cc(C(F)(F)F)c3)cc(C#CC3CCCCN3)c21]

### Step 1: tert-butyl 2-((3-amino-4-(2-chloro-5-fluorophenoxy)-5-(3-fluoro-5-(trifluoromethyl)benzamido)-1-methyl-1H-indazol-7-yl)ethynyl)piperidine-1-carboxylate

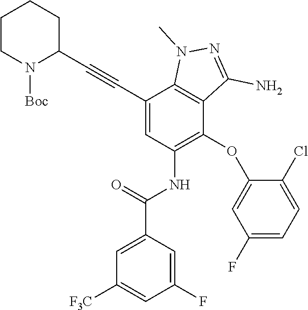 [SMILES: Cn1nc(N)c2c(Oc3cc(F)ccc3Cl)c(NC(=O)c3cc(F)cc(C(F)(F)F)c3)cc(C#CC3CCCCN3C(=O)OC(C)(C)C)c21]

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 30 mg, 0.052 mmol), Pd(PPh₃)₂Cl₂ (7.3 mg, 0.01 mmol) and cuprous iodide (2 mg, 0.01 mmol). The vial was sealed with a Teflon-lined septum, evacuated and backfilled with nitrogen (this process was repeated a total of three times). A mixture of tert-butyl 2-ethynylpiperidine-1-carboxylate (22 mg, 0.1 mmol) and triethylamine (0.7 mL) in N,N-Dimethylformamide (0.7 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 10% methanol in dichloromethane to afford the desired product as a brown yellow solid (30 mg, 81%). LCMS calculated for C₃₄H₃₂ClF₅N₅O₄ (M+H)⁺ m/z=704.2; found 704.4.

### Step 2: N-(3-amino-4-(2-chloro-5-fluorophenoxy)-1-methyl-7-(piperidin-2-ylethynyl)-1H-indazol-5-yl)-3-fluoro-5-(trifluoromethyl)benzamide

To the mixture of tert-butyl 2-((3-amino-4-(2-chloro-5-fluorophenoxy)-5-(3-fluoro-5-(trifluoromethyl)benzamido)-1-methyl-1H-indazol-7-yl)ethynyl)piperidine-1-carboxylate (30 mg, 0.04 mmol) in dichloromethane (5 mL) was added trifluoroacetic acid (1 mL) at room temperature. The reaction was stirred for 1 h. The mixture was concentrated under reduced pressure. The crude product was purified by Prep-HPLC with the following conditions (Column: XBridge Prep Phenyl OBD Column 19*250 mm, 5 m; Mobile Phase: acetonitrile in water (0.1% 2,2,2-trifluoroacetic acid); Flow rate: 60 mL/min; Gradient: 29% B to 47% B in 10 min; Detector: 254/220 nm). Eluted fractions were collected and lyophilized to afford the TFA salt of the desired product as an off-white solid. LCMS calculated for C₂₉H₂₄ClF₅N₅O₂ (M+H)⁺ m/z=604.2; found 604.2. ¹H NMR (400 MHz, DMSO-d₆) δ 10.16 (s, 1H), 7.92 (d, J=8.4 Hz, 1H), 7.76-7.69 (m, 2H), 7.51-7.43 (m, 2H), 6.92-6.84 (m, 1H), 6.52 (dd, J=9.6, 2.8 Hz, 1H), 5.16 (s, 2H), 4.68-4.51 (m, 1H), 4.09 (s, 3H), 3.30-2.99 (m, 2H), 2.17-2.07 (m, 1H), 1.96-1.56 (m, 5H).

### Example 79. N-(7-((1-Acetylpiperidin-2-yl)ethynyl)-3-amino-4-(2-chloro-5-fluorophenoxy)-1-methyl-1H-indazol-5-yl)-3-fluoro-5-(trifluoromethyl)benzamide

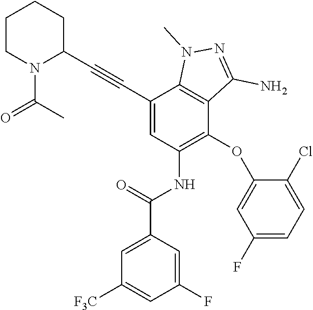 [SMILES: CC(=O)N1CCCCC1C#Cc1cc(NC(=O)c2cc(F)cc(C(F)(F)F)c2)c(Oc2cc(F)ccc2Cl)c2c(N)nn(C)c12]

### Step 1: 2-Ethynylpiperidine

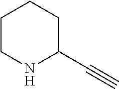

The mixture of tert-butyl 2-ethynylpiperidine-1-carboxylate (1 g, 4.78 mmol) and trifluoroacetic acid (3 mL) in dichloromethane (9 mL) was stirred at room temperature for 2 h. The resulting mixture was concentrated under reduced pressure. The crude product was used in the next step directly without further purification. LCMS calculated for C₇H₁₂N (M+H)⁺ m/z=110.1; found 110.2.

### Step 2: 1-(2-Ethynylpiperidin-1-yl)ethan-1-one

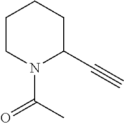

To the mixture of 2-ethynylpiperidine (200 mg, 1.83 mmol) and triethylamine (556 mg, 5.5 mmol) in dichloromethane (2 mL) was added acetic anhydride (243 mg, 2.38 mmol) at room temperature. The reaction was stirred at for 2 h. The resulting mixture was concentrated under reduced pressure. The residue was purified by silica gel column chromatography, eluted with 10% methanol in dichloromethane to afford the desired product as a yellow oil (100 mg, 36%). LCMS calculated for C₉H₁₄NO (M+H)⁺ m/z=152.1; found 152.0.

### Step 3: N-(7-((I-acetylpiperidin-2-yl)ethynyl)-3-amino-4-(2-chloro-5-fluorophenoxy)-1-methyl-1H-indazol-5-yl)-3-fluoro-5-(trifluoromethyl)benzamide

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 20 mg, 0.035 mmol), Pd(PPh₃)₂Cl₂ (4.9 mg, 0.007 mmol) and cuprous iodide (1.3 mg, 0.007 mmol). The vial was sealed with a Teflon-lined septum, evacuated and backfilled with nitrogen (this process was repeated a total of three times). 1-(2-Ethynylpiperidin-1-yl)ethan-1-one (10.5 mg, 0.07 mmol) and triethylamine (0.5 mL) in N,N-Dimethylformamide (0.5 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 10% methanol in dichloromethane to afford crude product which was further purified by reversed-phase flash chromatography with the following conditions (Column: Xselect CSH C18 OBD Column 30*150 mm; Mobile Phase: acetonitrile in water (0.1% formic acid); Flow rate: 60 mL/min; 42% B to 62% B in 10 min; Detector: 254/220 nm). Eluted fractions were collected and lyophilized to afford the desired product as a yellow solid. LCMS calculated for C₃₁H₂₄ClF₅N₅O₃ (M−H)⁻ m/z=644.1; found 644.2. ¹H NMR (400 MHz, DMSO-d₆) δ 10.13 (s, 1H), 7.93-7.86 (m, 1H), 7.77-7.70 (m, 2H), 7.50-7.42 (m, 2H), 6.91-6.83 (m, 1H), 6.51 (dd, J=9.6, 2.8 Hz, 1H), 5.81-5.75 (m, 1H), 5.09 (s, 2H), 4.06 (s, 3H), 3.85-3.75 (m, 1H), 2.15-1.86 (m, 5H), 1.84-1.58 (m, 4H), 1.53-1.28 (m, 1H).

### Example 80. 2-((3-Amino-4-(2-chloro-5-fluorophenoxy)-5-(3-fluoro-5-(trifluoromethyl)benzamido)-1-methyl-1H-indazol-7-yl)ethynyl)-N-methylpiperidine-1-carboxamide

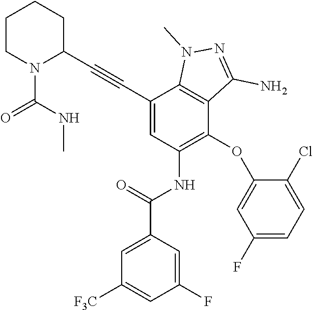 [SMILES: CNC(=O)N1CCCCC1C#Cc1cc(NC(=O)c2cc(F)cc(C(F)(F)F)c2)c(Oc2cc(F)ccc2Cl)c2c(N)nn(C)c12]

### Step 1: 2-Ethynyl-N-methylpiperidine-1-carboxamide

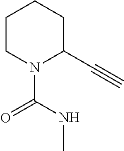

To the mixture of 2-ethynylpiperidine (Example 79, Step 1: 200 mg, 1.83 mmol) and triethylamine (0.28 mL, 2.0 mmol) in dichloromethane (2 mL) was added N-methylimidazole-1-carboxamide (229 mg, 1.83 mmol) at room temperature. The reaction was stirred for 2 h. The resulting mixture was concentrated under reduced pressure. The residue was purified by silica gel column chromatography, eluted with 10% methanol in dichloromethane to afford the desired product as a light-yellow solid (100 mg, 33%). LCMS calculated for C₉H₁₅N₂O (M+H)⁺ m/z=167.1; found 167.0.

### Step 2: 2-((3-Amino-4-(2-chloro-5-fluorophenoxy)-5-(3-fluoro-5-(trifluoromethyl)benzamido)-1-methyl-1H-indazol-7-yl)ethynyl)-N-methylpiperidine-1-carboxamide

To a screw-cap vial equipped with a magnetic stir bar were placed N-(3-amino-7-bromo-4-(2-chloro-5-fluorophenoxy)-1-methyl-1H-indazol-5-yl)-3-fluoro-5-(trifluoromethyl)benzamide (Example 25: 20 mg, 0.035 mmol), Pd(PPh₃)₂Cl₂ (4.9 mg, 0.007 mmol) and cuprous iodide (1.3 mg, 0.007 mmol). The vial was sealed with a Teflon-lined septum, evacuated and backfilled with nitrogen (this process was repeated a total of three times). 2-Ethynyl-N-methylpiperidine-1-carboxamide (11.5 mg, 0.07 mmol) and triethylamine (0.5 mL) in N,N-Dimethylformamide (0.5 mL) were added. The reaction was stirred at 80° C. for 2 h. Upon cooling to room temperature, the resulting mixture was diluted with water (20 mL). The resulting mixture was extracted with ethyl acetate (3×20 mL). The combined organic layers were washed with brine (50 mL), dried over anhydrous sodium sulfate. After filtration, the filtrate was concentrated under reduced pressure. The residue was purified by silica gel column chromatography, eluted with 50% ethyl acetate in petroleum ether to afford crude product which was further purified by reversed-phase flash chromatography with the following conditions (Column: Xselect CSH C18 OBD Column 30*150 mm; Mobile Phase: acetonitrile in water (0.1% formic acid); Flow rate: 60 mL/min; 40% B to 60% B in 10 min; Detector: 254/220 nm). Eluted fractions were collected and lyophilized to afford the desired product as a yellow solid. LCMS calculated for C₃₁H₂₇ClF₅N₆O₃ (M+H)⁺ m/z=661.2; found 661.2. ¹H NMR (400 MHz, DMSO-d₆) δ 10.13 (s, 1H), 7.93-7.87 (m, 1H), 7.78-7.71 (m, 2H), 7.46 (dd, J=8.8, 5.6 Hz, 1H), 7.41 (s, 1H), 6.92-6.83 (m, 1H), 6.66-6.59 (m, 1H), 6.51 (dd, J=9.6, 2.8 Hz, 1H), 5.41-5.35 (m, 1H), 5.08 (s, 2H), 4.05 (s, 3H), 3.82-3.73 (m, 1H), 3.02-2.90 (m, 1H), 2.60 (d, J=4.4 Hz, 3H), 1.90-1.82 (m, 1H), 1.75-1.63 (m, 4H), 1.44-1.28 (m, 1H).

### Example 81. N-(3-Amino-7-bromo-4-(2-chloro-5-fluorophenoxy)-1-(methylsulfonyl)-1H-indazol-5-yl)-3-fluoro-5-(trifluoromethyl)benzamide

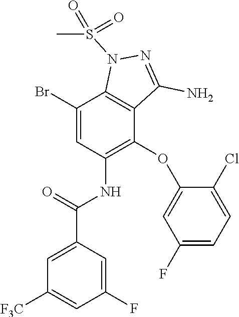 [SMILES: CS(=O)(=O)n1nc(N)c2c(Oc3cc(F)ccc3Cl)c(NC(=O)c3cc(F)cc(C(F)(F)F)c3)cc(Br)c21]

To the mixture of N-(3-amino-7-bromo-4-(2-chloro-5-fluorophenoxy)-1H-indazol-5-yl)-3-fluoro-5-(trifluoromethyl)benzamide (Example 11: 30 mg, 0.05 mmol) in tetrahydrofuran (1 mL) was added sodium hydride (60% in mineral oil, 3.20 mg, 0.08 mmol) at 0° C. The reaction was stirred for 30 min at the same temperature. To the above mixture was added methanesulfonyl chloride (7.34 mg, 0.06 mmol) dropwise at 0° C. The reaction was stirred for additional 1 h. The reaction was quenched with water at 0° C. The aqueous solution was extracted with ethyl acetate (3×5 mL). The combined organic layers were washed with brine (5 mL), dried over anhydrous sodium sulfate. After filtration, the filtrate was concentrated under reduced pressure. The residue was purified by silica gel column chromatography, eluted with 50% ethyl acetate in petroleum ether to afford crude product which was further purified by Prep-HPLC with the following conditions (Column: XBridge Prep Phenyl OBD Column 19*250 mm, 5 m; Mobile Phase: acetonitrile in water (0.1% formic acid); Flow rate: 60 mL/min; Gradient: 37% B to 52% B in 10 min; Wave Length: 254/220 nm). Eluted fractions were collected and lyophilized to afford the desired product as a white solid. LCMS calculated for C₂₂H₁₄BrClF₅N₄O₄S (M+H)⁺ m/z=639.0; found 639.0. ¹H NMR (400 MHz, DMSO-d₆) δ 10.34 (s, 1H), 7.93 (d, J=8.4 Hz, 1H), 7.89 (s, 1H), 7.70-7.68 (m, 2H), 7.42 (dd, J=8.8, 5.6 Hz, 1H), 6.91-6.88 (m, 1H), 6.81 (dd, J=9.6, 2.8 Hz, 1H), 6.22 (s, 2H), 3.30 (s, 3H).

### Example 82. N-(1-Acetyl-3-amino-7-bromo-4-(2-chloro-5-fluorophenoxy)-1H-indazol-5-yl)-3-fluoro-5-(trifluoromethyl)benzamide

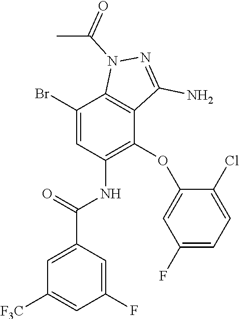 [SMILES: CC(=O)n1nc(N)c2c(Oc3cc(F)ccc3Cl)c(NC(=O)c3cc(F)cc(C(F)(F)F)c3)cc(Br)c21]

The mixture of acetic acid (4 mg, 0.07 mmol) and 2-(7-azabenzotriazol-1-yl)-N,N,N′,N′-tetramethyluronium hexafluorophosphate (24 mg, 0.06 mmol) in N,N-dimethylformamide (2 mL) was stirred for 30 min at room temperature. To the above mixture was added N-(3-amino-7-bromo-4-(2-chloro-5-fluorophenoxy)-1H-indazol-5-yl)-3-fluoro-5-(trifluoromethyl)benzamide (Example 11: 30 mg, 0.05 mmol) and N,N-diisopropylethylamine (14 mg, 0.11 mmol). The reaction was stirred for additional 1 h. The mixture was purified by Prep-HPLC with the following conditions (Column: Xselect CSH C18 OBD Column 30*150 mm 5 m; Mobile Phase: acetonitrile in water (0.1% formic acid); Flow rate: 60 mL/min; Gradient: 48% B to 68% B in 8 min; Wave Length: 254/220 nm). Eluted fractions were collected and lyophilized to afford the desired product as a yellow green solid. LCMS calculated for C₂₃H₁₄BrClF₅N₄O₃ (M+H)⁺ m/z=603.0; found 603.1. ¹H NMR (400 MHz, DMSO-d₆) δ 9.97 (s, 1H), 7.90 (d, J=8.4 Hz, 1H), 7.71-7.68 (m, 2H), 7.48 (s, 1H), 7.41 (dd, J=8.8, 5.6 Hz, 1H), 7.35 (s, 2H), 6.88-6.83 (m, 1H), 6.77 (dd, J=9.6, 2.8 Hz, 1H), 2.77 (s, 3H).

### Example A. pS473 AKT Assay

SKBR3 (PIK3CA WT), MCF7 (PIK3CA E545K), and T47D (PIK3CA H1047R) cells were cultured using 10-cm petri dishes with recommended medium. One day before the assay, cells were seeded in 96-well plates. After overnight incubation, cells were treated with different concentrations of PI3Kα inhibitors for 2 h. Cells were then fixed using 4% paraformaldehyde at room temperature for 20 min. Cells were aspirated with 4% paraformaldehyde, and washed using 1× regular phosphate buffered saline 3 times, 5 min each. Any residual phosphate buffered saline was aspirated and cells were blocked using 10% goat serum containing 1% bovine serum albumin and 0.3% Triton X-100 at room temperature for 1 h. Without any additional washing, primary antibodies (rabbit anti-pSer473 AKT) were diluted using blocking buffer and added at a final volume of 50 microliter per well. Assay plates with primary antibodies were maintained overnight at 4° C. Cells were washed using 1× regular phosphate buffered saline 3 times, 5 min each. After the final wash, cells were incubated with horseradish peroxidase-conjugated secondary antibodies (goat Anti-rabbit IgG) and diluted using the same blocking buffer at room temperature for 1 h. Cells were then washed thoroughly using 1× regular phosphate buffered saline 3 times, 5 min each, and any residual phosphate buffered saline was aspirated. Super-Signal ELISA Pico Chemiluminescent Substrate was then added at a final volume of 100 microliter per well. Plates were read on a i3x Multi-Mode Microplate Reader and IC₅₀ values calculated using GraphPad Prism software.

Results of the assay described above are presented in Table A. “++++” indicates an IC₅₀ less than 1000 nM; “+++” indicates an IC₅₀ greater than or equal to 1000 nM but less than 5000 nM; “++” indicates an IC₅₀ greater than or equal to 5000 nM but less than 10000 nM; and “+” indicates an IC₅₀ greater than or equal to 10000 nM.

TABLE A

| Example | SKBR3_IC₅₀ | T47D_IC₅₀ |
|---|---|---|
| No. | (nM) | (nM) |
| 1 | ++++ | ++++ |
| 2 | +++ | ++++ |
| 3 | + | ++++ |
| 4 | ++ | ++++ |
| 5 | ++ | +++ |
| 6 | + | +++ |
| 7 | ++++ | ++++ |
| 8 | ++++ | ++++ |
| 9 | +++ | ++++ |
| 10 | +++ | ++++ |
| 11 | ++++ | ++++ |
| 12 | ++++ | ++++ |
| 13 | ++++ | ++++ |
| 14 | +++ | ++++ |
| 15 | ++++ | ++++ |
| 16 | +++ | ++++ |
| 17 | +++ | ++++ |
| 18 | ++++ | ++++ |
| 19 | +++ | ++++ |
| 20 | +++ | ++++ |
| 21 | ++++ | ++++ |
| 22 | ++++ | ++++ |
| 23 | ++++ | ++++ |
| 24 | ++++ | ++++ |
| 25 | ++++ | ++++ |
| 26 | +++ | ++++ |
| 27 | +++ | ++++ |
| 28 | ++++ | ++++ |
| 29 | +++ | ++++ |
| 30 | ++++ | ++++ |
| 31 | ++++ | ++++ |
| 32 | ++++ | ++++ |
| 33 | +++ | ++++ |
| 34 | +++ | ++++ |
| 35 | +++ | ++++ |
| 36 | ++++ | ++++ |
| 37 | +++ | ++++ |
| 38 | +++ | ++++ |
| 39 | +++ | ++++ |
| 40 | ++++ | ++++ |
| 41 | +++ | ++++ |
| 42 | +++ | ++++ |
| 43 | ++++ | ++++ |
| 44 | ++++ | ++++ |
| 45 | ++++ | ++++ |
| 46 | +++ | ++++ |
| 47 | +++ | ++++ |
| 48 | +++ | ++++ |
| 49 | ++++ | ++++ |
| 50 | ++ | ++++ |
| 51 | + | ++++ |
| 52 | +++ | ++++ |
| 53 | + | ++++ |
| 54 | + | + |
| 55 | +++ | ++++ |
| 56 | +++ | ++++ |
| 57 | ++++ | ++++ |
| 58 | ++++ | ++++ |
| 59 | ++++ | ++++ |
| 60 | ++++ | ++++ |
| 61 | ++ | ++++ |
| 62 | +++ | ++++ |
| 63 | +++ | ++++ |
| 64 | +++ | ++++ |
| 65 | ++++ | ++++ |
| 66 | ++++ | ++++ |
| 67 | +++ | ++++ |
| 68 | ++++ | ++++ |
| 69 | ++++ | ++++ |
| 70 | + | ++++ |
| 71 | ++++ | ++++ |
| 72 | ++++ | ++++ |
| 73 | ++++ | ++++ |
| 74 | ++++ | ++++ |
| 75 | ++++ | ++++ |
| 76 | ++ | ++++ |
| 77 | +++ | ++++ |
| 78 | +++ | ++++ |
| 79 | +++ | ++++ |
| 80 | +++ | ++++ |
| 81 | +++ | ++++ |
| 82 | +++ | ++++ |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 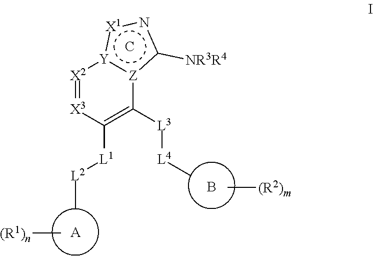
or a pharmaceutically acceptable salt thereof, wherein:
X¹ is CR⁵, O, N, or NR⁶;
X² is CR⁷ or N;
X³ is CR⁸ or N;
Y is C or N;
Z is C or N;
n is 0, 1, 2, 3, 4, 5, or 6;
m is 0, 1, 2, 3, 4, 5, or 6;
Ring A is C₃₋₁₄ cycloalkyl, C₆₋₁₀ aryl, 4-14 membered heterocycloalkyl, or 5-10 membered heteroaryl;
Ring B is C₃₋₁₄ cycloalkyl, C₆₋₁₀ aryl, 4-14 membered heterocycloalkyl, or 5-10 membered heteroaryl;
Ring C is a 5-membered heteroaryl having 2 to 3 heteroatoms as ring members selected from O and N;
L¹ and L³ are each independently selected from C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)-C₁₋₄ alkyl-, -(5-6 membered heteroarylene)-C₁₋₄ alkyl-, —O—, —S—, —N(R^{L})—, —C(O)—, —C(O)N(R^{L})—, —N(R^{L})C(O)—, —N(R^{L})C(O)N(R^{L})—, —N(R^{L})C(O)O—, —OC(O)N(R^{L})—, —S(O)₂—, —N(R^{L})S(O)₂—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)-C₁₋₄ alkyl, and (5-6 membered heteroarylene)-C₁₋₄ alkyl of L¹ and L³ are each optionally substituted with 1, 2, 3, or 4 independently selected R^{G} substituents;
L² and L⁴ are each independently selected from bond, C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)-C₁₋₄ alkyl-, -(5-6 membered heteroarylene)-C₁₋₄ alkyl-, —O—, —S—, —N(R^{L})—, —C(O)—, —C(O)N(R^{L})—, —N(R^{L})C(O)—, —N(R^{L})C(O)N(R^{L})—, —N(R^{L})C(O)O—, —OC(O)N(R^{L})—, —S(O)₂—, —N(R^{L})S(O)₂—, —S(O)₂N(R^{L})—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)-C₁₋₄ alkyl, and (5-6 membered heteroarylene)-C₁₋₄ alkyl of L² and L⁴ are each optionally substituted with 1, 2, 3, or 4 independently selected R^{G} substituents;
each R^{L} is independently selected from H, C₁₋₆ alkyl, and C₁₋₆ haloalkyl;
each R¹ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1}, —SR^{a1}, —NR^{c1}R^{d1}, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}C(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R¹ are each optionally substituted with 1, 2, 3, 4, 5, or 6 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1}, R^{b1}, R^{c1}, and R^{d1} are each optionally substituted with 1, 2, 3, 4, 5, or 6 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1} and R^{e1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{1A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a1A}, —SR^{a1A}, —NR^{c1A}R^{d1A}, —C(O)R^{a1A}, —C(O)OR^{a1A}, —C(O)NR^{c1A}R^{d1A}, —C(O)NR^{c1A}(OR^{a1A}), —OC(O)R^{a1A}, —OC(O)NR^{c1A}R^{d1A}, —OC(O)OR^{a1A}, —OS(O)₂R^{b1A}, —OS(O)₂NR^{c1A}R^{d1A}, —NR^{c1A}C(O)R^{a1A}, —NR^{c1A}C(O)OR^{a1A}, —NR^{c1A}C(O)NR^{c1A}R^{d1A}, —NR^{c1A}S(O)₂R^{b1A}, —NR^{c1A}S(O)₂NR^{c1A}R^{d1A}, —NR^{c1A}OR^{a1A}, —NR^{c1A}S(O)R^{b1A}, —NR^{c1A}S(O)NR^{c1A}R^{b1A}, —S(O)R^{b1A}, —S(O)₂R^{b1A}, —S(O)NR^{c1A}R^{d1A}, —S(O)₂NR^{c1A}R^{d1A}, —C(═NR^{e1A})R^{a1A}, —C(═NR^{e1A})NR^{c1A}R^{d1A}, —NR^{c1A}C(═NR^{e1A})R^{a1A}, —NR^{c1A}C(═NR^{e1A})NR^{c1A}R^{d1A}, —NR^{c1A}S(O)(═NR^{e1A})R^{b1A}, —NR^{c1A}S(O)(═NR^{e1A})NR^{c1A}R^{d1A}, —OS(O)(═NR^{e1A})R^{b1A}, —S(O)(═NR^{e1A})R^{b1A}, —S(O)(═NR^{e1A})NR^{c1A}R^{d1A}, —C(O)NR^{c1A}S(O)₂R^{b1A}, —C(O)NR^{c1A}S(O)₂NR^{c1A}R^{d1A}, —S(O)₂NR^{c1A}C(O)R^{b1A}, —NR^{c1A}S(O)NR^{g1A}C(O)R^{b1A}, and —P(O)R^{f1A}R^{g1A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{1A} are each optionally substituted with 1, 2, 3, 4, 5, or 6 independently selected R^{G} substituents;
each R^{a1A}, R^{b1A}, R^{c1A}, and R^{d1A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a1A}, R^{b1A}, R^{c1A}, and R^{d1A} are each optionally substituted with 1, 2, 3, 4, 5, or 6 independently selected R^{G} substituents;
or, any R^{c1A} and R^{d1A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e1A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f1A} and R^{g1A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R² is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R^{d2}, —C(O)NR^{c2}(OR^{a2}), —OC(O)R^{a2}, —OC(O)NR^{c2}R^{d2}, —OC(O)OR^{a2}, —OS(O)₂R^{b2}, —OS(O)₂NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —OS(O)(═NR^{e2})R^{b2}, —S(O)(═NR^{e2})R^{b2}, —S(O)(═NR^{e2})NR^{c2}R^{d2}, —C(O)NR^{c2}S(O)₂R^{b2}, —C(O)NR^{c2}S(O)₂NR^{c2}R^{d2}, —S(O)₂NR^{c2}C(O)R^{b2}, —NR^{c2}S(O)NR^{c2}C(O)R^{b2}, and —P(O)R^{f2}R^{g2}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R² are each optionally substituted with 1, 2, 3, 4, 5, or 6 independently selected R^{2A} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a2}, R^{b2}, R^{c2}, and R^{d2} are each optionally substituted with 1, 2, 3, 4, 5, or 6 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{2A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a2A}, —SR^{a2A}, —NR^{c2A}R^{d2A}, —C(O)R^{a2A}, —C(O)OR^{a2A}, —C(O)NR^{c2A}R^{d2A}, —C(O)NR^{c2A}(OR^{a2A}), —OC(O)R^{a2A}, —OC(O)NR^{a2A}R^{d2A}, —OC(O)OR^{a2A}, —OS(O)₂R^{b2A}, —OS(O)₂NR^{c2A}R^{d2A}, —NR^{c2A}C(O)R^{a2A}, —NR^{c2A}C(O)OR^{a2A}, —NR^{c2A}C(O)NR^{c2A}R^{d2A}, —NR^{b2A}S(O)₂R^{b2A}, —NR^{c2A}S(O)₂NR^{c2A}R^{d2A}, —NR^{c2A}OR^{a2A}, —NR^{c2A}S(O)R^{b2A}, —NR^{c2A}S(O)NR^{c2A}R^{d2A}, —S(O)R^{b2A}, —S(O)₂R^{b2A}, —S(O)NR^{c2A}R^{d2A}, —S(O)₂NR^{c2A}R^{d2A}, —C(═NR^{e2A})R^{a2A}, —C(═NR^{e2A})NR^{c2A}R^{d2A}, —NR^{c2A}C(═NR^{e2A})R^{a2A}, —NR^{c2A}C(═NR^{e2A})NR^{c2A}R^{d2A}, —NR^{c2A}S(O)(═NR^{e2A})R^{b2A}, —NR^{c2A}S(O)(═NR^{e2A})NR^{c2A}R^{d2A}, —OS(O)(═NR^{e2A})R^{b2A}, —S(O)(═NR^{e2A})R^{b2A}, —S(O)(═NR^{e2A})NR^{c2A}R^{d2A}, —C(O)NR^{c2A}S(O)₂R^{b2A}, —C(O)NR^{c2A}S(O)₂NR^{c2A}R^{d2A}, —S(O)₂NR^{c2A}C(O)R^{b2A}, —NR^{c2A}S(O)NR^{c2A}C(O)R^{b2A}, and —P(O)R^{f2A}R^{g2A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{2A} are each optionally substituted with 1, 2, 3, 4, 5, or 6 independently selected R^{G} substituents;
each R^{a2A}, R^{b2A}, R^{c2A}, and R^{d2A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a2A}, R^{b2A}, R^{c2A}, and R^{d2A} are each optionally substituted with 1, 2, 3, 4, 5, or 6 independently selected R^{G} substituents;
or, any R^{c2A} and R^{d2A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e2A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f2A} and R^{g2A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R³ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a3}, —C(O)OR^{a3}, —C(O)NR^{c3}R^{d3}, —S(O)₂R^{b3}, —S(O)₂NR^{c3}R^{d3}, —S(O)(═NR^{e3})R^{b3}, and —S(O)(═NR^{e3})NR^{c3}R^{d3}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R³ are each optionally substituted with 1, 2, 3, 4, 5, or 6 independently selected R^{G} substituents;
or, R³ and L³ together with the atoms to which they are attached form a 5-10 membered heterocycloalkyl group, wherein the 5-10 membered heterocycloalkyl group is optionally substituted with 1, 2, 3, 4, 5, or 6 independently selected R^{G} substituents;
each R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a3}, R^{b3}, R^{c3}, and R^{d3} are each optionally substituted with 1, 2, 3, 4, 5, or 6 independently selected R^{G} substituents;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁴ are each optionally substituted with 1, 2, 3, 4, 5, or 6 independently selected R^{G} substituents;
R⁵ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —CN, —OR^{a5}, —SR^{a5}, —NR^{c5}R^{d5}, —NO₂, —C(O)R^{a5}, —C(O)OR^{a5}, —C(O)NR^{c5}R^{d5}, —C(O)NR^{c5}(OR^{a5}), —OC(O)R^{a5}, —OC(O)NR^{c5}R^{d5}, —OC(O)OR^{a5}, —OS(O)₂R^{b5}, —OS(O)₂NR^{c5}R^{d5}, —NR^{c5}C(O)R^{a5}, —NR^{c5}C(O)OR^{a5}, —NR^{c5}C(O)NR^{c5}R^{d5}, —NR^{c5}S(O)₂R^{b5}, —NR^{c5}S(O)₂NR^{c5}R^{d5}, —NR^{c5}OR^{a5}, —NR^{c5}S(O)R^{b5}, —NR^{c5}S(O)NR^{c5}R^{d5}, —S(O)R^{b5}, —S(O)₂R^{b5}, —S(O)NR^{c5}R^{d5}, —S(O)₂NR^{c5}R^{d5}, —C(═NR^{e5})R^{a5}, —C(═NR^{e5})NR^{c5}R^{d5}, —NR^{c5}C(═NR^{e5})R^{a5}, —NR^{c5}C(═NR^{e5})NR^{c5}R^{d5}, —NR^{c5}S(O)(═NR^{e5})R^{b5}, —NR^{c5}S(O)(═NR^{e5})NR^{c5}R^{d5}, —OS(O)(═NR^{e5})R^{b5}, —S(O)(═NR^{e5})R^{b5}, —S(O)(═NR^{e5})NR^{c5}R^{d5}, —C(O)NR^{c5}S(O)₂R^{b5}, —C(O)NR^{c5}S(O)₂NR^{c5}R^{d5}, —S(O)₂NR^{c5}C(O)R^{b5}, —NR^{c5}S(O)NR^{c5}C(O)R^{b5}, and —P(O)R^{f5}R^{g5}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁵ are each optionally substituted with 1, 2, 3, 4, 5, or 6 independently selected R^{5A} substituents;
each R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R^{a5}, R^{b5}, R^{c5}, and R^{d5} are each optionally substituted with 1, 2, 3, 4, 5, or 6 independently selected R^{5A} substituents;
or, any R^{c5} and R^{d5} attached to the same N atom, together with the N atom to which they are attached, form a 4-14 membered heterocycloalkyl group, wherein the 4-14 membered heterocycloalkyl group is optionally substituted with 1, 2, 3, 4, 5, or 6 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{5A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a5A}, —SR^{a5A}, —NR^{c5A}R^{d5A}, —NO₂, —C(O)R^{a5A}, —C(O)OR^{a5A}, —C(O)NR^{c5A}R^{d5A}, —C(O)NR^{c5A}(OR^{a5A}), —OC(O)R^{a5A}, —OC(O)NR^{c5A}R^{d5A}, —OC(O)OR^{a5A}, —OS(O)₂R^{b5A}, —OS(O)₂NR^{c5A}R^{d5A}, —NR^{c5A}C(O)R^{a5A}, —NR^{c5A}C(O)OR^{a5A}, —NR^{c5A}C(O)NR^{c5A}R^{d5A}, —NR^{c5A}S(O)₂R^{b5A}, —NR^{c5A}S(O)₂NR^{c5A}R^{d5A}, NR^{c5A}OR^{a5A}, —NR^{c5A}S(O)R^{b5A}, —NR^{c5A}S(O)NR^{c5A}R^{d5A}, —S(O)R^{b5A}, —S(O)₂R^{b5A}, —S(O)NR^{c5A}R^{d5A}, —S(O)₂NR^{c5A}R^{d5A}, —C(═NR^{e5A})R^{a5A}, —C(═NR^{e5A})NR^{c5A}R^{d5A}, —NR^{c5A}C(═NR^{e5A})R^{a5A}, —NR^{c5A}C(═NR^{e5A})NR^{c5A}R^{d5A}, —NR^{c5A}S(O)(═NR^{e5A})R^{b5A}, —NR^{c5A}S(O)(═NR^{e5A})NR^{c5A}R^{d5A}, —OS(O)(═NR^{e5A})R^{b5A}, —S(O)(═NR^{e5A})R^{b5A}, —S(O)(═NR^{e5A})NR^{c5A}R^{d5A}, —C(O)NR^{c5A}S(O)₂R^{b5A}, —C(O)NR^{c5A}S(O)₂NR^{c5A}R^{d5A}, —S(O)₂NR^{c5A}C(O)R^{b5A}, —NR^{d5A}S(O)NR^{c5A}C(O)R^{b5A}, and —P(O)R^{f5A}R^{g5A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{5A} are each optionally substituted with 1, 2, 3, 4, 5, or 6 independently selected R^{G} substituents;
each R^{a5A}, R^{b5A}, R^{c5A}, and R^{d5A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a5A}, R^{b5A}, R^{c5A}, and R^{d5A} are each optionally substituted with 1, 2, 3, 4, 5, or 6 independently selected R^{G} substituents;
or, any R^{c5A} and R^{d5A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e5A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f5A} and R^{d5A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁶ is selected from H, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —C(O)R^{a6}, —C(O)OR^{a6}, —C(O)NR^{c6}R^{d6}, —C(O)NR^{c6}(OR^{a6}), —S(O)R^{b6}, —S(O)₂R^{b6}, —S(O)NR^{c6}R^{d6}, —S(O)₂NR^{c6}R^{d6}, —C(═NR^{e6})R^{a6}, —C(═NR^{e6})NR^{c6}R^{d6}, —S(O)(═NR^{e6})R^{b6}, —S(O)(═NR^{e6})NR^{c6}R^{d6}, —C(O)NR^{c6}S(O)₂R^{b6}, —C(O)NR^{c6}S(O)₂NR^{c6}R^{d6}, and —S(O)₂NR^{c6}C(O)R^{b6}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁶ are each optionally substituted with 1, 2, 3, 4, 5, or 6 independently selected R^{6A} substituents;
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a6}, R^{b6}, R^{c6}, and R^{d6} are each optionally substituted with 1, 2, 3, 4, 5, or 6 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{6A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)N^{c6A}R^{d6A}, —C(O)N^{c6A}(OR^{a6A}), —OC(O)R^{a6A}, —OC(O)N^{c6A}R^{d6A}, —OC(O)OR^{a6A}, —OS(O)₂R^{b6A}, —OS(O)₂N^{c6A}R^{d6A}, —NR^{c6A}C(O)R^{a6A}, —NR^{c6A}C(O)OR^{a6A}, —NR^{c6A}C(O)NR^{c6A}R^{d6A}, —NR^{c6A}S(O)₂R^{b6A}, —NR^{c6A}S(O)₂N^{c6A}R^{d6A}, —NR^{c6A}OR^{a6A}, —NR^{c6A}S(O)R^{b6A}, —NR^{c6A}S(O)NR^{c6A}R^{d6A}, —S(O)R^{b6A}, —S(O)₂R^{b6A}, —S(O)N^{c6A}R^{d6A}, —S(O)₂N^{c6A}R^{d6A}, —C(═NR^{e6A})R^{a6A}, —C(═NR^{e6A})N^{c6A}R^{d6A}, —NR^{c6A}C(═NR^{e6A})R^{a6A}, —NR^{c6A}C(═NR^{e6A})NR^{c6A}R^{d6A}, —NR^{c6A}S(O)(═NR^{e6A})R^{b6A}, —NR^{c6A}S(O)(═NR^{e6A})N^{c6A}R^{d6A}, —OS(O)(═NR^{e6A})R^{b6A}, —S(O)(═NR^{e6A})R^{b6A}, —S(O)(═NR^{e6A})NR^{c6A}R^{d6A}, —C(O)NR^{c6A}S(O)₂R^{b6A}, —C(O)NR^{c6A}S(O)₂N^{c6A}R^{d6A}, —S(O)₂NR^{c6A}C(O)R^{b6A}, —NR^{c6A}S(O)NR^{c6A}C(O)R^{b6A}, and —P(O)R^{f6A}R^{g6A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{6A} are each optionally substituted with 1, 2, 3, 4, 5, or 6 independently selected R^{G} substituents;
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a6A}, R^{b6A}, R^{c6A}, and R^{d6A} are each optionally substituted with 1, 2, 3, 4, 5, or 6 independently selected R^{G} substituents;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e6A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f6A} and R^{g6A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}, —NR^{c7}C(O)R^{a7}, —NR^{c7}C(O)OR^{a7}, —NR^{c7}C(O)NR^{c7}R^{d7}, —NR^{c7}S(O)₂R^{b7}, —NR^{c7}S(O)₂NR^{c7}R^{d7}, —NR^{c7}OR^{a7}, —NR^{c7}S(O)R^{b7}, —NR^{c7}S(O)NR^{c7}R^{d7}, —S(O)R^{b7}, —S(O)₂R^{b7}, —S(O)NR^{c7}R^{d7}, —S(O)₂NR^{c7}R^{d7}, —C(═NR^{e7})R^{a7}, —C(═NR^{e7})NR^{c7}R^{d7}, —NR^{c7}C(═NR^{e7})R^{a7}, —NR^{c7}C(═NR^{e7})NR^{c7}R^{d7}, —NR^{c7}S(O)(═NR^{e7})R^{b7}, —NR^{c7}S(O)(═NR^{e7})NR^{c7}R^{d7}, —OS(O)(═NR^{e7})R^{b7}, —S(O)(═NR^{e7})R^{b7}, —S(O)(═NR^{e7})NR^{c7}R^{d7}, —C(O)NR^{c7}S(O)₂R^{b7}, —C(O)NR^{c7}S(O)₂NR^{c7}R^{d7}, —S(O)₂NR^{c7}C(O)R^{b7}, —NR^{c7}S(O)NR^{c7}C(O)R^{b7}, and —P(O)R^{f7}R^{g7}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁷ are each optionally substituted with 1, 2, 3, 4, 5, or 6 independently selected R^{7A} substituents;
or, R⁵ and R⁷ together with the atoms to which they are attached form a C₅₋₁₄ cycloalkyl or 5-14 membered heterocycloalkyl group, wherein the C₅₋₁₄ cycloalkyl and 5-14 membered heterocycloalkyl group is optionally substituted with 1, 2, 3, 4, 5, or 6 independently selected R^{7A} substituents;
or, R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents;
each R^{a7}, R^{b7}, R^{c7}, and R^{d7} is independently selected from H, C₁₋₆ alkyl, C₁₋₆ haloalkyl, C₂₋₆ alkenyl, C₂₋₆ alkynyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl, wherein the C₁₋₆ alkyl, C₂₋₆ alkenyl, C₂₋₆ alkynyl, C₁₋₆ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R^{a7}, R^{b7}, R^{c7}, and R^{d7} are each optionally substituted with 1, 2, 3, 4, 5, or 6 independently selected R^{7A} substituents;
or, any R^{c7} and R^{d7} attached to the same N atom, together with the N atom to which they are attached, form a 4-14 membered heterocycloalkyl group, wherein the 4-14 membered heterocycloalkyl group is optionally substituted with 1, 2, 3, 4, 5, or 6 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7A}, —SR^{a7A}, —NR^{c7A}R^{d7A}, —NO₂, —C(O)R^{a7A}, —C(O)OR^{a7A}, —C(O)NR^{c7A}R^{d7A}, —C(O)NR^{c7A}(OR^{a7A}), —OC(O)R^{a7A}, —OC(O)NR^{c7A}R^{d7A}, —OC(O)OR^{a7A}, —OS(O)₂R^{b7A}, —OS(O)₂NR^{c7A}R^{d7A}, —NR^{c7A}C(O)R^{a7A}, —NR^{c7A}C(O)OR^{a7A}, —NR^{c7A}C(O)NR^{c7A}R^{d7A}, —NR^{c7A}S(O)₂R^{b7A}, —NR^{c7A}S(O)₂NR^{c7A}R^{d7A}, —NR^{C7A}OR^{a7A}, —NR^{c7A}S(O)R^{b7A}, —NR^{c7A}S(O)NR^{c7A}R^{d7A}, —S(O)R^{b7A}, —S(O)₂R^{b7A}, —S(O)NR^{c7A}R^{d7A}, —S(O)₂NR^{c7A}R^{d7A}, —C(═NR^{e7A})R^{a7A}, —C(═NR^{e7A})NR^{c7A}R^{d7A}, —NR^{c7A}C(═NR^{e7A})R^{a7A}, —NR^{c7A}C(═NR^{e7A})NR^{c7A}R^{d7A}, —NR^{c7A}S(O)(═NR^{e7A})R^{b7A}, —NR^{c7A}S(O)(═NR^{e7A})NR^{c7A}R^{d7A}, —OS(O)(═NR^{e7A})R^{b7A}, —S(O)(═NR^{e7A})R^{b7A}, —S(O)(═NR^{e7A})NR^{c7A}R^{d7A}, —C(O)NR^{c7A}S(O)₂R^{b7A}, —C(O)NR^{c7A}S(O)₂NR^{c7A}R^{d7A}, —S(O)₂NR^{c7A}C(O)R^{b7A}, —NR^{c7A}S(O)NR^{c7A}C(O)R^{b7A}, and —P(O)R^{f7A}R^{g7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{7B} substituents;
each R^{a7A}, R^{b7A}, R^{c7A}, and R^{d7A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7A}, R^{b7A}, R^{e7A}, and R^{d7A} are each optionally substituted with 1, 2, 3, 4, 5, or 6 independently selected R^{7B} substituents;
or, any R^{c7A} and R^{d7A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{7B} substituents;
each R^{e7A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7A} and R^{d7A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7B} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7B}, —SR^{a7B}, —NR^{c7B}R^{d7B}, —NO₂, —C(O)R^{a7B}, —C(O)OR^{a7B}, —C(O)NR^{c7B}R^{d7B}, —C(O)NR^{c7B} (OR^{a7B}), —OC(O)R^{a7B}, —OC(O)NR^{c7B}R^{d7B}, —OC(O)OR^{a7B}, —OS(O)₂R^{b7B}, —OS(O)₂NR^{c7B}R^{d7B}, —NR^{c7B}C(O)R^{a7B}, —NR^{c7B}C(O)OR^{a7B}, —NR^{c7B}C(O)NR^{c7B}R^{d7B}, —NR^{c7B}S(O)₂R^{b7B}, —NR^{c7B}S(O)₂NR^{c7B}R^{d7B}, —NR^{c7B}OR^{a7B}, —NR^{c7B}S(O)R^{b7B}, —NR^{c7B}S(O)NR^{c7B}R^{d7B}, —S(O)R^{b7B}, —S(O)₂R^{b7B}, —S(O)NR^{c7B}R^{d7B}, —S(O)₂NR^{c7B}R^{d7B}, —C(═NR^{e7B})R^{a7B}, —C(═NR^{e7B})NR^{c7B}R^{d7B}, —NR^{c7B}C(═NR^{e7B})R^{a7B}, —NR^{c7B}C(═NR^{e7B})NR^{c7B}R^{d7B}, —NR^{c7B}S(O)(═NR^{e7B})R^{b7B}, —NR^{c7B}S(O)(═NR^{e7B})NR^{c7B}R^{d7B}, —OS(O)(═NR^{e7B})R^{b7B}, —S(O)(═NR^{e7B})R^{b7B}, —S(O)(═NR^{e7B})NR^{c7B}R^{d7B}, —C(O)NR^{c7B}S(O)₂R^{b7B}, —C(O)NR^{c7B}S(O)₂NR^{c7B}R^{d7B}, —S(O)₂NR^{c7B}C(O)R^{b7B}, —NR^{c7B}S(O)NR^{c7B}C(O)R^{b7B}, and —P(O)R^{f7B}R^{d7B}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7B} are each optionally substituted with 1, 2, 3, 4, 5, or 6 independently selected R^{G} substituents;
each R^{a7B}, R^{b7B}, R^{c7B}, and R^{d7B} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7B}, R^{b7B}, R^{c7B}, and R^{d7B} are each optionally substituted with 1, 2, 3, 4, 5, or 6 independently selected R^{G} substituents;
or, any R^{c7B} and R^{d7B} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e7B} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7B} and R^{g7B} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
R⁸ is selected from H, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}, —C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}, —NR^{c8}C(O)R^{a8}, —NR^{c8}C(O)OR^{a8}, —NR^{c8}C(O)NR^{c8}R^{d8}, —NR^{c8}S(O)₂R^{b8}, —NR^{c8}S(O)₂NR^{c8}R^{d8}, —NR^{c8}OR^{a8}, —NR^{c8}S(O)R^{b8}, —NR^{c8}S(O)NR^{c8}R^{d8}, —S(O)R^{b8}, —S(O)₂R^{b8}, —S(O)NR^{c8}R^{d8}, —S(O)₂NR^{c8}R^{d8}, —C(═NR^{e8})R^{a8}, —C(═NR^{e8})NR^{c8}R^{d8}, —NR^{c8}C(═NR^{e8})R^{a8}, —NR^{c8}C(═NR^{e8})NR^{c8}R^{d8}, —NR^{c8}S(O)(═NR^{e8})R^{b8}, —NR^{c8}S(O)(═NR^{e8})NR^{c8}R^{d8}, —OS(O)(═NR^{e8})R^{b8}, —S(O)(═NR^{e8})R^{b8}, —S(O)(═NR^{e8})NR^{c8}R^{d8}, —C(O)NR^{c8}S(O)₂R^{b8}, —C(O)NR^{c8}S(O)₂NR^{c8}R^{d8}, —S(O)₂NR^{c8}C(O)R^{b8}, —NR^{c8}S(O)NR^{c8}C(O)R^{b8}, and —P(O)R^{f8}R^{g8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁸ are each optionally substituted with 1, 2, 3, 4, 5, or 6 independently selected R^{8A} substituents;
each R^{a8}, R^{b8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{b8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{c8A}R^{d8A}, —NO₂, —C(O)R^{a8A}, —C(O)OR^{a8A}, —C(O)NR^{c8A}R^{d8A}, —C(O)NR^{c8A}(OR^{a8A}), —OC(O)R^{a8A}, —OC(O)NR^{c8A}R^{d8A}, —OC(O)OR^{a8A}, —OS(O)₂R^{b8A}, —OS(O)₂NR^{c8A}R^{d8A}, —NR^{c8A}C(O)R^{a8A}, —NR^{c8A}C(O)OR^{a8A}, —NR^{c8A}C(O)NR^{c8A}R^{d8A}, —NR^{c8A}S(O)₂R^{b8A}, —NR^{c8A}S(O)₂NR^{c8A}R^{d8A}, —NR^{c8A}OR^{d8A}, —NR^{c8A}S(O)R^{b8A}, —NR^{c8A}S(O)NR^{c8A}R^{d8A}, —S(O)R^{b8A}, —S(O)₂R^{b8A}, —S(O)NR^{c8A}R^{d8A}, —S(O)₂NR^{c8A}R^{d8A}, —C(═NR^{e8A})R^{a8A}, —C(═NR^{e8A})NR^{c8A}R^{d8A}, —NR^{c8A}C(═NR^{e8A})R^{a8A}, —NR^{c8A}C(═NR^{e8A})NR^{c8A}R^{d8A}, —NR^{c8A}S(O)(═NR^{e8A})R^{b8A}, —NR^{c8A}S(O)(═NR^{e8A})NR^{c8A}R^{d8A}, —OS(O)(═NR^{e8A})R^{b8A}, —S(O)(═NR^{e8A})R^{b8A}, —S(O)(═NR^{e8A})NR^{c8A}R^{d8A}, —C(O)NR^{c8A}S(O)₂R^{b8A}, —C(O)NR^{c8A}S(O)₂NR^{c8A}R^{d8A}, —S(O)₂NR^{c8A}C(O)R^{b8A}, —NR^{c8A}S(O)NR^{c8A}C(O)R^{b8A}, and —P(O)R^{f8A}R^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{b8A}, R^{c8A}, and R^{d8A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8A}, R^{b8A}, R^{c8A}, and R^{d8A} are each optionally substituted with 1, 2, 3, 4, 5, or 6 independently selected R^{G} substituents;
or, any R^{c8A} and R^{d8A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{e8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl) carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein X¹ is N or NR⁶.

3. The compound of claim 1, or a pharmaceutically acceptable salt thereof, wherein R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —C(O)R^{a6}, and —S(O)₂R^{b6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents; and
each R^{a6} and R^{b6} is independently selected from H and C₁₋₆ alkyl.

4. The compound of claim 1, or a pharmaceutically acceptable salt thereof, wherein R⁶ is selected from H, C₁₋₆ alkyl, C₂₋₆ alkenyl, C₂₋₆ alkynyl, C₁₋₆ haloalkyl, —C(O)R^{a6}, and —S(O)₂R^{b6}, wherein the C₁₋₆ alkyl, C₂₋₆ alkenyl, C₂₋₆ alkynyl, and C₁₋₆ haloalkyl of R⁶ are each optionally substituted with 1, 2, 3, or 4 independently selected R^{6A} substituents; and
each R^{a6} and R^{b6} is independently selected from H and C₁₋₆ alkyl.

5. The compound of claim 1, or a pharmaceutically acceptable salt thereof, wherein R⁶ is selected from H, C₁₋₆ alkyl, —C(O)R^{a6}, and —S(O)₂R^{b6}, wherein the C₁₋₆ alkyl of R⁶ is optionally substituted with 1, 2, 3, or 4 independently selected R^{6A} substituents; and
each R^{a6} and R^{b6} is independently selected from H and C₁₋₃ alkyl.

6. The compound of claim 1, or a pharmaceutically acceptable salt thereof, wherein R⁶ is selected from H, methyl, ethyl, methycarbonyl, and methylsulfonyl, wherein the methyl and ethyl of R⁶ are optionally substituted with 1 or 2 independently selected R^{6A} substituents.

7. The compound of claim 1, or a pharmaceutically acceptable salt thereof, wherein each R^{6A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —NR^{c6A}C(O)N^{c6A}R^{d6A}, —NR^{c6A}S(O)₂R^{b6A}, —NR^{c6A}S(O)R^{b6A}, —NR^{c6A}S(O)N^{c6A}R^{d6A}, —S(O)R^{b6A}, —S(O)₂R^{b6A}, —S(O)N^{c6A}R^{d6A}, and —S(O)₂NR^{c6A}R^{d6A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{6A} are each optionally substituted with 1, 2, 3, or 4 independently selected R^{G} substituents; and
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

8. The compound of claim 1, or a pharmaceutically acceptable salt thereof, wherein each R^{6A} is independently selected from oxo, halo, C₁₋₆ alkyl, C₁₋₆ haloalkyl, 4-7 membered heterocycloalkyl, —CN, —OR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)NR^{c6A}R^{d6A}, —OC(O)OR^{a6A}, —NR^{c6A}C(O)R^{a6A}, —NR^{c6A}C(O)OR^{a6A}, —S(O)R^{b6A}, —S(O)₂R^{b6A}, —S(O)NR^{c6A}R^{d6A}, and —S(O)₂NR^{c6A}R^{d6A}, wherein the C₁₋₆ alkyl, C₁₋₆ haloalkyl, and 4-7 membered heterocycloalkyl of R^{6A} are each optionally substituted with 1, 2, 3, or 4 independently selected R^{G} substituents; and
each R^{a6A}, R^{b6A}, R^{c6A}, and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

9. The compound of claim 1, or a pharmaceutically acceptable salt thereof, wherein each R^{6A} is independently selected from CN, 4-7 membered heterocycloalkyl, and —C(O)NR^{c6A}R^{d6A}, and
each R^{c6A} and R^{d6A} is independently selected from H, C₁₋₆ alkyl, C₁₋₆ haloalkyl, C₂₋₆ alkenyl, and C₂₋₆ alkynyl;
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents.

10. The compound of claim 1, or a pharmaceutically acceptable salt thereof, wherein each R^{6A} is independently selected from CN, azetidinyl, aminocarbonyl, methylaminocarbonyl, and morpholinylcarbonyl.

11. The compound of claim 1, or a pharmaceutically acceptable salt thereof, wherein X² is CR⁷.

12. The compound of claim 1, or a pharmaceutically acceptable salt thereof, wherein R⁷ is selected from H, halo, C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, (5-14 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₁₄ cycloalkyl, C₆₋₁₀ aryl, 4-14 membered heterocycloalkyl, 5-14 membered heteroaryl, C₃₋₁₄ cycloalkyl-C₁₋₄ alkyl, C₆₋₁₀ aryl-C₁₋₄ alkyl, (4-14 membered heterocycloalkyl)-C₁₋₄ alkyl, and (5-14 membered heteroaryl)-C₁₋₄ alkyl of R⁷ are each optionally substituted with 1, 2, 3, or 4 independently selected R^{7A} substituents; and
each R^{c7} and R^{d7} is independently selected from H, C₁₋₆ alkyl, C₂₋₆ alkenyl, C₂₋₆ alkynyl, and C₁₋₆ haloalkyl.

13. The compound of claim 1, or a pharmaceutically acceptable salt thereof, wherein R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, and —C(O)NR^{c7}R^{d7}, wherein the C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁷ are each optionally substituted with 1, 2, 3, or 4 independently selected R^{7A} substituents; and
each R^{c7} and R^{d7} is independently selected from H, C₁₋₆ alkyl, C₂₋₆ alkenyl, C₂₋₆ alkynyl, and C₁₋₆ haloalkyl.

14. The compound of claim 1, or a pharmaceutically acceptable salt thereof, wherein R⁷ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, 5-6 membered heteroaryl, and —C(O)NR^{c7}R^{d7}, wherein the C₁₋₆ alkyl, C₂₋₆ alkenyl, C₂₋₆ alkynyl, C₁₋₆ haloalkyl, C₃₋₇ cycloalkyl, phenyl, 4-10 membered heterocycloalkyl, and 5-6 membered heteroaryl of R⁷ are each optionally substituted with 1, 2, 3, or 4 independently selected R^{7A} substituents; and
each R^{e7} and R^{d7} is independently selected from H, C₁₋₆ alkyl, C₂₋₆ alkenyl, C₂₋₆ alkynyl, and C₁₋₆ haloalkyl.

15. The compound of claim 1, or a pharmaceutically acceptable salt thereof, wherein R⁷ is selected from H, halo, C₁₋₆ alkyl, C₂₋₆ alkenyl, C₂₋₆ alkynyl, phenyl, 8-10 membered heterocycloalkyl, 5-6 membered heteroaryl, and —C(O)NR^{c7}R^{d7}, wherein the C₁₋₆ alkyl, C₂₋₆ alkenyl, C₂₋₆ alkynyl, phenyl, 8-10 membered heterocycloalkyl, and 5-6 membered heteroaryl of R⁷ are each optionally substituted with 1, 2, 3, or 4 independently selected R^{7A} substituents; and
each R^{c7} and R^{d7} is independently selected from H and C₁₋₆ alkyl.

16. The compound of claim 1, or a pharmaceutically acceptable salt thereof, wherein each R⁷ is H, chloro, bromo, methyl, ethyl, ethenyl, ethynyl, propynyl, dimethylpropynyl, 5,6,7,8-tetrahydro-[1,2,4]triazolo[1,5-a]pyrazinyl, phenyl, pyrazolyl, pyridinyl, triazolyl, pyrimidinyl, and ethylaminocarbonyl, wherein the methyl, ethyl, ethenyl, ethynyl, propynyl, dimethylpropynyl, 5,6,7,8-tetrahydro-[1,2,4]triazolo[1,5-a]pyrazinyl, phenyl, pyrazolyl, pyridinyl, triazolyl, and pyrimidinyl of R⁷ are each optionally substituted with 1, 2, 3, or 4 independently selected R^{7A} substituents.

17. The compound of claim 1, or a pharmaceutically acceptable salt thereof, wherein each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, CN, OR^{a7A}, —NR^{c7A}R^{d7A}, and C(O)NR^{c7A}R^{d7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, or 4 independently selected R^{7B} substituents; and
each R^{a7A}, R^{c7A}, and R^{d7A} is independently selected from H, C₁₋₆ alkyl, and C₁₋₆ haloalkyl.

18. The compound of claim 1, or a pharmaceutically acceptable salt thereof, wherein each R^{7A} is independently selected from halo, C₁₋₆ alkyl, C₂₋₆ alkenyl, C₂₋₆ alkynyl, C₁₋₆ haloalkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents; and
each R^{a7A}, R^{c7A}, and R^{d7A} is independently selected from H and C₁₋₆ alkyl.

19. The compound of claim 1, or a pharmaceutically acceptable salt thereof, wherein each R^{7A} is independently selected from C₁₋₆ alkyl, C₃₋₇ cycloalkyl, 4-7 membered heterocycloalkyl, 5-6 membered heteroaryl, CN, OR^{a7A}, —NR^{c7A}R^{d7A}, and C(O)NR^{c7A}R^{d7A}, wherein the C₁₋₆ alkyl, C₃₋₇ cycloalkyl, 4-7 membered heterocycloalkyl, and 5-6 membered heteroaryl of R^{7A} are each optionally substituted with 1, 2, 3, or 4 independently selected R^{7B} substituents; and
each R^{a7A}, R^{c7A}, and R^{d7A} is independently selected from H and C₁₋₃ alkyl.

20. The compound of claim 1, or a pharmaceutically acceptable salt thereof, wherein each R^{7A} is independently selected from methyl, isopropyl, cyclopropyl, cyclobutyl, oxetanyl, azetidinyl, tetrahydrofuranyl, tetrahydropyranyl, piperidinyl, imidazolyl, pyrazolyl, pyridinyl, cyano, hydroxy, amino, and aminocarbonyl, wherein the methyl, isopropyl, cyclopropyl, oxetanyl, azetidinyl, tetrahydrofuranyl, tetrahydropyranyl, piperidinyl, imidazolyl, pyrazolyl, and pyridinyl of R^{7A} are each optionally substituted with 1, 2, 3, or 4 independently selected R^{7B} substituents.

21. The compound of claim 1, or a pharmaceutically acceptable salt thereof, wherein each R^{7B} is independently selected from C₁₋₆ alkyl, OR^{a7B}, NR^{c7B}R^{d7B}, C(O)R^{a7B}, and —C(O)NR^{c7B}R^{d7B}, wherein the C₁₋₆ alkyl is optionally substituted by OH; and
each R^{a7B}, R^{c7B}, and R^{d7B} is independently selected from H and C₁₋₃ alkyl.

22. The compound of claim 1, or a pharmaceutically acceptable salt thereof, wherein each R^{7B} is independently selected from methyl, hydroxymethyl, hydroxy, amino, methylcarbonyl, and methylaminocarbonyl.

23. The compound of claim 1, or a pharmaceutically acceptable salt thereof, wherein R⁷ is selected from H, chloro, bromo, methyl, hydroxymethyl, cyanomethyl, hydroxyethyl, cyanoethyl, pyridinylethyl, cyanoethenyl, pyridinylethenyl, ethynyl, cyclopropylethynyl, (hydroxycyclopropyl)ethynyl, (hydroxymethylcyclopropyl)ethynyl, (aminocyclopropyl)ethynyl, (hydroxy)(dimethyl)propynyl, (amino)(dimethyl)propynyl, (methyloxetanyl)ethynyl, tetrahydropyranylethynyl, (methylcarbonylpiperidinyl)ethynyl, (methylaminocarbonylpiperidinyl)ethynyl, pyridinylethynyl, (methylpyrazolyl)ethynyl, (methylimidazolyl)ethynyl, 5,6,7,8-tetrahydro-[1,2,4]triazolo[1,5-a]pyrazinyl, pyrimidinyl, isopropylpyrazolyl, tetrahydrofuranylpyrazolyl, methyltriazolyl, phenyl, aminocarbonylphenyl, ethylaminocarbonyl, methylpyrazolyl, pyridinyl, and morpholinylmethyl.

24. The compound of claim 1, or a pharmaceutically acceptable salt thereof, wherein R⁷ is selected from H, chloro, bromo, methyl,

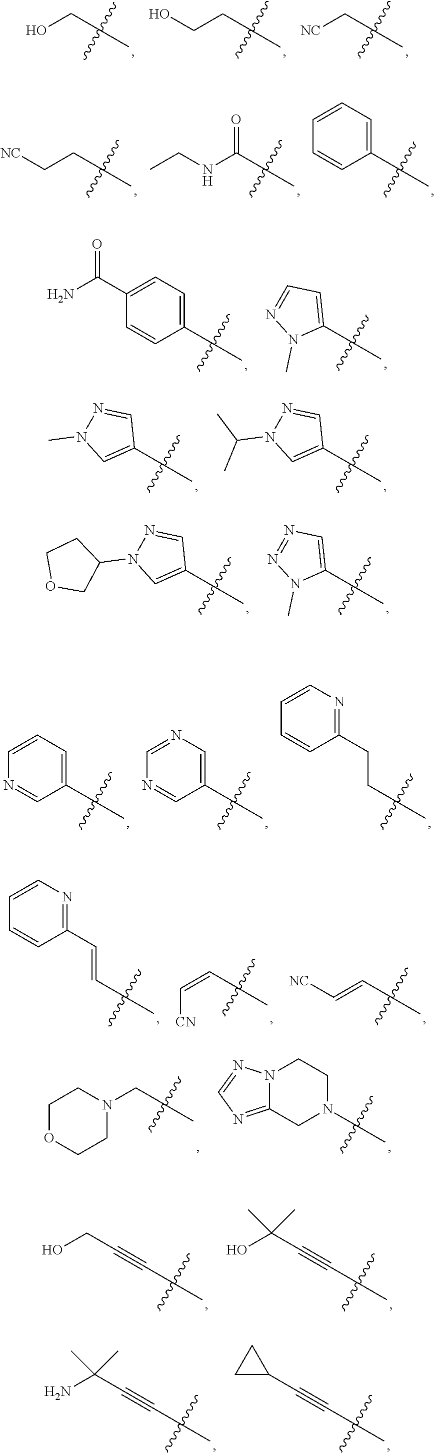

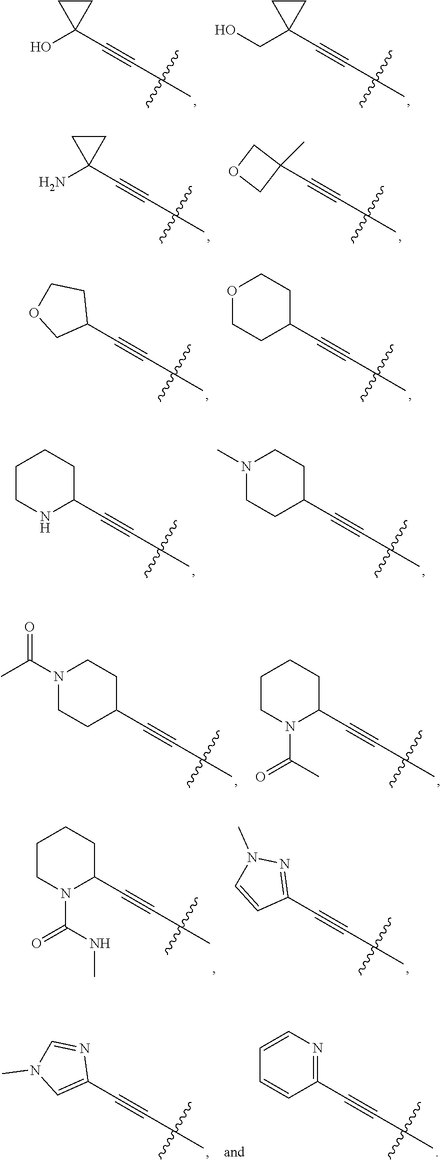

25. The compound of claim 1, or a pharmaceutically acceptable salt thereof, wherein R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents.

26. The compound of claim 1, or a pharmaceutically acceptable salt thereof, wherein R⁶ and R⁷ together with the atoms to which they are attached form a 5-14 membered heterocycloalkyl group selected from

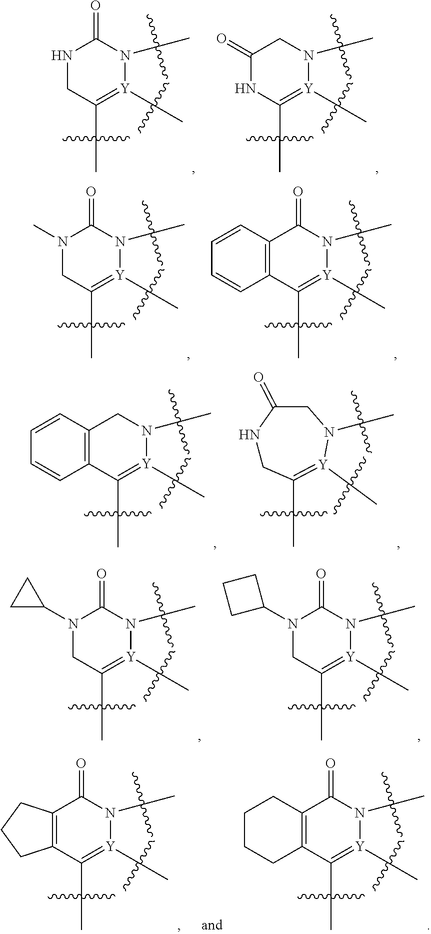

27. The compound of claim 1, or a pharmaceutically acceptable salt thereof, wherein X³ is CR⁸.

28. The compound of claim 1, or a pharmaceutically acceptable salt thereof, wherein R⁸ is selected from H, halo, C₁₋₆ alkyl, C₂₋₆ alkenyl, C₂₋₆ alkynyl, and C₁₋₆ haloalkyl.

29. The compound of claim 1, or a pharmaceutically acceptable salt thereof, X³ is CH.

30. The compound of claim 1, or a pharmaceutically acceptable salt thereof, wherein Y is C.

31. The compound of claim 1, or a pharmaceutically acceptable salt thereof, wherein Z is C.

32. The compound of claim 1, or a pharmaceutically acceptable salt thereof, wherein Z is N.

33. The compound of claim 1, or a pharmaceutically acceptable salt thereof, wherein R³ is selected from H, C₁₋₆ alkyl, C₁₋₆ haloalkyl, and —C(O)NR^{c3}R^{d3}; and
each R^{c3} and R^{d3} is independently selected from H and C₁₋₆ alkyl.

34. The compound of claim 1, or a pharmaceutically acceptable salt thereof, wherein R³ is selected from H, C₁₋₆ alkyl, and —C(O)NR^{c3}R^{d3}; and
each R^{c3} and R^{d3} is independently selected from H and C₁₋₃ alkyl.

35. The compound of claim 1, or a pharmaceutically acceptable salt thereof, wherein R³ is selected from H, methyl, and aminomethylcarbonyl.

36. The compound of claim 1, or a pharmaceutically acceptable salt thereof, wherein R⁴ is selected from H, C₁₋₆ alkyl, and C₁₋₆ haloalkyl.

37. The compound of claim 1, or a pharmaceutically acceptable salt thereof, wherein R⁴ is H or methyl.

38. The compound of claim 1, or a pharmaceutically acceptable salt thereof, wherein L¹ is —N(R^{L})C(O)— or —N(R^{L})—.

39. The compound of claim 1, or a pharmaceutically acceptable salt thereof, wherein L¹ is —NHC(O)—.

40. The compound of claim 1, or a pharmaceutically acceptable salt thereof, wherein L² is a bond.

41. The compound of claim 1, or a pharmaceutically acceptable salt thereof, wherein L³ is selected from C₁₋₆ alkylene, —O—, and —N(R^{L})—.

42. The compound of claim 1, or a pharmaceutically acceptable salt thereof, wherein L³ is selected from methylene, —O—, and —NH—.

43. The compound of claim 1, or a pharmaceutically acceptable salt thereof, wherein L⁴ is a bond or —O—.

44. The compound of claim 1, or a pharmaceutically acceptable salt thereof, wherein Ring A is C₃₋₁₀ cycloalkyl, C₆₋₁₀ aryl, 4-10 membered heterocycloalkyl, or 5-10 membered heteroaryl.

45. The compound of claim 1, or a pharmaceutically acceptable salt thereof, wherein Ring A is phenyl, 8-10 membered heterocycloalkyl, or 8-10 membered heteroaryl.

46. The compound of claim 1, or a pharmaceutically acceptable salt thereof, wherein Ring A is phenyl, benzo[b]thiophenyl, or indolinyl.

47. The compound of claim 1, or a pharmaceutically acceptable salt thereof, wherein n is 0, 1, 2, 3, or 4.

48. The compound of claim 1, or a pharmaceutically acceptable salt thereof, wherein each R¹ is independently selected from oxo, halo, C₁₋₆ alkyl, C₂₋₆ alkenyl, C₂₋₆ alkynyl, C₁₋₆ haloalkyl, —CN, and —OR^{a1}; and
each R^{a1} is independently selected from H, C₁₋₆ alkyl, C₂₋₆ alkenyl, C₂₋₆ alkynyl, and C₁₋₆ haloalkyl.

49. The compound of claim 1, or a pharmaceutically acceptable salt thereof, wherein each R¹ is independently selected from halo, C₁₋₆ alkyl, C₁₋₆ haloalkyl, and OR^{a1}; and
each R^{a1} is independently selected from H, C₁₋₆ alkyl, and C₁₋₆ haloalkyl.

50. The compound of claim 1, or a pharmaceutically acceptable salt thereof, wherein each R¹ is independently selected from fluoro, chloro, trifluoromethyl, and hydroxy.

51. The compound of claim 1, or a pharmaceutically acceptable salt thereof, wherein Ring B is C₃₋₇ cycloalkyl, phenyl, 4-7 membered heterocycloalkyl, or 5-6 membered heteroaryl.

52. The compound of claim 1, or a pharmaceutically acceptable salt thereof, wherein Ring B is phenyl or 5-6 membered heteroaryl.

53. The compound of claim 1, or a pharmaceutically acceptable salt thereof, wherein Ring B is phenyl or pyridinyl.

54. The compound of claim 1, or a pharmaceutically acceptable salt thereof, wherein m is 0, 1, 2, or 3.

55. The compound of claim 1, or a pharmaceutically acceptable salt thereof, wherein each R² is independently selected from halo, C₁₋₆ alkyl, C₂₋₆ alkenyl, C₂₋₆ alkynyl, and C₁₋₆ haloalkyl.

56. The compound of claim 1, or a pharmaceutically acceptable salt thereof, wherein each R² is independently selected from halo.

57. The compound of claim 1, or a pharmaceutically acceptable salt thereof, wherein each R² is independently chloro or fluoro.

58. The compound of claim 1, or a pharmaceutically acceptable salt thereof, wherein:
X¹ is CR⁵, N, or NR⁶;
X² is CR⁷ or N;
X³ is CR⁸ or N;
Y is C or N;
Z is C or N;
n is 0, 1, 2, 3, or 4;
m is 0, 1, 2, 3, or 4;
L¹ is —NHC(O)— or —NH—;
L² is a bond;
L³ is selected from C₁₋₆ alkylene, —O—, and —NH—;
L⁴ is a bond or —O—;
each R¹ is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a1}, —SR^{a1}, —NR^{c1}R^{d1}, —C(O)R^{a1}, —C(O)OR^{a1}, —C(O)NR^{c1}R^{d1}, —C(O)NR^{c1}(OR^{a1}), —OC(O)R^{a1}, —OC(O)NR^{c1}R^{d1}, —OC(O)OR^{a1}, —OS(O)₂R^{b1}, —OS(O)₂NR^{c1}R^{d1}, —NR^{c1}C(O)R^{a1}, —NR^{c1}C(O)OR^{a1}, —NR^{c1}C(O)NR^{c1}R^{d1}, —NR^{c1}S(O)₂R^{b1}, —NR^{c1}S(O)₂NR^{c1}R^{d1}, —NR^{c1}OR^{a1}, —NR^{c1}S(O)R^{b1}, —NR^{c1}S(O)NR^{c1}R^{d1}, —S(O)R^{b1}, —S(O)₂R^{b1}, —S(O)NR^{c1}R^{d1}, —S(O)₂NR^{c1}R^{d1}, —C(═NR^{e1})R^{a1}, —C(═NR^{e1})NR^{c1}R^{d1}, —NR^{c1}C(═NR^{e1})R^{a1}, —NR^{c1}C(═NR^{e1})NR^{c1}R^{d1}, —NR^{c1}S(O)(═NR^{e1})R^{b1}, —NR^{c1}S(O)(═NR^{e1})NR^{c1}R^{d1}, —OS(O)(═NR^{e1})R^{b1}, —S(O)(═NR^{e1})R^{b1}, —S(O)(═NR^{e1})NR^{c1}R^{d1}, —C(O)NR^{c1}S(O)₂R^{b1}, —C(O)NR^{c1}S(O)₂NR^{c1}R^{d1}, —S(O)₂NR^{c1}C(O)R^{b1}, —NR^{c1}S(O)NR^{c1}C(O)R^{b1}, and —P(O)R^{f1}R^{g1}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R¹ are each optionally substituted with 1, 2, 3, 4, 5, or 6 independently selected R^{1A} substituents;
each R^{a1}, R^{b1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a1}, R^{b1}, R^{c1}, and R^{d1} are each optionally substituted with 1, 2, 3, 4, 5, or 6 independently selected R^{1A} substituents;
or, any R^{e1} and R^{d1} attached to the same N atom, together with the N atom to which they are attached, form a 4-7 membered heterocycloalkyl group, wherein the 4-10 membered heterocycloalkyl group is optionally substituted with 1, 2, 3, 4, 5, or 6 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy;
each R^{f1} and R^{g1} are independently selected from H, C₁₋₆ alkyl, C₁₋₆ alkoxy, C₁₋₆ haloalkyl, C₁₋₆ haloalkoxy;
each R^{1A} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl) carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R² is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}, —NR^{c2}R^{d2}, —C(O)R^{a2}, —C(O)OR^{a2}, —C(O)NR^{c2}R⁴², —C(O)NR^{c2}(OR^{a2}), —OC(O)R^{a2}, —OC(O)NR^{c2}R^{d2}, —OC(O)OR^{a2}, —OS(O)₂R^{b2}, —OS(O)₂NR^{c2}R^{d2}, —NR^{c2}C(O)R^{a2}, —NR^{c2}C(O)OR^{a2}, —NR^{c2}C(O)NR^{c2}R^{d2}, —NR^{c2}S(O)₂R^{b2}, —NR^{c2}S(O)₂NR^{c2}R^{d2}, —NR^{c2}OR^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{e2})R^{a2}, —C(═NR^{e2})NR^{c2}R^{d2}, —NR^{c2}C(═NR^{e2})R^{a2}, —NR^{c2}C(═NR^{e2})NR^{c2}R^{d2}, —NR^{c2}S(O)(═NR^{e2})R^{b2}, —NR^{c2}S(O)(═NR^{e2})NR^{c2}R^{d2}, —OS(O)(═NR^{e2})R^{b2}, —S(O)(═NR^{e2})R^{b2}, —S(O)(═NR^{e2})NR^{c2}R^{d2}, —C(O)NR^{c2}S(O)₂R^{b2}, —C(O)NR^{c2}S(O)₂NR^{c2}R^{d2}, —S(O)₂NR^{c2}C(O)R^{b2}, —NR^{c2}S(O)NR^{c2}C(O)R^{b2}, and —P(O)R^{f2}R^{g2}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R² are each optionally substituted with 1, 2, 3, 4, 5, or 6 independently selected R^{2A} substituents;
each R^{a2}, R^{b2}, R^{e2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a2}, R^{b2}, R^{c2}, and R^{d2} are each optionally substituted with 1, 2, 3, 4, 5, or 6 independently selected R^{2A} substituents;
or, any R^{c2} and R^{d2} attached to the same N atom, together with the N atom to which they are attached, form a 4-7 membered heterocycloalkyl group, wherein the 4-10 membered heterocycloalkyl group is optionally substituted with 1, 2, 3, 4, 5, or 6 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy;
each R^{f2} and R^{g2} are independently selected from H, C₁₋₆ alkyl, C₁₋₆ alkoxy, C₁₋₆ haloalkyl, C₁₋₆ haloalkoxy;
each R^{2A} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl) carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R³ is selected from H, C₁₋₆ alkyl, C₁₋₆ haloalkyl, —C(O)R^{a3}, —C(O)OR^{a3}, —C(O)NR^{c3}R^{d3}, —S(O)₂R^{b3}, —S(O)₂NR^{c3}R^{d3}, —S(O)(═NR^{e3})R^{b3}, and —S(O)(═NR^{e3})NR^{c3}R^{d3}, wherein the C₁₋₆ alkyl, and C₁₋₆ haloalkyl of R³ are each optionally substituted with 1, 2, 3, 4, 5, or 6 independently selected R^{G} substituents;
or, R³ and L³ together with the atoms to which they are attached form a 5-10 membered heterocycloalkyl group, wherein the 5-10 membered heterocycloalkyl group is optionally substituted with 1, 2, 3, 4, 5, or 6 independently selected R^{G} substituents;
each R^{a3}, R^{b3}, R^{c3}, and R^{d3} is independently selected from H, C₁₋₆ alkyl, and C₁₋₆ haloalkyl;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, C₁₋₆ alkyl, and C₁₋₆ haloalkyl;
R⁸ is selected from H, halo, CN, C₁₋₆ alkyl, C₁₋₆ haloalkyl, C₁₋₆ alkoxyl and C₁₋₆ haloalkoxyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl) carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

59. The compound of claim 1, or a pharmaceutically acceptable salt thereof, wherein:
X¹ is N or NR⁶;
X² is CR⁷;
X³ is CR⁸;
Y is C or N;
Z is C or N;
n is 0, 1, 2, 3, or 4;
m is 0, 1, 2, 3, or 4;
Ring A is phenyl, 8-10 membered heterocycloalkyl, or 8-10 membered heteroaryl;
Ring B is phenyl or 5-6 membered heteroaryl;
Ring C is a 5-membered heteroaryl having 2 to 3 nitrogen atoms as ring members;
L¹ is —N(R^{L})C(O)— or —N(R^{L})—;
L² is a bond;
L³ is selected from C₁₋₆ alkylene, —O—, and —N(R^{L})—;
L⁴ is a bond or —O—;
each R^{L} is independently selected from H and C₁₋₆ alkyl;
each R¹ is independently selected from oxo, halo, C₁₋₆ alkyl, C₂₋₆ alkenyl, C₂₋₆ alkynyl, C₁₋₆ haloalkyl, —CN, and —OR^{a1};
each R^{a1} is independently selected from H, C₁₋₆ alkyl, C₂₋₆ alkenyl, C₂₋₆ alkynyl, and C₁₋₆ haloalkyl;
each R² is independently selected from halo, C₁₋₆ alkyl, C₂₋₆ alkenyl, C₂₋₆ alkynyl, and C₁₋₆ haloalkyl;
R³ is selected from H, C₁₋₆ alkyl, C₁₋₆ haloalkyl, and —C(O)NR^{c3}R^{d3};
each R^{c3} and R^{d3} is independently selected from H and C₁₋₆ alkyl;
R⁴ is selected from H, C₁₋₆ alkyl, and C₁₋₆ haloalkyl;
R⁶ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, —C(O)R^{a6}, —C(O)OR^{a6}, —C(O)NR^{c6}R^{d6}, —C(O)NR^{c6}(OR^{a6}), —S(O)R^{b6}, —S(O)₂R^{b6}, —S(O)NR^{c6}R^{d6}, —S(O)₂NR^{c6}R^{d6}, —C(═NR^{e6})R^{a6}, —C(═NR^{e6})NR^{c6}R^{d6}, —S(O)(═NR^{e6})R^{b6}, —S(O)(═NR^{e6})NR^{c6}R^{d6}, —C(O)NR^{c6}S(O)₂R^{b6}, —C(O)NR^{c6}S(O)₂NR^{c6}R^{d6}, and —S(O)₂NR^{c6}C(O)R^{b6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, or 4 independently selected R^{6A} substituents;
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a6}, R^{b6}, R^{c6}, and R^{d6} are each optionally substituted with 1, 2, 3, 4, 5, or 6 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, R⁶ and R⁷ together with the atoms to which they are attached form a 5-14 membered heterocycloalkyl group, wherein the 5-14 membered heterocycloalkyl group is optionally substituted with 1, 2, 3, 4, 5, or 6 independently selected R^{7A} substituents;
each R^{6A} is independently selected from oxo, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6A}, —SR^{a6A}, —NR^{c6A}R^{d6A}, —NO₂, —C(O)R^{a6A}, —C(O)OR^{a6A}, —C(O)NR^{c6A}R^{d6A}, —OC(O)R^{a6A}, —OC(O)N^{c6A}R^{46A}, —OC(O)OR^{a6A}, —NR^{c6A}C(O)R^{a6A}, —NR^{c6A}C(O)OR^{a6A}, —NR^{c6A}C(O)N^{c6A}R^{d6A}, —NR^{c6A}S(O)₂R^{b6A}, —NR^{c6A}S(O)R^{b6A}, —NR^{c6A}S(O)N^{c6A}R^{d6A}, —S(O)R^{b6A}, —S(O)₂R^{b6A}, —S(O)N^{c6A}R^{d6A}, and —S(O)₂NR^{c6A}R^{d6A}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{6A} are each optionally substituted with 1, 2, 3, or 4 independently selected R^{G} substituents;
each R^{a6A}, R^{b6A}, R^{c6A}, and R^{d6A} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6A}, R^{b6A}, R^{c6A}, and R^{d6A} are each optionally substituted with 1, 2, 3, or 4 independently selected R^{G} substituents; or
or, any R^{c6A} and R^{d6A} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{G} substituents;
R⁸ is selected from H, halo, C₁₋₆ alkyl, C₂₋₆ alkenyl, C₂₋₆ alkynyl, and C₁₋₆ haloalkyl; and
each R^{G} is independently selected from H, OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl) carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

60. The compound of claim 1, wherein the compound of Formula I is a compound of Formula II: 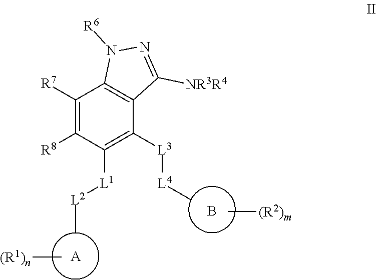
or a pharmaceutically acceptable salt thereof.

61. The compound of claim 1, wherein the compound of Formula I is a compound of Formula IIa: 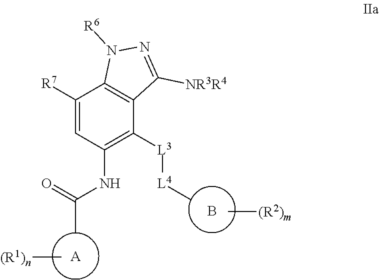
or a pharmaceutically acceptable salt thereof.

62. The compound of claim 1, wherein the compound of Formula I is a compound of Formula III: 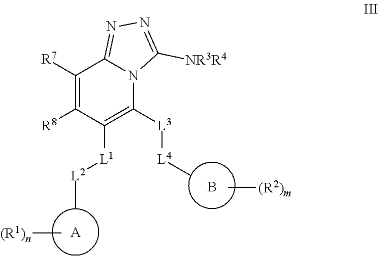
or a pharmaceutically acceptable salt thereof.

63. The compound of claim 1, wherein the compound of Formula I is a compound of Formula IIIa: 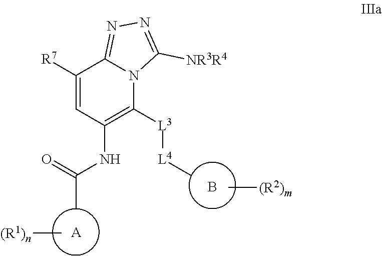
or a pharmaceutically acceptable salt thereof.

64. The compound of claim 1, wherein the compound of Formula I is a compound of Formula IV: 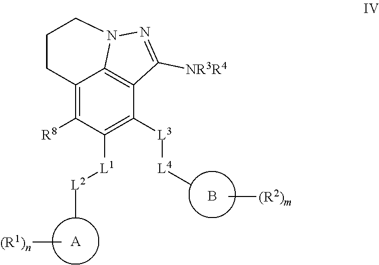
or a pharmaceutically acceptable salt thereof.

65. The compound of claim 1, wherein the compound of Formula I is a compound of Formula IVa: 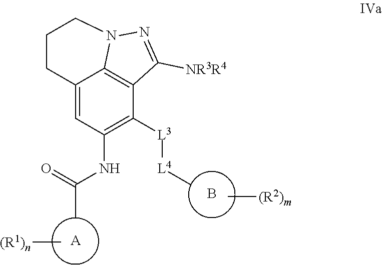
or a pharmaceutically acceptable salt thereof.

66. The compound of claim 1, which is selected from:
N-(3-amino-4-(2-chloro-5-fluorophenoxy)-1H-indazol-5-yl)-3-fluoro-5-(trifluoromethyl)benzamide;
N-(4-(2-chloro-5-fluorophenoxy)-3-(methylamino)-1H-indazol-5-yl)-3-fluoro-5-(trifluoromethyl)benzamide;
N-(3-amino-4-((5-chloropyridin-3-yl)oxy)-1H-indazol-5-yl)-3-fluoro-5-(trifluoromethyl)benzamide;
N-(3-amino-4-((2-chloro-5-fluorophenyl)amino)-1H-indazol-5-yl)-3-fluoro-5-(trifluoromethyl)benzamide;
N-(3-amino-4-(2-chloro-5-fluorobenzyl)-1H-indazol-5-yl)-3-fluoro-5-(trifluoromethyl)benzamide;
N-(3-amino-4-((2-chloro-5-fluorophenoxy)methyl)-1H-indazol-5-yl)-3-fluoro-5-(trifluoromethyl)benzamide;
N-(3-amino-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-ethyl-1H-indazol-5-yl)-3-fluoro-5-(trifluoromethyl)benzamide;
N-(3-amino-4-(2-chloro-5-fluorophenoxy)-1-(2-(methylamino)-2-oxoethyl)-1H-indazol-5-yl)-3-fluoro-5-(trifluoromethyl)benzamide;
N-(3-amino-4-(2-chloro-5-fluorophenoxy)-1-(2-morpholino-2-oxoethyl)-1H-indazol-5-yl)-3-fluoro-5-(trifluoromethyl)benzamide;
N-(3-amino-7-bromo-4-(2-chloro-5-fluorophenoxy)-1H-indazol-5-yl)-3-fluoro-5-(trifluoromethyl)benzamide;
N-(3-amino-7-chloro-4-(2-chloro-5-fluorophenoxy)-1H-indazol-5-yl)-3-fluoro-5-(trifluoromethyl)benzamide;
N-(3-amino-4-(2-chloro-5-fluorophenoxy)-7-methyl-1H-indazol-5-yl)-3-fluoro-5-(trifluoromethyl)benzamide;
N-(3-amino-4-(2-chloro-5-fluorophenoxy)-7-(1-methyl-1H-pyrazol-4-yl)-1H-indazol-5-yl)-3-fluoro-5-(trifluoromethyl)benzamide;
N-(3-amino-4-(2-chloro-5-fluorophenoxy)-7-(1-methyl-1H-pyrazol-5-yl)-1H-indazol-5-yl)-3-fluoro-5-(trifluoromethyl)benzamide;
N-(3-amino-4-(2-chloro-5-fluorophenoxy)-7-(pyridin-3-yl)-1H-indazol-5-yl)-3-fluoro-5-(trifluoromethyl)benzamide;
N-(3-amino-4-(2-chloro-5-fluorophenoxy)-7-phenyl-1H-indazol-5-yl)-3-fluoro-5-(trifluoromethyl)benzamide;
N-(3-amino-4-(2-chloro-5-fluorophenoxy)-1H-indazol-5-yl)benzo[b]thiophene-3-carboxamide;
N-(3-amino-4-(2-chloro-5-fluorophenoxy)-1H-indazol-5-yl)-5-fluoro-3-hydroxy-3-(trifluoromethyl) indoline-1-carboxamide;
N-(3-amino-5-(2-chloro-5-fluorophenoxy)-[1,2,4]triazolo[4,3-a]pyridin-6-yl)-3-fluoro-5-(trifluoromethyl)benzamide;
N-(3-amino-4-(2-chloro-5-fluorophenoxy)-7-(morpholinomethyl)-1H-indazol-5-yl)-3-fluoro-5-(trifluoromethyl)benzamide; and
N-(2-amino-3-(2-chloro-5-fluorophenoxy)-7,8-dihydro-6H-pyrazolo[4,5,1-ij]quinolin-4-yl)-3-fluoro-5-(trifluoromethyl)benzamide;
or a pharmaceutically acceptable salt thereof.

67. The compound of claim 1, which is selected from:
N-(3-amino-4-(2-chloro-5-fluorophenoxy)-7-(hydroxymethyl)-1H-indazol-5-yl)-3-fluoro-5-(trifluoromethyl)benzamide;
N-(3-amino-7-chloro-4-(2-chloro-5-fluorophenoxy)-1-methyl-1H-indazol-5-yl)-3-fluoro-5-(trifluoromethyl)benzamide;
N-(3-amino-7-bromo-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-7-(1-isopropyl-1H-pyrazol-4-yl)-1-methyl-1H-indazol-5-yl)-3-fluoro-5-(trifluoromethyl)benzamide;
N-(3-amino-7-(4-carbamoylphenyl)-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-methyl-7-(1-(tetrahydrofuran-3-yl)-1H-pyrazol-4-yl)-1H-indazol-5-yl)-3-fluoro-5-(trifluoromethyl)benzamide;
N-(3-amino-4-(2-chloro-5-fluorophenoxy)-1-methyl-7-(pyrimidin-5-yl)-1H-indazol-5-yl)-3-fluoro-5-(trifluoromethyl)benzamide;
N-(3-amino-4-(2-chloro-5-fluorophenoxy)-1-methyl-7-(1-methyl-1H-1,2,3-triazol-5-yl)-1H-indazol-5-yl)-3-fluoro-5-(trifluoromethyl)benzamide;
N-(3-amino-4-(2-chloro-5-fluorophenoxy)-7-(cyanomethyl)-1-methyl-1H-indazol-5-yl)-3-fluoro-5-(trifluoromethyl)benzamide;
N-(6-amino-7-(2-chloro-5-fluorophenoxy)-2-oxo-2,3-dihydro-1H-pyrazolo[1,5,4-de]quinoxalin-8-yl)-3-fluoro-5-(trifluoromethyl)benzamide;
N-(3-amino-1-(2-amino-2-oxoethyl)-7-bromo-4-(2-chloro-5-fluorophenoxy)-1H-indazol-5-yl)-3-fluoro-5-(trifluoromethyl)benzamide;
N-(3-amino-7-chloro-4-(2-chloro-5-fluorophenoxy)-1-(2-cyanoethyl)-1H-indazol-5-yl)-3-fluoro-5-(trifluoromethyl)benzamide;
N-(3-(2-aminoacetamido)-7-chloro-4-(2-chloro-5-fluorophenoxy)-1-methyl-1H-indazol-5-yl)-3-fluoro-5-(trifluoromethyl)benzamide;
N-(2-amino-3-(2-chloro-5-fluorophenoxy)-8-oxo-6,7,8,9-tetrahydro-[1,4]diazepino[6,7,1-hi]indazol-4-yl)-3-fluoro-5-(trifluoromethyl)benzamide;
N-(3-amino-4-(2-chloro-5-fluorophenoxy)-1-methyl-7-(pyridin-2-ylethynyl)-1H-indazol-5-yl)-3-fluoro-5-(trifluoromethyl)benzamide;
N-(3-amino-4-(2-chloro-5-fluorophenoxy)-1-methyl-7-((tetrahydro-2H-pyran-4-yl)ethynyl)-1H-indazol-5-yl)-3-fluoro-5-(trifluoromethyl)benzamide;
N-(3-amino-4-(2-chloro-5-fluorophenoxy)-7-(3-hydroxy-3-methylbut-1-yn-1-yl)-1-methyl-1H-indazol-5-yl)-3-fluoro-5-(trifluoromethyl)benzamide;
N-(3-amino-7-(3-amino-3-methylbut-1-yn-1-yl)-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-methyl-7-((tetrahydrofuran-3-yl)ethynyl)-1H-indazol-5-yl)-3-fluoro-5-(trifluoromethyl)benzamide;
N-(3-amino-4-(2-chloro-5-fluorophenoxy)-7-(cyclopropylethynyl)-1-methyl-1H-indazol-5-yl)-3-fluoro-5-(trifluoromethyl)benzamide;
N-(3-amino-4-(2-chloro-5-fluorophenoxy)-7-((1-hydroxycyclopropyl)ethynyl)-1-methyl-1H-indazol-5-yl)-3-fluoro-5-(trifluoromethyl)benzamide;
N-(3-amino-4-(2-chloro-5-fluorophenoxy)-7-((1-(hydroxymethyl)cyclopropyl)ethynyl)-1-methyl-1H-indazol-5-yl)-3-fluoro-5-(trifluoromethyl)benzamide;
N-(3-amino-7-((1-aminocyclopropyl)ethynyl)-4-(2-chloro-5-fluorophenoxy)-1-methyl-1H-indazol-5-yl)-3-fluoro-5-(trifluoromethyl)benzamide;
N-(3-amino-4-(2-chloro-5-fluorophenoxy)-1-methyl-7-((3-methyloxetan-3-yl)ethynyl)-1H-indazol-5-yl)-3-fluoro-5-(trifluoromethyl)benzamide;
N-(3-amino-4-(2-chloro-5-fluorophenoxy)-7-ethynyl-1-methyl-1H-indazol-5-yl)-3-fluoro-5-(trifluoromethyl)benzamide;
N-(3-amino-4-(2-chloro-5-fluorophenoxy)-1-methyl-7-((1-methyl-1H-pyrazol-3-yl)ethynyl)-1H-indazol-5-yl)-3-fluoro-5-(trifluoromethyl)benzamide;
N-(3-amino-4-(2-chloro-5-fluorophenoxy)-7-(3-hydroxyprop-1-yn-1-yl)-1-methyl-1H-indazol-5-yl)-3-fluoro-5-(trifluoromethyl)benzamide;
N-(3-amino-4-(2-chloro-5-fluorophenoxy)-1-methyl-7-((1-methyl-1H-imidazol-4-yl)ethynyl)-1H-indazol-5-yl)-3-fluoro-5-(trifluoromethyl)benzamide;
N-(3-amino-4-(2-chloro-5-fluorophenoxy)-1-methyl-7-((1-methylpiperidin-4-yl)ethynyl)-1H-indazol-5-yl)-3-fluoro-5-(trifluoromethyl)benzamide;
N-(7-((1-acetylpiperidin-4-yl)ethynyl)-3-amino-4-(2-chloro-5-fluorophenoxy)-1-methyl-1H-indazol-5-yl)-3-fluoro-5-(trifluoromethyl)benzamide;
N-(7-chloro-4-(2-chloro-5-fluorophenoxy)-1-methyl-3-(methylamino)-1H-indazol-5-yl)-3-fluoro-5-(trifluoromethyl)benzamide;
N-(7-chloro-4-(2-chloro-5-fluorophenoxy)-3-(dimethylamino)-1-methyl-1H-indazol-5-yl)-3-fluoro-5-(trifluoromethyl)benzamide;
N-(3-amino-4-(2-chloro-5-fluorophenoxy)-7-(5,6-dihydro-[1,2,4]triazolo[1,5-a]pyrazin-7 (8H)-yl)-1-methyl-1H-indazol-5-yl)-3-fluoro-5-(trifluoromethyl)benzamide;
N-(3-amino-1-(2-(azetidin-1-yl)ethyl)-7-chloro-4-(2-chloro-5-fluorophenoxy)-1H-indazol-5-yl)-3-fluoro-5-(trifluoromethyl)benzamide;
(E)-N-(3-amino-4-(2-chloro-5-fluorophenoxy)-7-(2-cyanovinyl)-1-methyl-1H-indazol-5-yl)-3-fluoro-5-(trifluoromethyl)benzamide;
(Z)—N-(3-amino-4-(2-chloro-5-fluorophenoxy)-7-(2-cyanovinyl)-1-methyl-1H-indazol-5-yl)-3-fluoro-5-(trifluoromethyl)benzamide;
N-(3-amino-4-(2-chloro-5-fluorophenoxy)-7-(2-cyanoethyl)-1-methyl-1H-indazol-5-yl)-3-fluoro-5-(trifluoromethyl)benzamide;
N-(3-amino-7-chloro-4-(2-chloro-5-fluorophenoxy)-1-methyl-1H-indazol-5-yl)-3-chloro-5-fluorobenzamide;
N-(3-amino-7-chloro-4-(2-chloro-5-fluorophenoxy)-1-methyl-1H-indazol-5-yl)-3-(trifluoromethyl)benzamide;
N-(3-amino-7-chloro-4-(2-chloro-5-fluorophenoxy)-1-methyl-1H-indazol-5-yl)-5-(trifluoromethyl) nicotinamide;
N-(3-amino-7-chloro-4-(2-chloro-5-fluorophenoxy)-1-methyl-1H-indazol-5-yl)benzo[b]thiophene-3-carboxamide;
N-(3-amino-7-chloro-4-(2-chloro-5-fluorophenoxy)-1-methyl-1H-indazol-5-yl) indoline-1-carboxamide;
3-Amino-4-(2-chloro-5-fluorophenoxy)-N-ethyl-5-(3-fluoro-5-(trifluoromethyl)benzamido)-1-methyl-1H-indazole-7-carboxamide;
N-(3-amino-4-(2-chloro-5-fluorophenoxy)-7-(2-hydroxyethyl)-1-methyl-1H-indazol-5-yl)-3-fluoro-5-(trifluoromethyl)benzamide;
N-(3-amino-4-(2-chloro-5-fluorophenoxy)-1-(2-hydroxyethyl)-7-(pyridin-2-ylethynyl)-1H-indazol-5-yl)-3-fluoro-5-(trifluoromethyl)benzamide;
N-(2-amino-3-(2-chloro-5-fluorophenoxy)-8-oxo-7,8-dihydro-6H-pyrazolo[4,5,1-ij]quinazolin-4-yl)-3-fluoro-5-(trifluoromethyl)benzamide;
N-(2-amino-3-(2-chloro-5-fluorophenoxy)-7-methyl-8-oxo-7,8-dihydro-6H-pyrazolo[4,5,1-ij]quinazolin-4-yl)-3-fluoro-5-(trifluoromethyl)benzamide;
N-(4-amino-3-(2-chloro-5-fluorophenoxy)-7H-pyrazolo[4,5,1-de]phenanthridin-2-yl)-3-fluoro-5-(trifluoromethyl)benzamide;
N-(2-amino-3-(2-chloro-5-fluorophenoxy)-7-cyclopropyl-8-oxo-7,8-dihydro-6H-pyrazolo[4,5,1-ij]quinazolin-4-yl)-3-fluoro-5-(trifluoromethyl)benzamide;
N-(2-amino-3-(2-chloro-5-fluorophenoxy)-7-cyclobutyl-8-oxo-7,8-dihydro-6H-pyrazolo[4,5,1-ij]quinazolin-4-yl)-3-fluoro-5-(trifluoromethyl)benzamide;
N-(4-amino-3-(2-chloro-5-fluorophenoxy)-7-oxo-7H-pyrazolo[4,5,1-de]phenanthridin-2-yl)-3-fluoro-5-(trifluoromethyl)benzamide;
N-(4-amino-3-(2-chloro-5-fluorophenoxy)-7-oxo-7,8,9,10-tetrahydrocyclopenta[c]pyrazolo[4,5,1-ij]quinolin-2-yl)-3-fluoro-5-(trifluoromethyl)benzamide;
N-(4-amino-3-(2-chloro-5-fluorophenoxy)-7-oxo-8,9,10,11-tetrahydro-7H-pyrazolo[4,5,1-de]phenanthridin-2-yl)-3-fluoro-5-(trifluoromethyl)benzamide;
(E)-N-(3-amino-4-(2-chloro-5-fluorophenoxy)-1-methyl-7-(2-(pyridin-2-yl) vinyl)-1H-indazol-5-yl)-3-fluoro-5-(trifluoromethyl)benzamide;
N-(3-amino-4-(2-chloro-5-fluorophenoxy)-1-methyl-7-(2-(pyridin-2-yl)ethyl)-1H-indazol-5-yl)-3-fluoro-5-(trifluoromethyl)benzamide;
N-(3-amino-4-(2-chloro-5-fluorophenoxy)-1-methyl-7-(piperidin-2-ylethynyl)-1H-indazol-5-yl)-3-fluoro-5-(trifluoromethyl)benzamide;
N-(7-((1-acetylpiperidin-2-yl)ethynyl)-3-amino-4-(2-chloro-5-fluorophenoxy)-1-methyl-1H-indazol-5-yl)-3-fluoro-5-(trifluoromethyl)benzamide;
2-((3-Amino-4-(2-chloro-5-fluorophenoxy)-5-(3-fluoro-5-(trifluoromethyl)benzamido)-1-methyl-1H-indazol-7-yl)ethynyl)-N-methylpiperidine-1-carboxamide;
N-(3-amino-7-bromo-4-(2-chloro-5-fluorophenoxy)-1-(methylsulfonyl)-1H-indazol-5-yl)-3-fluoro-5-(trifluoromethyl)benzamide; and
N-(1-acetyl-3-amino-7-bromo-4-(2-chloro-5-fluorophenoxy)-1H-indazol-5-yl)-3-fluoro-5-(trifluoromethyl)benzamide;
or a pharmaceutically acceptable salt thereof.

68. A pharmaceutical composition, comprising a compound of claim 1, or a pharmaceutically acceptable salt thereof, and a pharmaceutically acceptable carrier.

69. A method of inhibiting an activity of PI3Kα kinase, comprising contacting the kinase with a compound of claim 1, or a pharmaceutically acceptable salt thereof.

70. A method of treating a PI3Kα-mediated disease or disorder in a patient, comprising administering to the patient a therapeutically effective amount of a compound of claim 1, or a pharmaceutically acceptable salt thereof.

71. The method of claim 70, wherein the disease or disorder is a cancer.

72. The method of claim 71, wherein the cancer is selected from breast cancer, brain cancer, prostate cancer, endometrial cancer, gastric cancer, leukemia, lymphoma, sarcoma, colorectal cancer, lung cancer, ovarian cancer, skin cancer, and head and neck cancer.

73. The method of claim 70, wherein the disease or disorder is CLOVES syndrome (congenital lipomatous overgrowth, vascular malformations, epidermal naevi, scoliosis/skeletal and spinal syndrome), or PIK3CA-related overgrowth syndrome (PROS).
